SEQUENCE LISTING (302 sequences)

SEQ ID NO: 1 - protein, 387 aa, synthetic construct
  REGION 1..387: note = Synthetic Construct
EPGSSASFSKRRKERLKLGAIFLEGITVKGVTQVTTQPKLGEVQQKCHQFCGWEGSGFPGAQPVSMDKQNIRLLEQKPYKVSWKADGTRYMMLIDGTNEVFMIDRDNSVFHVSNLEFPFRKDLRMHLSNTLLDGEMIIDKVNGQAVPRYLIYDIIKFNAQPVGDCDFNIRLQCIEREIISPRHEKMKTGLIDKTQEPFSVRPKQFFDINISRKLLEGNFAKEVSHEMDGLIFQPIGKYKPGRCDDILKWKPPSLNSVDFRLKITRMGGEGLLPQNVGLLYVGGYERPFAQIKVTKELKQYDNKIIECKFENNSWVFMRQRIDKSFPNAYNTAMAVCNSISNPVTKEMLFEFIDRCAAAAQGQKRKYPLDPDTELMPPPPPKRLHRPT

SEQ ID NO: 2 - DNA, 1161 bp, synthetic construct
  misc_feature 1..1161: note = Synthetic Construct
  source 1..1161: mol_type = other DNA
gaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaacc

SEQ ID NO: 3 - DNA, 6559 bp, synthetic construct
  misc_feature 1..6559: note = Synthetic Construct
  source 1..6559: mol_type = other DNA
tcgcgcgtttcggtgatgacggtgaaaacctctgacacatgcagctcccggagacggtcacagcttgtcacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgtcaaccaaaccagagaagccaatatggatgtcacaagaagactatgaccgtcagtacggctcaattactggtgatgaatcgtccacagtctcaaaaaaagactccaaagttactgccaatgcgcctggtgatggtaatggatcattaccagttttgcaaagcagctctatccttacttctaaggtgtctgatctacccattgaggcagaatccgggtttaaaatccaaaagagaagacatgaaaggtatgatcaagaggaaagattacgcaaacagcgtgctcaaaaattgagagaagagcaactaaagagacatgaaatagagatgactgcaaatagatccataaatgttgaccagattgtccgagagcactataatgaacgtacaataatcgccaatcgcgctaagaggaatctatcgcctattattaagcttcgtaattttaacaatgcgattaagtatatgttaattgacaaatatacaaaacctggagatgtcgttttagaactcggatgtggcaaaggtggtgatttaagaaaatatggtgctgcaggtatttcacagtttattggtattgacatttccaatgcctctatccaagaagctcataagagataccggtccatgagaaatttggactaccaagttgtgctaattactggggattgctttggcgaatcattaggtgttgcagtagagcccttcccagactgtagatttccgtgtgatatcgtttcgacgcaattttgcttgcattatgcttttgagaccgaagaaaaagcaaggagagctttactgaatgtcgccaaatcactcaaaattggtggtcacttttttggcacgataccagattcagaattcattcgttataaactgaataaatttcccaaagaagtggaaaagccatcttggggaaactccatttataaagtgacatttgagaataattcataccaaaagaatgattatgagttcacatcgccttatggtcaaatgtatacttattggctagaagatgctatagataatgttcctgaatatgttgtgccattcgaaaccttaagaagtttggctgatgaatacggtttggaactagtatcgcaaatgccttttaataaattttttgtccaagaaattcccaaatggattgagaggttttcgcctaaaatgagggaaggtttacaaagatctgatggtagatatggtgttgaaggggatgaaaaggaagcggcatcatatttctataccatgtttgcattccgtaaggtaaaacagtatatcgaaccggaaagcgtaaagcccaactgatgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 4 - protein, 528 aa, SARS-CoV-2
MAENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSGSPCESHGKQVVSDIDYVPLKSATCITRCNLGGAVCRHHANEYRQYLDAYNMMISAGFSLWIYKQFDTYNLWNTFTRLQ

SEQ ID NO: 5 - DNA, 1584 bp, synthetic construct
  misc_feature 1..1584: note = Synthetic Construct
  source 1..1584: mol_type = other DNA
atggcagagaatgtaaccgggctattcaaggattgcagcaaaataattaccgggcttcatccgacgcaagcacccacacacctgagtgtcgatatcaagttcaagactgaaggactttgtgtcgacatacctgggattccgaaggacatgacctatcgtagactaatttctatgatgggattcaagatgaattatcaagttaacggctatccaaatatgttcataactagggaggaagcaatacgtcatgtacgtgcttggataggattcgacgtagaaggatgtcatgccacacgtgacgctgtgggtactaacttaccattacaattaggctttagcaccggggttaacttagttgcagttcccaccggctacgtcgacaccgagaataacacggagtttacaagggtaaatgccaagccgccgccaggtgatcagttcaagcacttgatccctttgatgtacaagggactaccctggaatgtagtaagaatcaagatagtacaaatgctatccgacaccctaaaaggattatccgaccgtgttgttttcgtcttgtgggcacacggctttgaactaacgtcaatgaaatactttgtcaaaattggaccagagagaacgtgctgcctttgtgataagcgtgcgacttgttttagcaccagctctgacacctatgcatgttggaatcattctgtagggtttgactacgtatacaacccatttatgatagacgtacaacaatggggttttacgggtaaccttcagtctaatcacgaccaacattgtcaagtacacggcaatgcgcacgtcgcatcctgtgacgccataatgactagatgtttggcagtacatgaatgtttcgtcaaacgtgtcgattggagcgtggaatacccgattattggtgatgaactgagggtcaactctgcttgtaggaaagtacagcacatggtagttaaatccgcgcttctagcggacaaattccctgttctacacgacatcggtaatccgaaagccataaaatgtgtaccacaagcagaagtcgaatggaagttttacgacgcccaaccttgctctgataaagcctacaagatcgaagagttgttctattcttacgcgacccaccatgataagtttacagatggtgtttgtttgttctggaactgtaacgtggatagataccctgccaacgccatagtttgcagattcgacacgagagtactgagtaatctgaaccttcccgggtgtgatggtgggagtttatacgtgaacaaacatgcctttcatacaccggcctttgataagagcgcatttaccaacttaaaacaactgccatttttttattactcaggtagtccgtgcgagagtcatgggaaacaggtagtcagtgatatagattacgtaccgctgaagtcagcaacttgtataaccagatgcaatcttggtggagctgtctgtcgtcatcatgctaacgaataccgtcaatacttagacgcatacaacatgatgatttccgcgggattctcattatggatttataaacaatttgatacatacaatttatggaatacctttacaaggctacag

SEQ ID NO: 6 - protein, 348 aa, Monkeypox virus
MKYINSEDYYGNQHNIIVEHLRDQSIKIGDVFNEDKLSDVGHQYAANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 7 - DNA, 1044 bp, synthetic construct
  misc_feature 1..1044: note = Synthetic Construct
  source 1..1044: mol_type = other DNA
atgaaatacattaatagcgaagattattacgggaatcaacacaatatcatcgtagagcatctaagggaccaatccataaagatcggggatgtcttcaacgaagataaactgagtgacgttggacatcaatacgccgcaaacaatgacaaatttagacttaatccggaagtaagttacttcacgaataaacgtacacgtgggccgttaggtatcttatctaactatgtgaagactctattgatctccatgtattgctcaaaaacattcttagacgattctaacaaaagaaaagtattagccatcgacttcggaaacggtgctgacttggagaaatacttttacggggaaatcgcgctgctggtagcaacagaccccgatgctgatgcaattgccagaggcaacgaaagatacaacaagctgaattcaggaataaaaacgaaatattataagtttgattacatacaggaaacaattcgttctgacacgttcgtgtctagcgttagagaagtattctactttggtaagttcaatatcatcgactggcagtttgccatccattactcatttcatccgaggcattatgcgacagtaatgaataatctgtctgaattaactgctagcggcgggaaagtgcttattactacgatggacggggacaagctaagtaagttgacggataagaagacttttatcatccataagaacttgcctagttccgagaactacatgagcgttgaaaaaatcgccgacgaccgtatagtagtatataatccatccacgatgtctacacccatgactgaatacataatcaagaaaaatgatattgtaagagttttcaacgagtatggatttgtattggtcgacaacgtagattttgctaccataatagaacgttccaaaaagttcataaacggtgcatcaacaatggaagacagacctagtactagaaactttttcgaacttaaccgtggcgccataaaatgcgagggcttggatgtagaggatttactttcatattatgtagtttacgtattcagcaagagg

SEQ ID NO: 8 - protein, 287 aa, Monkeypox virus
MDEIVKNIREGTHVLLPFYETLPELNLSLGKSPLPSLEYGANYFLQISRVNDLNRMPTDMLKLFTHDIMLPESDLDKVYEILKINSVKYYGRSTRADAVVADLSARNKLFKRERDAIKSNNHLTENNLYISDYKMLTFDVFRPLFDFVNEKYCIIKLPTLFGRGVIDTMRIYCSLFKNVRLLKCVSDSWLKDSAIMVASDVYKKNLDLFMSHVKSVTKSSSWKDVNTVQFSILNDPVDTEFINKFLEFSNRVYEALYYVHSLLYSSMTSDSKSIENKHQRRLVKLLL

SEQ ID NO: 9 - DNA, 861 bp, synthetic construct
  misc_feature 1..861: note = Synthetic Construct
  source 1..861: mol_type = other DNA
atggatgagattgtcaagaatattcgtgagggaacgcacgtgttgctgcctttctacgagactttgccggaattgaacctttcccttggaaagagccctcttcccagtctagaatatggagcaaattacttccttcagattagtagagtaaacgatctgaacaggatgccaaccgatatgttgaagctatttacacacgatattatgttacctgaatccgatttagacaaagtttacgaaatcctgaaaattaactcagtgaagtattacggccgtagcacgagggccgacgccgtagttgcagatctaagtgctaggaacaagctattcaagcgtgagagagacgctataaaaagtaacaaccacctgaccgagaacaatctttacataagtgactataagatgcttaccttcgacgtctttagaccattattcgactttgtgaatgaaaaatattgcatcataaagttaccgacgctttttggaagaggggttatagacacaatgagaatatactgctcacttttcaagaatgtgagactattaaagtgtgtctcagacagttggctgaaagactccgcaattatggttgcatcagatgtttacaagaagaacttagacctatttatgtctcacgttaagtctgttacgaagtccagtagttggaaagatgtaaacacagttcaattctccattttgaacgatcccgtagacacggaatttattaacaaattccttgagttctctaatagagtctacgaagccttgtactatgttcacagccttttatactccagtatgaccagtgattcaaaaagtatagaaaataaacatcagcgtaggctagttaaattactgcta

SEQ ID NO: 10 - protein, 868 aa, African Swine Fever virus
MASLENLVARYQRCFNDQSLKNSTIEFEIRFQHINFLLFKTVYEALVAQEIPSTISHSIRCIKKVHHENHCREKILPSDNFYFKKQPLMFFKFSEPASLGCKVSLAIEQPIRKFILDSSVLVRLKNRTTFQISELWKIELTVVKQLMGSEVSAKLTAFKTLLFDTPEQQTAKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQMYVVADQLYSLGTTGIEPLKPTILDGEFMPEKKEFYGFDVIMYEGNLLTQQGFETRIEALNKGIKVLQAFNIKAEMKPFISLTSADPNVLLKNFESVFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSDIDGKVLEMRCLKREVNYVSWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGIRHLVGIDKDQTALAELIYRKFSHATTRQHKHATNIYVLHQDLAEPAKEISEKVHQIYGFPKEGASSIVSNLFIHYLMKSSQQVENLAVLCHKLLQPGGMVWFTTMLGERVLELLHENRVELNEVWEARENEVVKFAIKRLFKEDVLQETGQEIGVLLPFSNGDFYNEYLVNTAFLIKIFKHHGFSLVQMQSFKDWIPEFQTFSKSLYKILTEADKTWTSLFGFICLRKN

SEQ ID NO: 11 - DNA, 2604 bp, synthetic construct
  misc_feature 1..2604: note = Synthetic Construct
  source 1..2604: mol_type = other DNA
atggctagtcttgagaatctggtagcacgttatcagagatgtttcaacgatcagtctctgaaaaattcaaccatcgaatttgaaatcaggttccagcacattaactttttattgtttaagacggtgtatgaagctctagtcgcacaagaaatcccaagtactatcagccactctatcaggtgtataaagaaggtccaccatgaaaatcactgcagagaaaaaatccttccgtctgacaatttctacttcaagaagcaaccgcttatgttttttaaatttagtgagcctgcttccttgggatgcaaagttagcctagccatcgagcaaccaattcgtaagttcatcttagactcttctgtgctggtcaggttaaaaaatagaaccacattccagattagcgaactttggaagatcgaactgaccgtcgtcaaacagcttatgggctctgaagtcagcgcaaaattaactgccttcaagactcttttgtttgacacaccggagcaacagactgcaaagaatatgatgaccttgattaatcctgacgatgagtacttgtacgagatcgagatagaatacacagggaaacctgaaagtttgactgctgcagacgtgatcaagatcaaaaacaccgttctaaccttaatatcacctaatcatcttatgctgacagcatatcatcaggctatcgagttcattgcttcacatattcttagttctgagattcttctggctagaataaaatctggaaagtgggggttaaaaagattgctgcctcaggtaaaatccatgactaaagcggattatatgaaattctacccaccggtaggatactatgttacagataaagcggatgggatacgtggcattgctgtcattcaggatacccaaatgtacgtggtcgcagatcaattatattcactgggaacgaccggtatagaacccctaaagcccactattcttgacggcgaattcatgcctgaaaagaaagaattttatggattcgatgtgattatgtacgagggcaatctgctaacgcagcagggttttgagacgagaatagaagcacttaacaaaggcatcaaagttttgcaggcctttaatataaaggcagaaatgaagcccttcataagtcttaccagcgcagaccccaatgtcctgttaaaaaattttgaaagcgttttcaaaaaaaaaactagaccgtactcaatagacggcataatactggtcgagccgggaaacagttatttaaacaccaataccttcaaatggaaacctacatgggacaataccttggacttcctggtcagaaaatgtcccgagtccctaaacgtccctgagtatgcacctaaaaagggcttctccctgcatctgctatttgtcggaatcagtggggagttattcaagaaattggcgctaaactggtgtcctggctacacgaagttattccctgttacccaacgtaatcagaactatttcccggtccaattccagccgtccgattttccattggcttttttatactatcatcccgacacgagtagtttcagtgacatagatggaaaggtactggaaatgagatgcctaaaaagggaggtaaattacgtctcatgggaaatcgtcaagattcgtgaagatagacaacaagatctaaagactgggggttatttcggtaacgattttaagactgcagagctgacatggttgaattacatggacccgttcagctttgaggaattagccaaaggaccatccggcatgtactttgctggggcgaagaccggcatctatcgtgcgcagacggcgttgatcagttttataaaacaagaaattatacagaaaatcagccatcaaagttgggtcatagatctgggaatcggaaagggtcaggatctagggcgttacctggatgcaggaattagacatctggttggtattgataaagaccagaccgcattggcggaattaatatataggaaattcagtcatgctacgacccgtcagcacaagcacgcgacgaacatctatgtattgcaccaagacttggcggaacctgctaaagaaatctctgaaaaggtgcatcaaatatatgggtttcccaaagaaggagctagtagtatagtaagcaatctgtttatacattatctgatgaagtccagccaacaggttgagaacttagcggtactatgtcataagttgttgcagcctggcgggatggtatggttcacaaccatgctgggtgagcgtgtcctggaattattacatgagaatagggtcgagctgaacgaagtctgggaagcaagggaaaatgaagtggttaagtttgccattaagaggctttttaaagaggatgtactgcaagagacggggcaagaaataggtgtactgcttccgttctctaacggcgatttctataacgaatatctggtaaacacagctttcctaatcaagatcttcaagcaccacggctttagcttggtccaaatgcaaagttttaaggattggatacccgaatttcagacgttcagcaaatcactttataagatactgacggaggcggacaaaacgtggacgtccctgttcgggtttatatgcttaaggaagaat

SEQ ID NO: 12 - protein, 301 aa, West Nile virus
MGGAKGRTLGEVWKERLNQMTKEEFTRYRKEAIIEVDRSAAKHARKEGNVTGGHPVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKIKNRIERLRREYSSTWHHDENH

SEQ ID NO: 13 - DNA, 350 bp, synthetic construct
  misc_feature 1..350: note = Synthetic Construct
  source 1..350: mol_type = other DNA
acaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 14 - protein, 480 aa, Homo sapiens
HKLKMANSAKAEEYEKMSLEQAKASVNSETESSFNINENTTASGTGLSEKTSVCRQVDIARKRKEFEDDLVKESSSCGKDTPSKKRKLDPEIVPEEKDCGDAEGNSKKRKRETEDVPKDKSSTGDGTQNKRKIALEDVPEKQKNLEEGHSSTVAAHYNELQEVGLEKRSQSRIFYLRNFNNWIKSVLIGEFLEKVRQKKKRDITVLDLGCGKGGDLLKWKKGRINKLVCTDIADVSVKQCQQRYEDMKNRRDSEYIFSAEFITADSSKELLIDKFRDPQMCFDICSCQFVCHYSFESYEQADMMLRNACERLSPGGYFIGTTPNSFELIRRLEASETESFGNEIYTVKFQKKGDYPLFGCKYDFNLEGVVDVPEFLVYFPLLNEMAKKYNMKLVYKKTFLEFYEEKIKNNENKMLLKRMQALEPYPANESSKLVSEKVDDYEHAAKYMKNSQVRLPLGTLSKSEWEATSIYLVFAFEKQQ

SEQ ID NO: 15 - DNA, 1428 bp, synthetic construct
  misc_feature 1..1428: note = Synthetic Construct
  source 1..1428: mol_type = other DNA
atggccaactccgcaaaggcagaggagtacgagaagatgagccttgaacaggctaaagcgtccgttaacagcgagaccgagtccagtttcaacatcaatgagaatacaacagctagtggcactggtttgtccgaaaagacgagtgtgtgtagacaggtagacattgctagaaagcgtaaggagttcgaggacgatttagtcaaggagtcatcttcctgcggaaaggatactccaagtaagaagcgtaagctagacccggagatagtgccagaagagaaagactgcggagatgcagagggtaatagcaaaaagagaaaaagagaaactgaggatgtgccgaaggacaagtcctcaactggtgatgggactcagaacaaaaggaagatagcattggaggacgtacctgagaagcagaaaaatcttgaggaaggtcattccagcacagttgcggcgcactataatgagctacaggaggtagggttggagaagcgttcccaatcaaggatattctatctaagaaactttaacaactggataaagtccgttctaataggagaattcttggagaaggtgagacaaaaaaaaaagagggatataacggttctagacttggggtgtgggaaagggggtgacttgttgaaatggaaaaaaggaaggataaataaacttgtctgtacggatatcgccgatgtttctgttaaacaatgtcaacaaaggtatgaagatatgaagaatcgtagagatagcgaatacattttttctgccgagttcataactgcggatagcagcaaagagcttctgatcgacaaattccgtgacccacaaatgtgctttgatatttgctcttgtcaattcgtttgtcattactcctttgagtcatacgaacaagcagacatgatgctgaggaatgcctgtgaaagactgtctcctggtggatacttcattgggacgaccccgaacagtttcgagttaatcagaaggttggaagccagtgaaaccgaatcatttggcaatgaaatatacacagtgaagtttcagaagaaaggagattacccgctgttcggctgtaagtatgactttaacttggagggagtagttgacgtgcccgagtttctagtctattttcctctgttgaacgagatggcgaagaagtataacatgaaattagtttacaaaaagacgttccttgagttctatgaagaaaagatcaagaacaatgaaaacaagatgttacttaaacgtatgcaggcacttgaaccgtacccggccaatgaaagtagtaagttagtcagcgaaaaagtcgacgattacgaacatgcggcaaaatatatgaagaattctcaagtaagattaccgttgggaacactatcaaagtccgaatgggaggcaacttccatctacttggttttcgcctttgagaagcaacag

SEQ ID NO: 16 - DNA, 9925 bp, synthetic construct
  misc_feature 1..9925: note = Synthetic Construct
  source 1..9925: mol_type = other DNA
tcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggcagagaatgtaaccgggctattcaaggattgcagcaaaataattaccgggcttcatccgacgcaagcacccacacacctgagtgtcgatatcaagttcaagactgaaggactttgtgtcgacatacctgggattccgaaggacatgacctatcgtagactaatttctatgatgggattcaagatgaattatcaagttaacggctatccaaatatgttcataactagggaggaagcaatacgtcatgtacgtgcttggataggattcgacgtagaaggatgtcatgccacacgtgacgctgtgggtactaacttaccattacaattaggctttagcaccggggttaacttagttgcagttcccaccggctacgtcgacaccgagaataacacggagtttacaagggtaaatgccaagccgccgccaggtgatcagttcaagcacttgatccctttgatgtacaagggactaccctggaatgtagtaagaatcaagatagtacaaatgctatccgacaccctaaaaggattatccgaccgtgttgttttcgtcttgtgggcacacggctttgaactaacgtcaatgaaatactttgtcaaaattggaccagagagaacgtgctgcctttgtgataagcgtgcgacttgttttagcaccagctctgacacctatgcatgttggaatcattctgtagggtttgactacgtatacaacccatttatgatagacgtacaacaatggggttttacgggtaaccttcagtctaatcacgaccaacattgtcaagtacacggcaatgcgcacgtcgcatcctgtgacgccataatgactagatgtttggcagtacatgaatgtttcgtcaaacgtgtcgattggagcgtggaatacccgattattggtgatgaactgagggtcaactctgcttgtaggaaagtacagcacatggtagttaaatccgcgcttctagcggacaaattccctgttctacacgacatcggtaatccgaaagccataaaatgtgtaccacaagcagaagtcgaatggaagttttacgacgcccaaccttgctctgataaagcctacaagatcgaagagttgttctattcttacgcgacccaccatgataagtttacagatggtgtttgtttgttctggaactgtaacgtggatagataccctgccaacgccatagtttgcagattcgacacgagagtactgagtaatctgaaccttcccgggtgtgatggtgggagtttatacgtgaacaaacatgcctttcatacaccggcctttgataagagcgcatttaccaacttaaaacaactgccatttttttattactcaggtagtccgtgcgagagtcatgggaaacaggtagtcagtgatatagattacgtaccgctgaagtcagcaacttgtataaccagatgcaatcttggtggagctgtctgtcgtcatcatgctaacgaataccgtcaatacttagacgcatacaacatgatgatttccgcgggattctcattatggatttataaacaatttgatacatacaatttatggaatacctttacaaggctacaggaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaacctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaag

SEQ ID NO: 17 - DNA, 8224 bp, synthetic construct
  misc_feature 1..8224: note = Synthetic Construct
  source 1..8224: mol_type = other DNA
tgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgaaatacattaatagcgaagattattacgggaatcaacacaatatcatcgtagagcatctaagggaccaatccataaagatcggggatgtcttcaacgaagataaactgagtgacgttggacatcaatacgccgcaaacaatgacaaatttagacttaatccggaagtaagttacttcacgaataaacgtacacgtgggccgttaggtatcttatctaactatgtgaagactctattgatctccatgtattgctcaaaaacattcttagacgattctaacaaaagaaaagtattagccatcgacttcggaaacggtgctgacttggagaaatacttttacggggaaatcgcgctgctggtagcaacagaccccgatgctgatgcaattgccagaggcaacgaaagatacaacaagctgaattcaggaataaaaacgaaatattataagtttgattacatacaggaaacaattcgttctgacacgttcgtgtctagcgttagagaagtattctactttggtaagttcaatatcatcgactggcagtttgccatccattactcatttcatccgaggcattatgcgacagtaatgaataatctgtctgaattaactgctagcggcgggaaagtgcttattactacgatggacggggacaagctaagtaagttgacggataagaagacttttatcatccataagaacttgcctagttccgagaactacatgagcgttgaaaaaatcgccgacgaccgtatagtagtatataatccatccacgatgtctacacccatgactgaatacataatcaagaaaaatgatattgtaagagttttcaacgagtatggatttgtattggtcgacaacgtagattttgctaccataatagaacgttccaaaaagttcataaacggtgcatcaacaatggaagacagacctagtactagaaactttttcgaacttaaccgtggcgccataaaatgcgagggcttggatgtagaggatttactttcatattatgtagtttacgtattcagcaagaggtgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaat

SEQ ID NO: 18 - DNA, 6184 bp, synthetic construct
  misc_feature 1..6184: note = Synthetic Construct
  source 1..6184: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttcacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggatgagattgtcaagaatattcgtgagggaacgcacgtgttgctgcctttctacgagactttgccggaattgaacctttcccttggaaagagccctcttcccagtctagaatatggagcaaattacttccttcagattagtagagtaaacgatctgaacaggatgccaaccgatatgttgaagctatttacacacgatattatgttacctgaatccgatttagacaaagtttacgaaatcctgaaaattaactcagtgaagtattacggccgtagcacgagggccgacgccgtagttgcagatctaagtgctaggaacaagctattcaagcgtgagagagacgctataaaaagtaacaaccacctgaccgagaacaatctttacataagtgactataagatgcttaccttcgacgtctttagaccattattcgactttgtgaatgaaaaatattgcatcataaagttaccgacgctttttggaagaggggttatagacacaatgagaatatactgctcacttttcaagaatgtgagactattaaagtgtgtctcagacagttggctgaaagactccgcaattatggttgcatcagatgtttacaagaagaacttagacctatttatgtctcacgttaagtctgttacgaagtccagtagttggaaagatgtaaacacagttcaattctccattttgaacgatcccgtagacacggaatttattaacaaattccttgagttctctaatagagtctacgaagccttgtactatgttcacagccttttatactccagtatgaccagtgattcaaaaagtatagaaaataaacatcagcgtaggctagttaaattactgctatgatttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 19 - DNA, 10945 bp, synthetic construct
  misc_feature 1..10945: note = Synthetic Construct
  source 1..10945: mol_type = other DNA
tcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggctagtcttgagaatctggtagcacgttatcagagatgtttcaacgatcagtctctgaaaaattcaaccatcgaatttgaaatcaggttccagcacattaactttttattgtttaagacggtgtatgaagctctagtcgcacaagaaatcccaagtactatcagccactctatcaggtgtataaagaaggtccaccatgaaaatcactgcagagaaaaaatccttccgtctgacaatttctacttcaagaagcaaccgcttatgttttttaaatttagtgagcctgcttccttgggatgcaaagttagcctagccatcgagcaaccaattcgtaagttcatcttagactcttctgtgctggtcaggttaaaaaatagaaccacattccagattagcgaactttggaagatcgaactgaccgtcgtcaaacagcttatgggctctgaagtcagcgcaaaattaactgccttcaagactcttttgtttgacacaccggagcaacagactgcaaagaatatgatgaccttgattaatcctgacgatgagtacttgtacgagatcgagatagaatacacagggaaacctgaaagtttgactgctgcagacgtgatcaagatcaaaaacaccgttctaaccttaatatcacctaatcatcttatgctgacagcatatcatcaggctatcgagttcattgcttcacatattcttagttctgagattcttctggctagaataaaatctggaaagtgggggttaaaaagattgctgcctcaggtaaaatccatgactaaagcggattatatgaaattctacccaccggtaggatactatgttacagataaagcggatgggatacgtggcattgctgtcattcaggatacccaaatgtacgtggtcgcagatcaattatattcactgggaacgaccggtatagaacccctaaagcccactattcttgacggcgaattcatgcctgaaaagaaagaattttatggattcgatgtgattatgtacgagggcaatctgctaacgcagcagggttttgagacgagaatagaagcacttaacaaaggcatcaaagttttgcaggcctttaatataaaggcagaaatgaagcccttcataagtcttaccagcgcagaccccaatgtcctgttaaaaaattttgaaagcgttttcaaaaaaaaaactagaccgtactcaatagacggcataatactggtcgagccgggaaacagttatttaaacaccaataccttcaaatggaaacctacatgggacaataccttggacttcctggtcagaaaatgtcccgagtccctaaacgtccctgagtatgcacctaaaaagggcttctccctgcatctgctatttgtcggaatcagtggggagttattcaagaaattggcgctaaactggtgtcctggctacacgaagttattccctgttacccaacgtaatcagaactatttcccggtccaattccagccgtccgattttccattggcttttttatactatcatcccgacacgagtagtttcagtgacatagatggaaaggtactggaaatgagatgcctaaaaagggaggtaaattacgtctcatgggaaatcgtcaagattcgtgaagatagacaacaagatctaaagactgggggttatttcggtaacgattttaagactgcagagctgacatggttgaattacatggacccgttcagctttgaggaattagccaaaggaccatccggcatgtactttgctggggcgaagaccggcatctatcgtgcgcagacggcgttgatcagttttataaaacaagaaattatacagaaaatcagccatcaaagttgggtcatagatctgggaatcggaaagggtcaggatctagggcgttacctggatgcaggaattagacatctggttggtattgataaagaccagaccgcattggcggaattaatatataggaaattcagtcatgctacgacccgtcagcacaagcacgcgacgaacatctatgtattgcaccaagacttggcggaacctgctaaagaaatctctgaaaaggtgcatcaaatatatgggtttcccaaagaaggagctagtagtatagtaagcaatctgtttatacattatctgatgaagtccagccaacaggttgagaacttagcggtactatgtcataagttgttgcagcctggcgggatggtatggttcacaaccatgctgggtgagcgtgtcctggaattattacatgagaatagggtcgagctgaacgaagtctgggaagcaagggaaaatgaagtggttaagtttgccattaagaggctttttaaagaggatgtactgcaagagacggggcaagaaataggtgtactgcttccgttctctaacggcgatttctataacgaatatctggtaaacacagctttcctaatcaagatcttcaagcaccacggctttagcttggtccaaatgcaaagttttaaggattggatacccgaatttcagacgttcagcaaatcactttataagatactgacggaggcggacaaaacgtggacgtccctgttcgggtttatatgcttaaggaagaatgaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaacctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaag

SEQ ID NO: 20 - DNA, 60 bp, synthetic construct
  misc_feature 1..60: note = Synthetic Construct
  source 1..60: mol_type = other DNA
tcaatctttttcaattttttgtttgtattcttttcttgcttaaatctataactacaaaaa

SEQ ID NO: 21 - DNA, 9772 bp, synthetic construct
  misc_feature 1..9772: note = Synthetic Construct
  source 1..9772: mol_type = other DNA
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaactccgcaaaggcagaggagtacgagaagatgagccttgaacaggctaaagcgtccgttaacagcgagaccgagtccagtttcaacatcaatgagaatacaacagctagtggcactggtttgtccgaaaagacgagtgtgtgtagacaggtagacattgctagaaagcgtaaggagttcgaggacgatttagtcaaggagtcatcttcctgcggaaaggatactccaagtaagaagcgtaagctagacccggagatagtgccagaagagaaagactgcggagatgcagagggtaatagcaaaaagagaaaaagagaaactgaggatgtgccgaaggacaagtcctcaactggtgatgggactcagaacaaaaggaagatagcattggaggacgtacctgagaagcagaaaaatcttgaggaaggtcattccagcacagttgcggcgcactataatgagctacaggaggtagggttggagaagcgttcccaatcaaggatattctatctaagaaactttaacaactggataaagtccgttctaataggagaattcttggagaaggtgagacaaaaaaaaaagagggatataacggttctagacttggggtgtgggaaagggggtgacttgttgaaatggaaaaaaggaaggataaataaacttgtctgtacggatatcgccgatgtttctgttaaacaatgtcaacaaaggtatgaagatatgaagaatcgtagagatagcgaatacattttttctgccgagttcataactgcggatagcagcaaagagcttctgatcgacaaattccgtgacccacaaatgtgctttgatatttgctcttgtcaattcgtttgtcattactcctttgagtcatacgaacaagcagacatgatgctgaggaatgcctgtgaaagactgtctcctggtggatacttcattgggacgaccccgaacagtttcgagttaatcagaaggttggaagccagtgaaaccgaatcatttggcaatgaaatatacacagtgaagtttcagaagaaaggagattacccgctgttcggctgtaagtatgactttaacttggagggagtagttgacgtgcccgagtttctagtctattttcctctgttgaacgagatggcgaagaagtataacatgaaattagtttacaaaaagacgttccttgagttctatgaagaaaagatcaagaacaatgaaaacaagatgttacttaaacgtatgcaggcacttgaaccgtacccggccaatgaaagtagtaagttagtcagcgaaaaagtcgacgattacgaacatgcggcaaaatatatgaagaattctcaagtaagattaccgttgggaacactatcaaagtccgaatgggaggcaacttccatctacttggttttcgcctttgagaagcaacaggaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaaccgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 22 - DNA, 9916 bp, synthetic construct
  misc_feature 1..9916: note = Synthetic Construct
  source 1..9916: mol_type = other DNA
tcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgtcccaaattgtcactggactgttcaaagactgctcccgtgagacttcaggcttgtcaccagcgtatgcaccaacgtacgtcagcgttgacgataagtataaaacttcagatgaattgtgtgtgaatctaaatcttccagcgaatgtgccttatagtagagttatatcacgtatgggctttaaactggacgcaactgttccaggctatccgaaactatttattacaagggaggaagcagtgcgtcaagtgaggtcatggataggtttcgacgtagaaggagcccatgcgagtagaaacgcctgcggaaccaatgttccgcttcaacttgggttctccaccggggtaaacttcgtggttcagcctgttggtgtcgttgatactgaatggggaaacatgttaacggggatagccgcgaggcctccgcccggagagcaatttaagcacctggtcccgcttatgcataaaggggctgcttggccaatcgtccgtaggcgtatcgttcagatgttgagcgatacccttgacaagctttccgattactgcacatttgtatgttgggcccatgggtttgaactaacgtcagcctcctacttttgcaaaattgggaaggagcaaaagtgttgtatgtgtaatcgtagagctgcagcatactcaagccccttgcagagttacgcctgttggactcattcatgtggctacgactacgtatacaatccattctttgtagacgtacagcagtggggctacgttggcaatttggcaacaaatcatgacagatattgttctgtgcatcaaggcgctcatgttgcgtccaatgatgctataatgactagatgcctggcgatccattcttgcttcatagaaagggtggactgggatattgaatacccgtacatatcacacgaaaagaaacttaattcttgttgccgtatcgtggagaggaatgtagtgagggcagctttgctggcgggatcatttgataaagtgtatgacatagggaaccctaagggtatccccattgtggatgatccggtggtagattggcactactttgatgcacaaccactaacaagaaaggttcaacagttattttacaccgaagatatggcatccagatttgctgatggcctttgtctattctggaactgtaatgtccccaaataccccaacaatgcgatcgtctgtcgttttgacacgagagtccattccgagtttaatttgccagggtgtgacggtgggtcactgtacgtgaataagcatgccttccatacaccagcctatgacgtttccgccttcagagatttgaaaccgctaccatttttctactactctacgactccgtgcgaggtacatggtaacggcagtatgatcgaagatatagattatgttcctctgaaaagcgccgtttgcataacagcttgcaaccttgggggagccgtttgcagaaaacatgccacagagtacagggaatatatggaagcctataatcttgtctcagcctctggattccgtctgtggtgctataaaacttttgatatatataacctttggagtacgtttacaaaagttcaggaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaacctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaag

SEQ ID NO: 23 - DNA, 8764 bp, synthetic construct
  misc_feature 1..8764: note = Synthetic Construct
  source 1..8764: mol_type = other DNA
tcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggcagagaatgtaaccgggctattcaaggattgcagcaaaataattaccgggcttcatccgacgcaagcacccacacacctgagtgtcgatatcaagttcaagactgaaggactttgtgtcgacatacctgggattccgaaggacatgacctatcgtagactaatttctatgatgggattcaagatgaattatcaagttaacggctatccaaatatgttcataactagggaggaagcaatacgtcatgtacgtgcttggataggattcgacgtagaaggatgtcatgccacacgtgacgctgtgggtactaacttaccattacaattaggctttagcaccggggttaacttagttgcagttcccaccggctacgtcgacaccgagaataacacggagtttacaagggtaaatgccaagccgccgccaggtgatcagttcaagcacttgatccctttgatgtacaagggactaccctggaatgtagtaagaatcaagatagtacaaatgctatccgacaccctaaaaggattatccgaccgtgttgttttcgtcttgtgggcacacggctttgaactaacgtcaatgaaatactttgtcaaaattggaccagagagaacgtgctgcctttgtgataagcgtgcgacttgttttagcaccagctctgacacctatgcatgttggaatcattctgtagggtttgactacgtatacaacccatttatgatagacgtacaacaatggggttttacgggtaaccttcagtctaatcacgaccaacattgtcaagtacacggcaatgcgcacgtcgcatcctgtgacgccataatgactagatgtttggcagtacatgaatgtttcgtcaaacgtgtcgattggagcgtggaatacccgattattggtgatgaactgagggtcaactctgcttgtaggaaagtacagcacatggtagttaaatccgcgcttctagcggacaaattccctgttctacacgacatcggtaatccgaaagccataaaatgtgtaccacaagcagaagtcgaatggaagttttacgacgcccaaccttgctctgataaagcctacaagatcgaagagttgttctattcttacgcgacccaccatgataagtttacagatggtgtttgtttgttctggaactgtaacgtggatagataccctgccaacgccatagtttgcagattcgacacgagagtactgagtaatctgaaccttcccgggtgtgatggtgggagtttatacgtgaacaaacatgcctttcatacaccggcctttgataagagcgcatttaccaacttaaaacaactgccatttttttattactcaggtagtccgtgcgagagtcatgggaaacaggtagtcagtgatatagattacgtaccgctgaagtcagcaacttgtataaccagatgcaatcttggtggagctgtctgtcgtcatcatgctaacgaataccgtcaatacttagacgcatacaacatgatgatttccgcgggattctcattatggatttataaacaatttgatacatacaatttatggaatacctttacaaggctacagtgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaag

SEQ ID NO: 24 - protein, 525 aa, MERS-CoV
MSQIVTGLFKDCSRETSGLSPAYAPTYVSVDDKYKTSDELCVNLNLPANVPYSRVISRMGFKLDATVPGYPKLFITREEAVRQVRSWIGFDVEGAHASRNACGTNVPLQLGFSTGVNFVVQPVGVVDTEWGNMLTGIAARPPPGEQFKHLVPLMHKGAAWPIVRRRIVQMLSDTLDKLSDYCTFVCWAHGFELTSASYFCKIGKEQKCCMCNRRAAAYSSPLQSYACWTHSCGYDYVYNPFFVDVQQWGYVGNLATNHDRYCSVHQGAHVASNDAIMTRCLAIHSCFIERVDWDIEYPYISHEKKLNSCCRIVERNVVRAALLAGSFDKVYDIGNPKGIPIVDDPVVDWHYFDAQPLTRKVQQLFYTEDMASRFADGLCLFWNCNVPKYPNNAIVCRFDTRVHSEFNLPGCDGGSLYVNKHAFHTPAYDVSAFRDLKPLPFFYYSTTPCEVHGNGSMIEDIDYVPLKSAVCITACNLGGAVCRKHATEYREYMEAYNLVSASGFRLWCYKTFDIYNLWSTFTKVQ

SEQ ID NO: 25 - DNA, 1575 bp, synthetic construct
  misc_feature 1..1575: note = Synthetic Construct
  source 1..1575: mol_type = other DNA
atgtcccaaattgtcactggactgttcaaagactgctcccgtgagacttcaggcttgtcaccagcgtatgcaccaacgtacgtcagcgttgacgataagtataaaacttcagatgaattgtgtgtgaatctaaatcttccagcgaatgtgccttatagtagagttatatcacgtatgggctttaaactggacgcaactgttccaggctatccgaaactatttattacaagggaggaagcagtgcgtcaagtgaggtcatggataggtttcgacgtagaaggagcccatgcgagtagaaacgcctgcggaaccaatgttccgcttcaacttgggttctccaccggggtaaacttcgtggttcagcctgttggtgtcgttgatactgaatggggaaacatgttaacggggatagccgcgaggcctccgcccggagagcaatttaagcacctggtcccgcttatgcataaaggggctgcttggccaatcgtccgtaggcgtatcgttcagatgttgagcgatacccttgacaagctttccgattactgcacatttgtatgttgggcccatgggtttgaactaacgtcagcctcctacttttgcaaaattgggaaggagcaaaagtgttgtatgtgtaatcgtagagctgcagcatactcaagccccttgcagagttacgcctgttggactcattcatgtggctacgactacgtatacaatccattctttgtagacgtacagcagtggggctacgttggcaatttggcaacaaatcatgacagatattgttctgtgcatcaaggcgctcatgttgcgtccaatgatgctataatgactagatgcctggcgatccattcttgcttcatagaaagggtggactgggatattgaatacccgtacatatcacacgaaaagaaacttaattcttgttgccgtatcgtggagaggaatgtagtgagggcagctttgctggcgggatcatttgataaagtgtatgacatagggaaccctaagggtatccccattgtggatgatccggtggtagattggcactactttgatgcacaaccactaacaagaaaggttcaacagttattttacaccgaagatatggcatccagatttgctgatggcctttgtctattctggaactgtaatgtccccaaataccccaacaatgcgatcgtctgtcgttttgacacgagagtccattccgagtttaatttgccagggtgtgacggtgggtcactgtacgtgaataagcatgccttccatacaccagcctatgacgtttccgccttcagagatttgaaaccgctaccatttttctactactctacgactccgtgcgaggtacatggtaacggcagtatgatcgaagatatagattatgttcctctgaaaagcgccgtttgcataacagcttgcaaccttgggggagccgtttgcagaaaacatgccacagagtacagggaatatatggaagcctataatcttgtctcagcctctggattccgtctgtggtgctataaaacttttgatatatataacctttggagtacgtttacaaaagttcag

SEQ ID NO: 26 - DNA, 11140 bp, synthetic construct
  misc_feature 1..11140: note = Synthetic Construct
  source 1..11140: mol_type = other DNA
tcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgagtgcagatgctcaatctttcctaaatagggtgtgtggtgtgagcgccgctagacttacaccttgcgggacggggacctctacggatgtcgtgtatagagcgttcgatatttataatgataaagtcgcaggttttgctaagtttctgaaaacaaattgttgtaggttccaggagaaagatgaagatgacaacttaattgattcatacttcgttgttaaaaggcataccttctctaattaccaacatgaagaaacaatttataatttgttgaaggactgtccagcggtcgcaaaacacgatttctttaaattcaggatagatggagatatggtaccacacatctctagacagaggcttacgaaatatacaatggccgatctagtctatgcattacgtcacttcgatgagggaaactgtgacacattaaaggaaatcttagtaacctataactgttgcgatgatgattattttaacaagaaggattggtacgatttcgttgaaaatccggatattttgcgtgtttacgcaaatttgggtgaaagagttagacaagccttattaaagaccgtccaattttgcgacgcaatgcgaaacgcgggtattgttggcgtactaacactcgataaccaagatttgaacggtaattggtacgactttggagatttcattcaaactaccccaggtagtggtgttccagtcgtagattcatattacagtctattgatgcctattttaacccttacaagagcactcactgccgaaagtcatgttgatacagacctgactaagccatacattaaatgggatttattgaagtatgacttcactgaagagaggctaaaacttttcgacagatactttaaatattgggaccaaacttaccatcctaattgtgtaaactgtttggatgacagatgtatattgcactgtgccaacttcaatgtccttttttctacagtctttccccccacaagctttggcccactggtgcgcaaaatatttgttgacggtgtgcctttcgttgtctccacaggttatcacttcagagaattaggagtagtacataatcaagatgtgaacttgcactcatctcggttatcgttcaaagagttactagtttatgctgctgatcctgccatgcatgcagcttctggcaatctattattggataagcgtactacttgcttttcagttgccgcactgactaataatgttgcttttcagacagtcaaaccaggcaattttaacaaggatttttatgactttgctgtatctaaaggcttcttcaaggagggttcctccgtggaattaaagcatttcttttttgcccaggatggtaacgccgcaatttctgactatgattattatagatacaatttacctaccatgtgcgacatcagacaattattgtttgtagttgaggtcgtggataaatactttgattgttacgatgggggctgcatcaacgctaatcaggtcatagttaataatttagacaaatccgcggggtttccctttaataagtggggtaaagcacgtctttactatgattcgatgtcttatgaagatcaagatgctttgtttgcatatacaaagcgtaatgttatacctactataacgcaaatgaacttgaaatacgctatttctgccaaaaatcgagctaggacagttgcaggtgtttcaatatgctccaccatgacaaaccgccaatttcatcaaaaactgttaaagagcatagcggcaacaagaggagccacggttgtgatcggtacttctaaattctatggtggatggcataatatgttgaaaacggtgtactcggatgttgaaaatccacatctaatgggctgggattaccctaaatgcgacagggcgatgccaaacatgttgagaatcatggccagcttagttcttgctagaaaacatacgacttgttgtagcctctcacatagattttatcggctcgcaaatgaatgcgcccaagtgttgtcggaaatggttatgtgtgggggttcattatatgtaaagccaggaggcacgtccagtggtgatgctactacagcttatgcgaattctgtttttaatatctgtcaggctgtaactgcaaatgttaacgcactattaagtacagacggtaataaaattgctgacaagtacgtcaggaaccttcagcatagactgtacgaatgtttatatcgtaaccgagacgttgacacggactttgtcaacgagttttatgcttatctgagaaaacacttttcaatgatgattctatcggacgatgctgtagtttgttttaactccacttatgctagtcaaggcttagtagcttcaatcaagaactttaaatccgtgttgtattaccaaaacaatgtttttatgtcagaagctaagtgctggaccgaaactgacttgactaaaggaccacatgagttctgttctcagcacaccatgctcgtaaaacaaggagacgattacgtctacttaccctatcctgatccaagtagaattcttggtgcaggttgcttcgtggatgacatagtaaaaactgacggaactcttatgatcgaaagattcgtttcactggctattgatgcttacccgctgaccaagcatccgaatcaggagtacgccgatgtttttcacttgtacctacaatatattagaaagttgcacgatgaattgactggtcatatgcttgatatgtacagcgtcatgttgactaatgataataccagtagatattgggaaccggaattttacgaagcaatgtatactccacataccgtgttgcaagaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaacctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaag

SEQ ID NO: 27 - DNA, 8083 bp, synthetic construct
  misc_feature 1..8083: note = Synthetic Construct
  source 1..8083: mol_type = other DNA
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaggtggagctaaggggaggacattgggggaagtttggaaggagagattaaaccaaatgaccaaagaggaatttacccgttacagaaaagaagccatcatcgaagtagaccgttcagctgccaagcatgctagaaaggagggcaatgtaacggggggccatccagtgagcagagggacggctaagttgcgttggcttgttgaaagaagatttcttgaaccagtggggaaagtcattgacttgggttgtggcaggggaggttggtgctactacatggcgacacagaaacgtgtccaggaagtaaggggatatactaaaggcggacccggacacgaagaacctcagctggttcaatcctatggctggaacatcgtcacgatgaagtcaggggttgacgtgttctatcgtccgtcagaatgttgcgacaccttactgtgtgatataggggagagctcaagctctgcggaggtggaggagcatagaacgatccgtgttctggagatggtcgaggattggttgcatcgtggcccaagagagttctgcgtcaaggtcctttgtccgtacatgcctaaggttatagaaaagatggagctgttacaacgtagatatgggggcggtttggtaagaaatccccttagcagaaacagcacgcacgaaatgtactgggtcagcagagcttcaggtaatgtggtgcatagcgtaaacatgactagccaggtactgttaggccgtatggagaaaaggacatggaagggaccgcaatatgaagaggatgtgaaccttggttctggaacaagggcggttggtaaacccctgctgaattctgacactagtaaaattaagaaccgtatagagaggttacgtagggagtacagctctacgtggcaccatgatgaaaatcacgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaagcatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 28 - DNA, 6628 bp, synthetic construct
  misc_feature 1..6628: note = Synthetic Construct
  source 1..6628: mol_type = other DNA
tcgcgcgtttcggtgatgacggtgaaaacctctgacacatgcagctcccggagacggtcacagcttgtcacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggttttagcaatggaaagtagagtggcaccggaaattcctgggctcattcaacctgggaatgtcacgcaagacttgaagatgatggtctgtaaattattgaattccccaaaacctacgaaaacattccctggttcccagcctgtgtcctttcagcattctgatgtggaagagaagctgcttgcgcatgattactacgtttgtgagaaaacagatggtctgcgggtgttgatgtttatagtgataaatcctgtgacgggtgagcaaggatgctttatgattgatagggaaaataactattatctggttaatggatttaggtttcccagattaccccaaaagaagaaagaagagctgctagagactcttcaagatggcaccttattagatggtgaacttgtcatacaaactaacccaatgacaaaattacaagagttgcgttatttaatgttcgattgtcttgctatcaatggtagatgtctcacacaatcaccaacaagttctagactagcccaccttggaaaagaattttttaaaccatacttcgatttaagagcagcgtaccctaatcgttgtactacttttccgttcaaaatttccatgaaacatatggatttcagttaccaattagtaaaagttgctaaaagtttagataaactaccacatctttctgatggtctgatatttactcctgtgaaggcaccttacactgccggcggaaaagattcattgttattaaaatggaagccagaacaagaaaacaccgtggacttcaaattgattttagatatcccaatggtggaggatccttctttgcctaaagatgatcggaacaggtggtattacaattatgacgttaagccagttttcagcttatatgtctggcaaggcggagctgatgtcaattcacgtttaaaacatttcgaccagcctttcgataggaaggaatttgaaatattagaaagaacatacagaaaatttgcagagttgagcgtttcagatgaggaatggcaaaatttgaagaacctagaacagccattaaatggtagaatagtagagtgcgcaaaaaaccaagagactggggcgtgggaaatgttaagattcagggatgataagttaaatggtaatcatacatcggtggtccagaaagttttggagagtatcaacgattcagtttcattggaggacctcgaggaaattgttggtgatattaaaaggtgctgggacgagagaagagcaaatatggctggtggtagtgggagaccactaccgtctcaaagtcaaaatgcgacattatctacctctaagccagtccattcacagcccccaagtaatgataaggagccaaaatatgtagacgaggatgattggtcggattagtgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 29 - protein, 933 aa, SARS-CoV-2
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 30 - DNA, 2799 bp, synthetic construct
  misc_feature 1..2799: note = Synthetic Construct
  source 1..2799: mol_type = other DNA
atgagtgcagatgctcaatctttcctaaatagggtgtgtggtgtgagcgccgctagacttacaccttgcgggacggggacctctacggatgtcgtgtatagagcgttcgatatttataatgataaagtcgcaggttttgctaagtttctgaaaacaaattgttgtaggttccaggagaaagatgaagatgacaacttaattgattcatacttcgttgttaaaaggcataccttctctaattaccaacatgaagaaacaatttataatttgttgaaggactgtccagcggtcgcaaaacacgatttctttaaattcaggatagatggagatatggtaccacacatctctagacagaggcttacgaaatatacaatggccgatctagtctatgcattacgtcacttcgatgagggaaactgtgacacattaaaggaaatcttagtaacctataactgttgcgatgatgattattttaacaagaaggattggtacgatttcgttgaaaatccggatattttgcgtgtttacgcaaatttgggtgaaagagttagacaagccttattaaagaccgtccaattttgcgacgcaatgcgaaacgcgggtattgttggcgtactaacactcgataaccaagatttgaacggtaattggtacgactttggagatttcattcaaactaccccaggtagtggtgttccagtcgtagattcatattacagtctattgatgcctattttaacccttacaagagcactcactgccgaaagtcatgttgatacagacctgactaagccatacattaaatgggatttattgaagtatgacttcactgaagagaggctaaaacttttcgacagatactttaaatattgggaccaaacttaccatcctaattgtgtaaactgtttggatgacagatgtatattgcactgtgccaacttcaatgtccttttttctacagtctttccccccacaagctttggcccactggtgcgcaaaatatttgttgacggtgtgcctttcgttgtctccacaggttatcacttcagagaattaggagtagtacataatcaagatgtgaacttgcactcatctcggttatcgttcaaagagttactagtttatgctgctgatcctgccatgcatgcagcttctggcaatctattattggataagcgtactacttgcttttcagttgccgcactgactaataatgttgcttttcagacagtcaaaccaggcaattttaacaaggatttttatgactttgctgtatctaaaggcttcttcaaggagggttcctccgtggaattaaagcatttcttttttgcccaggatggtaacgccgcaatttctgactatgattattatagatacaatttacctaccatgtgcgacatcagacaattattgtttgtagttgaggtcgtggataaatactttgattgttacgatgggggctgcatcaacgctaatcaggtcatagttaataatttagacaaatccgcggggtttccctttaataagtggggtaaagcacgtctttactatgattcgatgtcttatgaagatcaagatgctttgtttgcatatacaaagcgtaatgttatacctactataacgcaaatgaacttgaaatacgctatttctgccaaaaatcgagctaggacagttgcaggtgtttcaatatgctccaccatgacaaaccgccaatttcatcaaaaactgttaaagagcatagcggcaacaagaggagccacggttgtgatcggtacttctaaattctatggtggatggcataatatgttgaaaacggtgtactcggatgttgaaaatccacatctaatgggctgggattaccctaaatgcgacagggcgatgccaaacatgttgagaatcatggccagcttagttcttgctagaaaacatacgacttgttgtagcctctcacatagattttatcggctcgcaaatgaatgcgcccaagtgttgtcggaaatggttatgtgtgggggttcattatatgtaaagccaggaggcacgtccagtggtgatgctactacagcttatgcgaattctgtttttaatatctgtcaggctgtaactgcaaatgttaacgcactattaagtacagacggtaataaaattgctgacaagtacgtcaggaaccttcagcatagactgtacgaatgtttatatcgtaaccgagacgttgacacggactttgtcaacgagttttatgcttatctgagaaaacacttttcaatgatgattctatcggacgatgctgtagtttgttttaactccacttatgctagtcaaggcttagtagcttcaatcaagaactttaaatccgtgttgtattaccaaaacaatgtttttatgtcagaagctaagtgctggaccgaaactgacttgactaaaggaccacatgagttctgttctcagcacaccatgctcgtaaaacaaggagacgattacgtctacttaccctatcctgatccaagtagaattcttggtgcaggttgcttcgtggatgacatagtaaaaactgacggaactcttatgatcgaaagattcgtttcactggctattgatgcttacccgctgaccaagcatccgaatcaggagtacgccgatgtttttcacttgtacctacaatatattagaaagttgcacgatgaattgactggtcatatgcttgatatgtacagcgtcatgttgactaatgataataccagtagatattgggaaccggaattttacgaagcaatgtatactccacataccgtgttgcaa

SEQ ID NO: 31 - protein, 301 aa, West Nile virus; identical to SEQ ID NO: 12

SEQ ID NO: 32 - DNA, 903 bp, synthetic construct
  misc_feature 1..903: note = Synthetic Construct
  source 1..903: mol_type = other DNA
atgggtggagctaaggggaggacattgggggaagtttggaaggagagattaaaccaaatgaccaaagaggaatttacccgttacagaaaagaagccatcatcgaagtagaccgttcagctgccaagcatgctagaaaggagggcaatgtaacggggggccatccagtgagcagagggacggctaagttgcgttggcttgttgaaagaagatttcttgaaccagtggggaaagtcattgacttgggttgtggcaggggaggttggtgctactacatggcgacacagaaacgtgtccaggaagtaaggggatatactaaaggcggacccggacacgaagaacctcagctggttcaatcctatggctggaacatcgtcacgatgaagtcaggggttgacgtgttctatcgtccgtcagaatgttgcgacaccttactgtgtgatataggggagagctcaagctctgcggaggtggaggagcatagaacgatccgtgttctggagatggtcgaggattggttgcatcgtggcccaagagagttctgcgtcaaggtcctttgtccgtacatgcctaaggttatagaaaagatggagctgttacaacgtagatatgggggcggtttggtaagaaatccccttagcagaaacagcacgcacgaaatgtactgggtcagcagagcttcaggtaatgtggtgcatagcgtaaacatgactagccaggtactgttaggccgtatggagaaaaggacatggaagggaccgcaatatgaagaggatgtgaaccttggttctggaacaagggcggttggtaaacccctgctgaattctgacactagtaaaattaagaaccgtatagagaggttacgtagggagtacagctctacgtggcaccatgatgaaaatcac

SEQ ID NO: 33 - protein, 597 aa, Homo sapiens
MAHNKIPPRWLNCPRRGQPVAGRFLPLKTMLGPRYDSQVAEENRFHPSMLSNYLKSLKVKMGLLVDLTNTSRFYDRNDIEKEGIKYIKLQCKGHGECPTTENTETFIRLCERFNERNPPELIGVHCTHGFNRTGFLICAFLVEKMDWSIEAAVATFAQARPPGIYKGDYLKELFRRYGDIEEAPPPPLLPDWCFEDDEDEDEDEDGKKESEPGSSASFGKRRKERLKLGAIFLEGVTVKGVTQVTTQPKLGEVQQKCHQFCGWEGSGFPGAQPVSMDKQNIKLLDLKPYKVSWKADGTRYMMLIDGTNEVFMIDRDNSVFHVSNLEFPFRKDLRMHLSNTLLDGEMIIDRVNGQAVPRYLIYDIIKFNSQPVGDCDFNVRLQCIEREIISPRHEKMKTGLIDKTQEPFSVRNKPFFDICTSRKLLEGNFAKEVSHEMDGLIFQPTGKYKPGRCDDILKWKPPSLNSVDFRLKITRMGGEGLLPQNVGLLYVGGYERPFAQIKVTKELKQYDNKIIECKFENNSWVFMRQRTDKSFPNAYNTAMAVCNSISNPVTKEMLFEFIDRCTAASQGQKRKHHLDPDTELMPPPPPKRPRPLT

SEQ ID NO: 34 - protein, 114 aa, SARS-CoV-2
MNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 35 - DNA, 1311 bp, Saccharomyces cerevisiae
  source 1..1311: mol_type = unassigned DNA
atgtcaaccaaaccagagaagccaatatggatgtcacaagaagactatgaccgtcagtacggctcaattactggtgatgaatcgtccacagtctcaaaaaaagactccaaagttactgccaatgcgcctggtgatggtaatggatcattaccagttttgcaaagcagctctatccttacttctaaggtgtctgatctacccattgaggcagaatccgggtttaaaatccaaaagagaagacatgaaaggtatgatcaagaggaaagattacgcaaacagcgtgctcaaaaattgagagaagagcaactaaagagacatgaaatagagatgactgcaaatagatccataaatgttgaccagattgtccgagagcactataatgaacgtacaataatcgccaatcgcgctaagaggaatctatcgcctattattaagcttcgtaattttaacaatgcgattaagtatatgttaattgacaaatatacaaaacctggagatgtcgttttagaactcggatgtggcaaaggtggtgatttaagaaaatatggtgctgcaggtatttcacagtttattggtattgacatttccaatgcctctatccaagaagctcataagagataccggtccatgagaaatttggactaccaagttgtgctaattactggggattgctttggcgaatcattaggtgttgcagtagagcccttcccagactgtagatttccgtgtgatatcgtttcgacgcaattttgcttgcattatgcttttgagaccgaagaaaaagcaaggagagctttactgaatgtcgccaaatcactcaaaattggtggtcacttttttggcacgataccagattcagaattcattcgttataaactgaataaatttcccaaagaagtggaaaagccatcttggggaaactccatttataaagtgacatttgagaataattcataccaaaagaatgattatgagttcacatcgccttatggtcaaatgtatacttattggctagaagatgctatagataatgttcctgaatatgttgtgccattcgaaaccttaagaagtttggctgatgaatacggtttggaactagtatcgcaaatgccttttaataaattttttgtccaagaaattcccaaatggattgagaggttttcgcctaaaatgagggaaggtttacaaagatctgatggtagatatggtgttgaaggggatgaaaaggaagcggcatcatatttctataccatgtttgcattccgtaaggtaaaacagtatatcgaaccggaaagcgtaaagcccaactga

SEQ ID NO: 36 - protein, 436 aa, Saccharomyces cerevisiae
MSTKPEKPIWMSQEDYDRQYGSITGDESSTVSKKDSKVTANAPGDGNGSLPVLQSSSILTSKVSDLPIEAESGFKIQKRRHERYDQEERLRKQRAQKLREEQLKRHEIEMTANRSINVDQIVREHYNERTIIANRAKRNLSPIIKLRNFNNAIKYMLIDKYTKPGDVVLELGCGKGGDLRKYGAAGISQFIGIDISNASIQEAHKRYRSMRNLDYQVVLITGDCFGESLGVAVEPFPDCRFPCDIVSTQFCLHYAFETEEKARRALLNVAKSLKIGGHFFGTIPDSEFIRYKLNKFPKEVEKPSWGNSIYKVTFENNSYQKNDYEFTSPYGQMYTYWLEDAIDNVPEYVVPFETLRSLADEYGLELVSQMPFNKFFVQEIPKWIERFSPKMREGLQRSDGRYGVEGDEKEAASYFYTMFAFRKVKQYIEPESVKPN

SEQ ID NO: 37 - DNA, 1380 bp, Saccharomyces cerevisiae
  source 1..1380: mol_type = unassigned DNA
atggttttagcaatggaaagtagagtggcaccggaaattcctgggctcattcaacctgggaatgtcacgcaagacttgaagatgatggtctgtaaattattgaattccccaaaacctacgaaaacattccctggttcccagcctgtgtcctttcagcattctgatgtggaagagaagctgcttgcgcatgattactacgtttgtgagaaaacagatggtctgcgggtgttgatgtttatagtgataaatcctgtgacgggtgagcaaggatgctttatgattgatagggaaaataactattatctggttaatggatttaggtttcccagattaccccaaaagaagaaagaagagctgctagagactcttcaagatggcaccttattagatggtgaacttgtcatacaaactaacccaatgacaaaattacaagagttgcgttatttaatgttcgattgtcttgctatcaatggtagatgtctcacacaatcaccaacaagttctagactagcccaccttggaaaagaattttttaaaccatacttcgatttaagagcagcgtaccctaatcgttgtactacttttccgttcaaaatttccatgaaacatatggatttcagttaccaattagtaaaagttgctaaaagtttagataaactaccacatctttctgatggtctgatatttactcctgtgaaggcaccttacactgccggcggaaaagattcattgttattaaaatggaagccagaacaagaaaacaccgtggacttcaaattgattttagatatcccaatggtggaggatccttctttgcctaaagatgatcggaacaggtggtattacaattatgacgttaagccagttttcagcttatatgtctggcaaggcggagctgatgtcaattcacgtttaaaacatttcgaccagcctttcgataggaaggaatttgaaatattagaaagaacatacagaaaatttgcagagttgagcgtttcagatgaggaatggcaaaatttgaagaacctagaacagccattaaatggtagaatagtagagtgcgcaaaaaaccaagagactggggcgtgggaaatgttaagattcagggatgataagttaaatggtaatcatacatcggtggtccagaaagttttggagagtatcaacgattcagtttcattggaggacctcgaggaaattgttggtgatattaaaaggtgctgggacgagagaagagcaaatatggctggtggtagtgggagaccactaccgtctcaaagtcaaaatgcgacattatctacctctaagccagtccattcacagcccccaagtaatgataaggagccaaaatatgtagacgaggatgattggtcggattag

SEQ ID NO: 38 - protein, 459 aa, Saccharomyces cerevisiae
MVLAMESRVAPEIPGLIQPGNVTQDLKMMVCKLLNSPKPTKTFPGSQPVSFQHSDVEEKLLAHDYYVCEKTDGLRVLMFIVINPVTGEQGCFMIDRENNYYLVNGFRFPRLPQKKKEELLETLQDGTLLDGELVIQTNPMTKLQELRYLMFDCLAINGRCLTQSPTSSRLAHLGKEFFKPYFDLRAAYPNRCTTFPFKISMKHMDFSYQLVKVAKSLDKLPHLSDGLIFTPVKAPYTAGGKDSLLLKWKPEQENTVDFKLILDIPMVEDPSLPKDDRNRWYYNYDVKPVFSLYVWQGGADVNSRLKHFDQPFDRKEFEILERTYRKFAELSVSDEEWQNLKNLEQPLNGRIVECAKNQETGAWEMLRFRDDKLNGNHTSVVQKVLESINDSVSLEDLEEIVGDIKRCWDERRANMAGGSGRPLPSQSQNATLSTSKPVHSQPPSNDKEPKYVDEDDWSD

SEQ ID NO: 39 - DNA, 1650 bp, Saccharomyces cerevisiae
  source 1..1650: mol_type = unassigned DNA
atgagttacactgacaaccctcctcaaacaaaaagagctttatcgttagacgatctggtgaatcacgatgaaaatgaaaaggttaaattacaaaaattaagtgaggcggctaatggcagcagaccttttgccgaaaatttagaatctgatataaatcaaacggaaacgggccaagctgctccgattgacaattacaaggagagtactggtcatggctcgcactcacaaaaacctaaatcacgcaagtcatctaatgatgatgaagaaaccgatacggatgacgaaatgggtgcaagtggagaaattaattttgattcagaaatggactttgactatgataaacaacatagaaatttactatccaacggatcacctcctatgaatgatggtagtgatgccaatgcgaagttagaaaagccttctgatgattcaattcatcagaatagcaagagtgatgaagaacagagaataccgaaacaaggtaatgaagggaacattgccagcaactatataacccaagtacctctgcaaaagcagaagcaaactgagaagaagatagcgggaaatgcagtaggaagcgtggtcaagaaggaagaagaagcgaatgcagctgtagataatatttttgaagagaaagctactttacaatcaaaaaagaataatatcaagagagatttggaggttctgaatgaaatatctgcgtcttccaagcccagtaagtacaggaatgttccaatttgggcacaaaaatggaaacctactatcaaagctcttcaaagtataaatgtgaaagatctcaaaattgacccatcttttttaaacattattcccgatgatgacttaacaaagtcagtacaggactgggtttatgctacaatatactcaattgctcctgaactaagatccttcattgagttagaaatgaaatttggtgttattattgatgcgaaaggcccagatcgtgtaaatccaccagtttcttcacaatgtgttttcactgagcttgatgcccatctaacgcctaatattgatgcatctttgttcaaagagttgagcaaatatattcgtggtattagcgaagtcactgaaaatacaggtaaattcagtattattgaatcccagacaagagattccgtctatagagtcggactatccacgcaaagaccaaggtttttgagaatgagtacagatattaagactgggagggtaggacaatttatagagaaaagacatgtagcccaactactattatattcaccaaaagatagttacgacgttaaaatctccctaaacttggaattacctgtacctgacaacgatccgccagaaaaatataaatctcaaagcccaattagtgaaaggacgaaagaccgtgttagttacattcataatgattcctgtaccagaattgatattacaaaagtcgaaaatcataaccaaaattcaaaaagtagacaatcagagaccactcacgaagtggaactagaaatcaacacgcctgcactgttaaacgcctttgataacataacgaacgatagtaaagaatatgcatctcttattagaacgtttctgaataatggtacaattattagaagaaagttatcgtctttatcatatgaaatttttgaaggttcaaagaaagtcatgtaa

SEQ ID NO: 40 - protein, 549 aa, Saccharomyces cerevisiae [sequence omitted]

SEQ ID NO: 41 - DNA, 1581 bp, synthetic construct
  misc_feature 1..1581: note = Synthetic Construct
  source 1..1581: mol_type = other DNA
gctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcag

SEQ ID NO: 42 - DNA, 2764 bp, synthetic construct
  misc_feature 1..2764: note = Synthetic Construct
  source 1..2764: mol_type = other DNA
tcagctgatgcacaatcgtttttaaacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacag

SEQ ID NO: 43 - DNA, 339 bp, synthetic construct
  misc_feature 1..339: note = Synthetic Construct
  source 1..339: mol_type = other DNA
aataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaa

SEQ ID NO: 44 - DNA, 1572 bp, synthetic construct
  misc_feature 1..1572: note = Synthetic Construct
  source 1..1572: mol_type = other DNA
tctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaa

SEQ ID NO: 45 - DNA, 2607 bp, synthetic construct
  misc_feature 1..2607: note = Synthetic Construct
  source 1..2607: mol_type = other DNA
atggcttctttagataatttagtggcacgatatcagaggtgctttaatgaccagtctcttaaaaatagtactattgaacttgaaatacgttttcaacagataaattttttattattcaaaaccgtatatgaggcacttgtggcacaagagatccctagcaccatctcccacagcatccgctgcatcaaaaaagttcaccatgaaaaccactgccgggaaaaaattttgccgtcggaaaatctttacttcaaaaaacagcctctcatgttttttaagttttcagagcctgcatctctgggctgtaaggtctcgctggccatcgagcagcccattcgtaaatttatcttggactcctccgttctcgttcggctcaaaaatcgtacgacctttcgggtatctgaactttggaaaatagagcttaccattgtaaagcagctgatgggaagcgaggtctctgcaaaacttgccgctttcaaaacgcttctgtttgacaccccagagcaacaaacgacaaaaaatatgatgacgttaataaacccagatgacgaatatctttacgaaatagaaatagagtatacaggaaagcccgaatccctaacggcggcagatgttataaaaattaaaaacacggtgttgacacttatttctccgaaccatttaatgctaacagcctaccaccaggccattgaattcattgcctcccatatactgtcctcagaaatccttcttgctcgtattaagagcgggaagtgggggcttaaacgcctcctcccccaggtgaaatccatgaccaaagcggattacatgaaattttatccgcccgttggctactatgtaacggacaaagcagatggaattagaggcatcgccgtcattcaggacacgcaaatttatgtggttgcagaccagctatacagcctaggtaccaccggcattgaaccccttaaaccaaccattttggatggtgaatttatgcctgaaaaaaaagaattttatgggtttgacgtcatcatgtatgagggcaatctgttgacgcaacaggggtttgaaacaagaattgagtctttaagcaagggcattaaagtcttacaagcgtttaacataaaagcagaaatgaagccctttatttcgctaacaagtgcagatcccaacgtgctcctcaaaaactttgaaagcatttttaagaaaaaaactcgcccatattctattgatggcatcattttagtagaacctggcaattcttatctaaatacaaatacttttaagtggaagcccacctgggataacacattagactttttggtgcgaaaatgtccggagagtttaaacgtgccagagtacgcgcccaaaaaagggttttccctgcatctactatttgtaggcatctccggagagctttttaaaaaattagcgctaaattggtgtccaggatatacgaaactattccccgttacacagcgcaaccaaaactactttccagtacagtttcagccatcggattttccactggcatttctttattaccacccagatacctcgtcattttctaatatagatggaaaggtccttgaaatgcgttgtcttaagagagaaattaattacgtcagatgggaaattgtaaaaatccgggaggataggcagcaggatcttaaaaccggcgggtattttggcaatgatttcaaaacagccgaactcacatggcttaactatatggatcccttttcctttgaggaactggcaaagggcccttctggaatgtacttcgccggtgccaaaaccggcatataccgcgctcaaacagcacttatttcctttattaaacaagaaatcatccaaaaaataagtcaccaatcctgggttatcgatcttggaataggaaaagggcaggacctaggacgttacctggacgcaggggtaaggcatcttgttggaatcgataaggaccaaaccgcgctcgcggagcttgtttatcgaaaattttcgcatgctacaacccgacagcacaagcacgctaccaacatttacgtgttgcatcaagacctcgcagagcctgcgaaggaaatcagcgaaaaggtacaccaaatttacgggtttcccaaggagggagcttcttccattgttagcaacctgtttattcactatcttatgaaaaacacgcagcaggtggaaaacctggccgttctgtgccataagcttcttcagccggggggaatggtgtggtttaccaccatgttgggagaacaggtcttagaattacttcatgaaaatagaatagagctcaatgaagtatgggaggcacgtgaaaacgaggtggtcaaatttgctattaaacgtctctttaaagaggatatattacaggaaactgggcaagaaattggagtcctgttacccttcagcaatggcgacttctacaatgaatatcttgtgaacacagcgtttttaattaaaatatttaaacatcacggcttttccctagttcaaaagcagtcctttaaggactggattccagaatttcaaaactttagtaaaagtttgtataaaattcttacagaagccgataaaacttggacaagcctttttgggtttatttgtctgcgcaaaaattaa

SEQ ID NO: 46 - DNA, 2538 bp, synthetic construct
  misc_feature 1..2538: note = Synthetic Construct
  source 1..2538: mol_type = other DNA
atggatgccaacatagtatcatcttctactattgcgacgtatatagacgctttagcgaagaatgcttcagaattagaacagaggtctaccgcatacgaaataaataatgaattggaactagtatttattaaaccgccattgattacgttgacaaatgtagtaaatatctccacgattcaggaatcgtttattcgatttaccgttactaataaggaaggtatcaaaattagaactaagattccattatctaaggtacatggtctagatgtaaaaaatgtgcagttggtagatgctatagataacatagtttgggaaaagaaatcattagtgacggaaaatcgtcttcacaaagaatgcttgttgagactatcaacagaggaacgtcatatatttttggattacaagaaatatggatcctctatccgactagaattagtcaatcttattcaagcaaaaacaaaaaactttacgatagactttaagctaaaatattttctaggatctggcgctcaatctaaaagttctttattgcacgctattaatcatccaaagtcaaggcctaatacatctctggaaatagaatttacacctagagacaatgaaacagttccatatgataaactaataaaggaattgacgactctctcgcgtcatatatttatggcttctccagagaatgtaattctttctccacctattaacgcacctataaagacttttatgttgcctaaacaagatatagtaggtctggatctggaaaatctatatgccgtaactaagactgacggcattcctataactatcagagttacatcaaaagggttgtattgttattttacacatcttggttatattattagatatccagttaagagaacaatagattccgaagtagtagtctttggtgaggcagttaaggataagaactggaccgtatatctcattaagctaatagagcccgtaaatgcaatcagtgatagactagaagaaagtaagtatgttgaatctaaactagtggatatttgtgatcggatagtattcaagtcaaagaaatacgaaggtccgtttactacaactagtgaagtcgtcgatatgttatctacatatttaccaaagcaaccagaaggtgttattctgttctattcaaagggacctaaatctaacattgattttaaaatcaaaaaggagaatactatagaccaaactgcaaatgtagtatttaggtacatgtccagtgaaccaattatctttggagagtcgtctatctttatagagtataagaaatttaccaacgataaaggctttcctaaagaatatggttctggtaagattgtgttatataacggcgttaattatctaaataatatctattgtttggaatatattaatacacataatgaagtgggtattaagtccgttgttgtacctattaagtttatagcagaattcttagtcaatggagaaatacttaaacctagaatcgataaaaccatgaaatatattaactcagaagactattatggaaatcaacataatatcatagtcgaacatttaagagatcaaagcatcaaaataggagatgtctttaacgaggataaactatcggatgttggacatcaatacgctgccaacaacgataaatttagattaaatccagaagttagttattttactaataaacgaactagagggccgttgggaattttatcaaactacgtcaagactcttcttatttctatgtattgttccaaaacatttttagacgattccaacaaacgaaaggtattagcgattgattttggaaacggtgctgacctggaaaaatacttttatggagagattgcgttattggtagcgacggatccggatgctgatgctatagctagaggaaatgaaagatacaacaaattaaattctggaattaaaaccaagtactacaaatttgactacattcaggaaactattcgatccgatacatttgtctctagtgtcagagaagtattctattttggaaagtttaatatcattgactggcagttcgctattcattattcttttcatccaagacattatgctacagtcatgaataacttatccgaactaactgcttctggaggcaaggtattaattactaccatggatggagacaaattatcaaaattaaccgataaaaagacttttataattcataagaatctacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataatgttgatttcgctacaattatagaacgaagtaaaaagtttattaatggcgcatctacaatggaagatagaccgtctacaagaaactttttcgaactaaatagaggagccattaaatgtgaaggtttagatgtcgaagacttacttagttactatgttgtttatgtcttttctaagcggtaa

SEQ ID NO: 47 - DNA, 864 bp, synthetic construct
  misc_feature 1..864: note = Synthetic Construct
  source 1..864: mol_type = other DNA
atggatgaaattgtaaaaaatatccgggaggggacacatgtccttcttccattttatgaaacattgccagaacttaacttgtctttaggtaaaagcccattacctagtctggaatatggagctaattactttcttcagatttctagagttaatgatctaaatagaatgccgaccgacatgttaaaactttttacacatgatatcatgttaccagaaagcgatctagataaagtctatgaaattttaaagattaatagcgtaaagtattatgggaggagtactagagcggatgccgtagttgccgacctcagcgcacgcaataaactgttcaaacgtgaacgagatgctattaaatctaataatcatctcactgaaaacaatctatacattagcgattataagatgttaaccttcgacgtgtttcgaccattatttgattttgtaaacgaaaaatattgtattattaaacttcccactctattcggtagaggtgtaatcgatactatgagaatatattgtagtctctttaaaaatgttagactgttaaaatgcgtaagcgatagttggttgaaagatagcgccattatggtggctagtgatgtttataaaaaaaatttggatttatttatgtctcatgttaagtccgtcactaagtcttcgtcttggaaggatgtgaacactgttcaatttagtattttaaacgatccagtggatacggaattcattaataagttcttagagttttcgaatagagtatacgaagctctctattacgttcactcgctgctttattctagtatgacttctgattcaaaaagtatcgaaaacaaacatcagagaaggctagttaaactactgctgtga

SEQ ID NO: 48 - DNA, 2715 bp, synthetic construct
  misc_feature 1..2715: note = Synthetic Construct
  source 1..2715: mol_type = other DNA
ggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgtccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagatgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactatcacggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaacgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggagatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcacccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgtcatggatgttatctccagagaagatcagagggggagtggacaagttgtcacctacgccctaaacactttcaccaacctggccgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggactacatgagttcactaaagagatatgaagacacaactttggttgaggacacagtactg

SEQ ID NO: 49 - DNA, 8341 bp, synthetic construct
  misc_feature 1..8341: note = Synthetic Construct
  source 1..8341: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 50 - DNA, 9874 bp, synthetic construct
  misc_feature 1..9874: note = Synthetic Construct
  source 1..9874: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcaatccataatgagaatcccgcggaaatcatcatgttgtatgcgtctaagtattgacggtattcgttagcatgatgacgacagacagctccaccaagattgcatctggttatacaagttgctgacttcagcggtacgtaatctatatcactgactacctgtttcccatgactctcgcacggactacctgagtaataaaaaaatggcagttgttttaagttggtaaatgcgctcttatcaaaggccggtgtatgaaaggcatgtttgttcacgtataaactcccaccatcacacccgggaaggttcagattactcagtactctcgtgtcgaatctgcaaactatggcgttggcagggtatctatccacgttacagttccagaacaaacaaacaccatctgtaaacttatcatggtgggtcgcgtaagaatagaacaactcttcgatcttgtaggctttatcagagcaaggttgggcgtcgtaaaacttccattcgacttctgcttgtggtacacattttatggctttcggattaccgatgtcgtgtagaacagggaatttgtccgctagaagcgcggatttaactaccatgtgctgtactttcctacaagcagagttgaccctcagttcatcaccaataatcgggtattccacgctccaatcgacacgtttgacgaaacattcatgtactgccaaacatctagtcattatggcgtcacaggatgcgacgtgcgcattgccgtgtacttgacaatgttggtcgtgattagactgaaggttacccgtaaaaccccattgttgtacgtctatcataaatgggttgtatacgtagtcaaaccctacagaatgattccaacatgcataggtgtcagagctggtgctaaaacaagtcgcacgcttatcacaaaggcagcacgttctctctggtccaattttgacaaagtatttcattgacgttagttcaaagccgtgtgcccacaagacgaaaacaacacggtcggataatccttttagggtgtcggatagcatttgtactatcttgattcttactacattccagggtagtcccttgtacatcaaagggatcaagtgcttgaactgatcacctggcggcggcttggcatttacccttgtaaactccgtgttattctcggtgtcgacgtagccggtgggaactgcaactaagttaaccccggtgctaaagcctaattgtaatggtaagttagtacccacagcgtcacgtgtggcatgacatccttctacgtcgaatcctatccaagcacgtacatgacgtattgcttcctccctagttatgaacatatttggatagccgttaacttgataattcatcttgaatcccatcatagaaattagtctacgataggtcatgtccttcggaatcccaggtatgtcgacacaaagtccttcagtcttgaacttgatatcgacactcaggtgtgtgggtgcttgcgtcggatgaagcccggtaattattttgctgcaatccttgaatagcccggttacattctctgccatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 51 - DNA, 9214 bp, synthetic construct
  misc_feature 1..9214: note = Synthetic Construct
  source 1..9214: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcgacacgtttgacgaaacattcatgtactgccaaacatctagtcattatggcgtcacaggatgcgacgtgcgcattgccgtgtacttgacaatgttggtcgtgattagactgaaggttacccgtaaaaccccattgttgtacgtctatcataaatgggttgtatacgtagtcaaaccctacagaatgattccaacatgcataggtgtcagagctggtgctaaaacaagtcgcacgcttatcacaaaggcagcacgttctctctggtccaattttgacaaagtatttcattgacgttagttcaaagccgtgtgcccacaagacgaaaacaacacggtcggataatccttttagggtgtcggatagcatttgtactatcttgattcttactacattccagggtagtcccttgtacatcaaagggatcaagtgcttgaactgatcacctggcggcggcttggcatttacccttgtaaactccgtgttattctcggtgtcgacgtagccggtgggaactgcaactaagttaaccccggtgctaaagcctaattgtaatggtaagttagtacccacagcgtcacgtgtggcatgacatccttctacgtcgaatcctatccaagcacgtacatgacgtattgcttcctccctagttatgaacatatttggatagccgttaacttgataattcatcttgaatcccatcatagaaattagtctacgataggtcatgtccttcggaatcccaggtatgtcgacacaaagtccttcagtcttgaacttgatatcgacactcaggtgtgtgggtgcttgcgtcggatgaagcccggtaattattttgctgcaatccttgaatagcccggttacattctctgccatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 52 - DNA, 9055 bp, synthetic construct
  misc_feature 1..9055: note = Synthetic Construct
  source 1..9055: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcctgtagccttgtaaaggtattccataaattgtatgtatcaaattgtttataaatccataatgagaatcccgcggaaatcatcatgttgtatgcgtctaagtattgacggtattcgttagcatgatgacgacagacagctccaccaagattgcatctggttatacaagttgctgacttcagcggtacgtaatctatatcactgactacctgtttcccatgactctcgcacggactacctgagtaataaaaaaatggcagttgttttaagttggtaaatgcgctcttatcaaaggccggtgtatgaaaggcatgtttgttcacgtataaactcccaccatcacacccgggaaggttcagattactcagtactctcgtgtcgaatctgcaaactatggcgttggcagggtatctatccacgttacagttccagaacaaacaaacaccatctgtaaacttatcatggtgggtcgcgtaagaatagaacaactcttcgatcttgtaggctttatcagagcaaggttgggcgtcgtaaaacttccattcgacttctgcttgtggtacacattttatggctttcggattaccgatgtcgtgtagaacagggaatttgtccgctagaagcgcggatttaactaccatgtgctgtactttcctacaagcagagttgaccctcagttcatcaccaataatcgggtattccacgctccaatccatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 53 - DNA, 9181 bp, synthetic construct
  misc_feature 1..9181: note = Synthetic Construct
  source 1..9181: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcctgtagccttgtaaaggtattccataaattgtatgtatcaaattgtttataaatccataatgagaatcccgcggaaatcatcatgttgtatgcgtctaagtattgacggtattcgttagcatgatgacgacagacagctccaccaagattgcatctggttatacaagttgctgacttcagcggtacgtaatctatatcactgactacctgtttcccatgactctcgcacggactacctgagtaataaaaaaatggcagttgttttaagttggtaaatgcgctcttatcaaaggccggtgtatgaaaggcatgtttgttcacgtataaactcccaccatcacacccgggaaggttcagattactcagtactctcgtgtcgaatctgcaaactatggcgttggcagggtatctatccacgttacagttccagaacaaacaaacaccatctgtaaacttatcatggtgggtcgcgtaagaatagaacaactcttcgatcttgtaggctttatcagagcaaggttgggcgtcgtaaaacttccattcgacttctgcttgtggtacacattttatggctttcggattaccgatgtcgtgtagaacagggaatttgtccgctagaagcgcggatttaactaccatgtgctgtactttcctacaagcagagttgaccctcagttcatcaccaataatcgggtattccacgctccaatcgacacgtttgacgaaacattcatgtactgccaaacatctagtcattatggcgtcacaggatgcgacgtgcgcattgccgtgtacttgacaatgttggtcgtgattagactgaaggttacccgtaaacatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 54 - DNA, 9370 bp, synthetic construct
  misc_feature 1..9370: note = Synthetic Construct
  source 1..9370: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcctgtagccttgtaaaggtattccataaattgtatgtatcaaattgtttataaatccataatgagaatcccgcggaaatcatcatgttgtatgcgtctaagtattgacggtattcgttagcatgatgacgacagacagctccaccaagattgcatctggttatacaagttgctgacttcagcggtacgtaatctatatcactgactacctgtttcccatgactctcgcacggactacctgagtaataaaaaaatggcagttgttttaagttggtaaatgcgctcttatcaaaggccggtgtatgaaaggcatgtttgttcacgtataaactcccaccatcacacccgggaaggttcagattactcagtactctcgtgtcgaatctgcaaactatggcgttggcagggtatctatccacgttacagttccagaacaaacaaacaccatctgtaaacttatcatggtgggtcgcgtaagaatagaacaactcttcgatcttgtaggctttatcagagcaaggttgggcgtcgtaaaacttccattcgacttctgcttgtggtacacattttatggctttcggattaccgatgtcgtgtagaacagggaatttgtccgctagaagcgcggatttaactaccatgtgctgtactttcctacaagcagagttgaccctcagttcatcaccaataatcgggtattccacgctccaatcgacacgtttgacgaaacattcatgtactgccaaacatctagtcattatggcgtcacaggatgcgacgtgcgcattgccgtgtacttgacaatgttggtcgtgattagactgaaggttacccgtaaaaccccattgttgtacgtctatcataaatgggttgtatacgtagtcaaaccctacagaatgattccaacatgcataggtgtcagagctggtgctaaaacaagtcgcacgcttatcacaaaggcagcacgttctctctggtccaattttgacaaagtatttcattgacgttagttcaaagccgtgtgcccacatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 55 - DNA, 9793 bp, synthetic construct
  misc_feature 1..9793: note = Synthetic Construct
  source 1..9793: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcgacagctccaccaagattgcatctggttatacaagttgctgacttcagcggtacgtaatctatatcactgactacctgtttcccatgactctcgcacggactacctgagtaataaaaaaatggcagttgttttaagttggtaaatgcgctcttatcaaaggccggtgtatgaaaggcatgtttgttcacgtataaactcccaccatcacacccgggaaggttcagattactcagtactctcgtgtcgaatctgcaaactatggcgttggcagggtatctatccacgttacagttccagaacaaacaaacaccatctgtaaacttatcatggtgggtcgcgtaagaatagaacaactcttcgatcttgtaggctttatcagagcaaggttgggcgtcgtaaaacttccattcgacttctgcttgtggtacacattttatggctttcggattaccgatgtcgtgtagaacagggaatttgtccgctagaagcgcggatttaactaccatgtgctgtactttcctacaagcagagttgaccctcagttcatcaccaataatcgggtattccacgctccaatcgacacgtttgacgaaacattcatgtactgccaaacatctagtcattatggcgtcacaggatgcgacgtgcgcattgccgtgtacttgacaatgttggtcgtgattagactgaaggttacccgtaaaaccccattgttgtacgtctatcataaatgggttgtatacgtagtcaaaccctacagaatgattccaacatgcataggtgtcagagctggtgctaaaacaagtcgcacgcttatcacaaaggcagcacgttctctctggtccaattttgacaaagtatttcattgacgttagttcaaagccgtgtgcccacaagacgaaaacaacacggtcggataatccttttagggtgtcggatagcatttgtactatcttgattcttactacattccagggtagtcccttgtacatcaaagggatcaagtgcttgaactgatcacctggcggcggcttggcatttacccttgtaaactccgtgttattctcggtgtcgacgtagccggtgggaactgcaactaagttaaccccggtgctaaagcctaattgtaatggtaagttagtacccacagcgtcacgtgtggcatgacatccttctacgtcgaatcctatccaagcacgtacatgacgtattgcttcctccctagttatgaacatatttggatagccgttaacttgataattcatcttgaatcccatcatagaaattagtctacgataggtcatgtccttcggaatcccaggtatgtcgacacaaagtccttcagtcttgaacttgatatcgacactcaggtgtgtgggtgcttgcgtcggatgaagcccggtaattattttgctgcaatccttgaatagcccggttacattctctgccatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 56 - DNA, 27 bp, synthetic construct
  misc_feature 1..27: note = Synthetic Construct
  source 1..27: mol_type = other DNA
acagcttgtcacaccccttttctggca

SEQ ID NO: 57 - DNA, 44 bp, synthetic construct
  misc_feature 1..44: note = Synthetic Construct
  source 1..44: mol_type = other DNA
tggttgacatttttagtttatgtatgtgttttttgtagttatag

SEQ ID NO: 58 - DNA, 39 bp, synthetic construct
  misc_feature 1..39: note = Synthetic Construct
  source 1..39: mol_type = other DNA
taaactaaaaatgtcaaccaaaccagagaagccaatatg

SEQ ID NO: 59 - DNA, 32 bp, synthetic construct
  misc_feature 1..32: note = Synthetic Construct
  source 1..32: mol_type = other DNA
tccgcttacatcagttgggctttacgctttcc

SEQ ID NO: 60 - DNA, 27 bp, synthetic construct
  misc_feature 1..27: note = Synthetic Construct
  source 1..27: mol_type = other DNA
gcccaactgatgtaagcggatgccggg

SEQ ID NO: 61 - DNA, 30 bp, synthetic construct
  misc_feature 1..30: note = Synthetic Construct
  source 1..30: mol_type = other DNA
aaaggggtgtgacaagctgtgaccgtctcc

SEQ ID NO: 62 - DNA, 32 bp, synthetic construct
  misc_feature 1..32: note = Synthetic Construct
  source 1..32: mol_type = other DNA
tcttgaatcaggttggccgatgcagtcttttg

SEQ ID NO: 63 - DNA, 35 bp, synthetic construct
  misc_feature 1..35: note = Synthetic Construct
  source 1..35: mol_type = other DNA
acgtctacaggaaccagggtcaagtgcatccttta

SEQ ID NO: 64 - DNA, 42 bp, synthetic construct
  misc_feature 1..42: note = Synthetic Construct
  source 1..42: mol_type = other DNA
aaaggggtgtccaaacaaccaattacttgttgagaaatagag

SEQ ID NO: 65 - DNA, 37 bp, synthetic construct
  misc_feature 1..37: note = Synthetic Construct
  source 1..37: mol_type = other DNA
atcggccaacctgattcaagaaatatcttgaccgcag

SEQ ID NO: 66 - DNA, 40 bp, synthetic construct
  misc_feature 1..40: note = Synthetic Construct
  source 1..40: mol_type = other DNA
accctggttcgttaatagatcaaaaatcatcgcttcgctg

SEQ ID NO: 67 - DNA, 27 bp, synthetic construct
  misc_feature 1..27: note = Synthetic Construct
  source 1..27: mol_type = other DNA
tcggccaaccgagcggtctaaggcgcc

SEQ ID NO: 68 - DNA, 31 bp, synthetic construct
  misc_feature 1..31: note = Synthetic Construct
  source 1..31: mol_type = other DNA
atctattaacgaaccagggtcaagtgcatcc

SEQ ID NO: 69 - DNA, 32 bp, synthetic construct
  misc_feature 1..32: note = Synthetic Construct
  source 1..32: mol_type = other DNA
tagaccgctcggttggccgatgcagtcttttg

SEQ ID NO: 70 - DNA, 29 bp, synthetic construct
  misc_feature 1..29: note = Synthetic Construct
  source 1..29: mol_type = other DNA
gctatcggtaatccgaaagccataaaatg

SEQ ID NO: 71 - DNA, 23 bp, synthetic construct
  misc_feature 1..23: note = Synthetic Construct
  source 1..23: mol_type = other DNA
gtgtagaacagggaatttgtccg

SEQ ID NO: 72 - DNA, 27 bp, synthetic construct
  misc_feature 1..27: note = Synthetic Construct
  source 1..27: mol_type = other DNA
gaagcaacaggaaccagggtcaagtgc

SEQ ID NO: 73 - DNA, 45 bp, synthetic construct
  misc_feature 1..45: note = Synthetic Construct
  source 1..45: mol_type = other DNA
agttggccatttttagtttatgtatgtgttttttgtagttataga

SEQ ID NO: 74 - DNA, 28 bp, synthetic construct
  misc_feature 1..28: note = Synthetic Construct
  source 1..28: mol_type = other DNA
accctggttcctgttgcttctcaaaggc

SEQ ID NO: 75 - DNA, 24 bp, synthetic construct
  misc_feature 1..24: note = Synthetic Construct
  source 1..24: mol_type = other DNA
taaactaaaaatggccaactccgc

SEQ ID NO: 76 - DNA, 22 bp, synthetic construct
  misc_feature 1..22: note = Synthetic Construct
  source 1..22: mol_type = other DNA
aatccataatgagaatcccgcg

SEQ ID NO: 77 - DNA, 31 bp, synthetic construct
  misc_feature 1..31: note = Synthetic Construct
  source 1..31: mol_type = other DNA
taaactaaaaatggcagagaatgtaaccggg

SEQ ID NO: 78 - DNA, 20 bp, synthetic construct
  misc_feature 1..20: note = Synthetic Construct
  source 1..20: mol_type = other DNA
gaaccagggtcaagtgcatc

SEQ ID NO: 79 - DNA, 45 bp, synthetic construct
  misc_feature 1..45: note = Synthetic Construct
  source 1..45: mol_type = other DNA
tctctgccatttttagtttatgtatgtgttttttgtagttataga

SEQ ID NO: 80 - DNA, 45 bp, synthetic construct
  misc_feature 1..45: note = Synthetic Construct
  source 1..45: mol_type = other DNA
gatgcacttgaccctggttcgacacgtttgacgaaacattcatgt

SEQ ID NO: 81 - DNA, 23 bp, synthetic construct
  misc_feature 1..23: note = Synthetic Construct
  source 1..23: mol_type = other DNA
agtcacatcaagatcgtttatgg

SEQ ID NO: 82 - DNA, 23 bp, synthetic construct
  misc_feature 1..23: note = Synthetic Construct
  source 1..23: mol_type = other DNA
gcacggaatatgggactacttcg

SEQ ID NO: 83 - DNA, 23 bp, synthetic construct
  misc_feature 1..23: note = Synthetic Construct
  source 1..23: mol_type = other DNA
actccacttcaagtaagagtttg

SEQ ID NO: 84 - DNA, 35 bp, synthetic construct
  misc_feature 1..35: note = Synthetic Construct
  source 1..35: mol_type = other DNA
ttttagtttatgtatgtgttttttgtagttataga

SEQ ID NO: 85 - DNA, 20 bp, synthetic construct
  misc_feature 1..20: note = Synthetic Construct
  source 1..20: mol_type = other DNA
gaaccagggtcaagtgcatc

SEQ ID NO: 86 - DNA, 52 bp, synthetic construct
  misc_feature 1..52: note = Synthetic Construct
  source 1..52: mol_type = other DNA
actacaaaaaacacatacataaactaaaaatggattggagcgtggaataccc

SEQ ID NO: 87 - DNA, 44 bp, synthetic construct
  misc_feature 1..44: note = Synthetic Construct
  source 1..44: mol_type = other DNA
gatgcacttgaccctggttcctgtagccttgtaaaggtattcca

SEQ ID NO: 88 - DNA, 29 bp, synthetic construct
  misc_feature 1..29: note = Synthetic Construct
  source 1..29: mol_type = other DNA
ttttagtttatgtatgtgttttttgtagt

SEQ ID NO: 89 - DNA, 20 bp, synthetic construct
  misc_feature 1..20: note = Synthetic Construct
  source 1..20: mol_type = other DNA
gaaccagggtcaagtgcatc

SEQ ID NO: 90 - DNA, 42 bp, synthetic construct
  misc_feature 1..42: note = Synthetic Construct
  source 1..42: mol_type = other DNA
ttttagtttatgtatgtgttttttgtagttatagatttaagc

SEQ ID NO: 91 - DNA, 23 bp, synthetic construct
  misc_feature 1..23: note = Synthetic Construct
  source 1..23: mol_type = other DNA
atgtttacgggtaaccttcagtc

SEQ ID NO: 92 - DNA, 18 bp, synthetic construct
  misc_feature 1..18: note = Synthetic Construct
  source 1..18: mol_type = other DNA
atgtgggcacacggcttt

SEQ ID NO: 93 - DNA, 20 bp, synthetic construct
  misc_feature 1..20: note = Synthetic Construct
  source 1..20: mol_type = other DNA
gacagctccaccaagattgc

SEQ ID NO: 94 - DNA, 23 bp, synthetic construct
  misc_feature 1..23: note = Synthetic Construct
  source 1..23: mol_type = other DNA
tacagatggtctttgtttgttct

SEQ ID NO: 95 - DNA, 20 bp, synthetic construct
  misc_feature 1..20: note = Synthetic Construct
  source 1..20: mol_type = other DNA
aacttatcatggtgggtcgc

SEQ ID NO: 96 - DNA, 23 bp, synthetic construct
  misc_feature 1..23: note = Synthetic Construct
  source 1..23: mol_type = other DNA
tagataccctgtcaacgccatag

SEQ ID NO: 97 - DNA, 25 bp, synthetic construct
  misc_feature 1..25: note = Synthetic Construct
  source 1..25: mol_type = other DNA
tccacgttacagttccagaacaaac

SEQ ID NO: 98 - DNA, 24 bp, synthetic construct
  misc_feature 1..24: note = Synthetic Construct
  source 1..24: mol_type = other DNA
atgtcgacacaaagtccttcagtc

SEQ ID NO: 99 - DNA, 23 bp, synthetic construct
  misc_feature 1..23: note = Synthetic Construct
  source 1..23: mol_type = other DNA
acctgggattctgaaggacatga

SEQ ID NO: 100 - DNA, 23 bp, synthetic construct
  misc_feature 1..23: note = Synthetic Construct
  source 1..23: mol_type = other DNA
tctgaaccttcacgggtgtgatg

SEQ ID NO: 101 - DNA, 27 bp, synthetic construct
  misc_feature 1..27: note = Synthetic Construct
  source 1..27: mol_type = other DNA
ttactcagtactctcgtgtcgaatctg

SEQ ID NO: 102 - DNA, 33 bp, synthetic construct
  misc_feature 1..33: note = Synthetic Construct
  source 1..33: mol_type = other DNA
agagataaaacagatggcagagaatgtaaccgg

SEQ ID NO: 103 - DNA, 38 bp, synthetic construct
  misc_feature 1..38: note = Synthetic Construct
  source 1..38: mol_type = other DNA
cacctctagtactctgtagccttgtaaaggtattccat

SEQ ID NO: 104 - DNA, 30 bp, synthetic construct
  misc_feature 1..30: note = Synthetic Construct
  source 1..30: mol_type = other DNA
tctctgccatctgttttatctcttgtcccc

SEQ ID NO: 105 - DNA, 28 bp, synthetic construct
  misc_feature 1..28: note = Synthetic Construct
  source 1..28: mol_type = other DNA
aaggctacagagtactagaggtgatggg

SEQ ID NO: 106 - DNA, 28 bp, synthetic construct
  misc_feature 1..28: note = Synthetic Construct
  source 1..28: mol_type = other DNA
cttccttctgttcggagattaccgaatc

SEQ ID NO: 107 - DNA, 22 bp, synthetic construct
  misc_feature 1..22: note = Synthetic Construct
  source 1..22: mol_type = other DNA
cggtgatgacggtgaaaacctc

SEQ ID NO: 108 - DNA, 25 bp, synthetic construct
  misc_feature 1..25: note = Synthetic Construct
  source 1..25: mol_type = other DNA
aaatttaggtttcaatgatcttccc

SEQ ID NO: 109 - DNA, 25 bp, synthetic construct
  misc_feature 1..25: note = Synthetic Construct
  source 1..25: mol_type = other DNA
ttcgaatcaatagtttccatacctc

SEQ ID NO: 110 - DNA, 20 bp, synthetic construct
  misc_feature 1..20: note = Synthetic Construct
  source 1..20: mol_type = other DNA
aacgtggatagataccctgc

SEQ ID NO: 111 - DNA, 44 bp, synthetic construct
  misc_feature 1..44: note = Synthetic Construct
  source 1..44: mol_type = other DNA
ggcgttggcagggtatctatccacgttsvvgttccagaacaaac

SEQ ID NO: 112 - DNA, 50 bp, synthetic construct
  misc_feature 1..50: note = Synthetic Construct
  source 1..50: mol_type = other DNA
tgataaagcctacaagatcgaagagttgttcbbstcttacgcgacccacc

SEQ ID NO: 113 - DNA, 56 bp, synthetic construct
  misc_feature 1..56: note = Synthetic Construct
  source 1..56: mol_type = other DNA
atggaagttttacgacbbscaaccttgctctgataaagcctacaagatcgaagagt

SEQ ID NO: 114 - DNA, 25 bp, synthetic construct
  misc_feature 1..25: note = Synthetic Construct
  source 1..25: mol_type = other DNA
gtcgtaaaacttccattcgacttct

SEQ ID NO: 115 - DNA, 24 bp, synthetic construct
  misc_feature 1..24: note = Synthetic Construct
  source 1..24: mol_type = other DNA
caactcttcgatcttgtaggcttt

SEQ ID NO: 116 - DNA, 40 bp, synthetic construct
  misc_feature 1..40: note = Synthetic Construct
  misc_feature 25..26: note = n is a, c, g, or t
  source 1..40: mol_type = other DNA
aaagcctacaagatcgaagagttgnnktattcttacgcga

SEQ ID NO: 117 - DNA, 21 bp, synthetic construct
  misc_feature 1..21: note = Synthetic Construct
  source 1..21: mol_type = other DNA
atcgacacgtttgacgaaaca

SEQ ID NO: 118 - DNA, 37 bp, synthetic construct
  misc_feature 1..37: note = Synthetic Construct
  misc_feature 22..23: note = n is a, c, g, or t
  source 1..37: mol_type = other DNA
tgtttcgtcaaacgtgtcgatnnkagcgtggaatacc

SEQ ID NO: 119 - DNA, 26 bp, synthetic construct
  misc_feature 1..26: note = Synthetic Construct
  source 1..26: mol_type = other DNA
tgattcaagaaatatcttgaccgcag

SEQ ID NO: 120 - DNA, 28 bp, synthetic construct
  misc_feature 1..28: note = Synthetic Construct
  source 1..28: mol_type = other DNA
ctgtagccttgtaaaggtattccataaa

SEQ ID NO: 121 - DNA, 27 bp, synthetic construct
  misc_feature 1..27: note = Synthetic Construct
  source 1..27: mol_type = other DNA
ccattgttgtaccgctatcataaatgg

SEQ ID NO: 122 - DNA, 27 bp, synthetic construct
  misc_feature 1..27: note = Synthetic Construct
  source 1..27: mol_type = other DNA
ccatttatgatagcggtacaacaatgg

SEQ ID NO: 123 - DNA, 37 bp, synthetic construct
  misc_feature 1..37: note = Synthetic Construct
  source 1..37: mol_type = other DNA
tgtttcgtcaaacgtgtcgattttagcgtggaatacc

SEQ ID NO: 124 - DNA, 37 bp, synthetic construct
  misc_feature 1..37: note = Synthetic Construct
  source 1..37: mol_type = other DNA
ttcatgtactgccaaacatctagtcattatggcgtca

SEQ ID NO: 125 - DNA, 40 bp, synthetic construct
  misc_feature 1..40: note = Synthetic Construct
  source 1..40: mol_type = other DNA
aaagcctacaagatcgaagagttgaattattcttacgcga

SEQ ID NO: 126 - DNA, 25 bp, synthetic construct
  misc_feature 1..25: note = Synthetic Construct
  source 1..25: mol_type = other DNA
atcagagcaaggttgggcgtcgtaa

SEQ ID NO: 127 - DNA, 40 bp, synthetic construct
  misc_feature 1..40: note = Synthetic Construct
  misc_feature 25..26: note = n is a, c, g, or t
  source 1..40: mol_type = other DNA
gcagaagtcgaatggaagttttacnnkgcccaaccttgct

SEQ ID NO: 128 - DNA, 24 bp, synthetic construct
  misc_feature 1..24: note = Synthetic Construct
  source 1..24: mol_type = other DNA
gtaaaacttccattcgacttctgc

SEQ ID NO: 129 - DNA, 27 bp, synthetic construct
  misc_feature 1..27: note = Synthetic Construct
  source 1..27: mol_type = other DNA
ctttgtgtcgacgtacctgggattccg

SEQ ID NO: 130 - DNA, 28 bp, synthetic construct
  misc_feature 1..28: note = Synthetic Construct
  source 1..28: mol_type = other DNA
tccttcagtcttgaacttgatatcgaca

SEQ ID NO: 131 - DNA, 1584 bp, synthetic construct; identical to SEQ ID NO: 5
  misc_feature 1..1584: note = Synthetic Construct
  source 1..1584: mol_type = other DNA

SEQ ID NO: 132 - DNA, 1161 bp, synthetic construct; identical to SEQ ID NO: 2
  misc_feature 1..1161: note = Synthetic Construct
  source 1..1161: mol_type = other DNA

SEQ ID NO: 133 - DNA, 1311 bp, synthetic construct; identical to SEQ ID NO: 35
  misc_feature 1..1311: note = Synthetic Construct
  source 1..1311: mol_type = other DNA

SEQ ID NO: 134 - DNA, 350 bp, synthetic construct; identical to SEQ ID NO: 13
  misc_feature 1..350: note = Synthetic Construct
  source 1..350: mol_type = other DNA

SEQ ID NO: 135 - DNA, 8038 bp, synthetic construct
  misc_feature 1..8038: note = Synthetic Construct
  source 1..8038: mol_type = other DNA
atttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcacctcttgctgaatacgtaaactacataatatgaaagtaaatcctctacatccaagccctcgcattttatggcgccacggttaagttcgaaaaagtttctagtactaggtctgtcttccattgttgatgcaccgtttatgaactttttggaacgttctattatggtagcaaaatctacgttgtcgaccaatacaaatccatactcgttgaaaactcttacaatatcatttttcttgattatgtattcagtcatgggtgtagacatcgtggatggattatatactactatacggtcgtcggcgattttttcaacgctcatgtagttctcggaactaggcaagttcttatggatgataaaagtcttcttatccgtcaacttacttagcttgtccccgtccatcgtagtaataagcactttcccgccgctagcagttaattcagacagattattcattactgtcgcataatgcctcggatgaaatgagtaatggatggcaaactgccagtcgatgatattgaacttaccaaagtagaatacttctctaacgctagacacgaacgtgtcagaacgaattgtttcctgtatgtaatcaaacttataatatttcgtttttattcctgaattcagcttgttgtatctttcgttgcctctggcaattgcatcagcatcggggtctgttgctaccagcagcgcgatttccccgtaaaagtatttctccaagtcagcaccgtttccgaagtcgatggctaatacttttcttttgttagaatcgtctaagaatgtttttgagcaatacatggagatcaatagagtcttcacatagttagataagatacctaacggcccacgtgtacgcatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctccca

SEQ ID NO: 136 - DNA, 7948 bp, synthetic construct
  misc_feature 1..7948: note = Synthetic Construct
  source 1..7948: mol_type = other DNA
atttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcacctcttgctgaatacgtaaactacataatatgaaagtaaatcctctacatccaagccctcgcattttatggcgccacggttaagttcgaaaaagtttctagtactaggtctgtcttccattgttgatgcaccgtttatgaactttttggaacgttctattatggtagcaaaatctacgttgtcgaccaatacaaatccatactcgttgaaaactcttacaatatcatttttcttgattatgtattcagtcatgggtgtagacatcgtggatggattatatactactatacggtcgtcggcgattttttcaacgctcatgtagttctcggaactaggcaagttcttatggatgataaaagtcttcttatccgtcaacttacttagcttgtccccgtccatcgtagtaataagcactttcccgccgctagcagttaattcagacagattattcattactgtcgcataatgcctcggatgaaatgagtaatggatggcaaactgccagtcgatgatattgaacttaccaaagtagaatacttctctaacgctagacacgaacgtgtcagaacgaattgtttcctgtatgtaatcaaacttataatatttcgtttttattcctgaattcagcttgttgtatctttcgttgcctctggcaattgcatcagcatcggggtctgttgctaccagcagcgcgatttccccgtaaaagtatttctccaagtcagcaccgtttccgaagtcgatggctaatacttttcttttgttcatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctccca

SEQ ID NO: 137 - DNA, 8032 bp, synthetic construct
  misc_feature 1..8032: note = Synthetic Construct
  source 1..8032: mol_type = other DNA
atttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaaaatccatactcgttgaaaactcttacaatatcatttttcttgattatgtattcagtcatgggtgtagacatcgtggatggattatatactactatacggtcgtcggcgattttttcaacgctcatgtagttctcggaactaggcaagttcttatggatgataaaagtcttcttatccgtcaacttacttagcttgtccccgtccatcgtagtaataagcactttcccgccgctagcagttaattcagacagattattcattactgtcgcataatgcctcggatgaaatgagtaatggatggcaaactgccagtcgatgatattgaacttaccaaagtagaatacttctctaacgctagacacgaacgtgtcagaacgaattgtttcctgtatgtaatcaaacttataatatttcgtttttattcctgaattcagcttgttgtatctttcgttgcctctggcaattgcatcagcatcggggtctgttgctaccagcagcgcgatttccccgtaaaagtatttctccaagtcagcaccgtttccgaagtcgatggctaatacttttcttttgttagaatcgtctaagaatgtttttgagcaatacatggagatcaatagagtcttcacatagttagataagatacctaacggcccacgtgtacgtttattcgtgaagtaacttacttccggattaagtctaaatttgtcattgtttgcggcgtattgatgtccaacgtcactcagtttatcttcgttgaagacatccccgatctttatggattggtcccttagatgctctacgatgatattgtgttgattcccgtaataatcttcgctattaatgtatttcatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctccca

SEQ ID NO: 138 - DNA, 9214 bp, synthetic construct
  misc_feature 1..9214: note = Synthetic Construct
  source 1..9214: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttccaccctttctatgaagcaagaatggatcgccaggcatctagtcattatagcatcattggacgcaacatgagcgccttgatgcacagaacaatatctgtcatgatttgttgccaaattgccaacgtagccccactgctgtacgtctacaaagaatggattgtatacgtagtcgtagccacatgaatgagtccaacaggcgtaactctgcaaggggcttgagtatgctgcagctctacgattacacatacaacacttttgctccttcccaattttgcaaaagtaggaggctgacgttagttcaaacccatgggcccaacatacaaatgtgcagtaatcggaaagcttgtcaagggtatcgctcaacatctgaacgatacgcctacggacgattggccaagcagcccctttatgcataagcgggaccaggtgcttaaattgctctccgggcggaggcctcgcggctatccccgttaacatgtttccccattcagtatcaacgacaccaacaggctgaaccacgaagtttaccccggtggagaacccaagttgaagcggaacattggttccgcaggcgtttctactcgcatgggctccttctacgtcgaaacctatccatgacctcacttgacgcactgcttcctcccttgtaataaatagtttcggatagcctggaacagttgcgtccagtttaaagcccatacgtgatataactctactataaggcacattcgctggaagatttagattcacacacaattcatctgaagttttatacttatcgtcaacgctgacgtacgttggtgcatacgctggtgacaagcctgaagtctcacgggagcagtctttgaacagtccagtgacaatttgggacatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 139 - DNA, 9865 bp, synthetic construct
  misc_feature 1..9865: note = Synthetic Construct
  source 1..9865: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcgcaccacagacggaatccagaggctgagacaagattataggcttccatatattccctgtactctgtggcatgttttctgcaaacggctcccccaaggttgcaagctgttatgcaaacggcgcttttcagaggaacataatctatatcttcgatcatactgccgttaccatgtacctcgcacggagtcgtagagtagtagaaaaatggtagcggtttcaaatctctgaaggcggaaacgtcataggctggtgtatggaaggcatgcttattcacgtacagtgacccaccgtcacaccctggcaaattaaactcggaatggactctcgtgtcaaaacgacagacgatcgcattgttggggtatttggggacattacagttccagaatagacaaaggccatcagcaaatctggatgccatatcttcggtgtaaaataactgttgaacctttcttgttagtggttgtgcatcaaagtagtgccaatctaccaccggatcatccacaatggggatacccttagggttccctatgtcatacactttatcaaatgatcccgccagcaaagctgccctcactacattcctctccacgatacggcaacaagaattaagtttcttttcgtgtgatatgtacgggtattcaatatcccagtccaccctttctatgaagcaagaatggatcgccaggcatctagtcattatagcatcattggacgcaacatgagcgccttgatgcacagaacaatatctgtcatgatttgttgccaaattgccaacgtagccccactgctgtacgtctacaaagaatggattgtatacgtagtcgtagccacatgaatgagtccaacaggcgtaactctgcaaggggcttgagtatgctgcagctctacgattacacatacaacacttttgctccttcccaattttgcaaaagtaggaggctgacgttagttcaaacccatgggcccaacatacaaatgtgcagtaatcggaaagcttgtcaagggtatcgctcaacatctgaacgatacgcctacggacgattggccaagcagcccctttatgcataagcgggaccaggtgcttaaattgctctccgggcggaggcctcgcggctatccccgttaacatgtttccccattcagtatcaacgacaccaacaggctgaaccacgaagtttaccccggtggagaacccaagttgaagcggaacattggttccgcaggcgtttctactcgcatgggctccttctacgtcgaaacctatccatgacctcacttgacgcactgcttcctcccttgtaataaatagtttcggatagcctggaacagttgcgtccagtttaaagcccatacgtgatataactctactataaggcacattcgctggaagatttagattcacacacaattcatctgaagttttatacttatcgtcaacgctgacgtacgttggtgcatacgctggtgacaagcctgaagtctcacgggagcagtctttgaacagtccagtgacaatttgggacatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 140 - DNA, 9046 bp, synthetic construct
  misc_feature 1..9046: note = Synthetic Construct
  source 1..9046: mol_type = other DNA
cttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcctgaacttttgtaaacgtactccaaaggttatatatatcaaaagttttatagcaccacagacggaatccagaggctgagacaagattataggcttccatatattccctgtactctgtggcatgttttctgcaaacggctcccccaaggttgcaagctgttatgcaaacggcgcttttcagaggaacataatctatatcttcgatcatactgccgttaccatgtacctcgcacggagtcgtagagtagtagaaaaatggtagcggtttcaaatctctgaaggcggaaacgtcataggctggtgtatggaaggcatgcttattcacgtacagtgacccaccgtcacaccctggcaaattaaactcggaatggactctcgtgtcaaaacgacagacgatcgcattgttggggtatttggggacattacagttccagaatagacaaaggccatcagcaaatctggatgccatatcttcggtgtaaaataactgttgaacctttcttgttagtggttgtgcatcaaagtagtgccaatctaccaccggatcatccacaatggggatacccttagggttccctatgtcatacactttatcaaatgatcccgccagcaaagctgccctcactacattcctctccacgatacggcaacaagaattaagtttcttttcgtgtgatatgtacgggtattcaatatcccagtccatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattga

SEQ ID NO: 141 - DNA, 27 bp, synthetic construct
  misc_feature 1..27: note = Synthetic Construct
  source 1..27: mol_type = other DNA
acagcttgtcacaccccttttctggca

SEQ ID NO: 142 - DNA, 44 bp, synthetic construct; identical to SEQ ID NO: 57
  misc_feature 1..44: note = Synthetic Construct
  source 1..44: mol_type = other DNA

SEQ ID NO: 143 - DNA, 39 bp, synthetic construct; identical to SEQ ID NO: 58
  misc_feature 1..39: note = Synthetic Construct
  source 1..39: mol_type = other DNA

SEQ ID NO: 144 - DNA, 32 bp, synthetic construct; identical to SEQ ID NO: 59
  misc_feature 1..32: note = Synthetic Construct
  source 1..32: mol_type = other DNA

SEQ ID NO: 145 - DNA, 27 bp, synthetic construct
  misc_feature 1..27: note = Synthetic Construct
  source 1..27: mol_type = other DNA
gcccaactgatgtaagcggatgccggg

SEQ ID NO: 146 - DNA, 30 bp, synthetic construct; identical to SEQ ID NO: 61
  misc_feature 1..30: note = Synthetic Construct
  source 1..30: mol_type = other DNA

SEQ ID NO: 147 - DNA, 27 bp, synthetic construct
  misc_feature 1..27: note = Synthetic Construct
  source 1..27: mol_type = other DNA
gaagcaacaggaaccagggtcaagtgc

SEQ ID NO: 148 - DNA, 45 bp, synthetic construct; identical to SEQ ID NO: 73
  misc_feature 1..45: note = Synthetic Construct
  source 1..45: mol_type = other DNA

SEQ ID NO: 149 - DNA, 28 bp, synthetic construct
  misc_feature 1..28: note = Synthetic Construct
  source 1..28: mol_type = other DNA
accctggttcctgttgcttctcaaaggc

SEQ ID NO: 150 - DNA, 24 bp, synthetic construct
  misc_feature 1..24: note = Synthetic Construct
  source 1..24: mol_type = other DNA
taaactaaaaatggccaactccgc

SEQ ID NO: 151 - DNA, 49 bp, synthetic construct
  misc_feature 1..49: note = Synthetic Construct
  source 1..49: mol_type = other DNA
actacaaaaaacacatacataaactaaaaatgtcccaaattgtcactgg

SEQ ID NO: 152 - DNA, 45 bp, synthetic construct
  misc_feature 1..45: note = Synthetic Construct
  source 1..45: mol_type = other DNA
gatgcacttgaccctggttcctgaacttttgtaaacgtactccaa

SEQ ID NO: 153 - DNA, 29 bp, synthetic construct
  misc_feature 1..29: note = Synthetic Construct
  source 1..29: mol_type = other DNA
ttttagtttatgtatgtgttttttgtagt

SEQ ID NO: 154 - DNA, 20 bp, synthetic construct
  misc_feature 1..20: note = Synthetic Construct
  source 1..20: mol_type = other DNA
gaaccagggtcaagtgcatc

SEQ ID NO: 155 - DNA, 38 bp, synthetic construct
  misc_feature 1..38: note = Synthetic Construct
  source 1..38: mol_type = other DNA
gatgcacttgaccctggttcgcaccacagacggaatcc

SEQ ID NO: 156 - DNA, 45 bp, synthetic construct
  misc_feature 1..45: note = Synthetic Construct
  source 1..45: mol_type = other DNA
gatgcacttgaccctggttccaccctttctatgaagcaagaatgg

SEQ ID NO: 157 - DNA, 56 bp, synthetic construct
  misc_feature 1..56: note = Synthetic Construct
  source 1..56: mol_type = other DNA
actacaaaaaacacatacataaactaaaaatggactgggatattgaatacccgtac

SEQ ID NO: 158 - DNA, 24 bp, synthetic construct
  misc_feature 1..24: note = Synthetic Construct
  source 1..24: mol_type = other DNA
cactgctgtaccgctacaaagaat

SEQ ID NO: 159 - DNA, 24 bp, synthetic construct
  misc_feature 1..24: note = Synthetic Construct
  source 1..24: mol_type = other DNA
attctttgtagcggtacagcagtg

SEQ ID NO: 160 - DNA, 26 bp, synthetic construct
  misc_feature 1..26: note = Synthetic Construct
  source 1..26: mol_type = other DNA
agggttccctatcgcatacactttat

SEQ ID NO: 161 - DNA, 26 bp, synthetic construct
  misc_feature 1..26: note = Synthetic Construct
  source 1..26: mol_type = other DNA
ataaagtgtatgcgatagggaaccct

SEQ ID NO: 162 - DNA, 46 bp, synthetic construct
  misc_feature 1..46: note = Synthetic Construct
  source 1..46: mol_type = other DNA
gatgcacttgaccctggttcattcttccttaagcatataaacccga

SEQ ID NO: 163 - DNA, 52 bp, synthetic construct
  misc_feature 1..52: note = Synthetic Construct
  source 1..52: mol_type = other DNA
actacaaaaaacacatacataaactaaaaatggctagtcttgagaatctggt

SEQ ID NO: 164 - DNA, 53 bp, synthetic construct
  misc_feature 1..53: note = Synthetic Construct
  source 1..53: mol_type = other DNA
gctattaatgtatttcatttttagtttatgtatgtgttttttgtagttataga

SEQ ID NO: 165 - DNA, 50 bp, synthetic construct
  misc_feature 1..50: note = Synthetic Construct
  source 1..50: mol_type = other DNA
cacatacataaactaaaaatgaaatacattaatagcgaagattattacgg

SEQ ID NO: 166 - DNA, 40 bp, synthetic construct
  misc_feature 1..40: note = Synthetic Construct
  source 1..40: mol_type = other DNA
tattcagcaagaggtgattcaagaaatatcttgaccgcag

SEQ ID NO: 167 - DNA, 39 bp, synthetic construct
  misc_feature 1..39: note = Synthetic Construct
  source 1..39: mol_type = other DNA
tatttcttgaatcacctcttgctgaatacgtaaactaca

SEQ ID NO: 168 - DNA, 51 bp, synthetic construct
  misc_feature 1..51: note = Synthetic Construct
  source 1..51: mol_type = other DNA
tgacaatctcatccatttttagtttatgtatgtgttttttgtagttataga

SEQ ID NO: 169 - DNA, 44 bp, synthetic construct
  misc_feature 1..44: note = Synthetic Construct
  source 1..44: mol_type = other DNA
catacataaactaaaaatggatgagattgtcaagaatattcgtg

SEQ ID NO: 170 - DNA, 45 bp, synthetic construct
  misc_feature 1..45: note = Synthetic Construct
  source 1..45: mol_type = other DNA
ctagttaaattactgctatgatttttctattactcttggcctcct

SEQ ID NO: 171 - DNA, 43 bp, synthetic construct
  misc_feature 1..43: note = Synthetic Construct
  source 1..43: mol_type = other DNA
agagtaatagaaaaatcatagcagtaatttaactagcctacgc

SEQ ID NO: 172 - DNA, 27 bp, synthetic construct
  misc_feature 1..27: note = Synthetic Construct
  source 1..27: mol_type = other DNA
ctggtagcaacagcccccgatgctgat

SEQ ID NO: 173 - DNA, 25 bp, synthetic construct
  misc_feature 1..25: note = Synthetic Construct
  source 1..25: mol_type = other DNA
cagcgcgatttccccgtaaaagtat

SEQ ID NO: 174 - DNA, 27 bp, synthetic construct
  misc_feature 1..27: note = Synthetic Construct
  source 1..27: mol_type = other DNA
caggaaacaattgcttctgacacgttc

SEQ ID NO: 175 - DNA, 36 bp, synthetic construct
  misc_feature 1..36: note = Synthetic Construct
  source 1..36: mol_type = other DNA
tatgtaatcaaacttataatatttcgtttttattcc

SEQ ID NO: 176 - DNA, 29 bp, synthetic construct
  misc_feature 1..29: note = Synthetic Construct
  source 1..29: mol_type = other DNA
ccggaagtaagtttcttcacgaataaacg

SEQ ID NO: 177 - DNA, 28 bp, synthetic construct
  misc_feature 1..28: note = Synthetic Construct
  source 1..28: mol_type = other DNA
attaagtctaaatttgtcattgtttgcg

SEQ ID NO: 178 - DNA, 29 bp, synthetic construct
  misc_feature 1..29: note = Synthetic Construct
  source 1..29: mol_type = other DNA
ccggaagtaagtgccttcacgaataaacg

SEQ ID NO: 179 - DNA, 27 bp, synthetic construct
  misc_feature 1..27: note = Synthetic Construct
  source 1..27: mol_type = other DNA
gtattagccatcgccttcggaaacggt

SEQ ID NO: 180 - DNA, 30 bp, synthetic construct
  misc_feature 1..30: note = Synthetic Construct
  source 1..30: mol_type = other DNA
ttttcttttgttagaatcgtctaagaatgt

SEQ ID NO: 181 - DNA, 27 bp, synthetic construct
  misc_feature 1..27: note = Synthetic Construct
  source 1..27: mol_type = other DNA
tttgccatccatgcctcatttcatccg

SEQ ID NO: 182 - DNA, 25 bp, synthetic construct
  misc_feature 1..25: note = Synthetic Construct
  source 1..25: mol_type = other DNA
ctgccagtcgatgatattgaactta

SEQ ID NO: 183 - DNA, 27 bp, synthetic construct
  misc_feature 1..27: note = Synthetic Construct
  source 1..27: mol_type = other DNA
gccgcaaacaatgccaaatttagactt

SEQ ID NO: 184 - DNA, 23 bp, synthetic construct
  misc_feature 1..23: note = Synthetic Construct
  source 1..23: mol_type = other DNA
gtattgatgtccaacgtcactca

SEQ ID NO: 185 - DNA, 30 bp, synthetic construct
  misc_feature 1..30: note = Synthetic Construct
  source 1..30: mol_type = other DNA
ccggaagtaagttacgccacgaataaacgt

SEQ ID NO: 186 - DNA, 53 bp, synthetic construct; identical to SEQ ID NO: 164
  misc_feature 1..53: note = Synthetic Construct
  source 1..53: mol_type = other DNA

SEQ ID NO: 187 - DNA, 50 bp, synthetic construct; identical to SEQ ID NO: 165
  misc_feature 1..50: note = Synthetic Construct
  source 1..50: mol_type = other DNA

SEQ ID NO: 188 - DNA, 20 bp, synthetic construct
  misc_feature 1..20: note = Synthetic Construct
  source 1..20: mol_type = other DNA
atggatggcaaactgccagt

SEQ ID NO: 189 - DNA, 35 bp, synthetic construct
  misc_feature 1..35: note = Synthetic Construct
  misc_feature 21..22: note = n is a, c, g, or t
  source 1..35: mol_type = other DNA
actggcagtttgccatccatnnktcatttcatccg

SEQ ID NO: 190 - DNA, 40 bp, synthetic construct; identical to SEQ ID NO: 166
  misc_feature 1..40: note = Synthetic Construct
  source 1..40: mol_type = other DNA

SEQ ID NO: 191 - DNA, 39 bp, synthetic construct; identical to SEQ ID NO: 167
  misc_feature 1..39: note = Synthetic Construct
  source 1..39: mol_type = other DNA

SEQ ID NO: 192 - DNA, 40 bp, synthetic construct
  misc_feature 1..40: note = Synthetic Construct
  misc_feature 23..24: note = n is a, c, g, or t
  source 1..40: mol_type = other DNA
cgccgcaaacaatgacaaatttnnkcttaatccggaagta

SEQ ID NO: 193 - DNA, 22 bp, synthetic construct
  misc_feature 1..22: note = Synthetic Construct
  source 1..22: mol_type = other DNA
aaatttgtcattgtttgcggcg

SEQ ID NO: 194 - DNA, 35 bp, synthetic construct
  misc_feature 1..35: note = Synthetic Construct
  source 1..35: mol_type = other DNA
caatctgtttatacatgctctgatgaagtccagcc

SEQ ID NO: 195 - DNA, 35 bp, synthetic construct
  misc_feature 1..35: note = Synthetic Construct
  source 1..35: mol_type = other DNA
caatctgtttatacatttactgatgaagtccagcc

SEQ ID NO: 196 - DNA, 29 bp, synthetic construct
  misc_feature 1..29: note = Synthetic Construct
  source 1..29: mol_type = other DNA
cttactatactactagctccttctttggg

SEQ ID NO: 197 - DNA, 35 bp, synthetic construct
  misc_feature 1..35: note = Synthetic Construct
  source 1..35: mol_type = other DNA
gtatagtaagcaatctggctatacattatctgatg

SEQ ID NO: 198 - DNA, 35 bp, synthetic construct
  misc_feature 1..35: note = Synthetic Construct
  source 1..35: mol_type = other DNA
gtatagtaagcaatctgttaatacattatctgatg

SEQ ID NO: 199 - DNA, 24 bp, synthetic construct
  misc_feature 1..24: note = Synthetic Construct
  source 1..24: mol_type = other DNA
tactagctccttctttgggaaacc

SEQ ID NO: 200 - DNA, 35 bp, synthetic construct
  misc_feature 1..35: note = Synthetic Construct
  source 1..35: mol_type = other DNA
gtatagtaagcaatctgtggatacattatctgatg

SEQ ID NO: 201 - DNA, 26 bp, synthetic construct
  misc_feature 1..26: note = Synthetic Construct
  source 1..26: mol_type = other DNA
gtattgcaccaactcttggcggaacc

SEQ ID NO: 202 - DNA, 22 bp, synthetic construct
  misc_feature 1..22: note = Synthetic Construct
  source 1..22: mol_type = other DNA
atagatgttcgtcgcgtgcttg

SEQ ID NO: 203 - DNA, 39 bp, synthetic construct
  misc_feature 1..39: note = Synthetic Construct
  source 1..39: mol_type = other DNA
gcgcagacggcgttgatcgcttttataaaacaagaaatt

SEQ ID NO: 204 - DNA, 25 bp, synthetic construct
  misc_feature 1..25: note = Synthetic Construct
  source 1..25: mol_type = other DNA
acgatagatgccggtcttcgcccca

SEQ ID NO: 205 - DNA, 34 bp, synthetic construct
  misc_feature 1..34: note = Synthetic Construct
  source 1..34: mol_type = other DNA
tatgtattgcaccaagcattggcggaacctgcta

SEQ ID NO: 206 - DNA, 24 bp, synthetic construct
  misc_feature 1..24: note = Synthetic Construct
  source 1..24: mol_type = other DNA
gatgttcgtcgcgtgcttgtgctg

SEQ ID NO: 207 - DNA, 34 bp, synthetic construct
  misc_feature 1..34: note = Synthetic Construct
  source 1..34: mol_type = other DNA
tatgtattgcaccaaaatttggcggaacctgcta

SEQ ID NO: 208 - DNA, 34 bp, synthetic construct
  misc_feature 1..34: note = Synthetic Construct
  source 1..34: mol_type = other DNA
tatgtattgcaccaagaattggcggaacctgcta

SEQ ID NO: 209 - DNA, 40 bp, synthetic construct
  misc_feature 1..40: note = Synthetic Construct
  source 1..40: mol_type = other DNA
acatctggttggtattgattatgaccagaccgcattggcg

SEQ ID NO: 210 - DNA, 27 bp, synthetic construct
  misc_feature 1..27: note = Synthetic Construct
  source 1..27: mol_type = other DNA
ctaattcctgcatccaggtaacgccct

SEQ ID NO: 211 - DNA, 35 bp, synthetic construct
  misc_feature 1..35: note = Synthetic Construct
  source 1..35: mol_type = other DNA
acatctggttggtattgctaaagaccagaccgcat

SEQ ID NO: 212 - DNA, 35 bp, synthetic construct
  misc_feature 1..35: note = Synthetic Construct
  source 1..35: mol_type = other DNA
acatctggttggtattgaaaaagaccagaccgcat

SEQ ID NO: 213 - DNA, 26 bp, synthetic construct
  misc_feature 1..26: note = Synthetic Construct
  source 1..26: mol_type = other DNA
tgattcaagaaatatcttgaccgcag

SEQ ID NO: 214 - DNA, 35 bp, synthetic construct
  misc_feature 1..35: note = Synthetic Construct
  source 1..35: mol_type = other DNA
catttttagtttatgtatgtgttttttgtagttat

SEQ ID NO: 215 - DNA, 23 bp, synthetic construct
  misc_feature 1..23: note = Synthetic Construct
  source 1..23: mol_type = other DNA
cgtacacgtgggccgttaggtat

SEQ ID NO: 216 - DNA, 28 bp, synthetic construct
  misc_feature 1..28: note = Synthetic Construct
  source 1..28: mol_type = other DNA
caatacaaatccatactcgttgaaaact

SEQ ID NO: 217 - DNA, 28 bp, synthetic construct
  misc_feature 1..28: note = Synthetic Construct
  source 1..28: mol_type = other DNA
aacaaaagaaaagtattagccatcgact

SEQ ID NO: 218 - DNA, 35 bp, synthetic construct
  misc_feature 1..35: note = Synthetic Construct
  source 1..35: mol_type = other DNA
gaaaggttcaacagttaggttacaccgaagatatg

SEQ ID NO: 219 - DNA, 32 bp, synthetic construct
  misc_feature 1..32: note = Synthetic Construct
  source 1..32: mol_type = other DNA
catagaaagggtggacttcgatattgaatacc

SEQ ID NO: 220 - DNA, 32 bp, synthetic construct
  misc_feature 1..32: note = Synthetic Construct
  source 1..32: mol_type = other DNA
gattggcactactttgctgcacaaccactaac

SEQ ID NO: 221 - DNA, 32 bp, synthetic construct
  misc_feature 1..32: note = Synthetic Construct
  source 1..32: mol_type = other DNA
gattggcactactttactgcacaaccactaac

SEQ ID NO: 222 - DNA, 22 bp, synthetic construct
  misc_feature 1..22: note = Synthetic Construct
  source 1..22: mol_type = other DNA
taccaccggatcatccacaatg

SEQ ID NO: 223 - DNA, 26 bp, synthetic construct
  misc_feature 1..26: note = Synthetic Construct
  source 1..26: mol_type = other DNA
ttgttagtggttgtgcatcaaagtag

SEQ ID NO: 224 - DNA, 20 bp, synthetic construct
  misc_feature 1..20: note = Synthetic Construct
  source 1..20: mol_type = other DNA
aagcaagaatggatcgccag

SEQ ID NO: 225 - DNA, 35 bp, synthetic construct
  misc_feature 1..35: note = Synthetic Construct
  source 1..35: mol_type = other DNA
gaaaggttcaacagttaaattacaccgaagatatg

SEQ ID NO: 226 - DNA, 35 bp, synthetic construct
  misc_feature 1..35: note = Synthetic Construct
  source 1..35: mol_type = other DNA
gaaaggttcaacagttattatacaccgaagatatg

SEQ ID NO: 227 - DNA, 35 bp, synthetic construct
  misc_feature 1..35: note = Synthetic Construct
  source 1..35: mol_type = other DNA
gaaaggttcaacagttagattacaccgaagatatg

SEQ ID NO: 228 - DNA, 18 bp, synthetic construct
  misc_feature 1..18: note = Synthetic Construct
  source 1..18: mol_type = other DNA
cgtgtagccaggacacca

SEQ ID NO: 229 - DNA, 18 bp, synthetic construct
  misc_feature 1..18: note = Synthetic Construct
  source 1..18: mol_type = other DNA
tggtgtcctggctacacg

SEQ ID NO: 230 - DNA, 47 bp, synthetic construct
  misc_feature 1..47: note = Synthetic Construct
  misc_feature 33..34: note = n is a, c, g, or t
  source 1..47: mol_type = other DNA
aagaaggagctagtagtatagtaagcaatctgnnkatacattatctg

SEQ ID NO: 231 - DNA, 41 bp, synthetic construct
  misc_feature 1..41: note = Synthetic Construct
  source 1..41: mol_type = other DNA
gattgcttactatactactagctccttctttgggaaaccca

SEQ ID NO: 232 - DNA, 42 bp, synthetic construct
  misc_feature 1..42: note = Synthetic Construct
  misc_feature 29..30: note = n is a, c, g, or t
  source 1..42: mol_type = other DNA
ctcaacctgttggctggacttcatcagmnnatgtataaacag

SEQ ID NO: 233 - DNA, 34 bp, synthetic construct
  misc_feature 1..34: note = Synthetic Construct
  source 1..34: mol_type = other DNA
aagtccagccaacaggttgagaacttagcggtac

SEQ ID NO: 234 - DNA, 1161 bp, synthetic construct; identical to SEQ ID NO: 2
  misc_feature 1..1161: note = Synthetic Construct
  source 1..1161: mol_type = other DNA

SEQ ID NO: 235 - DNA, 1575 bp, synthetic construct; identical to SEQ ID NO: 25
  misc_feature 1..1575: note = Synthetic Construct
  source 1..1575: mol_type = other DNA

SEQ ID NO: 236 - DNA, 2604 bp, synthetic construct; identical to SEQ ID NO: 11
  misc_feature 1..2604: note = Synthetic Construct
  source 1..2604: mol_type = other DNA

SEQ ID NO: 237 - DNA, 1044 bp, synthetic construct; identical to SEQ ID NO: 7
  misc_feature 1..1044: note = Synthetic Construct
  source 1..1044: mol_type = other DNA

SEQ ID NO: 238 - DNA, 861 bp, synthetic construct; identical to SEQ ID NO: 9
  misc_feature 1..861: note = Synthetic Construct
  source 1..861: mol_type = other DNA

SEQ ID NO: 239 - DNA, 1311 bp, synthetic construct; identical to SEQ ID NO: 35
  misc_feature 1..1311: note = Synthetic Construct
  source 1..1311: mol_type = other DNA

SEQ ID NO: 240 - DNA, 350 bp, synthetic construct; identical to SEQ ID NO: 13
  misc_feature 1..350: note = Synthetic Construct
  source 1..350: mol_type = other DNA

SEQ ID NO: 241 - DNA, 9247 bp, synthetic construct
  misc_feature 1..9247: note = Synthetic Construct
  source 1..9247: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcggttggccgatgcagtcttttgggcggtgggggcggcatgagctccgtgtcagggtccaggggatacttccgcttctgtccctgggcggctgctgcacatctgtcaatgaattcaaacagcatctccttggtgacagggttcgagatgctattgcacacagccatggctgtgttgtaggcatttgggaaacttttgtctatcctctgtctcatgaagacccagctattgttctcaaatttgcattctataattttgttgtcatactgttttagttcttttgtcaccttgatttgtgcaaagggtctttcataacctccaacatagagaaggccaacattctgtggaagcaacccttctcctcccattcttgttatcttaagtcgaaaatccacagagtttagactgggaggtttccatttcaaaatgtcatcacatcgaccaggcttgtattttccaataggctgaaaaataagtccatccatttcatggctgacttctttggcaaaatttccttccagaagctttcttgaaatattgatgtcaaaaaattgtttaggtctgacactaaatggttcctgtgttttgtcaatgagtccagtcttcattttttcatgtcgtggacttataatttcacgttcaatacactgtagacgaatattaaaatcacagtctccaactggttgtgcattgaacttaattatgtcatagatcaaatatcttgggacggcctgtccatttactttgtcaatgatcatttccccatccaaaagagtatttgataaatgcatgcgaagatctttacgaaatggaaattccagatttgaaacatgaaaaacggaattatctctgtcaatcataaaaacttcatttgtgccatcaatcaacatcatgtaacgagtgccgtctgctttccagcttactttataaggcttctgctctaaaagtctaatattttgcttgtccatggagacaggctgtgctccagggaacccagacccttcccagccacagaactgatgacatttctgctgcacctctcctaacttgggttgagttgttacttgagttacacctttaacagttatgccttccaagaaaatagctcccagttttaaccgctcttttctccttttactaaaggatgcacttgaccctggttcgtgattttcatcatggtgccacgtagagctgtactccctacgtaacctctctatacggttcttaattttactagtgtcagaattcagcaggggtttaccaaccgcccttgttccagaaccaaggttcacatcctcttcatattgcggtcccttccatgtccttttctccatacggcctaacagtacctggctagtcatgtttacgctatgcaccacattacctgaagctctgctgacccagtacatttcgtgcgtgctgtttctgctaaggggatttcttaccaaaccgcccccatatctacgttgtaacagctccatcttttctataaccttaggcatgtacggacaaaggaccttgacgcagaactctcttgggccacgatgcaaccaatcctcgaccatctccagaacacggatcgttctatgctcctccacctccgcagagcttgagctctcccctatatcacacagtaaggtgtcgcaacattctgacggacgatagaacacgtcaacccctgacttcatcgtgacgatgttccagccataggattgaaccagctgaggttcttcgtgtccgggtccgcctttagtatatccccttacttcctggacacgtttctgtgtcgccatgtagtagcaccaacctcccctgccacaacccaagtcaatgactttccccactggttcaagaaatcttctttcaacaagccaacgcaacttagccgtccctctgctcactggatggccccccgttacattgccctcctttctagcatgcttggcagctgaacggtctacttcgatgatggcttcttttctgtaacgggtaaattcctctttggtcatttggtttaatctctccttccaaacttcccccaatgtcctccccttagctccacccatttttagtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 242 - DNA, 7840 bp, synthetic construct
  misc_feature 1..7840: note = Synthetic Construct
  source 1..7840: mol_type = other DNA
gggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttcacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgggtgaaagaatggatgaaccaatacccgctggttttgaacctgaaatgctaagaaaaaagcaaattacggttcttgacttgcaccccggagctggtaaaactcgacgtattttacctcaaattatcaaggaagcgatcaacaggaggttacgtaccgccgttctggcccctactagagttgtagcggctgaaatggctgaagccttaagaggcttaccaatacgataccaaacatctgctgtgcctcgggaacataatggaaacgagatcgtagacgtaatgtgccacgctactcttacacatcgtctaatgtcaccacacagagtacccaattacaatctttttgtcatggatgaagcacattttactgatcctgcctccattgcggcaaggggatatataagtactaaagtggagttgggcgaagccgccgcaatctttatgacagccacaccacccggaacttcagatccattcccggagtccaattcacctatctctgacttacaaacggagatccctgatagagcatggaactcgggctacgaatggataaccgaatacacaggcaaaaccgtctggttcgtcccaagtgtcaaaatgggcaacgaaattgccttgtgtctacagcgtgctgggaaaaaagtagttcaattgaataggaagtcttatgaaacggaatatccaaagtgtaaaaacgatgactgggattttgtaataactactgatatcagtgaaatgggtgcaaacttcaaagcttcgagggttattgattccagaaagtcagttaaaccaaccataattactgagggcgaagggagagttattctcggtgaaccgtctgctgtgacggctgcaagcgcagcgcaacgtcgaggtagaattggaagaaatccatctcaagttggtgacgaatattgttatggtggtcacactaatgaggatgatagtaatttcgcccattggaccgaggctagaatcatgttagacaatattaatatgccaaatgggctcattgcacagttttatcagccagaacgggaaaaggtctataccatggatggtgagtacagattgagaggtgaagaaagaaaaaactttttggaacttctgagaacagcagacctgccggtgtggttagcttacaaagtcgcagcagcaggtgtttcttaccatgatagaagatggtgcttcgacggtcctaggacaaacacaatactagaagataataacgaggtggaggttattacaaagttgggtgagaggaagattttacgccctagatggatagatgctagggtttatagcgaccatcaagctttgaaggcttttaaagatttcgcttcaggaaagcgcgaaccagggtcaagtgcatcctttagtaaaaggagaaaagagcggttaaaactgggagctattttcttggaaggcataactgttaaaggtgtaactcaagtaacaactcaacccaagttaggagaggtgcagcagaaatgtcatcagttctgtggctgggaagggtctgggttccctggagcacagcctgtctccatggacaagcaaaatattagacttttagagcagaagccttataaagtaagctggaaagcagacggcactcgttacatgatgttgattgatggcacaaatgaagtttttatgattgacagagataattccgtttttcatgtttcaaatctggaatttccatttcgtaaagatcttcgcatgcatttatcaaatactcttttggatggggaaatgatcattgacaaagtaaatggacaggccgtcccaagatatttgatctatgacataattaagttcaatgcacaaccagttggagactgtgattttaatattcgtctacagtgtattgaacgtgaaattataagtccacgacatgaaaaaatgaagactggactcattgacaaaacacaggaaccatttagtgtcagacctaaacaattttttgacatcaatatttcaagaaagcttctggaaggaaattttgccaaagaagtcagccatgaaatggatggacttatttttcagcctattggaaaatacaagcctggtcgatgtgatgacattttgaaatggaaacctcccagtctaaactctgtggattttcgacttaagataacaagaatgggaggagaagggttgcttccacagaatgttggccttctctatgttggaggttatgaaagaccctttgcacaaatcaaggtgacaaaagaactaaaacagtatgacaacaaaattatagaatgcaaatttgagaacaatagctgggtcttcatgagacagaggatagacaaaagtttcccaaatgcctacaacacagccatggctgtgtgcaatagcatctcgaaccctgtcaccaaggagatgctgtttgaattcattgacagatgtgcagcagccgcccagggacagaagcggaagtatcccctggaccctgacacggagctcatgccgcccccaccgcccaaaagactgcatcggccaacctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgtt

SEQ ID NO: 243 - DNA, 6898 bp, synthetic construct
  misc_feature 1..6898: note = Synthetic Construct
  source 1..6898: mol_type = other DNA
tcgcgcgtttcggtgatgacggtgaaaacctctgacacatgcagctcccggagacggtcacagcttgtcacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgagttacactgacaaccctcctcaaacaaaaagagctttatcgttagacgatctggtgaatcacgatgaaaatgaaaaggttaaattacaaaaattaagtgaggcggctaatggcagcagaccttttgccgaaaatttagaatctgatataaatcaaacggaaacgggccaagctgctccgattgacaattacaaggagagtactggtcatggctcgcactcacaaaaacctaaatcacgcaagtcatctaatgatgatgaagaaaccgatacggatgacgaaatgggtgcaagtggagaaattaattttgattcagaaatggactttgactatgataaacaacatagaaatttactatccaacggatcacctcctatgaatgatggtagtgatgccaatgcgaagttagaaaagccttctgatgattcaattcatcagaatagcaagagtgatgaagaacagagaataccgaaacaaggtaatgaagggaacattgccagcaactatataacccaagtacctctgcaaaagcagaagcaaactgagaagaagatagcgggaaatgcagtaggaagcgtggtcaagaaggaagaagaagcgaatgcagctgtagataatatttttgaagagaaagctactttacaatcaaaaaagaataatatcaagagagatttggaggttctgaatgaaatatctgcgtcttccaagcccagtaagtacaggaatgttccaatttgggcacaaaaatggaaacctactatcaaagctcttcaaagtataaatgtgaaagatctcaaaattgacccatcttttttaaacattattcccgatgatgacttaacaaagtcagtacaggactgggtttatgctacaatatactcaattgctcctgaactaagatccttcattgagttagaaatgaaatttggtgttattattgatgcgaaaggcccagatcgtgtaaatccaccagtttcttcacaatgtgttttcactgagcttgatgcccatctaacgcctaatattgatgcatctttgttcaaagagttgagcaaatatattcgtggtattagcgaagtcactgaaaatacaggtaaattcagtattattgaatcccagacaagagattccgtctatagagtcggactatccacgcaaagaccaaggtttttgagaatgagtacagatattaagactgggagggtaggacaatttatagagaaaagacatgtagcccaactactattatattcaccaaaagatagttacgacgttaaaatctccctaaacttggaattacctgtacctgacaacgatccgccagaaaaatataaatctcaaagcccaattagtgaaaggacgaaagaccgtgttagttacattcataatgattcctgtaccagaattgatattacaaaagtcgaaaatcataaccaaaattcaaaaagtagacaatcagagaccactcacgaagtggaactagaaatcaacacgcctgcactgttaaacgcctttgataacataacgaacgatagtaaagaatatgcatctcttattagaacgtttctgaataatggtacaattattagaagaaagttatcgtctttatcatatgaaatttttgaaggttcaaagaaagtcatgtaatgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 244 - DNA, 8628 bp, synthetic construct
  misc_feature 1..8628: note = Synthetic Construct
  source 1..8628: mol_type = other DNA
tcgcgcgtttcggtgatgacggtgaaaacctctgacacatgcagctcccggagacggtcacagcttgtcacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgagttacactgacaaccctcctcaaacaaaaagagctttatcgttagacgatctggtgaatcacgatgaaaatgaaaaggttaaattacaaaaattaagtgaggcggctaatggcagcagaccttttgccgaaaatttagaatctgatataaatcaaacggaaacgggccaagctgctccgattgacaattacaaggagagtactggtcatggctcgcactcacaaaaacctaaatcacgcaagtcatctaatgatgatgaagaaaccgatacggatgacgaaatgggtgcaagtggagaaattaattttgattcagaaatggactttgactatgataaacaacatagaaatttactatccaacggatcacctcctatgaatgatggtagtgatgccaatgcgaagttagaaaagccttctgatgattcaattcatcagaatagcaagagtgatgaagaacagagaataccgaaacaaggtaatgaagggaacattgccagcaactatataacccaagtacctctgcaaaagcagaagcaaactgagaagaagatagcgggaaatgcagtaggaagcgtggtcaagaaggaagaagaagcgaatgcagctgtagataatatttttgaagagaaagctactttacaatcaaaaaagaataatatcaagagagatttggaggttctgaatgaaatatctgcgtcttccaagcccagtaagtacaggaatgttccaatttgggcacaaaaatggaaacctactatcaaagctcttcaaagtataaatgtgaaagatctcaaaattgacccatcttttttaaacattattcccgatgatgacttaacaaagtcagtacaggactgggtttatgctacaatatactcaattgctcctgaactaagatccttcattgagttagaaatgaaatttggtgttattattgatgcgaaaggcccagatcgtgtaaatccaccagtttcttcacaatgtgttttcactgagcttgatgcccatctaacgcctaatattgatgcatctttgttcaaagagttgagcaaatatattcgtggtattagcgaagtcactgaaaatacaggtaaattcagtattattgaatcccagacaagagattccgtctatagagtcggactatccacgcaaagaccaaggtttttgagaatgagtacagatattaagactgggagggtaggacaatttatagagaaaagacatgtagcccaactactattatattcaccaaaagatagttacgacgttaaaatctccctaaacttggaattacctgtacctgacaacgatccgccagaaaaatataaatctcaaagcccaattagtgaaaggacgaaagaccgtgttagttacattcataatgattcctgtaccagaattgatattacaaaagtcgaaaatcataaccaaaattcaaaaagtagacaatcagagaccactcacgaagtggaactagaaatcaacacgcctgcactgttaaacgcctttgataacataacgaacgatagtaaagaatatgcatctcttattagaacgtttctgaataatggtacaattattagaagaaagttatcgtctttatcatatgaaatttttgaaggttcaaagaaagtcatgtaatgtaagcggatgccgggagcagacaagcccgtcagggcgcgtcagcgggtgttggcgggtgtacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggttttagcaatggaaagtagagtggcaccggaaattcctgggctcattcaacctgggaatgtcacgcaagacttgaagatgatggtctgtaaattattgaattccccaaaacctacgaaaacattccctggttcccagcctgtgtcctttcagcattctgatgtggaagagaagctgcttgcgcatgattactacgtttgtgagaaaacagatggtctgcgggtgttgatgtttatagtgataaatcctgtgacgggtgagcaaggatgctttatgattgatagggaaaataactattatctggttaatggatttaggtttcccagattaccccaaaagaagaaagaagagctgctagagactcttcaagatggcaccttattagatggtgaacttgtcatacaaactaacccaatgacaaaattacaagagttgcgttatttaatgttcgattgtcttgctatcaatggtagatgtctcacacaatcaccaacaagttctagactagcccaccttggaaaagaattttttaaaccatacttcgatttaagagcagcgtaccctaatcgttgtactacttttccgttcaaaatttccatgaaacatatggatttcagttaccaattagtaaaagttgctaaaagtttagataaactaccacatctttctgatggtctgatatttactcctgtgaaggcaccttacactgccggcggaaaagattcattgttattaaaatggaagccagaacaagaaaacaccgtggacttcaaattgattttagatatcccaatggtggaggatccttctttgcctaaagatgatcggaacaggtggtattacaattatgacgttaagccagttttcagcttatatgtctggcaaggcggagctgatgtcaattcacgtttaaaacatttcgaccagcctttcgataggaaggaatttgaaatattagaaagaacatacagaaaatttgcagagttgagcgtttcagatgaggaatggcaaaatttgaagaacctagaacagccattaaatggtagaatagtagagtgcgcaaaaaaccaagagactggggcgtgggaaatgttaagattcagggatgataagttaaatggtaatcatacatcggtggtccagaaagttttggagagtatcaacgattcagtttcattggaggacctcgaggaaattgttggtgatattaaaaggtgctgggacgagagaagagcaaatatggctggtggtagtgggagaccactaccgtctcaaagtcaaaatgcgacattatctacctctaagccagtccattcacagcccccaagtaatgataaggagccaaaatatgtagacgaggatgattggtcggattagcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 245 - DNA, 1161 bp, synthetic construct; identical to SEQ ID NO: 2
  misc_feature 1..1161: note = Synthetic Construct
  source 1..1161: mol_type = other DNA

SEQ ID NO: 246 - DNA, 2799 bp, synthetic construct; identical to SEQ ID NO: 30
  misc_feature 1..2799: note = Synthetic Construct
  source 1..2799: mol_type = other DNA

SEQ ID NO: 247 - DNA, 922 bp, synthetic construct
  misc_feature 1..922: note = Synthetic Construct
  source 1..922: mol_type = other DNA
cataaactaaaaggtggagctaaggggaggacattgggggaagtttggaaggagagattaaaccaaatgaccaaagaggaatttacccgttacagaaaagaagccatcatcgaagtagaccgttcagctgccaagcatgctagaaaggagggcaatgtaacggggggccatccagtgagcagagggacggctaagttgcgttggcttgttgaaagaagatttcttgaaccagtggggaaagtcattgacttgggttgtggcaggggaggttggtgctactacatggcgacacagaaacgtgtccaggaagtaaggggatatactaaaggcggacccggacacgaagaacctcagctggttcaatcctatggctggaacatcgtcacgatgaagtcaggggttgacgtgttctatcgtccgtcagaatgttgcgacaccttactgtgtgatataggggagagctcaagctctgcggaggtggaggagcatagaacgatccgtgttctggagatggtcgaggattggttgcatcgtggcccaagagagttctgcgtcaaggtcctttgtccgtacatgcctaaggttatagaaaagatggagctgttacaacgtagatatgggggcggtttggtaagaaatccccttagcagaaacagcacgcacgaaatgtactgggtcagcagagcttcaggtaatgtggtgcatagcgtaaacatgactagccaggtactgttaggccgtatggagaaaaggacatggaagggaccgcaatatgaagaggatgtgaaccttggttctggaacaagggcggttggtaaacccctgctgaattctgacactagtaaaattaagaaccgtatagagaggttacgtagggagtacagctctacgtggcaccatgatgaaaatcacgcctgattca

SEQ ID NO: 248 - DNA, 27 bp, synthetic construct
  misc_feature 1..27: note = Synthetic Construct
  source 1..27: mol_type = other DNA
atggttttagcaatggaaagtagagtg

SEQ ID NO: 249 - DNA, 23 bp, synthetic construct
  misc_feature 1..23: note = Synthetic Construct
  source 1..23: mol_type = other DNA
ctaatccgaccaatcatcctcgt

SEQ ID NO: 250 - DNA, 53 bp, synthetic construct
  misc_feature 1..53: note = Synthetic Construct
  source 1..53: mol_type = other DNA
cattgctaaaaccatttttagtttatgtatgtgttttttgtagttatagattt

SEQ ID NO: 251 - DNA, 27 bp, synthetic construct
  misc_feature 1..27: note = Synthetic Construct
  source 1..27: mol_type = other DNA
gtcggattagtgtaagcggatgccggg

SEQ ID NO: 252 - DNA, 32 bp, synthetic construct
  misc_feature 1..32: note = Synthetic Construct
  source 1..32: mol_type = other DNA
gaccctggttcttgcaacacggtatgtggagt

SEQ ID NO: 253 - DNA, 32 bp, synthetic construct
  misc_feature 1..32: note = Synthetic Construct
  source 1..32: mol_type = other DNA
ccgtgttgcaagaaccagggtcaagtgcatcc

SEQ ID NO: 254 - DNA, 45 bp, synthetic construct
  misc_feature 1..45: note = Synthetic Construct
  source 1..45: mol_type = other DNA
agcatctgcactcatttttagtttatgtatgtgttttttgtagtt

SEQ ID NO: 255 - DNA, 37 bp, synthetic construct
  misc_feature 1..37: note = Synthetic Construct
  source 1..37: mol_type = other DNA
atacataaactaaaaatgagtgcagatgctcaatctt

SEQ ID NO: 256 - DNA, 28 bp, synthetic construct
  misc_feature 1..28: note = Synthetic Construct
  source 1..28: mol_type = other DNA
gcctgattcaagaaatatcttgaccgca

SEQ ID NO: 257 - DNA, 28 bp, synthetic construct
  misc_feature 1..28: note = Synthetic Construct
  source 1..28: mol_type = other DNA
gtgattttcatcatggtgccacgtagag

SEQ ID NO: 258 - DNA, 22 bp, synthetic construct
  misc_feature 1..22: note = Synthetic Construct
  source 1..22: mol_type = other DNA
acccgttacagaaaagaagcca

SEQ ID NO: 259 - DNA, 38 bp, synthetic construct
  misc_feature 1..38: note = Synthetic Construct
  misc_feature 24..25: note = n is a, c, g, or t
  source 1..38: mol_type = other DNA
tggcttcttttctgtaacgggtmnnttcctctttggtc

SEQ ID NO: 260 - DNA, 22 bp, synthetic construct
  misc_feature 1..22: note = Synthetic Construct
  source 1..22: mol_type = other DNA
gtgattttcatcatggtgccac

SEQ ID NO: 261 - DNA, 22 bp, synthetic construct
  misc_feature 1..22: note = Synthetic Construct
  source 1..22: mol_type = other DNA
gtggcaccatgatgaaaatcac

SEQ ID NO: 262 - DNA, 40 bp, synthetic construct
  misc_feature 1..40: note = Synthetic Construct
  misc_feature 25..26: note = n is a, c, g, or t
  source 1..40: mol_type = other DNA
accttactgtgtgatataggggagnnktcaagctctgcgg

SEQ ID NO: 263 - DNA, 28 bp, synthetic construct
  misc_feature 1..28: note = Synthetic Construct
  source 1..28: mol_type = other DNA
ctcccctatatcacacagtaaggtgtcg

SEQ ID NO: 264 - DNA, 35 bp, synthetic construct
  misc_feature 1..35: note = Synthetic Construct
  source 1..35: mol_type = other DNA
aaatcactaagcctgattcaagaaatatcttgacc

SEQ ID NO: 265 - DNA, 45 bp, synthetic construct
  misc_feature 1..45: note = Synthetic Construct
  source 1..45: mol_type = other DNA
tagctccaccttttagtttatgtatgtgttttttgtagttataga

SEQ ID NO: 266 - DNA, 30 bp, synthetic construct
  misc_feature 1..30: note = Synthetic Construct
  source 1..30: mol_type = other DNA
accctggttcgtgattttcatcatggtgcc

SEQ ID NO: 267 - DNA, 27 bp, synthetic construct
  misc_feature 1..27: note = Synthetic Construct
  source 1..27: mol_type = other DNA
tgaaaatcacgaaccagggtcaagtgc

SEQ ID NO: 268 - DNA, 28 bp, synthetic construct
  misc_feature 1..28: note = Synthetic Construct
  source 1..28: mol_type = other DNA
taaactaaaaatgggtggagctaagggg

SEQ ID NO: 269 - DNA, 48 bp, synthetic construct
  misc_feature 1..48: note = Synthetic Construct
  source 1..48: mol_type = other DNA
tagctccacccatttttagtttatgtatgtgttttttgtagttataga

SEQ ID NO: 270 - DNA, 27 bp, synthetic construct
  misc_feature 1..27: note = Synthetic Construct
  source 1..27: mol_type = other DNA
ctacggatgtcgcgtatagagcgttcg

SEQ ID NO: 271 - DNA, 25 bp, synthetic construct
  misc_feature 1..25: note = Synthetic Construct
  source 1..25: mol_type = other DNA
aggtccccgtcccgcaaggtgtaag

SEQ ID NO: 272 - DNA, 35 bp, synthetic construct
  misc_feature 1..35: note = Synthetic Construct
  source 1..35: mol_type = other DNA
ctacggatgtcgtgtatgcagcgttcgatatttat

SEQ ID NO: 273 - DNA, 35 bp, synthetic construct
  misc_feature 1..35: note = Synthetic Construct
  source 1..35: mol_type = other DNA
ctacggatgtcgtgtatttagcgttcgatatttat

SEQ ID NO: 274 - DNA, 35 bp, synthetic construct
  misc_feature 1..35: note = Synthetic Construct
  source 1..35: mol_type = other DNA
ctacggatgtcgtgtataaagcgttcgatatttat

SEQ ID NO: 275 - DNA, 34 bp, synthetic construct
  misc_feature 1..34: note = Synthetic Construct
  source 1..34: mol_type = other DNA
gtttctgaaaacaaatgcttgtaggttccaggag

SEQ ID NO: 276 - DNA, 35 bp, synthetic construct
  misc_feature 1..35: note = Synthetic Construct
  source 1..35: mol_type = other DNA
ttagcaaaacctgcgactttatcattataaatatc

SEQ ID NO: 277 - DNA, 34 bp, synthetic construct
  misc_feature 1..34: note = Synthetic Construct
  source 1..34: mol_type = other DNA
gttttgctaagtttctggcaacaaattgttgtag

SEQ ID NO: 278 - DNA, 30 bp, synthetic construct
  misc_feature 1..30: note = Synthetic Construct
  source 1..30: mol_type = other DNA
ctgcgactttatcattataaatatcgaacg

SEQ ID NO: 279 - DNA, 35 bp, synthetic construct
  misc_feature 1..35: note = Synthetic Construct
  source 1..35: mol_type = other DNA
gaaaacaaattgttgtgcgttccaggagaaagatg

SEQ ID NO: 280 - DNA, 25 bp, synthetic construct
  misc_feature 1..25: note = Synthetic Construct
  source 1..25: mol_type = other DNA
agaaacttagcaaaacctgcgactt

SEQ ID NO: 281 - DNA, 35 bp, synthetic construct
  misc_feature 1..35: note = Synthetic Construct
  source 1..35: mol_type = other DNA
gaaaacaaattgttgtaagttccaggagaaagatg

SEQ ID NO: 282 - DNA, 35 bp, synthetic construct
  misc_feature 1..35: note = Synthetic Construct
  source 1..35: mol_type = other DNA
gaaaacaaattgttgtttgttccaggagaaagatg

SEQ ID NO: 283 - DNA, 33 bp, synthetic construct
  misc_feature 1..33: note = Synthetic Construct
  source 1..33: mol_type = other DNA
aattgattcatacttcgctgttaaaaggcatac

SEQ ID NO: 284 - DNA, 30 bp, synthetic construct
  misc_feature 1..30: note = Synthetic Construct
  source 1..30: mol_type = other DNA
aagttgtcatcttcatctttctcctggaac

SEQ ID NO: 285 - protein, 464 aa, Sus scrofa
MENSAKAEECEKLSLKEAKTSIDSETESSFSIDENTTSPGTRLSEKAPTCGLVGTPKKRKMAFEDDLVKESSSCGEDTPSKKRKLDAGIVPEEKGSGDDEGISRKRRMESDDGTQKKRKIELEDVPEKRKNLEEGHSSAVAAHYNELQEVGLEKRSQSRIFYLRNFNNWMKSVLIGEFLEKVRQKKKRDITVLDLGCGKGGDLLKWKKGRIDKLVCTDIADVSVKQCQQRYEDMKNRCRDNEYIFSAEFITADCSKELLIDKFRDPEMCFDICSCQFVCHYSFESYEQADIMLRNACERLSPGGYFIGTTPNSFELIRRLEASETESFGNEIYTVKFQKKGDYPLFGCKYDFNLEGVVDVPEFLVYFPLLNEMAKKYNMKLVYQKTFLEFYEEKIKNNENKMLLKRMQALEPYPASENSRLASEKADDYEHAAKYMKNSQVRLPLVSFHFLTRIYLVFAFEKQQ

SEQ ID NO: 286 - protein, 478 aa, Sus scrofa
  SITE 117: note = misc_feature - Xaa can be any naturally occurring amino acid
MENSAKAEECEKLSLKEAKTSIDSETESSFSIDENTTSPGTRLSEKAPTCGLVGTPKKRKMAFEDDLVKESSSCGEDTPSKKRKLDAGIVPEEKGSGDDEGISRKRRMESDGFTKDXTSTGDGTQKKRKIELEDVPEKRKNLEEGHSSAVAAHYNELQEVGLEKRSQSRIFYLRNFNNWMKSVLIGEFLEKVRQKKKRDITVLDLGCGKGGDLLKWKKGRIDKLVCTDIADVSVKQCQQRYEDMKNRCRDNEYIFSAEFITADCSKELLIDKFRDPEMCFDICSCQFVCHYSFESYEQADIMLRNACERLSPGGYFIGTTPNSFELIRRLEASETESFGNEIYTVKFQKKGDYPLFGCKYDFNLEGVVDVPEFLVYFPLLNEMAKKYNMKLVYQKTFLEFYEEKIKNNENKMLLKRMQALEPYPASENSRLASEKADDYEHAAKYMKNSQVRLPLGTLSKSEWEATSIYLVFAFEKQQ

SEQ ID NO: 287 - protein, 597 aa, Sus scrofa
MAHNKIPPRWLNCPRRGQPVAGRFLPLKTMLGPRYDSQVAEENRFHPSMLSNYLKSLKVKMGLLVDLTNTSRFYDRNDIEKEGIKYIKLQCKGHGECPTTENTETFIRLCERFNERNPPELIGVHCTHGFNRTGFLICAFLVEKMDWSIEAAVATFAQARPPGIYKGDYLKELFRRYGDIEEAPPPPLLPDWCFEDDEDEDEEEDGKKESEPGSSASFGKRRKERLKLGAIFLEGVTVKGVTQVTTQPKLGEVQQKCHQFCGWEGSGFPGAQPVSMDKQNIKLLEQKPYKVSWKADGTRYMMLIDGTNEVFMIDRDNSVFHVSNLEFPFRKDLRIHLSNTLLDGEMIIDRVNGLAVPRYLIYDIIKFNAQPVGDCDFNIRLQCIEREIISPRHEKMKAGLIDKTQEPFSVRNKPFFDIYTSRKLLEGNFAKEVSHEMDGLIFQPTGKYKPGRCDDILKWKPPSLNSVDFRLKITRMGGEGLLPQNVGLLYVGGYERPFAQIKVTKELKQYDNKIIECKFENNSWVFMRQRTDKSFPNAYNTAMAVCNSISNPVTKEMLFEFIDRCVAASQGQKRKHHPDPDAELMPPPPPKRPRPLT

SEQ ID NO: 288 - protein, 845 aa, Monkeypox virus
MDANIVSSSTIATYIDALAKNASELEQRSTAYEINNELELVFIKPPLITLTNVVNISTIQESFIRFTVTNKEGIKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDKLIKELTTLSRHIFMASPENVILSPPINAPIKTFMLPKQDIVGLDLENLYAVTKTDGIPITIRVTSKGLYCYFTHLGYIIRYPVKRTIDSEVVVFGEAVKDKNWTVYLIKLIEPVNAISDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFIEYKKFTNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDVFNEDKLSDVGHQYAANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 289 - protein, 527 aa, SARS-CoV-2
AENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSGSPCESHGKQVVSDIDYVPLKSATCITRCNLGGAVCRHHANEYRQYLDAYNMMISAGFSLWIYKQFDTYNLWNTFTRLQ

SEQ ID NO: 290 - protein, 844 aa, Monkeypox virus
DANIVSSSTIATYIDALAKNASELEQRSTAYEINNELELVFIKPPLITLTNVVNISTIQESFIRFTVTNKEGIKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDKLIKELTTLSRHIFMASPENVILSPPINAPIKTFMLPKQDIVGLDLENLYAVTKTDGIPITIRVTSKGLYCYFTHLGYIIRYPVKRTIDSEVVVFGEAVKDKNWTVYLIKLIEPVNAISDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFIEYKKFTNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDVFNEDKLSDVGHQYAANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 291 - protein, 932 aa, SARS-CoV-2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 292 - protein, 300 aa, West Nile virus
GGAKGRTLGEVWKERLNQMTKEEFTRYRKEAIIEVDRSAAKHARKEGNVTGGHPVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKIKNRIERLRREYSSTWHHDENH

SEQ ID NO: 293 - protein, 527 aa, synthetic construct
  REGION 1..527: note = Synthetic Construct
AENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSDSPCESHGKQVVSDIDYVPLKSATCITRCNLGGAVCRHHANEYRQYLDAYNMMISAGFSLWIYKQFDTYNLWNTFTRLQ

SEQ ID NO: 294 - protein, 527 aa, synthetic construct
  REGION 1..527: note = Synthetic Construct
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 295 - protein, 527 aa, synthetic construct
  REGION 1..527: note = Synthetic Construct
AENVTGLFKDCSKVINGLHPTQALTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFTRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTVEYPIIGDELKINAACRKVQHMVVKAALLADKFSVLHDIGNPKAIKCVPQAEVEWKFYDAQPCSDKAYKIEELYYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 296 - protein, 527 aa, synthetic construct
  REGION 1..527: note = Synthetic Construct
AENVTGLFKDCSKVITGLHPTQAPTHLSVDTKFKTEGLCVDIPGIPKDMTYRRLISMMGFKMNYQVNGYPNMFITREEAVRHVRAWIGFDVEGCHATREAIGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDKRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 297 - protein, 508 aa, synthetic construct
  REGION 1..508: note = Synthetic Construct
TSLYKRCSFEYSGQHPAHALTWHDCGAEYRCDEPLAKLVGVADGTLISYKTLVSALGFLPSLKIDTYHNMFLTRDACRAYVQSWIGIDVEAAHAIKPNVGTNLPLQVGFSTGKNFSVVPEGIWVNAHGFCTEPVPAKIPPGDEFKHLKKDMRQARPWKVIRYEIVAHLADVAPQTDYICFVTWAHQLELATMRYFVKLGIEERCYCGRRACFTNGTEFACKAHHALTVPQCDYVYNPFLVDVATWGFSGRLSTNHDAVCTYHSNAHVASADAIMTVCLAIHELFSSVDWALEFPVTPEQSQLNKACRLVQANYLNILLTTTKATVVHDIGNPKGIPIVRRPGVKYHYYDQAPIVKHVQKLKYKPEMEARFIDGLTMFWNCNVDTYPANALVCRYDTHRQKHLIGPNGAALYVNKHAFLTPEMHTYATHKLTLAPLVYYSTTDCSAEQPIVVTYRDCVTRCNTGTTICPTHALEYQDFIAAYNLMARHGFNVYIPRNVNVYNCWLTFTN

SEQ ID NO: 298 - protein, 518 aa, synthetic construct
  REGION 1..518: note = Synthetic Construct
TGLFKICNKDFAGVHPAHAVTTYALSSNYKVGEGLAALVNANVGDVIPYKHLISLLGFKMNVDVECCHSMFITRDSAIRNVRGWIGFDVEATHACGTNIGTNLPFQIGFSTGADFVAVPEGLVDTPSGSQFEPVQSKTPPGEQFNHLRSLFKLAKPWSVLRPRIIQMLADNLSGVSECVVFVTWCHGLELTTMRYFCKIGPEKRCHCGQRATCMSSSQDSYSCWKHSLGCDFVYNPYIVDIQQWGYSGNLQSNHDLYCNVHGHAHVASADATMTRCLAIYNAFCQDVNWEITYPHIVNEDEINSSCRLLQRYFLNACIDALKPSTIYDIGNPKGIKCVRRSDVNFRFFDKQPIVHNVKQLTYDFNEHSNKFKDGLCLFWNCNVDCYPDNSLVCRFDTRNLSVLNLPGCNGGSLYVNKHAFHTPRFDRTSFKYLKPMPFFFYDSSPCDTIQIDGVSQDLVSLSTKDCITRCNIGGAVCKKHAQLYAEFVSAYNVAVTAGFTFWVANGFDPYNLWKQFTS

SEQ ID NO: 299 - protein, 509 aa, synthetic construct
  REGION 1..509: note = Synthetic Construct
CGLFKDCAKVEHYIPPAYATTYISLSDSFKTNNGFAVDVGNKPVTYASVISYMGFRFDANIPGYHTLFCTRDFAIRNVRAWLGFDVESAHVCGDNIGTNVPLQLGFSNGVDFVVQPEGCVVTDKGSSICPVKSRAPPGEQFAHLIPLMRRGQAWSIVRRRIVQMVCDYFDGLSDVVIFVLWAGGLELTTMRYFVKIGVPCKCDCGVVATCYNSAQGTYHCMKHALGCDYLYNPYCIDIQQWGYTGSLSMNHHEVCNVHRNEHVASGDATMTRCLAVHDCFVKRVDWSITYPFIDNEATINKAGRVVQSHVVRAAIKVLNPSAIHDVGNPKGIRCVTTPLPWFCYDKDPINSNVRCLDYDFITHGQMPGLMLFWNCNVDMYPECSVVCRFDTRTRSKLSLEGCNGGALYVNNHAFHTEAYDRRAFAKLKPLPFFFFDDSTCELVDGQPNYVPLKSNVCITRCNIGGAVCKKHAALYRKYVEDYNTFVQAGFTIWGPSNFDTYTLWQGFVN

SEQ ID NO: 300 - protein, 517 aa, synthetic construct
  REGION 1..517: note = Synthetic Construct
TGLYKDCSREVAGLSPAYAPTYMSVDAKFKTNDELCVNLNIPTNVSYSRVISRMGFKLDASIPGYPKLFITKEEAIRQVRSWIGFDVEGAHVSRNACGTNVPLQLGFSTGVNFVVQTVGAVDTDWGTMLTNIAARPPPGEQFKHLVPLMNKGAAWPVVRRRIVQMLSDTLDKLSDYCTFVCWAHGFELTSASYFCKIGKEQKCCMCNRRAGAYSSPLHSYACWTHSSGYDYVYNPFFVDVQQWGYIGNLATNHDRYCTLHQGAHVASNDAIMTRCLAIYSCFIERVDWDVEYPYISHERQLNAFCRVVERNVVRAALLAGSFERVYDIGNPKGIPIVEDPVVDWHYYDAQPLRKNVKQLFYTDDLASKFNDGLCLFWNCNVPKYPNNAVVCRFDTRVHSEFNLPGCNGGSLYVNKHAFHTPAYDVSAFRDLKPMPFFYYSTTPCEMHGNGNMLEDIDYVPLKSAVCVTACNLGGAVCRKHAAEYRDYVEAYNLVSAAGFRLWCYKTFDIYNLWSSFT

SEQ ID NO: 301 - protein, 528 aa, SARS-CoV-1
MAENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSDSPCESHGKQVVSDIDYVPLKSATCITRCNLGGAVCRHHANEYRQYLDAYNMMISAGFSLWIYKQFDTYNLWNTFTRLQ

SEQ ID NO: 302 - protein, 528 aa, SARS-CoV-2
MAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

(19) United States
(12) Patent Application Publication
(10) Pub. No.: US 2024/0327780 A1
(43) Pub. Date: Oct. 3, 2024

(54) SYNTHETIC BIOLOGY APPROACHES TO TARGET RNA-CAPPING ENZYMES FROM VIRUSES

(71) Applicant: The Board of Trustees of the University of Illinois, Urbana, IL (US)

(72) Inventors: Angad P. Mehta, Champaign, IL (US); Marya Y. Ornelas, Urbana, IL (US); Angela Y. Thomas, Champaign, IL (US)

(73) Assignee: The Board of Trustees of the University of Illinois, Urbana, IL (US)

(21) Appl. No.: 18/622,394

(22) Filed: Mar. 29, 2024

Related U.S. Application Data
(60) Provisional application No. 63/493,639, filed on Mar. 31, 2023.

(51) Int. Cl. C12N 1/18; A61K 39/215; C12N 7/00; C12N 9/10; C12N 9/12; C12N 9/18

(52) U.S. Cl. CPC C12N 1/185; A61K 39/215; C12N 7/00; C12N 9/1007; C12N 9/1241; C12N 9/18; C12N 2510/00; C12N 2710/24122; C12N 2770/20022; C12N 2770/24122; C12Y 207/0705; C12Y 301/03033

(57) ABSTRACT

The emergence of zoonotic pathogenic viruses has accentuated the need to develop broad-spectrum antivirals and vaccines. Highly modular yeast-based phenotypic platforms for characterization and targeting of RNA capping enzymes from emerging pathogens including coronaviruses, MPV, ASFV, and WNV, are disclosed herein. This platform can identify key amino acid residues and protein domains. Inactivation and attenuation mutations in viral enzymes are also disclosed herein. This platform is applied to vertebrate RNA capping enzymes, demonstrating use for high-throughput phenotypic screening. The disclosed platforms are highly modular and can be adapted for RNA capping enzymes from viruses and variants that emerge in the future.

### CROSS REFERENCE TO RELATED APPLICATIONS

This application claims priority to U.S. Provisional Application No. 63/493,639, filed Mar. 31, 2023, which is incorporated by reference in its entirety.

### ACKNOWLEDGMENT OF GOVERNMENT SUPPORT

This invention was made with government support under GM136629 awarded by the National Institutes of Health and 2037695 awarded by the National Science Foundation. The government has certain rights in the invention.

### INCORPORATION OF ELECTRONIC SEQUENCE LISTING

The Sequence Listing is submitted as an XML file in the form of the file named “7950-109668-02_ST26.xml” (˜692,165 bytes), which was created on Mar. 21, 2024 which is incorporated by reference herein.

### FIELD

This relates to a modular yeast synthetic biology platform which allows characterization and targeting of viral, and other, RNA enzymes, as well as attenuated viruses generated using the same.

### BACKGROUND

Pathogenic RNA viruses are a threat to human health and world economy. This is clear from the COVID-19 pandemic which resulted in more than 470 million infections and more than 6 million deaths worldwide in a relatively short period of time. Several other pathogenic RNA virus related outbreaks have occurred in the last 20 years, e.g., SARS-CoV in 2003, MERS-CoV in 2012, and Ebola virus from 2013 to 2016. The emergence of new deadly pathogenic RNA viruses, including pathogenic coronavirus, accentuates the need to develop novel broad-spectrum antivirals and vaccines. Broad-spectrum antiviral development strategies have targeted mechanisms that are highly conserved in RNA viruses, e.g., viral replication. These small molecules have been repurposed for COVID-19 and have shown potency for targeting SARS-CoV-2. One example is Remdesivir, a broad-spectrum antiviral that targets RNA dependent RNA polymerases. Remdesivir was developed to treat Ebola, Hepatitis C and other viral infections, and was then repurposed to combat the COVID-19 pandemic. There is a need to develop antivirals that target other conserved mechanisms involved in RNA virus replication like viral genome encoded RNA capping enzymes and viral proteases. Essential viral enzymes are also targeted to develop attenuated strains of viruses as potential live attenuated vaccine strains.

### SUMMARY

This disclosure provides tractable and modular synthetic biology platforms to characterize and target viral genome encoded RNA capping enzymes from RNA viruses. SARS-CoV-2 is a strain of coronavirus that causes coronavirus disease 2019 (COVID-19). The relatively large RNA genome of coronaviruses (˜30 kb) encodes several non-structural proteins (nsp) like RNA polymerase (nsp12), helicase (nsp13), proof-reading exonucleases (nsp14), RNA capping enzymes (e.g., nsp10, nsp14) and proteases (M^{pro}) which are involved in high fidelity replication, translation and packaging of the viral genomes, as well as evading innate immune responses. One feature of the coronavirus replication is the replication of the viral genome catalyzed by viral RdRP and the incorporation of the RNA-cap structure at the 5′-position of the viral RNA genome which is catalyzed by viral genome encoded RNA capping enzyme.³⁰ These viral RNA capping enzymes are components of the coronavirus genome replication and translation, and are implicated in immune evasion.^{25,31}The final viral RNA cap-0 structure of the coronavirus genome is identical to that of eukaryotic host mRNAs, i.e., it consists of a 5′-end cap structure that consists of 7-methylguanosine (m7G) linked to first nucleotide of the transcript mRNA via 5′-5′ triphosphate bridge.³² Conventional RNA cap maturation involves three steps: (i) RNA triphosphatase which catalyzes the cleavage of the B-phosphate and γ-phosphate, (ii) Guanylyltransferase that catalyzes the linkage of guanosine (G) linked to first nucleotide of the transcript mRNA via 5′-5′ triphosphate bridge, and (iii) Methyltransferase that catalyzes the methylation at the N7 position of guanine at cap-0 and 2′-O methylation at cap-1 position using S-adenosylmethionine (AdoMet) as a cofactor. In eukaryotic cells, the mRNA cap structure is implicated in efficient recognition of the mRNA by the eukaryotic translation initiation factor 4E (elF4E) for translation initiation.³³ RNA molecules lacking 5′-end cap structure are rapidly degraded in cytoplasmic P-bodies.³⁴

During the course of viral/host adaptation and evolution, viral RNA-cap structures identical to the host mRNAs have been selected for, to promote efficient viral RNA translation and propagation. Essentially, the virus disguises its RNA genome to resemble the host mRNA by incorporating a 5′-end cap structure that is identical to the host mRNA 5′-end cap structure. RNA viruses typically encode their own RNA capping enzymes that are involved in the maturation of the 5′-end RNA cap structure. One reason why RNA viruses, including coronaviruses, encode their own capping enzymes is that they replicate in the host cytoplasm, whereas the host RNA capping enzymes are localized in the host nucleus.³² Compared to the conserved enzymatic steps involved in the eukaryotic mRNA capping reactions, the mechanistic details of the enzymatic steps of viral RNA capping processes can be highly diverse.³⁵ In addition to differences in enzyme structure and reaction mechanisms, the viral RNA capping is diverse in terms of genetic components and protein domain organization. Most RNA viruses encode RNA capping enzymes that first incorporate a guanosine linked to first nucleotide of the transcript mRNA via 5′-5′ triphosphate bridge followed by a series of methylation reactions at cap-0 and cap-1 position (FIGS. 1A-1B).³⁵ Certain RNA viruses, like influenza viruses, incorporate a 5′-end cap by a mechanism called as cap-snatching mechanism, where viral enzymes essentially “snatch” and excise the 5′-end of host mRNAs and append it to the viral RNA genome (FIG. 1).³⁶ Certain viruses encode a complex RNA secondary structure which facilitates cap-independent translation of the viral mRNA sequence.³⁷ SARS-CoV-2 encodes several RNA capping enzymes. Studies on RNA viruses, like coronavirus (e.g., SARS-CoV-1, which was the cause of SARS, 2002) have demonstrated that nsp14 is one component involved in maintaining replication fidelity and therefore, pathogenicity.^{38,39} Studies with pathogenic replicons of SARS-CoV-1 indicate that nsp14 was involved in high-fidelity replication and efficient viral propagation. Inactivation mutations in the methyl transferase domain of nsp14 of SARS-CoV-1 resulted in impaired replication/translation of the virus. Further, these functionally inactive point mutations in the methyltransferase resulted in attenuation in pathogenesis in RNA viruses.^{40,41} Typically, in vitro enzyme assays and/or pathogenic replicons of viruses have been used to screen for inhibitors of RNA capping enzymes,^{42,43} however, such platforms are not suitable for a high-throughput screening methodologies. Developing platforms that are compatible with high-throughput screening approaches in standard laboratory setting is therefore likely to be beneficial for screening potential antivirals and identifying attenuation mutations.

Phenotypic yeast-based complementation platforms are disclosed herein, which can be used in standard laboratory setting for functional characterization and targeting of N7-MTase (Yeast platform for RNA Cap 0 N7-Methyltransferase—YeRCOM). In some examples, this platform is used for functional characterization of SARS-CoV-2 nsp14, domains and residues in nsp14 were linked to N7-MTase activity. YeRCOM further encompasses nsp14 variants observed in emerging variants of SARS-CoV-2 (e.g., delta variant of SARS-CoV-2 encodes nsp14 A394V and nsp14 P46L). YeRCOM was combined with directed evolution to identify attenuation mutations in SARS-CoV-2 nsp14. Because of high sequence similarity of nsp14 in emerging coronaviruses, these observations have implications on live attenuated vaccine development strategies. In some examples, YeRCOM is used for characterization and targeting RNA capping enzymes from other emerging pathogens including MERS-CoV, Monkeypox virus and African Swine Fever virus. In some examples, YeRCOM is used for characterization and targeting of human RNA capping enzymes and to demonstrate the feasibility of this approach to perform phenotypic screening to identify inhibitors of RNA capping enzymes. In some examples, a yeast platform for RNA Cap 0 Guanylyltransferases is used for characterization and targeting of a of RNA capping enzyme in emerging pathogens, such as West Nile virus and SARS-CoV-2. In some examples, a yeast platform for RNA 5′-triphosphatase is used for characterization and targeting of RNA capping enzymes in emerging pathogens. The disclosed platforms can be readily used in combination with high-throughput screening and medicinal chemistry approaches to rapidly develop inhibitors of SARS-CoV-2 methyltransferase as potential antivirals.

Provided is an isolated non-native yeast, which includes at least one of (a) a genetically inactivated Abd1 gene and a heterologous methyltransferase; (b) a genetically inactivated Ceg1p gene and a heterologous guanylyltransferase; and/or (c) a genetically inactivated Cet1p and a heterologous RNA triphosphatase, as well as compositions and methods of using the same, such as for screening for compounds which reduce or inhibit expression and/or activity of the heterologous methyltransferase, heterologous guanylyltransferase, and/or heterologous RNA triphosphatase. Also provided are isolated and attenuated viruses, such as SARS-CoV-2, MERS-CoV, ASFV, MPV, and/or WNV, and methods of their use to stimulate an immune response.

### REFERENCES FOR SUMMARY

(25) Chen, Y.; Guo, D. Molecular Mechanisms of Coronavirus RNA Capping and Methylation. Virol. Sin. 2016, 31 (1), 3-11. https://doi.org/10.1007/s12250-016-3726-4.
(30) Sawicki, S. G. Coronavirus Genome Replication. Viral Genome Replication 2008, 25-39.
(31) Kasuga, Y.; Zhu, B.; Jang, K.-J.; Yoo, J.-S. Innate Immune Sensing of Coronavirus and Viral Evasion Strategies. Exp Mol Med 2021, 53 (5), 723-736.
(32) Ramanathan, A.; Robb, G. B.; Chan, S.-H. MRNA Capping: Biological Functions and Applications. Nucleic Acids Res 2016, 44 (16), 7511-7526.
(33) Filipowicz, W.; Furuichi, Y.; Sierra, J. M.; Muthukrishnan, S.; Shatkin, A. J.; Ochoa, S. A Protein Binding the Methylated 5′-Terminal Sequence, M7GpppN, of Eukaryotic Messenger RNA. Proceedings of the National Academy of Sciences 1976, 73 (5), 1559-1563.
(34) Sheth, U.; Parker, R. Decapping and Decay of Messenger RNA Occur in Cytoplasmic Processing Bodies. Science 2003.
(35) Decroly, E.; Ferron, F.; Lescar, J.; Canard, B. Conventional and Unconventional Mechanisms for Capping Viral MRNA. Nat Rev Microbiol 2012, 10 (1), 51-65.
(36) De Vlugt, C.; Sikora, D.; Pelchat, M. Insight into Influenza: A Virus Cap-Snatching. Viruses 2018, 10 (11), 641.
(37) Martinez-Salas, E.; Francisco-Velilla, R.; Fernandez-Chamorro, J.; Embarek, A. M. Insights into Structural and Mechanistic Features of Viral IRES Elements. Frontiers in Microbiology 2018, 8.
(38) Eckerle, L. D.; Becker, M. M.; Halpin, R. A.; Li, K.; Venter, E.; Lu, X.; Scherbakova, S.; Graham, R. L.; Baric, R. S.; Stockwell, T. B. Infidelity of SARS-CoV Nsp14-Exonuclease Mutant Virus Replication Is Revealed by Complete Genome Sequencing. PLoS pathogens 2010, 6 (5).
(39) Eckerle, L. D.; Lu, X.; Sperry, S. M.; Choi, L.; Denison, M. R. High Fidelity of Murine Hepatitis Virus Replication Is Decreased in Nsp14 Exoribonuclease Mutants. Journal of virology 2007, 81 (22), 12135-12144.
(40) Chen, Y.; Cai, H.; Xiang, N.; Tien, P.; Ahola, T.; Guo, D. Functional Screen Reveals SARS Coronavirus Nonstructural Protein Nsp14 as a Novel Cap N7 Methyltransferase. Proceedings of the National Academy of Sciences 2009, 106 (9), 3484-3489.
(41) Sperry, S. M.; Kazi, L.; Graham, R. L.; Baric, R. S.; Weiss, S. R.; Denison, M. R. Single-Amino-Acid Substitutions in Open Reading Frame (ORF) 1b-Nsp14 and ORF 2a Proteins of the Coronavirus Mouse Hepatitis Virus Are Attenuating in Mice. Journal of virology 2005, 79 (6), 3391-3400.
(42) Otava, T.; Sila, M.; Li, F.; Fanfrlik, J.; Devkota, K.; Perveen, S.; Chau, I.; Pakarian, P.; Hobza, P.; Vedadi, M.; Boura, E.; Nencka, R. The Structure-Based Design of SARS-CoV-2 Nsp14 Methyltransferase Ligands Yields Nanomolar Inhibitors. ACS Infect. Dis. 2021, 7 (8), 2214-2220.
(43) Devkota, K.; Schapira, M.; Perveen, S.; Khalili Yazdi, A.; Li, F.; Chau, I.; Ghiabi, P.; Hajian, T.; Loppnau, P.; Bolotokova, A.; Satchell, K. J. F.; Wang, K.; Li, D.; Liu, J.; Smil, D.; Luo, M.; Jin, J.; Fish, P. V.; Brown, P. J.; Vedadi, M. Probing the SAM Binding Site of SARS-CoV-2 Nsp14 In Vitro Using SAM Competitive Inhibitors Guides Developing Selective Bisubstrate Inhibitors. SLAS DISCOVERY: Advancing the Science of Drug Discovery 2021, 26 (9), 1200-1211.

The foregoing and other features of this disclosure will become more apparent from the following detailed description of several aspects which proceeds with reference to the accompanying figures.

### BRIEF DESCRIPTION OF THE FIGURES

FIGS. 1A-1B shows viral RNA capping. Several families of viruses including coronaviruses, flaviviruses and filoviruses encode enzymes necessary for the maturation of cap-0 and cap-1 structures. Some viruses like influenza A, utilize alternate mechanisms for cap maturation, i.e., cap-snatching mechanism. Some viruses like hepatitis C virus, utilize internal ribosomal entry site (IRES) for cap-independent translation (1A). Enzymatic reactions involved in conventional RNA capping transformations described. For SARS-CoV-2, the methyltransferase reaction is catalyzed by nsp14 and in some examples, phenotypic yeast platforms for functional characterization and targeting of SARS-CoV-2 nsp14 were developed (1B).

FIGS. 2A-2J show vectors: pMO1 (2A), pMO2 (2B), pMO3 (2C), pMO32 (2D), pROS1 (2E), pROS2 (2F), pROS8 (2G), pROS12 (2H), pROS13 (2I), and pROS14 (2J). Plasmid details are listed in Table 2.

FIGS. 3A-3D show engineering YeRCOM for functional screening of RNA cap-0 methyltransferase activity. (3A) Scheme of workflow to generate S. cerevisiae abd1::kanMX4 pMO1 haploid. S. cerevisiae abd1::kanMX4 pMO1 diploid was sporulated and dissected which led to two possible populations of strains surviving on selection media: S. cerevisiae abd1::kanMX4 pMO1 diploid and S. cerevisiae abd1::kanMX4 pMO1 haploid. S. cerevisiae abd1::kanMX4 pMO1 diploid can grow in the presence of 5-FOA because abd1 is expressed from the genome. S. cerevisiae abd1::kanMX4 pMO1 haploid cannot grow in the presence of 5-FOA because growth is dependent on plasmid based expression of abd1; curing pMO1 results in no yeast growth. (3B) Both S. cerevisiae abd1::kanMX4 pMO1 diploid and S. cerevisiae abd1::kanMX4 pMO1 haploid grow in the absence of 5-FOA, but only S. cerevisiae abd1::kanMX4 pMO1 diploid grows in the presence of 5-FOA due to chromosomal expression of abd1. Data represent 3 biological replicates. (3C) Total growth after 48 hours of S. cerevisiae abd1::kanMX4 pMO1 haploid (Haploid) and S. cerevisiae abd1::kanMX4 pMO1 diploid (Diploid) in the presence of 5-FOA. Each circle represents an individual replicate. Bars represent the average of 3 replicates and error bars represent mean±SD. (3D) Enzymatic scheme demonstrating 5′-capping of yeast mRNAs using native yeast enzymes (Ceg1, Cet1, Abd1).

FIGS. 4A-4B show characterization of S. cerevisiae abd1::kanMX4 haploid pMO1 strain: (4A) Lane 1: PCR amplification of the S. cerevisiae mating allele followed by gel electrophoresis showing diploid MATa and MATα amplification from S. cerevisiae abd1::kanMX4 diploid pMO1; Lane 2: PCR amplification of the S. cerevisiae mating allele followed by gel electrophoresis showing amplification of just MATa from S. cerevisiae abd1::kanMX4 haploid pMO1. (4B) Total DNA was isolated from S. cerevisiae abd1::kanMX4 haploid pMO1 and defined gene segments were PCR amplified and analyzed by gel electrophoresis for characterization of S. cerevisiae abd1::kanMX4 haploid pMO1. Lane 1: abd1 fragment from pMO1; Lane 2: S. cerevisiae mating allele, MATa; Lane 3: kanMX4 locus from chromosome II.

FIGS. 5A-5D show using YeRCOM for functional characterization of SARS-CoV-2 nsp14 activity. (5A) SARS-CoV-2 nsp14 plasmid expression is able to recover S. cerevisiae abd1::kanMX4 haploid growth in the presence of 5FOA. (5B) The ability of WT nsp14 and a series of mutants and truncations of nsp14 (see Table 5 for details) to catalyze the N7-methylation of yeast mRNAs was studied by transforming their respective plasmid construct in S. cerevisiae abd1::kanMX4 pMO1 haploid and monitoring their ability to recover growth in the presence of 5-FOA. Data represent 3 biological replicates. (5C) Representative strains listed in panel (5B) were grown in liquid selection medium in the presence of 5-FOA and the total growth after 48 hours was measured (OD600). Each circle represents an individual replicate. Bars represent the average of 4 replicates and error bars represent mean±SD. Note: In the figure legend of the bar-charts, pMO2 corresponds to S. cerevisiae abd1::kanMX4 pMO1 pMO2 haploid, pMO3 corresponds to S. cerevisiae abd1::kanMX4 pMO1 pMO3 haploid and so on. (5D) Enzymatic scheme demonstrating 5′-capping of yeast mRNAs using yeast enzymes (Ceg1, Cet1) and SARS-CoV-2 nsp14.

FIG. 6 shows characterization of the growth rates of S. cerevisiae abd1::kanMX4 pMO1 haploid and S. cerevisiae abd1::kanMX4 pMO2 haploid. The effect of constitutive plasmid expression of abd1 or nsp14 on recovery of S. cerevisiae abd1::kanMX4 haploid growth. Each data point represents the mean of 3 biological replicates. Error bars represent mean±SD.

FIGS. 7A-7D depict building YeRCOM platforms to study the methyltransferase activity of nsp14 mutations observed in variants of SARS-CoV-2. (7A) SARS-CoV-2 nsp14 plasmid expression is able to recover S. cerevisiae abd1::kanMX4 haploid growth in the presence of 5FOA. (7B) The ability of variants of nsp14 to catalyze the N7-methylation of yeast mRNAs was studied by transforming their respective plasmid construct in S. cerevisiae abd1::kanMX4 pMO1 haploid and monitoring their ability to recover growth in the presence of 5-FOA. Data represent 3 biological replicates. (7C) Representative strains listed in panel (7B) were grown in liquid selection medium in the presence of 5-FOA and the total growth after 48 hours was measured (OD600). Each circle represents an individual replicate. Bars represent the average of 4 replicates and error bars represent mean±SD. Note: In the figure legend of the bar-charts, V381L corresponds to S. cerevisiae abd1::kanMX4 pMO1 pMO15 haploid, A394V corresponds to S. cerevisiae abd1::kanMX4 pMO1 pMO16 haploid and so on. (7D) Enzymatic scheme demonstrating 5′-capping of yeast mRNAs using yeast enzymes (Ceg1, Cet1) and nsp14 variants encoded by SARS-CoV-2 variants.

FIG. 8 shows characterization of the growth rates of variants of SARS-CoV-2 nsp14. Variants A394V (label B), I42V (label C), P412H (label D), P46L (label E), and V381L (label F) were studied for their effects on nsp14 methyltransferase function and thus yeast growth rates in comparison to WT nsp14 (label A). Each data point represents the mean of 3 biological replicates. Error bars represent mean±SD. Strains were grown in liquid selection medium in the presence of 5-FOA and the total growth after 48 hours was measured (OD600). Each circle represents an individual replicate. Bars represent the mean of 3 replicates and error bars represent mean±SD.

FIGS. 9A-9B show sequence alignment of nsp14 protein sequences from a diverse set of coronaviruses across two panels (9A and 9B). Amino acid residues (D331, D243) are highly conserved residues and their mutation to alanine is known to abolish methyltransferase function (D331A) and exonuclease function (D243A) of nsp14. Amino acid residues (W293, A354, F368, Y369, and C388) are highly conserved residues targeted for random mutagenesis herein. Amino acid residues (P46, V381, A394, and P412) are mutations found in SARS-CoV-2 variants and studied for their effects on nsp14 methyltransferase function in this study. GenBank Accession numbers for representative nsp14 sequences include: SARS-CoV-1 (SEQ ID NO: 293 is depicted) (AY278741), SARS-CoV-2 (SEQ ID NO: 294 is depicted) (MT318827.1), Pangolin CoV (SEQ ID NO: 295 is depicted) (MT121216.1), Bat CoV (SEQ ID NO: 296 is depicted) (MN996532), Magpie-robin-CoV (SEQ ID NO: 297 is depicted) (005352853), Canada Goose CoV (SEQ ID NO: 298 is depicted) (QCB65096), Mink CoV (SEQ ID NO: 299 is depicted) (009019180), Erinaceus hedgehog (SEQ ID NO: 300 is depicted) (QGA70691), each of which is incorporated by reference herein in their entirety. Sequence alignment was performed using Clustal Omega EMBL-EBI Multiple Sequence Alignment tool.

FIGS. 10A-10E show the use of YeRCOM for directed evolution of nsp14 to identify inactivation and attenuation mutations. (10A) Libraries of nsp14 were generated and transformed into S. cerevisiae abd1::kanMX4 pMO1 haploid. The libraries were then screened in the presence of 5-FOA to identify inactivated or attenuated variants of nsp14. (10B) List of nsp14 variants that are inactive. (10C) Representative strains listed in panel (10B) were grown in liquid selection medium in the presence of 5-FOA and the total growth after 48 hours was measured (OD600). Each circle represents an individual replicate. Bars represent the mean of 4 replicates and error bars represent mean±SD. (10D) Growth curves of the identified attenuated mutant of nsp14 (label D), nsp14 W293F (label B), nsp14 F368N (label C), and WT nsp14 (label A) to elucidate the activity of each point mutation. Each data point represents the mean of 3 biological replicates. Error bars represent mean±SD. (10E) Growth curves of additional attenuation mutants of nsp14 identified in this disclosure: F368L (label B), D353T (label C), and D353A (label D). Each data point represents the mean of 3 biological replicates. Error bars represent mean±SD.

FIGS. 11A-11D show sequence and structural analysis of WT and attenuated nsp14 mutant. (11A) Sequence alignment of nsp14 protein sequences from a diverse set of alpha, beta, and gamma coronaviruses. Complete sequence alignment is shown in FIG. 9. Amino acid residues (D331, D243) are highly conserved residues and their mutation to alanine abolishes methyltransferase function (D331A) and exonuclease function (D243A) of nsp14. Amino acid residues (W293, A354, F368, Y369, and C388) are highly conserved residues targeted for random mutagenesis in this disclosure. Amino acid residues (P46, V381, A394, and P412) are mutations found in SARS-CoV-2 variants and studied for their effects on nsp14 methyltransferase function. Accession numbers for representative nsp14 sequences: SARS-CoV-1 (AY278741), SARS-CoV-2 (MT318827.1), Pangolin CoV (MT121216.1), Bat CoV (MN996532), Magpie-robin-CoV (005352853), Canada Goose CoV (QCB65096), Mink CoV (009019180), Erinaceus hedgehog (QGA70691). Sequence alignment was performed using Clustal Omega EMBL-EBI Multiple Sequence Alignment tool. For Magpie-robin-Cov residues 1-56 and 233-405 of SEQ ID NO: 297 are depicted. For Mink-CoV residues 1-53 and 229-402 of SEQ ID NO: 299 are depicted. For Canada-Goose-CoV residues 1-56 and 232-408 of SEQ ID NO: 298 are depicted. For Erinaceus-hedgehog-CoV residues 1-54 and 231-406 of SEQ ID NO: 300 are depicted. For SARS-CoV-1 residues 1-58 and 235-414 of SEQ ID NO: 293 are depicted. For Pangolin-CoV residues 1-58 and 235-414 of SEQ ID NO: 295 are depicted. For SARS-CoV-2 residues 1-58 and 235-414 of SEQ ID NO: 294 are depicted. For Bat-CoV residues 1-58 and 235-414 of SEQ ID NO: 296 are depicted. (11B) Evolutionary relationships between strains used in sequence alignment analysis predicted using Clustal Omega. (11C) Superimposition of WT nsp14 crystal structure (PDB code: 5C8T) (ribbon and sticks) with predicted WT nsp14 SWISS-MODEL structure (ribbon and sticks). (11D) Superimposition of WT nsp14 crystal structure (PDB: 5C8T) (ribbon and sticks) with predicted nsp14 SWISS-MODEL structure (ribbon and sticks) for attenuated nsp14 mutant (F368N, W293F). Zoomed in active site images show residues interacting with ligand S-adenosyl methionine (SAM). Residues mutated in this disclosure (C388, A354, F368. Y369, W293) are shown.

FIGS. 12A-12B show modeling mutations that result in attenuated nsp14 activity using AlphaFold. (12A) Superimposition of WT nsp14 crystal structure (PDB code: 5C8T) (ribbon and sticks) with predicted WT nsp14 AlphaFold structure (ribbon and sticks). (12B) Superimposition of WT nsp14 crystal structure (PDB: 5C8T) (ribbon and sticks) with predicted nsp14 Alphafold structure (ribbon and sticks) for attenuated nsp14 mutant (F368N, W293F). Zoomed in active site images show residues interacting with ligand S-adenosyl methionine (SAM). Residues mutated in this study (C388, A354, F368. Y369, W293) are shown. Modeling was performed using Alphafold with 5 minimum models, 24 recycles, 0.5 Å tolerance, and 1 sample.

FIGS. 13A-13L show vectors pMO1 (13A), pMO5 (13B), pMO41 (13C), pMO42 (13D), pIJ11 (13E), pIJ12 (13F), pIJ13 (13G), pAT15 (13H), pMO65 (13I), pMO66 (13J), pMO67 (13K), and pMO68 (13L). Plasmid details are listed in Table 7.

FIGS. 14A-14E show the use of YeRCOM for functional characterization of MERS nsp14 activity. (14A) Schematic showing the workflow of complementation assays. S. cerevisiae abd1::kanMX4 is transformed with pMO1 encoding native S. cerevisiae N7-MTase, abd1, and a plasmid encoding the viral N7-MTase. The yeast strain is subjected to 5-FOA treatment to cure the abd1 expressing plasmid. If the viral N7-MTase can catalyze the RNA cap-0 N7-methylation of S. cerevisiae mRNAs, then S. cerevisiae abd1::kanMX4 haploid strain will survive in the presence of 5-FOA. If the viral N7-MTase cannot catalyze the RNA cap-0 N7-methylation of S. cerevisiae mRNAs, then the strain will not survive in the presence of 5-FOA. (14B) The ability of WT MERS nsp14 and a series of mutants and truncations of MERS nsp14 were studied by transforming their respective plasmid construct in S. cerevisiae abd1::kanMX4 pMO1 haploid and monitoring their ability to recover growth in the presence of 5-FOA. Data represent three biological replicates. Plasmid name and details are listed. (14C) Representative strains listed in panel (14B) were grown in liquid selection medium in the presence of 5-FOA and the total growth after 96 hours was measured (OD600). Each circle represents an individual replicate. Bars represent the average of 4 replicates and error bars represent mean±SD. (14D) Enzymatic scheme demonstrating 5′ capping of S. cerevisiae mRNAs using S. cerevisiae enzymes (Ceg1, Cet1) and MERS Nsp14. (14E) Structure of MERS nsp14 predicted by SWISS model. The MERS nsp14 N7-methyltransferase (N7-MTase) domain, the exonuclease (ExoN) domain, and the C-terminal 17 amino acids are shown as labeled. Zinc ions are shown as spheres.

FIG. 15 shows MERS, SARS-CoV-1 and SARS-CoV-2 nsp14 protein sequence alignment. Amino acid residues (W293, D353, F365) are highly conserved residues that were targeted for mutagenesis of MERS nsp14. Sequence alignment was performed using Clustal Omega EMBL-EBI Multiple Sequence Alignment tool. See Madeira et al., Search and sequence analysis tools services from EMBL-EBI in 2022. Nucleic Acids Research. 2022, 50, 276-279. Genbank accession numbers for representative nsp14 sequences: MERS (NC_019843.3), SARS-CoV-1 (AY278741), and SARS-CoV-2 (MT318827.1), each of which is incorporated by reference herein in their entirety. For MERS SEQ ID NO: 24 is depicted. For SARS-CoV-1 SEQ ID NO: 301 is depicted. For SARS-CoV-2 SEQ ID NO: 302 is depicted.

FIGS. 16A-16D show the use of YeRCOM to test the activity of point mutations hypothesized to attenuate MERS nsp14 function. (16A) The ability of WT MERS nsp14 and these mutants of nsp14 to catalyze the N7-methylation of yeast mRNAs were studied by transforming their respective plasmid constructs in S. cerevisiae abd1::kanMX4 pMO1 haploid and monitoring their ability to recover growth in the presence of 5-FOA. Data represent three biological replicates. Plasmid name and details are listed. (16B) Representative strains listed in panel (16A) were grown in liquid selection medium in the presence of 5-FOA and the total growth after 96 hours was measured (OD600). Each circle represents an individual replicate. Bars represent the average of 4 replicates and error bars represent mean±SD. (16C) Growth curves of S. cerevisiae abd1::kanMX4 complemented by MERS nsp14 attenuated mutant identified in this study F365G nsp14 (label B) and WT nsp14 (label A). Both strains were cured prior to growth rate analysis. Each data point represents the mean of four biological replicates. Error bars represent mean±SD. (16D) MERS nsp14 homology model predicted by SWISS MODEL overlayed with SARS-CoV nsp14 crystal structure (pdb: 5C8T). Residues targeted for mutagenesis in this study are labeled: W293, D353, F365. ADOMET coenzyme shown as labeled.

FIGS. 17A-17C show the use of YeRCOM to test the N7-MTase activity of various truncations of MPV vD1. (17A) The ability of WT MPV vD1 and truncations to catalyze the N7-methylation of yeast mRNAs were studied by transforming their respective plasmid construct in S. cerevisiae abd1::kanMX4 pMO1 pMO42 haploid and monitoring their ability to recover growth in the presence of 5-FOA. Data represent three biological replicates. Plasmid name and details are listed. (17B) Representative strains listed in panel (17A) were grown in liquid selection medium in the presence of 5-FOA and the total growth after 48 hours was measured (OD600). Each circle represents an individual replicate. Bars represent the average of 4 replicates and error bars represent mean±SD. (17C) Crystal structure of VACV vD1+vD12 complex (pdb: 2VDW). vD12, vD1 (amino acids 783-844), vD1 (amino acids 498-590), and the rest of vD1 are labeled.

FIGS. 18A-18D show the use of YeRCOM for functional characterization of MPV vD1 N7-MTase activity. (18A) The ability of WT MPV vD1 and a series of mutants of vD1 to catalyze the N7-methylation of yeast mRNAs were studied by transforming their respective plasmid construct in S. cerevisiae abd1::kanMX4 pMO1 pMO42 haploid and monitoring their ability to recover growth in the presence of 5-FOA. Data represent three biological replicates. (18B) Representative strains listed in panel (18A) were grown in liquid selection medium in the presence of 5-FOA and the total growth after 48 hours was measured (OD600). Each circle represents an individual replicate. Bars represent the average of 4 replicates and error bars represent mean±SD. (18C) Growth curves of S. cerevisiae abd1::kanMX4 pMO42 complemented by MPV vD1 attenuation mutants identified in this study (D545A (label B), R548A (label C), R548K (label D), and Y683V (label E)) and WT MPV vD1 (label A). Each data point represents the mean of three biological replicates. Error bars represent mean±SD. (18D) Active site images of VACV vD1 (pdb: 2VDW) with ADOMET coenzyme. Residues targeted for MPV vD1 mutagenesis in this study are labeled: Y683, R548, R655, D620, and D598.

FIG. 19 shows attenuated activity of MPV vD1 D598A. S. cerevisiae abd1::kanMX4 pMO1 pMO42 pMO41 (WT vD1) haploid and S. cerevisiae abd1::kanMX4 pMO1 pMO42 pMO50 (vD1 598A) were grown in liquid selection medium in the presence of 5-FOA and the total growth after 96 hours was measured (OD600). Each circle represents an individual replicate. Bars represent the average of 4 replicates and error bars represent mean±SD.

FIGS. 20A-20F show the use of YeRCOM for functional characterization of ASFV NP868R N7-MTase activity. (20A-20B) The ability of WT ASFV NP868R and a series of mutants of NP868R to catalyze the N7-methylation of yeast mRNAs were studied by transforming their respective plasmid construct in S. cerevisiae abd1::kanMX4 pMO1 pMO42 haploid and monitoring their ability to recover growth in the presence of 5-FOA. Data represent three biological replicates. (20C) Representative strains listed in panel (20B) were grown in liquid selection medium in the presence of 5-FOA and the total growth after 120 hours was measured (OD600). Each circle represents an individual replicate. Bars represent the average of 3 replicates and error bars represent mean±SD. (20D) Additional biochemical characterization experiments were performed. The indicated mutants were transformed into S. cerevisiae abd1::kanMX4 pMO1 pMO42 haploid and grown in liquid selection medium in the presence of 5-FOA. Total growth after 120 hours was measured (OD600). Each circle represents an individual replicate. Bars represent the average of 3 replicates and error bars represent mean±SD. (20E) Growth curves of S. cerevisiae abd1::kanMX4 complemented by ASFV NP868R attenuation mutants identified in this study (K647Y (label B), F711W (label C), and D680L (label D)) in comparison to WT ASFV NP868R (label A). Each data point represents the mean of three biological replicates. Error bars represent mean±SD. (20F) Active site of ASFV NP868R N7-MTase (pdb: 7D8U) with ADOMET coenzyme shown. Residues targeted for MPV vD1 mutagenesis in this study are labeled: Y714, F711, D680, D646, and K647.

FIGS. 21A-21C show the use of YeRCOM to identify viral N7-MTase specific inhibitors. (21A) Human N7-MTase rnmt plasmid expression is able to recover S. cerevisiae abd1::kanMX4 haploid growth in the presence of 5-FOA. Data represent three biological replicates. (21B) This platform can be used to identify small molecule inhibitors of viral N7 MTases. S. cerevisiae abd1::kanMX4 pMO5 haploid can be used as a negative control to ensure small molecule inhibitors only inhibit viral N7 MTase activity, not RNMT activity. (21C) Growth rate perturbation of S. cerevisiae abd1::kanMX4 pMO65 (YeRCOM-MERS), S. cerevisiae abd1::kanMX4 pMO41 pMO42 (YeRCOM-MPV), and S. cerevisiae abd1::kanMX4 pMO5 (YeRCOM-human) in the presence of 25 and 50 uM disulfiram. Cultures were grown in liquid selection medium and the total growth after 48 hours was measured (OD600). Each circle represents an individual replicate. Bars represent the average of 4 replicates and error bars represent mean±SD.

FIGS. 22A-22B depict confirmation of the absence of abd1 expression (pMO1) from YeRCOM-human strain. (22A) S. cerevisiae abd1::kanMX4 pMO1 pMO5 was grown in the presence of 5-FOA for 48 hours and the resulting cured strain, S. cerevisiae abd1::kanMX4 pMO5, was grown in the presence and absence of uracil to verify the absence of pMO1. Without pMO1, uracil supplementation is required for growth since pMO1 contains an uracil biosynthetic gene marker. The total growth after 48 hours was measured (OD600). Each circle represents an individual replicate. Bars represent the average of 4 replicates and error bars represent mean±SD. (22B) PCR analysis was also used to verify the absence of pMO1 in cured S. cerevisiae abd1::kanMX4 pMO5 strain. Plasmids were isolated from S. cerevisiae abd1::kanMX4 pMO5 strain and amplified using primers specific to both rnmt gene (lane 1) and abd1 gene (lane 2). Only amplification using primers specific to rnmt gene yielded a PCR product. As a control, the abd1 expressing plasmid (pMO1) was also amplified using primers specific to both abd1 gene (lane 3) and rnmt gene (lane 4). Only amplification using primers specific to abd1 gene yielded a PCR product. Leftmost lane contains DNA ladder. rnmt size=1440 base pairs. abd1 size=1311 base pairs.

FIGS. 23A-23C depict (23A) The ability of WT WNV ns5 to catalyze the formation of the guanine cap on yeast mRNAs were studied by transforming pAM206 in S. cerevisiae ceg1::kanMX4 pAT4 haploid and monitoring its ability to recover growth in the presence of 5-FOA. S. cerevisiae ceg1::kanMX4 pAT4 pAT13 shows that mce1 is a needed domain in order for yeast to recover growth. Data represent three biological replicates. (23B) S. cerevisiae ceg1::kanMX4 pAT4 pAM206 (WT WNV) haploid and S. cerevisiae ceg1::kanMX4 pAT4 pAM206-F24X or pAM206-S150X were grown in liquid selection medium in the presence of 5-FOA and the total growth after 72 hours was measured (OD600). Each circle represents an individual replicate. Bars represent the average of 4 replicates and error bars represent mean±SD (23C) Growth curves of S. cerevisiae ceg1::kanMX4 pAT4 complemented by WNV ns5 attenuation mutant identified in this study (F24L (label B), WT WNV ns5 (label A). Each data point represents the mean of four biological replicates. Error bars represent mean±SD.

FIGS. 24A-24C depict (24A) The ability of WT SARS-Cov-2 nsp12 and a series of nsp12 mutants to catalyze the formation of the guanine cap on yeast mRNAs were studied by transforming their respective plasmid construct in S. cerevisiae ceg1::kanMX4 pAT4 haploid and monitoring their ability to recover growth in the presence of 5-FOA. Data represent three biological replicates. (24B) S. cerevisiae ceg1::kanMX4 pAT4 haploid expressing WT SARS-CoV-2 nsp12 and mutants were grown in liquid selection medium in the presence of 5-FOA and the total growth after 72 hours was measured (OD600). Each circle represents an individual replicate. Bars represent the average of 4 replicates and error bars represent mean±SD (24C) Summarization of the plasmids used in SARS-CoV-2 nsp12 biochemical characterization.

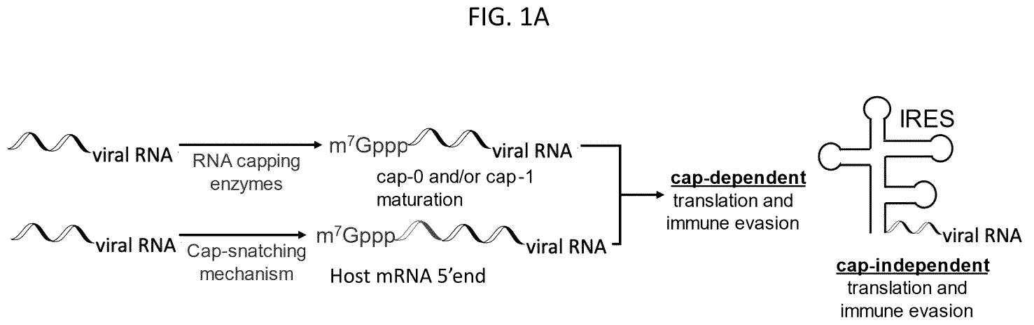

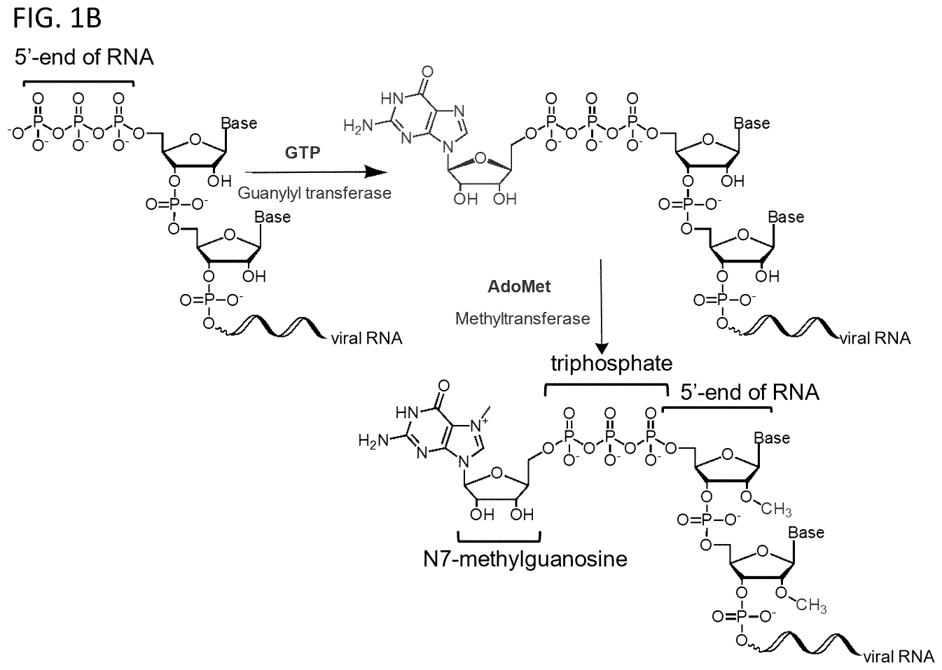

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.

In the accompanying sequence listing:
SEQ ID NO: 1 is an exemplary amino acid sequence for the Mce1 GTase domain.
SEQ ID NO: 2 is an exemplary nucleotide sequence encoding the Mce1 GTase domain.
SEQ ID NO: 3 is an exemplary nucleotide sequence encoding the pMO1 plasmid.
SEQ ID NO: 4 is an exemplary amino acid sequence for SARS-CoV-2 nsp14, with a N-terminal M.
SEQ ID NO: 5 is an exemplary nucleotide sequence encoding SARS-CoV-2 nsp14.
SEQ ID NO: 6 is an exemplary amino acid sequence for MPV D1.
SEQ ID NO: 7 is an exemplary nucleotide sequence encoding MPV D1.
SEQ ID NO: 8 is an exemplary amino acid sequence for MPV D12.
SEQ ID NO: 9 is an exemplary nucleotide sequence encoding MPV D12.
SEQ ID NO: 10 is an exemplary amino acid sequence for ASFV NP868R.
SEQ ID NO: 11 is an exemplary nucleotide sequence encoding ASFV NP868R.
SEQ ID NO: 12 is an exemplary amino acid sequence for WNV ns5.
SEQ ID NO: 13 is an exemplary nucleotide sequence encoding a pTpl1 promoter.
SEQ ID NO: 14 is an exemplary amino acid sequence for RNMT.
SEQ ID NO: 15 is an exemplary nucleotide sequence encoding RNMT.
SEQ ID NO: 16: is an exemplary nucleotide sequence encoding the pMO2 plasmid.
SEQ ID NO: 17 is an exemplary nucleotide sequence encoding the pMO41 plasmid.
SEQ ID NO: 18 is an exemplary amino acid sequence encoding the pMO42 plasmid.
SEQ ID NO: 19 is an exemplary nucleotide sequence encoding the pAT15 plasmid.
SEQ ID NO: 20 is an exemplary nucleotide sequence encoding an aspect of the pTpl1 promoter.
SEQ ID NO: 21 is an exemplary nucleotide sequence encoding the pMO5 plasmid.
SEQ ID NO: 22 is an exemplary nucleotide sequence encoding the pMO65 plasmid.
SEQ ID NO: 23 is an exemplary nucleotide sequence encoding the pMO32 plasmid.
SEQ ID NO: 24 is an exemplary amino acid sequence for MERS-CoV nsp14.
SEQ ID NO: 25 is an exemplary nucleotide sequence encoding MERS-CoV nsp14.
SEQ ID NO: 26 is an exemplary nucleotide sequence encoding the pAT8 plasmid.
SEQ ID NO: 27: is an exemplary nucleotide sequence encoding pAT13.
SEQ ID NO: 28 is an exemplary nucleotide sequence encoding the pAT4 plasmid.
SEQ ID NO: 29 is an exemplary amino acid sequence for SARS-CoV-2 nsp12, with a N-terminal M.
SEQ ID NO: 30 is an exemplary nucleotide sequence for SARS-CoV-2 nsp12.
SEQ ID NO: 31 is an exemplary amino acid sequence for WNV ns5, with a N-terminal M.
SEQ ID NO: 32 is an exemplary nucleotide sequence encoding WNV ns5.
SEQ ID NO: 33 is an exemplary amino acid sequence for MCE1.
SEQ ID NO: 34 is an exemplary amino acid sequence for SARS-CoV-2 nsp9.
SEQ ID NO: 35 is an exemplary nucleotide sequence for Saccharomyces cerevisiae abd1.
SEQ ID NO: 36 is an exemplary amino acid sequence for Saccharomyces cerevisiae Abd1.
SEQ ID NO: 37 is an exemplary nucleotide sequence encoding Saccharomyces cerevisiae ceg1.
SEQ ID NO: 38 is an exemplary amino acid sequence for Saccharomyces cerevisiae Ceg1p.
SEQ ID NO: 39 is an exemplary nucleotide sequence encoding Saccharomyces cerevisiae cet1.
SEQ ID NO: 40 is an exemplary amino acid sequence for Saccharomyces cerevisiae Cet1p.
SEQ ID NO: 41 is an exemplary nsp14 SARS-CoV-2 nucleotide sequence, taken from GenBank MT318827.1
SEQ ID NO: 42 is an exemplary nsp12 SARS-CoV-2 nucleotide sequence, taken from GenBank MT318827.1
SEQ ID NO: 43 is an exemplary nsp9 SARS-CoV-2 nucleotide sequence, taken from GenBank MT318827.1
SEQ ID NO: 44 is an exemplary nsp14 MERS-CoV nucleotide sequence, taken from NCBI NC_019843.3.
SEQ ID NO: 45 is an exemplary NP868R ASFV nucleotide sequence, taken from NCBI NC_001659.2.
SEQ ID NO: 46 is an exemplary D1 MPV nucleotide sequence, taken from GenBank MT903343.1
SEQ ID NO: 47 is an exemplary D12 MPV nucleotide sequence, taken from GenBank MT903343.1
SEQ ID NO: 48 is an exemplary ns5 WNV nucleotide sequence, taken from NCBI NC_009942.1
SEQ ID NO: 49 is an exemplary nucleotide sequence encoding the pMO3 plasmid.
SEQ ID NO: 50 is an exemplary nucleotide sequence encoding the pROS1 plasmid.
SEQ ID NO: 51 is an exemplary nucleotide sequence encoding the pROS2 plasmid.
SEQ ID NO: 52 is an exemplary nucleotide sequence encoding the pROS8 plasmid.
SEQ ID NO: 53 is an exemplary nucleotide sequence encoding the pROS12 plasmid.
SEQ ID NO: 54 is an exemplary nucleotide sequence encoding the pROS13 plasmid.
SEQ ID NO: 55 is an exemplary nucleotide sequence encoding the pROS14 plasmid.
SEQ ID NOs: 56-134 are listed in Example 1
SEQ ID NO: 135 is an exemplary nucleotide sequence encoding the pIJI1 plasmid.
SEQ ID NO: 136 is an exemplary nucleotide sequence encoding the pIJ12 plasmid.
SEQ ID NO: 137 is an exemplary nucleotide sequence encoding the pIJ13 plasmid.
SEQ ID NO: 138 is an exemplary nucleotide sequence encoding the pMO66 plasmid.
SEQ ID NO: 139 is an exemplary nucleotide sequence encoding the pMO67 plasmid.
SEQ ID NO: 140 is an exemplary nucleotide sequence encoding the pMO68 plasmid.
SEQ ID NOs: 141-240 can be found in example 7.
SEQ ID NO: 241 is an exemplary nucleotide sequence encoding the pAM206 plasmid.
SEQ ID NO: 242 is an exemplary nucleotide sequence encoding the pAT37 plasmid.
SEQ ID NO: 243 is an exemplary nucleotide sequence encoding the pMO35 plasmid.
SEQ ID NO: 244 is an exemplary nucleotide sequence encoding the pMO37 plasmid.
SEQ ID NOs: 245-284 can be found in Example 13
SEQ ID NO: 285 is an exemplary amino acid sequence for Sus scrofa RNMT.
SEQ ID NO: 286 is an exemplary amino acid sequence for Sus scrofa RNMT.
SEQ ID NO: 287 is an exemplary amino acid sequence for Sus scrofa MCE1.
SEQ ID NO: 288 is an exemplary amino acid sequence for MPV D1, with a N-terminal M.
SEQ ID NO: 289 is an exemplary amino acid sequence for SARS-CoV-2 nsp14.
SEQ ID NO: 290 is an exemplary amino acid sequence for MPV D1.
SEQ ID NO: 291 is an exemplary amino acid sequence for SARS-CoV-2 nsp12.
SEQ ID NO: 292 is an exemplary amino acid sequence for WNV ns5.
SEQ ID NO: 293 is an exemplary amino acid sequence for an aspect of SARS-CoV-1 nsp14, taken from AY278741.
SEQ ID NO: 294 is an exemplary amino acid sequence for an aspect of SARS-CoV-2 nsp14, taken from MT318827.1.
SEQ ID NO: 295 is an exemplary amino acid sequence for an aspect of Pangolin CoV nsp14, taken from MT121216.1.
SEQ ID NO: 296 is an exemplary amino acid sequence for an aspect of Bat CoV nsp14, taken from MN996532.
SEQ ID NO: 297 is an exemplary amino acid sequence for an aspect of Magpie-robin-CoV nsp14, taken from 005352853.
SEQ ID NO: 298 is an exemplary amino acid sequence for an aspect of Canada Goose CoV nsp14, taken from QCB65096.
SEQ ID NO: 299 is an exemplary amino acid sequence for an aspect of Mink CoV nsp14, taken from 009019180.
SEQ ID NO: 300 is an exemplary amino acid sequence for an aspect of Erinaceus hedgehog CoV nsp14, taken from QGA70691.
SEQ ID NO: 301 is an exemplary amino acid sequence for SARS-CoV-1 nsp14, with a N-terminal M.
SEQ ID NO: 302 is an exemplary amino acid sequence for SARS-CoV-2 nsp14, with a N-terminal M.

Exemplary amino acid sequences including an N-terminal M are disclosed herein. Of these examples, sequences which lack the N-terminal M are also contemplated herein.

### DETAILED DESCRIPTION

### I. Introduction

Zoonotic pathogenic viruses and animal viruses pose a threat to human and animal health. There is an increasing risk of emerging infectious diseases being transmitted from wild and domesticated animals to humans. In addition to this, viral host jumps from wild animals hosts to domesticated animals is a significant threat to domestic and food industry. Several factors are suggested to have contributed to the increasing risks, including growth of human population, geographic expansion of human habitats, climate change, expansion of agriculture and deforestation amongst others.^{1,2} Over the past several decades, around 60% to 70% of the incidences related to emerging infectious diseases in humans are zoonotic in nature and are caused by zoonotic viruses or drug-resistant pathogens.^{2,3} Often, this sudden emergence of zoonotic pathogens leads to disease outbreaks that are challenging to contain. One reason for this is the absence of effective control measures such as broad-spectrum therapeutics or vaccines targeting these zoonotic pathogens. Broad-spectrum antiviral development approaches often target pathways that are highly conserved in viral replication and propagation. This is because the antivirals that are developed using this approach can often be repurposed to combat viral pathogens that have recently made zoonotic jump. For example, Remdesivir was initially used to treat Ebola, Hepatitis C and other viral infections, but was then repurposed to combat SARS-CoV-2. Similarly, Paxlovid (i.e., Nirmatrevir) was derived from Pfizer's 3CL protease inhibitor development pipeline. Prior to COVID-19 pandemic, 3CL protease inhibitors had shown promise against feline coronaviruses. These broad-spectrum inhibitor development efforts eventually resulted in the identification and development of Paxlovid for COVID-19 treatment. There is an increasing need to develop similar broad-spectrum antivirals targeting other viral replication and propagation mechanisms. This disclosure provides yeast-based phenotypic platforms for characterization and targeting of viral genome encoded essential RNA capping enzymes from emerging viral pathogens. These platforms can be used to identify and develop broad-spectrum antivirals targeting highly conserved RNA capping enzymes as well as identify attenuation mutations having implications on virus biocontainment as well as live attenuated vaccine development.

Most RNA virus genomes contain a 5′ RNA cap structure that is identical to their eukaryotic host mRNA cap. This 5′-end cap structure consists of 7-methylguanosine (m7G) linked to first nucleotide of the transcript mRNA via 5′-5′ triphosphate bridge.⁴ Conventional steps in RNA cap maturation involve RNA triphosphatase mediated cleavage of the B-phosphate and γ-phosphate, guanylyl transferase catalyzed linkage of guanosine linked to first nucleotide of the transcript mRNA via 5′-5′ triphosphate bridge and importantly, the methyltransferase that catalyzes the methylation at the N7 position of guanine at cap-0 and 2′-O methylation at cap-1 position using S-adenosylmethionine (AdoMet) as a cofactor. This cap structure is implicated in efficient recognition of the viral genomic RNA or viral mRNA transcripts by the eukaryotic translation initiation factor 4E (elF4E) for translation initiation.⁵⁶ During the course of viral/host evolution the RNA viruses evolved a 5′ RNA genome cap structure that is identical to the host mRNAs to disguise the viral genome as mRNA molecules and ensure viral genome translation and evade innate immune responses. In addition to the presence of RNA capping enzymes in emerging RNA virus pathogens, RNA capping enzymes have also been annotated in several families of emerging pathogenic DNA virus families like poxviridae. In case of DNA viruses, the RNA capping enzymes cap most (if not all) of the DNA virus mRNA transcripts with a cap structure that is identical to the host mRNAs. Therefore, unlike RNA viruses where the RNA capping enzymes are involved in capping the viral genome, in case of DNA viruses, the viral RNA capping enzymes are involved in capping of the viral mRNA transcripts. In both DNA and RNA viruses, the viral genome encoded RNA capping enzymes are essential for viral propagation. Even though the viral genome or mRNA cap structure is identical to the host mRNAs, the mechanistic and structural details of viral genome encoded, RNA capping enzymes are often distinct from their host RNA capping enzymes.^{7,8} Therefore, these viral genome-encoded proteins have been a long-standing target of antiviral development efforts.⁹ Usually in vitro enzyme assays or pathogenic replicons of viruses have been used to screen for inhibitors of RNA capping enzymes.^{10,11} However, these translational efforts are often hindered due to the unavailability of robust platforms that can be used in standard laboratory setting and are compatible with high-throughput screening technologies.

In the instant disclosure, a synthetic biology approach was used to develop yeast-based phenotypic platforms (such as YeRCOM, platforms for guanylyltransferases, and platforms for RNA triphosphatases) for characterization and targeting RNA capping enzymes from several emerging pathogens including MERS-CoV, Monkeypox virus, SARS-CoV-2, WNV, and African Swine Fever virus. To make these platforms applicable to identify broad-spectrum inhibitors that selectively target viral RNA capping enzymes over human RNA capping enzymes, the yeast-based phenotypic platforms were expanded to human RNA capping enzymes to demonstrate the feasibility of this approach to perform phenotypic screening to identify inhibitors of RNA capping enzymes.

These platforms are also used for directed evolution of RNA capping enzymes emerging pathogens to identify attenuation mutations in RNA capping viral enzymes. Because of high sequence similarity of RNA capping enzymes in these emerging pathogenic viruses, these observations have implications on broad-spectrum antiviral development, live attenuated vaccine development and virus biocontainment.

### REFERENCES INTRODUCTION

1. Jones, K. E. et al. Global trends in emerging infectious diseases. Nature 451, 990-993 (2008).
2. Bean, A. G. et al. Studying immunity to zoonotic diseases in the natural host—keeping it real. Nat. Rev. Immunol. 13, 851-861 (2013).
3. Salyer, S. J., Silver, R., Simone, K. & Behravesh, C. B. Prioritizing zoonoses for global health capacity building—themes from One Health zoonotic disease workshops in 7 countries, 2014-2016. Emerg. Infect. Dis. 23, S55 (2017).
4. Ramanathan, A., Robb, G. B. & Chan, S.-H. mRNA capping: biological functions and applications. Nucleic Acids Res. 44, 7511-7526 (2016).
5. Filipowicz, W. et al. A protein binding the methylated 5′-terminal sequence, m7GpppN, of eukaryotic messenger RNA. Proc. Natl. Acad. Sci. 73, 1559-1563 (1976).
6. Sheth, U. & Parker, R. Decapping and decay of messenger RNA occur in cytoplasmic processing bodies. Science (2003).
7. Otava, T. et al. The Structure-Based Design of SARS-CoV-2 nsp14 Methyltransferase Ligands Yields Nanomolar Inhibitors. ACS Infect. Dis. 7, 2214-2220 (2021).
8. Devkota, K. et al. Probing the SAM Binding Site of SARS-CoV-2 Nsp14 In Vitro Using SAM Competitive Inhibitors Guides Developing Selective Bisubstrate Inhibitors. SLAS Discov. Adv. Sci. Drug Discov. 26, 1200-1211 (2021).
9. Chen, Y. et al. Functional screen reveals SARS coronavirus nonstructural protein nsp14 as a novel cap N7 methyltransferase. Proc. Natl. Acad. Sci. U.S.A 106, 3484-3489 (2009).
10. Ogando, N. S. et al. The Enzymatic Activity of the nsp14 Exoribonuclease Is Critical for Replication of MERS-CoV and SARS-CoV-2. J. Virol. 94, e01246-20 (2020).
11. Lauring, A. S., Jones, J. O. & Andino, R. Rationalizing the development of live attenuated virus vaccines. Nat. Biotechnol. 28, 573-579 (2010).

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

About: Unless context indicated otherwise, “about” refers to plus or minus 5% of a reference value. For example, “about” 100 refers to 95 to 105.

Abd1: mRNA (guanine-N7)-methyltransferase. A yeast methyltransferase which catalyzes the transfer of a methyl group from S-adenosylmethionine to the GpppN terminus of capped mRNA. Gene name abd1. S. cerevisiae NCBI Gene ID: 852538; Exemplary sequence NCBI Ref Seq: NP_009795.3. SEQ ID NO: 34 provides an exemplary nucleotide sequence. SEQ ID NO: 35 provides an exemplary amino acid sequence.

Adjuvant: A component used to enhance antigenicity, for example of the attenuated viruses provided herein. In some examples, an adjuvant can include a suspension of minerals (alum, aluminum hydroxide, or phosphate) on which antigen is adsorbed; or water-in-oil emulsion, for example, in which antigen solution is emulsified in mineral oil (Freund's incomplete adjuvant), sometimes with the inclusion of killed mycobacteria (Freund's complete adjuvant) to further enhance antigenicity (inhibits degradation of antigen and/or causes influx of macrophages). In some examples, the adjuvant used in an immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants of use include the QS21 purified plant extract, Matrix M, AS01, MF59, and ALFQ adjuvants.

Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists.

Administration: To provide or give a subject an agent, such as an attenuated virus described herein, by any effective route. Administration can be local or systemic. Exemplary routes of administration include, but are not limited to, injection (such as subcutaneous, intramuscular, intradermal, intraperitoneal, intratumoral, intraprostatic, intrathecal, intraosseous, and intravenous), oral, sublingual, rectal, transdermal, intranasal, vaginal and inhalation routes.

African Swine Fever Virus (ASFV): A highly contagious virus that causes viral hemorrhagic disease in swine with high mortality rates. ASFV is a linear double stranded DNA virus in the Asfarviridae family. ASFV is classified as a nucleocytoplasmic large DNA virus (NCLDV) because it replicates in the cytosol of mammalian cells, particularly monocyte/macrophage lineages of domestic and wild pigs. ASFV has a genome size of about 170-190 kb (depending on the strain) encoding 151-167 open reading frames with limited associated known or predicted functions. ASFV encodes RNA capping enzymes, such as NP868R.

Amino acid substitution: The replacement of one amino acid in a polypeptide (such as a viral protein, such as a SARS-CoV-2 protein, such as an RNA cap-0 (guanine-N7)-methyltransferase protein, such as a nsp14 protein) with a different amino acid, such as replacement of a tryptophan with a phenylalanine. In some examples, such a replacement is achieved by altering the coding sequence at the appropriate codon.

Attenuated: A virus that is “attenuated” or that has an “attenuated phenotype” refers to a virus that has decreased virulence compared to a reference virus under similar conditions of infection. Attenuation usually is associated with decreased virus replication as compared to replication of a reference wild-type virus under similar conditions of infection, and thus “attenuation” and “restricted replication” often are used synonymously. In some hosts (typically non-natural hosts, including experimental animals), disease is not evident during infection with a reference virus in question, and restriction of virus replication can be used as a surrogate marker for attenuation.

Ceg1p: mRNA guanylyltransferase. A yeast guanylyltransferase which plays a role in mRNA 5′ capping. Ceg1p forms a heterotetramer with Cet1p. Gene name ceg1. S. cerevisiae NCBI Gene ID: 852747; Exemplary sequence NCBI Ref Seq: NP_011385.3. SEQ ID NO: 37 provides an exemplary nucleotide sequence. SEQ ID NO: 38 provides an exemplary amino acid sequence.

Cet1p: Polynucleotide 5′-phosphatase. A yeast RNA 5′-triphosphatase which plays a role in mRNA 5′ capping. Cet1p forms a heterotetramer with Ceg1p. Gene name cet1. S. cerevisiae NCBI Gene ID: 855873; Exemplary sequence NCBI Ref Seq: NP_015096.1 SEQ ID NO: 39 provides an exemplary nucleotide sequence. SEQ ID NO: 40 provides an exemplary amino acid sequence.

Contacting: Placement in direct physical association; includes both in solid and liquid form, which can take place either in vivo or in vitro. Contacting includes contact between one molecule and another molecule, for example between an antiviral compound and a cell, such as a stable cell clone harboring an isolated non-native coronavirus genome disclosed herein.

Control: A reference standard. In some examples, the control is a negative control sample, such as an untreated yeast cell including a heterologous methyltransferase. In other examples, the control is a positive control sample, such as a yeast cell including a heterologous methyltransferase treated with a molecule having a known activity, such as a known compound which inhibits methyltransferase activity. In some examples the control is a historical control or standard reference value or range of values (such as a previously tested control sample).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus (CoV): A large family of positive-sense, single-stranded RNA viruses that can infect humans and non-human animals. Coronaviruses are part of the phylogenetic family Coronaviridae. Coronaviruses have been organized into four groups: alphacoronaviruses (α-CoVs), betacoronaviruses (β-CoVs), gammacoronaviruses (γ-CoVs), and deltacoronaviruses (Δ-CoVs). Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronavirus is the Swine Delta Coronavirus (SDCV).

Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV-1), SARS-CoV-2, and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), and human coronavirus NL63 (NL63-CoV).

A coronavirus genome may be non-native, such as a non-native SARS-CoV-2 genome. A non-native coronavirus genome is genetically modified from a corresponding wild-type (native) coronavirus genome. For example, a non-native SARS-CoV-2 genome may include additional genes not present in a corresponding wild-type SARS-CoV-2 genome, may include genetically inactivated SARS-CoV-2 genes, and/or may include substitutions, such as a nsp14 gene encoding W293F, F368N, F368L, D353T, and/or D353A substitutions. Another example of a coronavirus genome is a non-native MERS-CoV genome, such as a non-native MERS-CoV genome with a nsp14 gene encoding F365G. Another example coronavirus genome is a non-native SARS-CoV-2 genome with a nsp12 gene encoding K73A, D218A, D760A, V30A, R33A, R33L, R33K, R55A, R55L, R55K, K50A, C53A, R116A, V71A, N209A, L119A, and/or Y217F.

A coronavirus genome, such as a non-native coronavirus genome, may replicate autonomously inside a cell. In some examples, a non-native SARS-CoV-2 genome is a variant of SARS-CoV-2 (such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (such as original lineage: B.1.1.529 and lineages: BA.2, BA.4, BA.5, BQ.1, BQ.1.1, BA.4.6, and BF.7)).

COVID-19: A contagious disease caused by severe acute respiratory syndrome coronavirus 2 (SARS-CoV-2). Symptoms of COVID-19 are variable, but often include fever, cough, fatigue, breathing difficulties, and loss of smell and taste. Symptoms can begin one to fourteen days after exposure to the virus. Around one in five infected individuals do not develop any symptoms. While most people have mild symptoms, some people develop acute respiratory distress syndrome (ARDS). ARDS can be precipitated by cytokine storms, multi-organ failure, septic shock, and blood clots. Longer-term damage to organs (in particular, the lungs and heart) has been observed. A significant number of patients recover from the acute phase of the disease but continue to experience a range of effects—known as long COVID—for months afterwards. These effects include severe fatigue, memory loss and other cognitive issues, low-grade fever, muscle weakness, and breathlessness.

D1 and D12: Also known as vD1 and vD12, these proteins form a heterodimer which carries out three steps in mRNA capping. D1 encodes the methyltransferase active site but requires the D12 stimulatory subunit for efficient activity.

Exemplary MPV D1 sequences include GenBank: MT903343.1 locus tag MPXV-UK_P1-096. An additional exemplary MPV D1 sequence is SEQ ID NO: 6. Exemplary MPV D12 sequences include GenBank: MT903343.1 locus tag MPXV-UK_P1-107. An additional exemplary MPV D12 sequence is SEQ ID NO: 8.

Effective amount: The amount of an agent (such as an attenuated virus disclosed herein) that is sufficient to effect beneficial or desired results. An effective amount (also referred to as a therapeutically effective amount) may vary depending upon one or more of: the subject and disease condition being treated, the weight and age of the subject, the severity of the disease condition, the manner of administration and the like. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one aspect, an “effective amount” of a therapeutic agent (e.g., an attenuated virus) is an amount sufficient to reduce viral infection, reduce viral load upon infection, reduce symptoms of viral infection, or reduce viral transmissibility upon infection by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent).

In one aspect, an “effective amount” of a therapeutic agent is an amount sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed therapeutic agent can be the amount of the immunogen sufficient to elicit a priming immune response in a subject that can be subsequently boosted with the same or a different immunogen to elicit a protective immune response.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. Regulation of expression can include controls on transcription, translation, RNA transport and processing, degradation of intermediary molecules such as mRNA, or through activation, inactivation, compartmentalization or degradation of specific protein molecules after they are produced.

Flavivirus: A genus of viruses in the family Flaviviridae. Flaviviruses have a positive-sense, single-stranded RNA genome, an icosahedral capsid and an outer envelope. Most flaviviruses are transmitted via the bite of a mosquito or tick. Translation of the viral genome produces a single polyprotein. The genome of most flaviviruses encodes three structural proteins (capsid, premembrane and envelope) and seven non-structural proteins (NS1, NS2A, NS2B, NS3, NS4A, NS4B and NS5). Flaviviruses include, but are not limited to, dengue virus (DENV), Zika virus (ZIKV), yellow fever virus (YFV), West Nile virus (WNV), Japanese encephalitis virus (JEV), Powassan virus (POWV), St. Louis encephalitis virus (SLEV), Usutu vims (USUV), Rocio virus (ROCV), and Ilheus virus (ILHV). Exemplary Flavivirus genomes include GenBank Accession Nos. KR052012.1 (DENV), MW015936.1 (ZIKV), NC_002031.1 (YFV), and L48961.1 (JEV).

Genetic inactivation or down-regulation: When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene down-regulation or deactivation includes processes that decrease transcription of a gene or translation of mRNA.

For example, a mutation, such as a substitution, partial or complete deletion, insertion, or other variation, can be made to a gene sequence that significantly reduces (and in some cases eliminates) production of the gene product or renders the gene product substantially or completely non-functional. For example, a genetic inactivation of a gene encoding a yeast Abd1 gene results in the yeast having a non-functional or non-detectable native Abd1 protein. Genetic inactivation is also referred to herein as “functional deletion”.

Guanylyltransferase (or guanylyl transferase): An enzyme that attaches a guanosine mono phosphate (GMP) group to its substrate. In some examples the guanylyltransferase is an RNA guanylyltransferase. In some examples the guanylyltransferase is a guanylyltransferase involved in RNA capping. In some examples, a guanylyltransferase attaches a GMP group to the 5′-end of an RNA, such as an mRNA. In some examples the guanylyltransferase catalyzes linkage of guanosine linked to first nucleotide of the transcript mRNA via 5′-5′ triphosphate bridge. Exemplary guanylyltransferases include Ceg1p, nsp12, ns5, NP868R, D1, and MCE1.

Exemplary guanylyltransferase amino acid sequences include GenBank Accession Nos. YP_459941.1 (HCoV-HKU1 nsp12), NP_003791.3 (H. sapiens MCE1 isoform a), NP_001273355.1 (H. sapiens MCE1 isoform b), NP_036014.1 (M. musculus MCE1 isoform 1), NP_001292202.1 (M. musculus MCE1 isoform 2), and XP_020945593.1 (S. scrofa MCE1). Further exemplary guanylyltransferase amino acid sequences include SEQ ID NO: 29 (SARS-CoV-2 nsp12), SEQ ID NO: 31 (WNV ns5), SEQ ID NO: 292 (WNV ns5), and SEQ ID NO: 287 (Sus scrofa MCE1).

Heterologous: A heterologous protein or polypeptide refers to a protein or polypeptide derived from a different source or species. A heterologous nucleic acid molecule refers to a nucleic acid molecule derived from a different source or species. Thus, a heterologous protein, polypeptide, or nucleic acid molecule in a cell, refers to a protein, polypeptide, or nucleic acid molecule not naturally found in the cell in nature (e.g., an exogenous protein, polypeptide, or nucleic acid molecule). In some examples, a cell expressing a heterologous protein, polypeptide, or nucleic acid molecule is transgenic. In some examples, a heterologous protein or polypeptide includes a non-native protein. A “non-native” protein or polypeptide is one which cannot be found in a naturally occurring instance of the host organism.

Immune response: A response of a cell of the immune system, such as a B cell, T cell, or monocyte, to a stimulus. In one example, the response is specific for a particular antigen (an “antigen-specific response”). In one example, an immune response is a T cell response, such as a CD4+ response or a CD8+ response. In another example, the response is a B cell response, and results in the production of specific antibodies.

Immunogenic composition: A preparation of immunogenic material capable of stimulating an immune response, which in some examples can be administered for the prevention, amelioration, or treatment of infectious or other types of disease. The immunogenic material may include attenuated or killed microorganisms (such as bacteria or viruses), or antigenic proteins, peptides or DNA derived from them. Immunogenic compositions comprise an antigen (such as an attenuated virus provided herein) that induces a measurable T cell response against the antigen, or induces a measurable B cell response (such as production of antibodies) against the antigen. In one example, an immunogenic composition is an attenuated virus provided herein, which induces a measurable CTL response against the isolated virus, or another virus, or induces a measurable B cell response (such as production of antibodies) against the isolated virus, or another virus when administered to a subject. For in vivo use, the immunogenic composition typically includes a recombinant virus in a pharmaceutically acceptable carrier and may also include other agents, such as an adjuvant.

Increase or Decrease: A positive or negative change, respectively, in quantity from a control value (such as a value representing no therapeutic agent). An increase is a positive change, such as an increase at least 25%, at least 50%, at least 100%, at least 200%, at least 300%, at least 400% or at least 500%, as compared to the control value. A decrease is a negative change, such as a decrease of at least 10%, at least 20%, at least 30%, at least 40%, at least 50%, at least 60%, at least 70%, at least 75%, at least 80%, at least 85%, at least 90%, at least 95%, at least 98%, at least 99%, or at least 100% decrease as compared to a control value. In some examples, the increase or decrease is statistically significant relative to a suitable control.

Isolated: A biological component (such as a nucleic acid molecule, protein, virus, or cell) that has been substantially separated, produced apart from, or purified away from other biological components in the cell of the organism (or in the organism) in which the component occurs, such as other chromosomal and extra-chromosomal DNA and RNA, and proteins. Thus, isolated nucleic acid molecules, viruses, and proteins include nucleic acid molecules, viruses, and proteins purified by standard purification methods. Similarly, an isolated host cell (or populations of cells) includes cells purified by standard purification methods from the organism or tissue in which they typically reside. The term also embraces nucleic acids and proteins prepared by recombinant expression in a host cell, as well as chemically synthesized nucleic acids and proteins. An isolated nucleic acid molecule, virus, protein, or host cell, such as a non-native yeast provided herein or host cell containing such, can be at least 50%, at least 60%, at least 70%, at least 80%, at least 90%, at least 95%, at least 96%, at least 97%, at least 98%, at least 99%, at least 99.9%, or at least 99.99% pure.

Marker: A marker gene as used herein, such as a selectable marker, is a gene, which when introduced into a cell, confers a trait suitable for artificial selection of cells exhibiting the trait. Positive markers are selectable markers that confer selective advantage to the host cell, such as antibiotic resistance. An antibiotic or antifungal resistance gene (the selectable marker gene) produces a protein that provides cells expressing the protein with resistance to a particular antibiotic or antifungal. An antibiotic resistance gene may confer resistance to neomycin (such as a neomycin phosphotransferase gene), kanamycin, geneticin, ampicillin, or another antibiotic. Exemplary selectable marker genes include Neo (confers resistance to geneticin), bsd (confers resistance to blasticidin), hygB d (confers resistance to hygromycin B), pac (confers resistance to puromycin), and Sh bla (confers resistance to zeocin). An antifungal resistance gene may confer resistance to Zeocin™ or cycloheximide, kanamycin, or many common antibiotics such as those listed above. Any of such can be present in a non-native yeast disclosed herein, for example in place of Abd1 or Ceg1p genes.

Negative (or counterselectable) markers are selectable markers that eliminate or inhibit growth of the host cell upon selection, while positive and negative selectable markers can serve as both a positive and a negative marker by conferring an advantage to the host cell under one condition, and inhibiting growth under a different condition. For example, the ura3 gene encodes the enzyme ODCase, which is important for the synthesis of ribonucleotides in yeast, and permits growth in the absence of uracil or uridine. However, if 5-Fluoroorotic acid (5-FOA) is present ODCase will catalyze its conversion into the toxic compound 5-fluorouracil, causing cell death.

An antibiotic or antifungal resistance cassette is a nucleic acid sequence encoding a selectable marker which confers resistance to that antibiotic in a host cell in which the nucleic acid is translated. Examples of antibiotic or antifungal resistance cassettes include, but are not limited to: kanamycin, ampicillin, tetracycline, chloramphenicol, neomycin, hygromycin and zeocin.

Methyltransferase: An enzyme which transfers a methyl group to its substrate. In some examples the methyltransferase is a methyltransferase involved in RNA capping. In some examples the methyltransferase is an RNA methyltransferase. In some examples the methyltransferase is an RNA guanine-N7 methyltransferase. In some examples, a methyltransferase catalyzes methylation at the N7 position of guanine at cap-0 and 2′-O methylation at cap-1 position. In some examples, S-adenosylmethionine (AdoMet) is a methyltransferase cofactor. In some examples the methyltransferase is a domain in a protein. Exemplary methyltransferases include: Abd1, nsp14, D1, D12, D1 and D12, NP868R, ns5, and mRNA cap methyltransferase (RNMT).

Exemplary methyltransferase amino acid sequences include GenBank Accession Nos. CAA85199.1 (S. cerevisiae Abd1), WBR50393.1 (ASFV NP868R), NP_001295192.1 (H. sapiens RNMT isoform 1), NP_003790.1 (H. sapiens RNMT isoform 2), NP_001365061.1 (H. sapiens RNMT isoform 3), NP_080716.1 (M. musculus RNMT isoform 1), NP_001164424.1 (M. musculus RNMT isoform 2), and XP_020951674.1 (S. scrofa RNMT). Further exemplary methyltransferase amino acid sequences include SEQ ID NO: 4 (SARS-CoV-2 nsp14), SEQ ID NO: 289 (SARS-CoV-2 nsp14), SEQ ID NO: 6 (MPV D1), SEQ ID NO: 8 (MVP D12), SEQ ID NO: 285 (S. scrofa RNMT), and SEQ ID NO: 286 (S. scrofa RNMT).

MCE1: Provides mRNA guanylyltransferase activity and triphosphatase activity to 7-methylguanosine mRNA capping. Gene name MCE1. H. sapiens Gene ID: 8732. Exemplary guanylyltransferase amino acid sequences include GenBank Accession Nos. NP_003791.3 (H. sapiens MCE1 isoform a), NP_001273355.1 (H. sapiens MCE1 isoform b), NP_036014.1 (M. musculus MCE1 isoform 1), NP_001292202.1 (M. musculus MCE1 isoform 2), XP_020945593.1 (S. scrofa MCE1). Further exemplary guanylyltransferase amino acid sequences include SEQ ID NO: 287 (Sus scrofa MCE1).

Middle East respiratory syndrome-related coronavirus (MERS-CoV): A positive-sense, single stranded RNA virus of the genus Betacoronavirus which causes a highly fatal cause of severe acute respiratory infection. The viral genome is capped, polyadenylated, and covered with nucleocapsid proteins. The MERS-CoV virion includes a viral envelope with large spike glycoproteins. The MERS-CoV genome, like most coronaviruses, has a common genome organization with the replicase gene included in the 5′-two thirds of the genome, and structural genes included in the 3′-third of the genome. The MERS-CoV genome encodes the canonical set of structural protein genes in the order 5′-spike (S)-envelope (E)-membrane (M) and nucleocapsid (N)-3′. MERS-CoV genomes are currently classified as Clade A or Clade B MERS-CoV. Examples of Clade A and Clade B viruses include the JordanN3/2012 (GenBank ID: KC776174) and England_Qatar/2012 (GenBank ID: KC667074) strains, respectively.

Monkeypox virus (MPV): A member of the Poxviridae family, which are double stranded DNA viruses. Poxviridae DNA genomes typically encode highly conserved RNA capping enzymes for capping viral RNA transcripts. MPV's genome is around 200 kb, and encodes approximately 200 proteins. Exemplary MPV genomes include GenBank Accession Nos. KJ642619.1, KJ642618.1, KJ642617.1 and KJ642616.1.

ns5: Nonstructural protein 5, derived from the larger flavivirus genome polyprotein. The genome polyprotein is subject to proteolysis forming nonstructural proteins that have various viral functions. ns5 has methyltransferase and guanylyltransferase activity. SEQ ID NO: 12 is an exemplary WNV ns5 amino acid sequence.

nsp9, nsp12, and nsp14: Nonstructural Proteins, derived from the larger polyprotein (ORFlab in SARS-CoV-2). The larger polyprotein is subject to proteolysis forming nonstructural proteins that have various viral functions. SARS-CoV-2 ORFlab Gene ID: 43740578. nsp9 has RNA triphosphatase activity. SEQ ID NO: 34 is an exemplary nsp9 amino acid sequence. nsp12 has guanylyltransferase activity. SEQ ID NO: 29 is an exemplary nsp12 amino acid sequence. nsp14 has methyltransferase activity. SEQ ID NOs: 4, 289 and 24 are exemplary nsp14 amino acid sequence.

Nucleic acid molecule: A deoxyribonucleotide or ribonucleotide polymer or combination thereof including without limitation, DNA or RNA, such as cDNA, genomic DNA, subgenomic DNA (sgDNA), mRNA, rRNA, tNRA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (for example, a promoter that drives expression of the heterologous nucleic acid sequence disclosed herein). Generally, operably linked DNA sequences are contiguous and, where necessary to join two protein coding regions, are in the same reading frame.

Pharmaceutically acceptable carriers: Remington's Pharmaceutical Sciences, by E. W. Martin, Mack Publishing Co., Easton, PA, 19th Edition, 1995, describes compositions and formulations suitable for pharmaceutical compositions, which include an isolated virus, such as an isolated SARS-CoV-2 virus with one or more substitutions in nsp14 and/or nsp12.

Examples of fluid carriers include pharmaceutically and physiologically acceptable fluids such as water, physiological saline, balanced salt solutions, aqueous dextrose, glycerol or the like as a vehicle. Examples of solid carriers include pharmaceutical grades of mannitol, lactose, starch, or magnesium stearate.

In addition to biologically neutral carriers, pharmaceutical compositions which include an isolated virus can contain minor amounts of non-toxic auxiliary substances, such as wetting or emulsifying agents, preservatives, and pH buffering agents and the like, for example, sodium acetate or sorbitan monolaurate. In particular examples, the carrier may be sterile.

Such compositions may be present in a sealed vial, for lyophilized for subsequent solubilization.

Plasmid: A construction of genetic material designed to direct transformation of a targeted cell. Plasmids include a construction of extrachromosomal genetic material, usually of a circular duplex of DNA which can replicate independently of chromosomal DNA. A plasmid generally contains multiple genetic elements positional and sequentially oriented with other necessary genetic elements such that the nucleic acid in a nucleic acid cassette can be transcribed and when necessary, translated in the transfected cells. Plasmids include nucleic acids that are DNA derived from a plasmid vector, cosmids, or phagemids wherein one or more fragments of nucleic acid may be inserted or cloned which encode for particular genes of interest. The plasmid can have a linear or circular configuration.

Plasmids generally contain one or more unique restriction sites. In addition, a plasmid can confer some well-defined phenotype on the host organism which is either selectable or readily detected. Thus, the plasmid can include an expression cassette, wherein a polypeptide is encoded. Expression includes the efficient transcription of an inserted gene, nucleic acid sequence, or nucleic acid cassette with the plasmid. Expression products can be proteins, polypeptides or RNA.

In one aspect, when a circular plasmid is transferred into a yeast cell, it can be an autonomously replicating, extra-chromosomal DNA molecule, distinct from the normal yeast genome and nonessential for yeast cell survival under nonselective conditions. The term “persistent expression” as used herein refers to introduction of genes into the cell together with genetic elements which enable episomal (extra-chromosomal) replication and/or maintenance of the genetic material in the cell. This can lead to apparently stable transformation of the cell without the integration of the novel genetic material into the chromosome of the host cell.

A plasmid can also introduce genetic material into chromosomes of the targeted cell where it integrates and becomes a permanent component of the genetic material in that cell. Gene expression after stable introduction can permanently alter the characteristics of the cell and its progeny arising by replication leading to stable transformation.

Poxvirus: Any virus belonging to the family Poxviridae. Poxviridae are characterized by, at least, a relatively large, double-stranded DNA genome (ranging from approximately 130 to 400 kbp). Virions are enveloped, slightly pleomorphic, ovoid, or brick shaped (approximately 140-260 nm in diameter and 220-450 nm long). Virions are composed of an external coat containing lipid and tubular or globular protein structures enclosing one or two lateral bodies and a core, which contains the genome. Particular poxviruses may belong to the chordopoxyirinae or entomopoxyirinae subfamily, which infect vertebrate or insect hosts, respectively. A poxvirus of the chordopoxyirinae subfamily may further belong to the genus Orthopoxvirus (including, e.g., monkeypox virus, vaccinia virus, buffalopoxvirus, camelpox virus, cowpox virus, elephantpox virus, variola virus (such as variola major and/or variola minor viruses), volepox virus, ectromelia virus, raccoonpox virus, skunkpox virus, or taterapox virus), Parapoxvirus (including, e.g., bovine papular stomatitis virus, Orf virus, psuedocowpox virus, sealpox virus, or Auzduk disease virus), Avipoxvirus (including, e.g., fowlpox virus), Capripoxvirus (including, e.g., sheeppox virus, lumpy skin disease virus, or goatpox virus), Leporipoxvirus (including, e.g., myxoma virus, or Shope fibroma virus), Suipoxvirus (including, e.g., swinepox virus), Mollusciposvirus (including, e.g., molluscum contagiosum virus), or Yatapoxvirus (including, e.g., tanapox virus or Yaba monkey tumor virus). Viruses of the Othropoxvirus and Parapoxvirus genera can be further characterized as zoonotic (including, e.g., monkeypox virus, vaccinia virus, buffalopoxvirus, camelpox virus, cowpox virus, elephantpox virus, bovine papular stomatitis virus, Orf virus, psuedocowpox virus, or sealpox virus) or nonzoonotic (including, e.g., variola virus, volepox virus, ectromelia virus, raccoonpox virus, skunkpox virus, taterapox virus, or Auzduk disease virus). Zoonotic viruses can infect multiple species of hosts (e.g., humans and animals), while nonzoonotic viruses are believed to infect only a single host species (e.g., humans, fowl, or monkey, etc.). The complete genomes (including cross-references to individual genes included therein and proteins encoded thereby) of over 25 poxviruses are known and publicly available (see, for instance, GenBank Accession Nos. M35027, U94848, AF095689, L22579, X69198, Y16780, AF380138, AF012825, AY009089, AF438165, AF482758, AF170726, AF170722, AF198100, AF325528, AY077835, AY077836, AY077832, AY077833, AY077834, AF410153, U60315, AJ293568, AF063866, and/or AF250284).

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In one example a promoter is constitutive. In another example a promoter is inducible.

Examples of promoters include, but are not limited to the SV40 promoter, the CMV enhancer-promoter, the CMV enhancer/β-actin promoter, EF1a promoter, or PGK promoter. In one example, expression is driven by a polymerase III promoter, such as U6 or H1, such as human or mouse U6 or H1 promoter. Also included are those promoter elements that are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Recombinant: A nucleic acid molecule or polypeptide that is not naturally occurring or has a sequence that is made by an artificial combination of two otherwise separated segments of nucleotide or amino acid sequence. This artificial combination can be accomplished, for example, by chemical synthesis or by the artificial manipulation of isolated segments of nucleic acids, e.g., by genetic engineering techniques. The term “recombinant” includes nucleic acids or polypeptides that have been altered solely by addition, substitution, or deletion of a portion of a natural nucleic acid molecule or peptide.

Reporter: Reporter genes are genes whose products can be assayed (i.e., observed or detected) subsequent to their introduction into a cell or organism, for example in a mammalian cell. Reporters can be used as markers for screening successfully transfected host cells (e.g., those transfected with a non-native viral genome provided herein), for studying regulation of gene expression, or can serve as controls for standardizing transfection efficiencies. Reporter gene expression can be either constitutive or inducible, with an external intervention such as, for example, the introduction of IPTG in the β-galactosidase system. Reporter genes can be expressed under their own promoter independent from that of the introduced gene or genes of interest, allowing the screening of successfully transfected cells even when the gene or genes of interest are expressed only under certain specific conditions.

For example, a reporter can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein or a small molecule (for example, molecules having a molecular weight of less than 10,000 amu). A reporter gene may encode a fluorescent molecule (such as a fluorescent protein, such as green fluorescent protein, red fluorescent protein, or yellow fluorescent protein) or a bioluminescent molecule (such as luciferase or nanoluciferase) that can be visualized. The amount of fluorescence or bioluminescence emitted from a fluorescent or bioluminescent molecule can be measured, such as the amount of fluorescence emitted from an intrinsically fluorescent molecule (for example green fluorescent protein, yellow fluorescent protein, or red fluorescent protein, among others) or a fluorophore complexed to a protein or nucleic acid. Fluorescence and bioluminescence detection methods suitable for use in the disclosed methods include fluorometry, microscopy, flow cytometry, and spectroscopy. For high throughput screening, laser scanning imaging and microplate readers are also suitable.

RNA triphosphatase: An enzyme which cleaves the B-phosphate and γ-phosphate in RNA. In some examples the RNA triphosphatase is an RNA triphosphatase involved in RNA capping. Exemplary RNA triphosphatases include: Cet1p and MCE1.

Exemplary RNA triphosphatase amino acid sequences include GenBank Accession Nos. NP_003791.3 (H. sapiens MCE1 isoform a), NP_001273355.1 (H. sapiens MCE1 isoform b), NP_036014.1 (M. musculus MCE1 isoform 1), NP_001292202.1 (M. musculus MCE1 isoform 2), XP_020945593.1 (S. scrofa MCE1). Further exemplary RNA triphosphatase amino acid sequences include SEQ ID NO: 287 (Sus scrofa MCE1).

RNA guanine-7 methyltransferase (RNMT): Enzyme with mRNA (guanine-N7-)-methyltransferase activity, involved in 7-methylguanosine mRNA capping. Gene name RNMT. H. sapiens Gene ID: 8731. Exemplary RNMT amino acid sequences include GenBank Accession Nos. CNP_001295192.1 (H. sapiens RNMT isoform 1), NP_003790.1 (H. sapiens RNMT isoform 2), NP_001365061.1 (H. sapiens RNMT isoform 3), NP_080716.1 (M. musculus RNMT isoform 1), NP_001164424.1 (M. musculus RNMT isoform 2), XP_020951674.1 (S. scrofa RNMT), SEQ ID NO: 285 (S. scrofa RNMT), and SEQ ID NO: 286 (S. scrofa RNMT).

SARS-CoV-2: Also known as 2019-nCoV or 2019 novel coronavirus, SARS-CoV-2 is a positive-sense, single stranded RNA virus of the genus betacoronavirus and is the causative agent of Coronavirus Disease 2019 (COVID-19). The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (NP)-3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

In one example, a SARS-CoV-2 is a naturally occurring variant thereof, such as alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (such as original lineage: B.1.1.529 and lineages: BA.2, BA.4, BA.5, BQ.1, BQ.1.1, BA.4.6, BF.7, and B.1.1.529). AT.1 is another exemplary naturally occurring variant of SARS-CoV-2.

Exemplary SARS-CoV-2 genomes include GenBank Accession Nos. NC_045512.2, OQ458113.1, and OQ223417.1.

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals, such as non-human primates, pigs, camels, bats, sheep, cows, dogs, cats, rodents, and the like. In a particular example, the subject is a human. In a particular example, the subject is a pig. In an additional example, a subject is selected that is in need of inhibiting an infection, such as SARS-CoV-2, MERS-CoV, ASFV, MPV or WNV infection. For example, the subject is either uninfected and at risk of the SARS-CoV-2, MERS-CoV, ASFV, MPV or WNV infection, or is infected and in need of treatment.

Transfection and Transduction: A transfected cell is a cell into which has been introduced a nucleic acid molecule by molecular biology techniques. Transfection encompasses all techniques by which a nucleic acid molecule might be introduced into such a cell, including transfection with plasmid vectors, and introduction of DNA by electroporation, liposome-mediated transfection (lipofection), non-liposomal transfection, dendrimer-based transfection, particle bombardment, and microinjection. Transduction as used herein includes virus-mediated gene delivery.

West Nile Virus: A member of the viral family Flaviviridae and the genus Flavivirus. WNV was first isolated from a woman in the West Nile district of Uganda in 1937. The virus was later identified in birds in the Nile delta region in 1953. Human infections attributable to WNV have been reported in many countries for over 50 years. In 1999, a WNV circulating in Israel and Tunisia was imported into New York, producing a large and dramatic outbreak that spread throughout the continental United States in the following years. Human infection is most often the result of bites from infected mosquitoes, but may also be transmitted through contact with other infection animals, their blood or other tissues. Infection with WNV is asymptomatic in about 80% of infected people, but about 20% develop West Nile fever.

Symptoms include fever, headache, fatigue, body aches, nausea, vomiting, swollen lymph glands and in some cases, a skin rash. Approximately 1 in 150 of infected individuals develop severe, neuroinvasive disease, such as encephalitis, meningitis or poliomyelitis. Treatment of WNV infection is supportive, such as administration of intravenous fluids, respiratory support and prevention of secondary infections. There is currently no approved vaccine available for humans.

Exemplary WNV genomes include GenBank Accession Nos. KR868734.1, LC318700.1, and MF797870.1.

Yeast: Unicellular microorganisms that belong to one of three classes: Ascomycetes, Basidiomycetes and Fungi Imperfecti. A yeast can be a non-pathogenic strain such as Saccharomyces cerevisiae. Yeast strains include Saccharomyces, Candida, Cryptococcus, Hansenula, Kluyveromyces, Pichia, Rhodotorula, Schizosaccharomyces and Yarrowia. Yeast genera include Saccharomyces, Candida, Hansenula, Pichia or Schizosaccharomyces. Species of yeast strains include Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica. “Non-native yeast” are those that are genetically modified from a corresponding wild type (native) yeast.

### III. Overview

To combat emerging pathogenic RNA viruses, there is a need to develop broadly applicable platforms that can be rapidly and rationally modulated based on emerging pathogens. This disclosure provides readily tractable and modular synthetic biology platforms targeting another essential and highly conserved, but relatively less explored, step in coronavirus translation and replication, capping of the viral RNA genome.^{18,35} Typically, in vitro assays⁶¹ or in vivo studies^{39,62} with viral replicons of pathogenic human RNA viruses have been used for studying and targeting RNA capping mechanisms. Such platforms using viral replicons of pathogenic human RNA viruses are often not compatible for use in standard Biosafety level 2 setting and can also be significantly challenging to genetically manipulate, making them intractable for basic molecular virology studies and high-throughput translational efforts targeting RNA capping enzymes. Here, a synthetic phenotypic yeast platform for molecular characterization and targeting of SARS-CoV-2 genome encoded enzymes involved in capping of the viral RNA genome is disclosed.

The disclosed synthetic, phenotypic yeast-based complementation platforms, such as YeRCOM, can be used in standard laboratory setting. In some examples, the phenotypic yeast-based complementation platforms can be used for functional characterization and targeting of SARS-CoV-2 RNA cap-0 (guanine-N7)-methyltransferase. These platforms were used to perform molecular characterization of SARS-CoV-2 nsp14 and identify domains and residues in nsp14 that impact N7-MTase activity. These platforms can include nsp14 variants observed in emerging variants of SARS-CoV-2 (e.g., delta variant of SARS-CoV-2 encodes nsp14 A394V and nsp14 P46L). In some examples, these platforms are combined with directed evolution to identify attenuation mutations in N7-MTase which has been shown to result in attenuation of coronaviruses; this can be used to generate low-reversion variants of viruses as live attenuated vaccine candidates.⁶³ In some examples, these platforms are modular and readily adaptable to any RNA virus.

### REFERENCES

(18) Ferron, F.; Decroly, E.; Selisko, B.; Canard, B. The Viral RNA Capping Machinery as a Target for Antiviral Drugs. Antiviral Res 2012, 96 (1), 21-31.
(35) Decroly, E.; Ferron, F.; Lescar, J.; Canard, B. Conventional and Unconventional Mechanisms for Capping Viral MRNA. Nat Rev Microbiol 2012, 10 (1), 51-65.
(39) Eckerle, L. D.; Lu, X.; Sperry, S. M.; Choi, L.; Denison, M. R. High Fidelity of Murine Hepatitis Virus Replication Is Decreased in Nsp14 Exoribonuclease Mutants. Journal of virology 2007, 81 (22), 12135-12144.
(61) Bouvet, M.; Debarnot, C.; Imbert, I.; Selisko, B.; Snijder, E. J.; Canard, B.; Decroly, E. In Vitro Reconstitution of SARS-Coronavirus MRNA Cap Methylation. PLOS Pathogens 2010, 6 (4), e1000863.
(62) Graham, R. L.; Becker, M. M.; Eckerle, L. D.; Bolles, M.; Denison, M. R.; Baric, R. S. A Live, Impaired-Fidelity Coronavirus Vaccine Protects in an Aged, Immunocompromised Mouse Model of Lethal Disease. Nature medicine 2012, 18 (12), 1820.
(63) Menachery, V. D.; Yount, B. L.; Josset, L.; Gralinski, L. E.; Scobey, T.; Agnihothram, S.; Katze, M. G.; Baric, R. S. Attenuation and Restoration of Severe Acute Respiratory Syndrome Coronavirus Mutant Lacking 2′-O-Methyltransferase Activity. J Virol 2014, 88 (8), 4251-4264.

Several zoonotic pathogenic viruses have emerged in the last 20 years, e.g., SARS-CoV, MERS-CoV, West Nile Virus, Monkeypox virus, and so on. MERS coronavirus related infections continue to be detected in human populations with a high case-fatality ratio of 34.5%. The 2022 Monkeypox virus outbreak resulted in more than 29,000 confirmed infections in the United States alone. This disclosure expands beyond human viruses. African Swine Fever Virus is a big threat to domestic pigs and food industry. ASFV may be derived from viruses of soft ticks. These ticks may be infecting wild swine population. Though ASFV results in asymptomatic infection in wild hosts, ASFV related outbreaks in domestic pigs lead to massive loss of domestic pigs due to high mortality rate in domesticated pigs.

There is an increasing need to develop broad-spectrum antivirals targeting viral replication and propagation mechanisms. The instant disclosure provides readily tractable and highly modular synthetic biology platforms targeting capping of the viral RNA genome.⁸⁹ In one example, YeRCOM is used for characterization and targeting of SARS-CoV-2 genome N7-MTase. In another example, YeRCOM is rapidly adapted to RNA capping enzymes from emerging pathogenic viruses that are a significant threat to human health and food supply. In some examples, YeRCOM derived platforms are engineered for targeting of RNA capping enzymes from emerging pathogens including MERS-CoV, Monkeypox virus and African Swine Fever virus which each pose a significant threat to human health and global economy. These phenotypic platforms can be used for broad-spectrum antiviral development, and live attenuated vaccine development for viral pathogens.

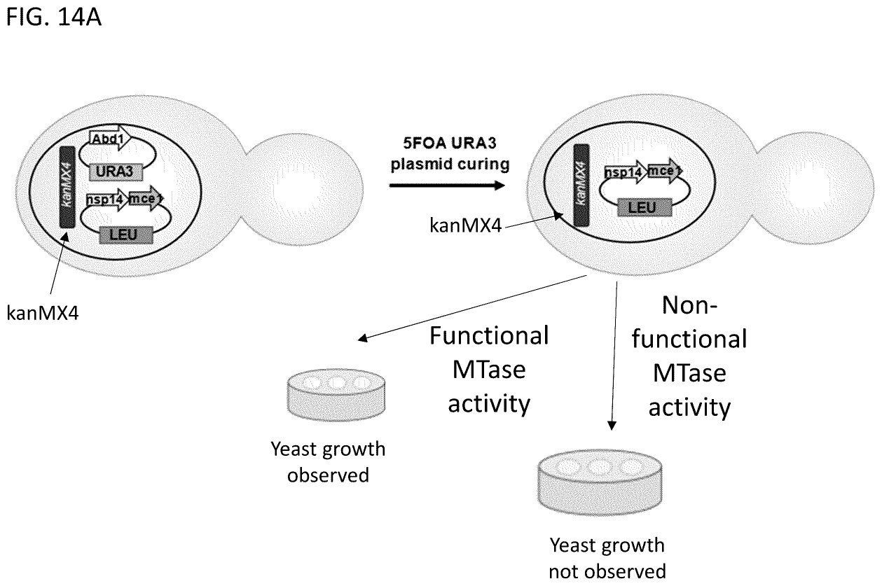

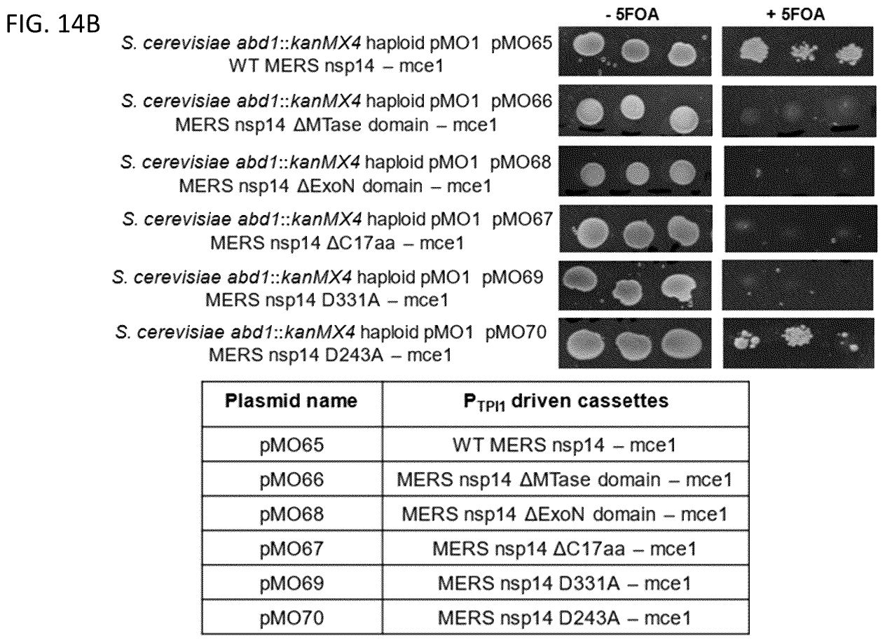

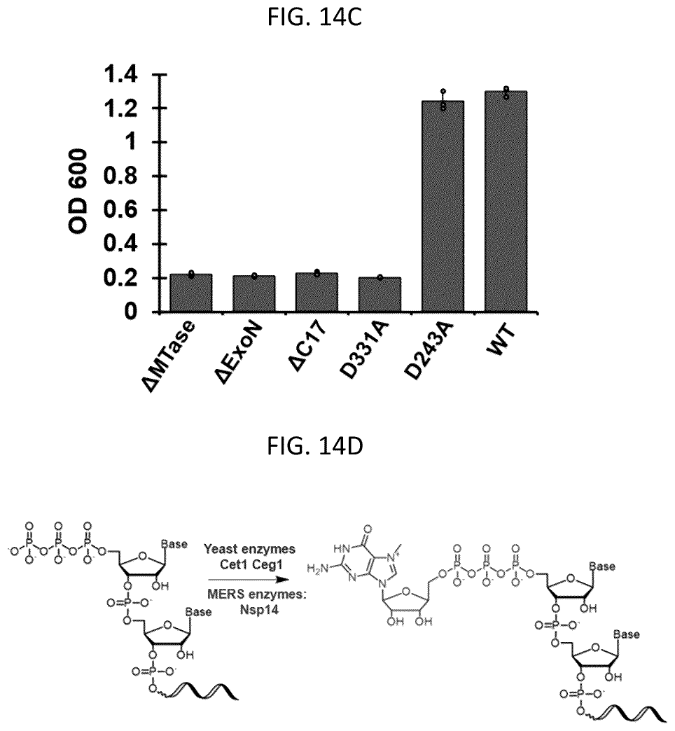

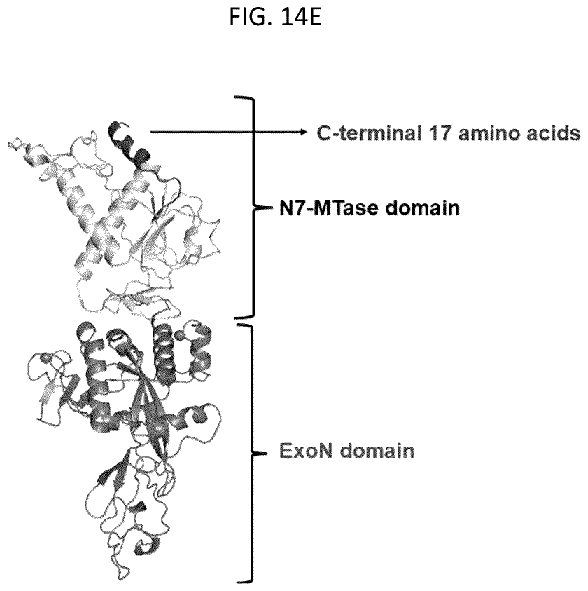

One reason RNA viruses and DNA viruses encode their own capping enzymes is because they replicate in the host cytoplasm, whereas the host RNA capping enzymes are localized in the host nucleus.⁴ As compared to the conserved enzymatic steps involved in the eukaryotic mRNA capping reactions, the mechanistic details of the enzymatic steps involved in viral RNA capping processes can be highly diverse.⁸ In addition to the differences in enzyme structure and reaction mechanisms, the viral RNA capping is also diverse in terms of genetic components and protein domain organization. In some examples, viruses encode RNA capping enzymes that first incorporate a guanosine linked to first nucleotide of the transcript mRNA via 5′-5′ triphosphate bridge followed by a series of methylation reactions at cap-0 and cap-1 position (FIG. 14D).⁸ Certain RNA viruses like influenza viruses, incorporate a 5′-end cap by a mechanism called as cap-snatching mechanism, where viral enzymes essentially “snatch” and excise the 5′-end of host mRNAs and append it to the viral RNA genome (FIG. 14B).⁴³ Certain viruses (e.g., Picornavirus) encode a complex RNA secondary structure which facilitates cap-independent translation of the viral mRNA sequence.⁴⁴

The molecular implications of targeting RNA capping enzymes can be distinct between RNA and DNA viruses. In case of RNA viruses, only the 5′end of the viral RNA genome is capped by the RNA capping enzymes. This disguises the entire viral genome as the host mRNA. On the other hand, DNA viruses like ASFV can have a large double stranded DNA genome with hundreds of open reading frames for RNA transcription. Most of these DNA virus mRNA transcripts (if not all) are capped at their 5′end with a cap structure that is identical to the host mRNAs. Therefore, unlike RNA viruses where the RNA capping enzymes are involved in capping the viral genome, in case of DNA viruses, the viral RNA capping enzymes are involved in capping of the viral mRNA transcripts. Regardless, in both cases, the RNA capping enzymes are implicated in viral propagation. Though the final RNA cap structure of the viral RNA genome or the viral mRNA transcript is identical to the host mRNAs, the proteins, protein domains and the mechanistic details of the viral RNA capping system often are distinct from human mRNA capping enzymes. Lack of RNA capping mechanisms results in significant attenuation of these strains and loss of pathogenesis. All of this makes viral RNA capping system a target for combating emerging pathogens. Despite this, the translational efforts targeting viral RNA capping enzymes have not been as successful as those targeting viral polymerases or proteases. One reason for this is that the screening methods for targeting viral RNA capping system typically involve low activity radioactive assays or using pathogenic replicons of virus that are incompatible for use in standard Biosafety Level 2 setting. By contrast, the instant disclosure utilizes synthetic biology approaches to develop yeast-based platforms for characterization and targeting of essential viral RNA capping enzymes from emerging pathogenic RNA and DNA viruses. In some examples the disclosed platforms are compatible for characterization and targeting of RNA capping enzymes from RNA viruses as well as DNA viruses. In some examples, these platforms are used for phenotypic screening to identify selective viral RNA capping enzyme inhibitors as well as to identify attenuation mutations in these essential viral enzymes. In some examples this platform is applied to human RNA capping enzymes, to enable these platforms to identify broad-spectrum inhibitors that selectively target viral RNA capping enzymes over human RNA capping enzymes. In some examples, these platforms are used for directed evolution of RNA capping enzymes from the above emerging pathogens, to permit the identification of attenuation mutations in viral enzymes.

### REFERENCES

4. Ramanathan, A., Robb, G. B. & Chan, S.-H. mRNA capping: biological functions and applications. Nucleic Acids Res. 44, 7511-7526 (2016).
8. Decroly, E., Ferron, F., Lescar, J. & Canard, B. Conventional and unconventional mechanisms for capping viral mRNA. Nat. Rev. Microbiol. 10, 51-65 (2012).
9. Ferron, F., Decroly, E., Selisko, B. & Canard, B. The viral RNA capping machinery as a target for antiviral drugs. Antiviral Res. 96, 21-31 (2012).
43. De Vlugt, C., Sikora, D. & Pelchat, M. Insight into influenza: a virus cap-snatching. Viruses 10, 641 (2018).
44. Martinez-Salas, E., Francisco-Velilla, R., Fernandez-Chamorro, J. & Embarek, A. M. Insights into Structural and Mechanistic Features of Viral IRES Elements. Front. Microbiol. 8, (2018).

Disclosed herein is an isolated non-native yeast including a genetically inactivated Abd1 gene and a heterologous methyltransferase. Also disclosed herein is an isolated non-native yeast including a genetically inactivated Ceg1p gene and a heterologous guanylyltransferase. Also disclosed herein is an isolated non-native yeast including a genetically inactivated Cet1p and a heterologous RNA triphosphatase.

In some examples, the isolated non-native yeast has an Abd1 gene replaced with an antibiotic resistance gene. In some examples the heterologous methyltransferase is a full-length methyltransferase. In some examples the heterologous methyltransferase is a functional methyltransferase fragment. In some examples the heterologous methyltransferase is a codon optimized methyltransferase for expression in yeast. In some examples the heterologous methyltransferase is encoded by a plasmid. In some examples the plasmid encoding the heterologous methyltransferase includes a promoter operably linked to the heterologous methyltransferase. In some examples the plasmid encoding the heterologous methyltransferase has at least 80%, at least 85%, at least 90%, at least 95%, at least 96%, at least 97%, at least 98%, at least 99%, or 100% sequence identity to SEQ ID NO: 16, 17, 18, 19, 21, 22, or 23. In some examples the heterologous methyltransferase is fused to an RNA polymerase complex targeting gene. In some examples, the RNA polymerase complex targeting gene includes a Mce1 GTase domain gene. In some examples, the Mce1 GTase domain gene is a full length Mce1 GTase domain. In some examples the Mce1 GTase domain gene is a functional Mce1 GTase domain fragment. In some examples the Mce1 GTase domain gene is a codon optimized Mce1 GTase domain gene for expression in yeast. In some examples the amino acid sequence of the Mce1 GTase domain gene has at least 80%, at least 85%, at least 90%, at least 95%, at least 96%, at least 97%, at least 98%, at least 99%, or 100% sequence identity to SEQ ID NO: 1. In some examples the nucleotide sequence of the Mce1 GTase domain gene has at least 80%, at least 85%, at least 90%, at least 95%, at least 96%, at least 97%, at least 98%, at least 99%, or 100% sequence identity to SEQ ID NO: 2. In some examples the isolated non-native yeast includes an Adb1 plasmid including a second Abd1 gene operably linked to a promoter. In some examples, the Adb1 plasmid includes a yeast Ura3 gene; and the Adb1 plasmid is curable. In some examples the nucleotide sequence of the Adb1 plasmid has at least 80%, at least 85%, at least 90%, at least 95%, at least 96%, at least 97%, at least 98%, at least 99%, or 100% sequence identity to SEQ ID NO: 3. In some examples the heterologous methyltransferase includes nsp14, D1, D12, D1 and D12, NP868R, ns5, or mRNA cap methyltransferase (RNMT). In some examples, the nsp14 has a protein sequence of at least 80%, at least 85%, at least 90%, at least 95%, at least 96%, at least 97%, at least 98%, at least 99%, or 100% sequence identity to SEQ ID NO: 4 or 24. In some examples the nsp14 has a protein sequence including the V381L, A394V, P46L, P412H, L157F and/or I42V substitutions within SEQ ID NO: 289. In some examples, the nsp14 is encoded by a nucleic acid sequence having at least 80%, at least 85%, at least 90%, at least 95%, at least 96%, at least 97%, at least 98%, at least 99%, or 100% sequence identity to SEQ ID NO: 5 or 25. In some examples the D1 protein sequence has at least 80%, at least 85%, at least 90%, at least 95%, at least 96%, at least 97%, at least 98%, at least 99%, or 100% sequence identity to SEQ ID NO: 6. In some examples, the D1 is encoded by a nucleic acid sequence having at least 80%, at least 85%, at least 90%, at least 95%, at least 96%, at least 97%, at least 98%, at least 99%, or 100% sequence identity to SEQ ID NO: 7. In some examples, the D12 is encoded by a protein sequence having at least 80%, at least 85%, at least 90%, at least 95%, at least 96%, at least 97%, at least 98%, at least 99%, or 100% sequence identity to SEQ ID NO: 8. In some examples, the D12 is encoded by a nucleic acid sequence having at least 80%, at least 85%, at least 90%, at least 95%, at least 96%, at least 97%, at least 98%, at least 99%, or 100% sequence identity to SEQ ID NO: 9. In some examples, the NP868R is encoded by a protein sequence having at least 80%, at least 85%, at least 90%, at least 95%, at least 96%, at least 97%, at least 98%, at least 99%, or 100% sequence identity to SEQ ID NO: 10. In some examples the NP868R is encoded by a nucleic acid sequence having at least 80%, at least 85%, at least 90%, at least 95%, at least 96%, at least 97%, at least 98%, at least 99%, or 100% sequence identity to SEQ ID NO: 11. In some examples the ns5 is encoded by a protein sequence including at least 80%, at least 85%, at least 90%, at least 95%, at least 96%, at least 97%, at least 98%, at least 99%, or 100% sequence identity to SEQ ID NO: 12. In some examples the RNMT is encoded by a protein sequence having at least 80%, at least 85%, at least 90%, at least 95%, at least 96%, at least 97%, at least 98%, at least 99%, or 100% sequence identity to SEQ ID NO: 14. In some examples the RNMT is encoded by a nucleic acid sequence having at least 80%, at least 85%, at least 90%, at least 95%, at least 96%, at least 97%, at least 98%, at least 99%, or 100% sequence identity to SEQ ID NOs: 15, 285, or 286. In some examples the heterologous methyltransferase includes a Coronaviridae methyltransferase, a Poxviridae methyltransferase, an Asfarviridae methyltransferase, a Flaviviridae methyltransferase, a Filoviridae methyltransferase, a vertebrate methyltransferase, or a mammalian methyltransferase. In some examples, the heterologous methyltransferase is nsp14, having a catalytically inactive ExoN domain.

In some examples, the Ceg1p gene is replaced by an antibiotic resistance gene. In some examples, the heterologous guanylyltransferase is a full length guanylyltransferase. In some examples the heterologous guanylyltransferase is a functional guanylyltransferase fragment. In some examples the heterologous guanylyltransferase is a codon optimized guanylyltransferase for expression in yeast. In some examples the heterologous guanylyltransferase is encoded by a plasmid. In some examples the plasmid encoding the heterologous guanylyltransferase includes a promoter operably linked to the heterologous guanylyltransferase. In some examples, the plasmid encoding the heterologous guanylyltransferase has at least 80%, at least 85%, at least 90%, at least 95%, at least 96%, at least 97%, at least 98%, at least 99%, or 100% sequence identity to SEQ ID NOs: 17, 18, 19, 26 or 27. In some examples, the heterologous guanylyltransferase is fused to an RNA polymerase complex targeting gene. In some examples, the RNA polymerase complex targeting gene includes a Mce1 GTase domain gene. In some examples, the Mce1 GTase domain gene is a full length Mce1 GTase domain. In some examples, the Mce1 GTase domain gene is a functional Mce1 GTase domain fragment. In some examples the Mce1 GTase domain gene is a codon optimized Mce1 GTase domain gene for expression in yeast. In some examples, the amino acid sequence of the Mce1 GTase domain gene has at least 80%, at least 85%, at least 90%, at least 95%, at least 96%, at least 97%, at least 98%, at least 99%, or 100% sequence identity to SEQ ID NO: 1. In some examples, the nucleotide sequence of the mcel GTase domain gene has at least 80%, at least 85%, at least 90%, at least 95%, at least 96%, at least 97%, at least 98%, at least 99%, or 100% sequence identity to SEQ ID NO: 2. In some examples, the isolated non-native yeast further includes a Ceg1p plasmid having a second Ceg1p gene operably linked to a promoter. In some examples, the Ceg1p plasmid further includes a yeast ura3 gene, and the Ceg1p plasmid is curable. In some examples, the nucleotide sequence of the Ceg1p plasmid has at least 80%, at least 85%, at least 90%, at least 95%, at least 96%, at least 97%, at least 98%, at least 99%, or 100% sequence identity to SEQ ID NO: 28. In some examples, the heterologous guanylyltransferase includes nsp12, ns5, NP868R, D1, D12, D1 and D12, or MCE1. In some examples, the nsp12 is encoded by a protein sequence having at least 80%, at least 85%, at least 90%, at least 95%, at least 96%, at least 97%, at least 98%, at least 99%, or 100% sequence identity to SEQ ID NO: 29. In some examples the nsp12 is encoded by a nucleic acid sequence having at least 80%, at least 85%, at least 90%, at least 95%, at least 96%, at least 97%, at least 98%, at least 99%, or 100% sequence identity to SEQ ID NO: 30. In some examples, the ns5 is encoded by a protein sequence having at least 80%, at least 85%, at least 90%, at least 95%, at least 96%, at least 97%, at least 98%, at least 99%, or 100% sequence identity to SEQ ID NO: 31. In some examples, the ns5 is encoded by a nucleic acid sequence having at least 80%, at least 85%, at least 90%, at least 95%, at least 96%, at least 97%, at least 98%, at least 99%, or 100% sequence identity to SEQ ID NO: 32. In some examples, the D1 is encoded by a protein having at least 80%, at least 85%, at least 90%, at least 95%, at least 96%, at least 97%, at least 98%, at least 99%, or 100% sequence identity to SEQ ID NO: 6. In some examples, the D1 is encoded by a nucleic acid sequence having at least 80%, at least 85%, at least 90%, at least 95%, at least 96%, at least 97%, at least 98%, at least 99%, or 100% sequence identity to SEQ ID NO: 7. In some examples, the D12 is encoded by a protein sequence having at least 80%, at least 85%, at least 90%, at least 95%, at least 96%, at least 97%, at least 98%, at least 99%, or 100% sequence identity to SEQ ID NO: 8. In some examples, the D12 is encoded by a nucleic acid sequence having at least 80%, at least 85%, at least 90%, at least 95%, at least 96%, at least 97%, at least 98%, at least 99%, or 100% sequence identity to SEQ ID NO: 9. In some examples, the NP868R is encoded by a protein sequence having at least 80%, at least 85%, at least 90%, at least 95%, at least 96%, at least 97%, at least 98%, at least 99%, or 100% sequence identity to SEQ ID NO: 10. In some examples, the NP868R is encoded by a nucleic acid sequence having at least 80%, at least 85%, at least 90%, at least 95%, at least 96%, at least 97%, at least 98%, at least 99%, or 100% sequence identity to SEQ ID NO: 11. In some examples, the MCE1 is encoded by a protein sequence having at least 80%, at least 85%, at least 90%, at least 95%, at least 96%, at least 97%, at least 98%, at least 99%, or 100% sequence identity to SEQ ID NOs: 33 or 287. In some examples, the heterologous guanylyltransferase includes a Coronaviridae guanylyltransferase, a Poxviridae guanylyltransferase, an Asfarviridae guanylyltransferase, a Flaviviridae guanylyltransferase, a Filoviridae guanylyltransferase, a vertebrate guanylyltransferase, or a mammalian guanylyltransferase.

In some examples, the Cet1p gene is replaced by an antibiotic resistance gene. In some examples, the heterologous RNA triphosphatase is a full length RNA triphosphatase. In some examples, the heterologous RNA triphosphatase is a functional RNA triphosphatase fragment. In some examples, the heterologous RNA triphosphatase is a codon optimized RNA triphosphatase for expression in yeast. In some examples, the heterologous RNA triphosphatase is encoded by a plasmid. In some examples, the plasmid encoding the heterologous RNA triphosphatase includes a promoter operably linked to the heterologous RNA triphosphatase. In some examples, the plasmid encoding the heterologous RNA triphosphatase has at least 80%, at least 85%, at least 90%, at least 95%, at least 96%, at least 97%, at least 98%, at least 99%, or 100% sequence identity to SEQ ID NOs: 17, 18, or 19. In some examples, the heterologous RNA triphosphatase is fused to an RNA polymerase complex targeting gene. In some examples, the RNA polymerase complex targeting gene includes a Mce1 GTase domain gene. In some examples, the Mce1 GTase domain gene is a full length Mce1 GTase domain. In some examples, the Mce1 GTase domain gene is a functional Mce1 GTase domain fragment. In some examples, the Mce1 GTase domain gene is a codon optimized Mce1 GTase domain gene for expression in yeast. In some examples, the amino acid sequence of the Mce1 GTase domain gene has at least 80%, at least 85%, at least 90%, at least 95%, at least 96%, at least 97%, at least 98%, at least 99%, or 100% sequence identity to SEQ ID NO: 1. In some examples the nucleotide sequence of the mce1 GTase domain gene has at least 80%, at least 85%, at least 90%, at least 95%, at least 96%, at least 97%, at least 98%, at least 99%, or 100% sequence identity to SEQ ID NO: 2. In some examples, the isolated non-native yeast further includes a Cet1p plasmid having a second Cet1p gene operably linked to a promoter. In some examples, the Cet1p plasmid further includes a yeast Ura3 gene, and the Cet1p plasmid is curable. In some examples, the heterologous RNA triphosphatase includes nsp9, NP868R, D1, D12, D1 and D12, or MCE1. In some examples, the nsp9 is encoded by a protein sequence having at least 80%, at least 85%, at least 90%, at least 95%, at least 96%, at least 97%, at least 98%, at least 99%, or 100% sequence identity to SEQ ID NO: 34. In some examples, the D1 is encoded by a protein sequence having at least 80%, at least 85%, at least 90%, at least 95%, at least 96%, at least 97%, at least 98%, at least 99%, or 100% sequence identity to SEQ ID NO: 6. In some examples, the D1 is encoded by a nucleic acid sequence having at least 80%, at least 85%, at least 90%, at least 95%, at least 96%, at least 97%, at least 98%, at least 99%, or 100% sequence identity to SEQ ID NO: 7. In some examples, the D12 is encoded by a protein sequence having at least 80%, at least 85%, at least 90%, at least 95%, at least 96%, at least 97%, at least 98%, at least 99%, or 100% sequence identity to SEQ ID NO: 8. In some examples, the D12 is encoded by a nucleic acid sequence having at least 80%, at least 85%, at least 90%, at least 95%, at least 96%, at least 97%, at least 98%, at least 99%, or 100% sequence identity to SEQ ID NO: 9. In some examples, the NP868R is encoded by a protein sequence having of at least 80%, at least 85%, at least 90%, at least 95%, at least 96%, at least 97%, at least 98%, at least 99%, or 100% sequence identity to SEQ ID NO: 10. In some examples, the NP868R is encoded by a nucleic acid sequence having at least 80%, at least 85%, at least 90%, at least 95%, at least 96%, at least 97%, at least 98%, at least 99%, or 100% sequence identity to SEQ ID NO: 11. In some examples, the MCE1 is encoded by a protein having at least 80%, at least 85%, at least 90%, at least 95%, at least 96%, at least 97%, at least 98%, at least 99%, or 100% sequence identity to SEQ ID NOs: 33 or 287.

In some examples, the heterologous methyltransferase; heterologous guanylyltransferase; or heterologous RNA triphosphatase is from a virus which encodes an RNA capping enzyme. In some examples, the virus which encodes the RNA capping enzyme is a Coronavirus, an African Swine Fever Virus, a Pox virus, a Flavivirus, a Coronaviridae virus, an Asfarviridae virus, a Poxviridae virus, a Filoviridae virus, or a Flaviviridae virus. In some examples, the Coronavirus or the Coronaviridae virus is SARS-CoV-1, SARS-CoV-2, MERS-CoV, 229E, NL63, OC43, or HKU1. In some examples, the African Swine Fever Virus or the Asfarviridae virus is African Swine Fever Virus. In some examples, the Poxvirus or the Poxviridae virus is smallpox virus, vaccinia virus, monkeypox virus, molluscum contagiosum virus, milker's nodes virus, Orf virus, cowpox virus, or tanapox virus. In some examples, the Filoviridae virus is Ebola virus or Marburg virus. In some examples, the Flavivirus or the Flaviviridae virus is dengue virus, yellow fever virus, Zika virus, Japanese encephalitis virus, West Nile virus, Kyasanur forest disease, Alkhurma disease, or Omsk hemorrhagic fever. In some examples, the yeast is S. cerevisiae. In some examples, the isolated non-native yeast is haploid.

Disclosed herein is a composition including the previously described isolated non-native yeasts, and a carrier. In some examples, the carrier is a solid or liquid medium. In some examples, the carrier includes synthetic defined media, optionally where the synthetic defined media includes yeast nitrogen base including glucose, lysine, methionine, histidine, and leucine, optionally, where the yeast nitrogen base lacks amino acids. In some examples, the composition further includes 5-FOA.

In some examples, disclosed herein is a kit with a plasmid including a methyltransferase gene, where incorporating the plasmid into a yeast including genetically inactivated Abd1 results in an isolated non-native yeast including a genetically inactivated Abd1 gene and a heterologous methyltransferase. In some examples, disclosed herein is kit with a plasmid having a guanylyltransferase gene, where incorporating the plasmid into a yeast having genetically inactivated Ceg1p results in an isolated non-native yeast including a genetically inactivated Ceg1p gene and a heterologous guanylyltransferase. In some examples, disclosed herein is a kit with a plasmid having an RNA triphosphatase gene, where incorporating the plasmid into a yeast having genetically inactivated Cet1p results in the isolated non-native yeast including a genetically inactivated Cet1p and a heterologous RNA. In some examples the kit also includes yeast with genetically inactivated Abd1, yeast with genetically inactivated Ceg1p, and/or yeast with genetically inactivated Cet1p. In some examples, the kit includes instructions for using the kit. In some examples the kit includes a culture medium.

Further disclosed herein is a kit including a plasmid including a methyltransferase gene, where incorporating the plasmid into a yeast having genetically inactivated Abd1 results in an isolated non-native yeast including a genetically inactivated Abd1 gene and a heterologous methyltransferase.

Also disclosed is a kit including a plasmid including a guanylyltransferase gene, where incorporating the plasmid into a yeast having genetically inactivated Ceg1p results in an isolated non-native yeast including a genetically inactivated Ceg1p gene and a heterologous guanylyltransferase. Also disclosed herein is a kit including a plasmid including an RNA triphosphatase gene, where incorporating the plasmid into a yeast having genetically inactivated Cet1p results in the isolated non-native yeast including a genetically inactivated Cet1p and a heterologous RNA. In some examples, the yeast including genetically inactivated Abd1 further includes a curable Abd1 plasmid. In some examples, the yeast including genetically inactivated Ceg1p further includes a curable Ceg1p plasmid. In some examples, the yeast including genetically inactivated Cet1p further includes a curable Cet1p plasmid. In some examples, the kits disclosed herein further include a culture medium where the culture medium optionally includes G418, optionally lacks uracil, and optionally includes 5-FOA.

In some examples, disclosed herein is a kit including a first strain of the isolated non-native yeasts described herein and a culture medium, optionally: where the culture medium includes G418, where the culture medium lacks uracil, where the culture medium includes 5-FOA, and instructions for using the kit. In some examples, further included is second strain of the isolated non-native yeasts disclosed herein, optionally, where the second strain includes: a vertebrate heterologous methyltransferase, heterologous guanylyltransferase, and/or heterologous RNA triphosphatase. In some examples, further included is a first control small molecule which inhibits activity of the heterologous methyltransferase, the heterologous guanylyltransferase, or the heterologous RNA triphosphatase of the first strain, and which inhibits activity of the heterologous methyltransferase, the heterologous guanylyltransferase, or the heterologous RNA triphosphatase of the second strain. In some examples, further included is a second control small molecule which inhibits activity of the heterologous methyltransferase, the heterologous guanylyltransferase, or the heterologous RNA triphosphatase of the first strain, and which does not inhibit activity of the heterologous methyltransferase, the heterologous guanylyltransferase, or the heterologous RNA triphosphatase of the second strain. Optionally further included is a third control small molecule which does not inhibit activity of the heterologous methyltransferase, the heterologous guanylyltransferase, or the heterologous RNA triphosphatase of the first strain, and which inhibits activity of the heterologous methyltransferase, the heterologous guanylyltransferase, or the heterologous RNA triphosphatase of the second strain. Optionally further included is a fourth control small molecule which does not inhibit activity of the heterologous methyltransferase, the heterologous guanylyltransferase, or the heterologous RNA triphosphatase of the first strain, and which does not inhibit activity of the heterologous methyltransferase, the heterologous guanylyltransferase, or the heterologous RNA triphosphatase of the second strain.

Also disclosed herein is a method of identifying one or more attenuating mutations in the heterologous viral methyltransferase; heterologous viral guanylyltransferase; or heterologous viral RNA triphosphatase of the isolated non-native yeasts disclosed herein. In some examples, this includes altering one or more amino acid residues in the heterologous viral methyltransferase; heterologous viral guanylyltransferase; or heterologous viral RNA triphosphatase of the isolated non-native yeast to generate an experimental strain. In some examples, this includes culturing the experimental strain. In some examples this includes measuring growth of the experimental strain. In some examples, this includes comparing the growth of the experimental strain to a control. In some examples, the control is historical non-native yeast growth data or the isolated non-native yeast. In some examples, when the experimental strain grows slower than the control, this indicates the presence of an attenuating mutation in the experimental strain,

Also disclosed herein is a method of screening for methyltransferase, guanylyltransferase, or RNA triphosphatase inhibitors using the isolated non-native yeasts disclosed herein. In some examples, this includes culturing a first strain of the isolated non-native yeast in media having at least one screening compound. In some examples, this includes measuring growth of the first strain. In some examples, this includes comparing the growth of the first strain to a control. In some examples, the control includes historical non-native yeast growth data, the isolated non-native yeast grown in the absence of the screening compound, or the isolated non-native yeast grown in the presence of a control compound. In some examples, this method includes culturing a second strain of the isolated non-native yeast in the media having the screening compound, the second strain including a vertebrate methyltransferase gene, a vertebrate guanylyltransferase gene, or a vertebrate RNA triphosphatase gene; measuring the growth of the second strain and comparing the growth of the second strain to a control, or to the growth of the first strain. In some examples, the media further includes 5-FOA or G418, and optionally the media lacks uracil. In some examples, the first strain growing slower than the control indicates the at least one screening compound is a methyltransferase inhibitor, a guanylyltransferase inhibitor, or an RNA triphosphatase inhibitor. In some examples, the first strain growing slower than both the control and the second strain indicates the at least one screening compound is a specific methyltransferase inhibitor, a specific guanylyltransferase inhibitor, or a specific RNA triphosphatase inhibitor.

In some examples, the vertebrate methyltransferase gene is a human methyltransferase gene. In some examples, the vertebrate guanylyltransferase gene is a human guanylyltransferase gene. In some examples, the vertebrate RNA triphosphatase gene is a human RNA triphosphatase gene. In some examples, the vertebrate guanylyltransferase gene is a pig guanylyltransferase gene. In some examples the vertebrate RNA triphosphatase gene is a pig RNA triphosphatase gene.

Further disclosed herein is an isolated virus. In some examples, the virus includes SARS-CoV-2 nsp14 including one or more of the following substitutions: nsp14 W293F, F368N, F368L, D353T, or D353A, where the substitutions refer to SEQ ID NO: 4. In some examples, the virus includes MERS-CoV nsp14 including the following substitution: F365G, where the substitution refers to SEQ ID NO: 24. In some examples, the virus includes ASFV NP868R including one or more of the following substitutions: Y714L, F711L, F711W, D680L, K647Y, or S604A, where the substitutions refer to SEQ ID NO: 10. In some examples the virus includes MPV D1 including one or more of the following substitutions: Y555F, R655A, D545A, D598A, Y683V, or R548A, R548K where the substitutions refer to SEQ ID NO: 290. In some examples, the virus includes SARS-CoV-2 nsp12 including one or more of the following substitutions: K73A, D218A, D760A, V30A, R33A, R33L, R33K, R55A, R55L, R55K, K50A, C53A, R116A, V71A, N209A, L119A, or Y217F where the substitutions refer to SEQ ID NO: 291. In some examples, the virus includes WNV ns5 including one or more of the following substitutions: F24L, F24E, or S150C where the substitutions refer to SEQ ID NO: 292. In some examples, the isolated virus including SARS-CoV-2 nsp14 is SARS-CoV-2. In some examples it is another virus. In some examples the isolated virus including MERS-CoV nsp14 is MERS-CoV. In some examples it is another virus. In some examples the isolated virus including ASFV NP868R is ASFV. In some examples it is another virus. In some examples the isolated virus including SARS COV 2 nsp12 is SARS-CoV-2. In some examples it is another virus. In some examples the isolated virus including WNV ns5 is WNV. In some examples it is another virus. Further disclosed herein, is an isolated virus as described above, and a carrier, optionally further including an adjuvant. In some examples, the viruses described herein can be used in a method of stimulating an immune response in a subject. In some examples, this includes administering a therapeutically effective amount of the isolated virus or the composition of to the subject, thereby stimulating an immune response in the subject.

### IV. Genetically Modified Yeast

The present disclosure provides isolated yeast having its native methyltransferase, guanylyltransferase, and/or RNA triphosphatase genes inactivated. In some examples, inactivation decreases expression and/or activity by at least 50%, at least 75%, at least 90%, at least 95%, at least 99%, at least 99.9% or even 100% (e.g., not detectable). In some examples the native methyltransferase gene is Abd1. In some examples the native guanylyltransferase gene is ceg1. In some examples the native RNA triphosphatase is cet1. Contemplated herein are isolated yeast containing a genetic inactivation (e.g., functional deletion) of an abd1, ceg1, and/or cet1 gene, or homolog thereof. Any yeast can be used, such as Saccharomyces, Candida, Cryptococcus, Hansenula, Kluyveromyces, Pichia, Rhodotorula, Schizosaccharomyces and Yarrowia. In some examples the yeast is Saccharomyces cerevisiae or Saccharomyces carlsbergensis. In particular examples, the disclosed yeast with inactivated abd1 is American Type Culture Collection (ATCC) no. 4033376. In particular examples, the disclosed yeast with inactivated ceg1 is Horizon Discovery accession #YGL130W, such as Clone ID: 24497. In particular examples, the disclosed yeast with inactivated cet1 is Horizon Discovery accession #YPL228W, such as Clone ID: 21064.

Yeast strains can be cultured by a variety of microbiological techniques. Yeast can be grown on solid, or liquid media, such as YPD media (ThermoFisher Cat #A1374501). In some examples, yeast is grown in synthetic defined media containing 0.67% nitrogen base without amino acids, 2% glucose, 0.02 mg/mL lysine, 0.02 mg/mL methionine, 0.02 mg/mL histidine, and 0.1 mg/ml carbenicillin, optionally with Leucine 0.012 mg/mL. General protocols and media for culturing yeast can be found in Lawrence Bergman, Methods in Molecular Biology, Two-Hybrid Systems: Methods and Protocols Chapters 2 and 3, Vol. 177.

In some examples, the isolated yeast includes a marker, for example to isolate or identify yeast strains that carry the marker. A marker can be a selectable marker, such as a resistance gene, such as an antibiotic resistance gene or an antifungal resistance gene. In some examples the marker replaces a native yeast gene, permitting identification or selection of yeast strains with genetic inactivation of a native yeast gene. In some examples, the marker is present on a plasmid, allowing identification or selection of yeast strains containing the plasmid.

### Genetic Inactivation

Disclosed herein is an isolated yeast cell. In some examples the yeast cell is a non-native yeast, having genetic inactivation of one or more genes. In some examples, the abd1 gene is inactivated in the yeast cell. In some examples, the ceg1 gene is inactivated in the yeast cell. In some examples the cet1 gene is inactivated in the yeast cell.

The genetic inactivation of abd1, ceg1, and/or cet1 gene, or homolog thereof can be on one or more strands of the yeast's genome. In some examples the disclosed yeast is a diploid strain, in other examples the disclosed yeast is a haploid strain. In some examples the yeast can undergo sporulation (generating haploid yeast spores from a diploid yeast strain) or mating (generating diploid yeast from a haploid yeast strain). In some examples, a diploid yeast has one native chromosome and one chromosome with genetic inactivation of a gene such as abd1, ceg1, and/or cet1. In some examples, sporulation of such a diploids strain can result in yeast with genetic inactivation of its only copy of a gene such as abd1, ceg1, and/or cet1. Yeast sporulation can be accomplished by methods such as Paulissen and Huang, Efficient Sporulation of Saccharomyces Cerevisiae in a 96 Multiwell Format, JoVE (2016) 115: e54584, incorporated by reference herein in its entirety. Yeast mating can be accomplished by methods such as Lawrence Bergman, Methods in Molecular Biology, Two-Hybrid Systems: Methods and Protocols Chapters 2 and 3, Vol. 177.

Any method for genetic inactivation can be used, as long as the expression of the abd1, ceg1 and/or cet1 gene(s) are significantly reduced or eliminated (e.g., a reduction of at least 50%, at least 75%, at least 90%, at least 95%, at least 99%, at least 99.9% or even 100% (e.g., not detectable)), and/or the function of the expressed ABD1, CEG1 and/or CET1 protein(s) are significantly reduced or eliminated (e.g., a reduction of at least 50%, at least 75%, at least 90%, at least 95%, at least 99%, at least 99.9% or even 100% (e.g., not detectable)), such as knocked out (or otherwise made inoperative). For example, the abd1, ceg1 and/or cet1 gene(s) can be manipulated to include one or more nucleotide substitutions, insertions, deletions, or combinations thereof, which result in inactivation of the gene. In particular examples, the abd1, ceg1 and/or cet1 gene(s) are genetically inactivated by a complete or partial deletion mutation, by insertional mutation, or both. In some examples, the abd1, ceg1 and/or cet1 gene(s) are genetically inactivated by the introduction of a stop codon, thereby disrupting expression of the protein. In some examples, the abd1, ceg1 and/or cet1 gene(s) can be inactivated using CRISPR/Cas9. CRISPR/Cas9 inactivation of genes in yeast can be accomplished by methods such as Novarina et al., A user-friendly and streamlined protocol for CRISPR/Cas9 genome editing in budding yeast, STAR Protocols (2022) 3:101358.

In some examples, genetic inactivation need not be 100% genetic inactivation. In some examples, genetic inactivation refers to at least 50%, at least 60%, at least 70%, at least 80%, at least 90%, or at least 95% gene or protein inactivation. The term “reduced” or “decreased” as used herein with respect to a particular gene or protein activity refers to a lower level of activity than that measured in a comparable cell of the same species. For example, a particular yeast having decreased abd1, ceg1, and/or cet1 activity has reduced abd1, ceg1, and/or cet1 activity if a comparable yeast not having an abd1, ceg1, and/or cet1 genetic mutation has detectable abd1, ceg1, and/or cet1 activity. Abd1 sequences are disclosed herein, and others are publicly available, for example from GenBank or EMBL. In some examples, the abd1 gene genetically inactivated includes a nucleic acid molecule having at least 80%, at least 90%, at least 95%, at least 97%, at least 98%, at least 99%, or 100% sequence identity to SEQ ID NO: 35 prior to its inactivation. In some examples, the abd1 gene genetically inactivated encodes a protein comprising at least 80%, at least 90%, at least 95%, at least 97%, at least 98%, at least 99%, or 100% sequence identity to SEQ ID NO: 36 prior to the inactivation.

Ceg1 sequences are disclosed herein, and others are publicly available, for example from GenBank or EMBL. In some examples, the ceg1 gene genetically inactivated includes a nucleic acid molecule having at least 80%, at least 90%, at least 95%, at least 97%, at least 98%, at least 99%, or 100% sequence identity to SEQ ID NO: 37 prior to its inactivation. In some examples, the ceg1 gene genetically inactivated encodes a protein comprising at least 80%, at least 90%, at least 95%, at least 97%, at least 98%, at least 99%, or 100% sequence identity to SEQ ID NO: 38 prior to the inactivation.

cet1 sequences are disclosed herein and others are publicly available, for example from GenBank or EMBL. In some examples, the cet1 gene genetically inactivated includes a nucleic acid molecule having at least 80%, at least 90%, at least 95%, at least 97%, at least 98%, at least 99%, or 100% sequence identity to SEQ ID NO: 39 prior to its inactivation. In some examples, the cet1 gene genetically inactivated encodes a protein comprising at least 80%, at least 90%, at least 95%, at least 97%, at least 98%, at least 99%, or 100% sequence identity to SEQ ID NO: 40 prior to the inactivation.

As used herein, an “inactivated” or “functionally deleted” gene means that the gene has been mutated, for example by insertion, deletion, or substitution (or combinations thereof) of one or more nucleotides such that the mutation substantially reduces (and in some cases abolishes) expression or biological activity of the encoded gene product. The mutation can act through affecting transcription or translation of the gene or its mRNA, or the mutation can affect the polypeptide product itself in such a way as to render it substantially inactive.

Genetic inactivation of one or more genes (can be referred to as functional deletion) can be performed using any method. In one example, a strain of Saccharomyces cerevisiae or other yeast is transformed with a vector which has the effect of down-regulating or otherwise inactivating an abd1, ceg1, and/or cet1 gene or homolog thereof. For example, control elements such as promoters and the like which control gene expression can be mutated, the coding region of the gene can be mutated so that any protein expressed is substantially inactive, or the abd1, ceg1, and/or cet1 gene or homolog thereof can be deleted entirely. For example, an abd1, ceg1, and/or cet1 gene or homolog thereof can be functionally deleted by complete or partial deletion mutation (for example by deleting a portion of the coding region of the gene), by insertional mutation (for example by inserting nucleotides into the coding region of the gene, such as a molecule of about 1-5000 nucleotides), or combinations thereof. Thus, the disclosure in some examples provides transformed or recombinant yeast that include at least one exogenous nucleic acid molecule which genetically inactivates an abd1, ceg1, and/or cet1 gene (such as mutates the nucleic acid sequence of SEQ ID NOs: 35, 37, and/or 39) or a homolog thereof.

In one example, an insertional mutation includes introduction of a nucleic acid molecule that is in multiples of three bases (e.g., a sequence of 3, 9, 12, or 15 nucleotides) to reduce the possibility that the insertion will affect downstream genes. Mutations can also be generated through insertion of a foreign gene sequence, for example the insertion of a gene encoding antibiotic resistance (such as hygromycin, bleomycin, G418, neomycin, or kanamycin) into the abd1, ceg1, and/or cet1 gene or homolog thereof.

In one example, genetic inactivation is achieved by deletion of a portion of the coding region of the abd1, ceg1, and/or cet1 gene(s) or homolog thereof. For example, some, most (such as at least 50%) or virtually the entire coding region can be deleted. In particular examples, about 5% to about 100% of the gene is deleted, such as at least 20% of the gene, at least 40% of the gene, at least 75% of the gene, or at least 90% of the abd1, ceg1, and/or cet1 gene(s). In particular examples, about 5% to about 100% of the coding region is deleted, such as at least 20% of the coding region, at least 40% of the coding region, at least 75% of the coding region, or at least 90% of the abd1, ceg1, and/or cet1 coding region(s).

In one example, allelic exchange is employed to genetically inactivate one or more genes, such as abd1, ceg1, and/or cet1.

In one example, counterselectable markers (such as pyrG) are employed (e.g., see Reyrat et al., Infec. Immun. 66:4011-4017, 1998). In this technique, a double selection strategy is employed wherein a plasmid is constructed encoding both a selectable and counterselectable marker, with flanking DNA sequences derived from both sides of the desired deletion. The selectable marker is used to select for fungi in which the plasmid has integrated into the genome in the appropriate location and manner. The counterselectable marker is used to select for the small percentage of fungi that have spontaneously eliminated the integrated plasmid. A fraction of these fungi will then contain only the desired deletion with no other foreign DNA present.

In another technique, the cre-lox system is used for site specific recombination of DNA (for example see Steiger et al., Appl. Environ. Microbiol. 77(1):114, 2011). The system includes 34 base pair lox sequences that are recognized by the bacterial cre recombinase gene. If the lox sites are present in the DNA in an appropriate orientation, DNA flanked by the lox sites will be excised by the cre recombinase, resulting in the deletion of all sequences except for one remaining copy of the lox sequence. Using recombination techniques, the targeted gene (e.g., abd1, ceg1, and/or cet1 or homolog thereof) can be deleted in the Saccharomyces genome and replaced with a selectable marker (for example a gene coding for kanamycin or other antibiotic resistance) that is flanked by the lox sites. Transient expression (e.g., by electroporation of a suicide plasmid containing the cre gene under control of a promoter that functions in Saccharomyces or other fungus) of the cre recombinase efficiently eliminates the lox flanked marker. This process can produce mutant fungi containing the desired deletion mutation of abd1, ceg1, and/or cet1 or homolog thereof and one copy of the lox sequence.

In another example, an abd1, ceg1, and/or cet1 gene sequence (or homolog thereof) in a fungal genome is replaced with a marker gene, such as one encoding for green fluorescent protein, 0-galactosidase, or luciferase. In this technique, DNA segments flanking a desired deletion are prepared by PCR and cloned into a suicide (non-replicating) vector for the fungus. An expression cassette, containing a promoter active in the fungus and the appropriate marker gene, is cloned between the flanking sequences. The plasmid is introduced into wild-type fungi. Fungi that incorporate and express the marker gene are isolated and examined for the appropriate recombination event (replacement of the wild type abd1, ceg1, and/or cet1 gene with the marker gene).

Thus, for example, a fungal cell can be engineered to have a disrupted abd1, ceg1, and/or cet1 gene (or homolog thereof) using mutagenesis or knock-out technology. (Methods in Yeast Genetics (1997 edition), Adams, Gottschling, Kaiser, and Sterns, Cold Spring Harbor Press, 1998; Datsenko and Wanner, Proc. Natl. Acad. Sci. USA 97: 6640-5, 2000; and Dai et al., Appl. Environ. Microbiol. 70(4):2474-85, 2004).

In another example, antisense technology is used to reduce or eliminate the activity of abd1, ceg1, and/or cet1 (or homolog thereof). For example, a fungal cell can be engineered to contain a cDNA that encodes an antisense molecule that prevents abd1, ceg1, and/or cet1 from being translated. The term “antisense molecule” encompasses any nucleic acid molecule or nucleic acid analog (e.g., peptide nucleic acids) that contains a sequence that corresponds to the coding strand of an endogenous abd1, ceg1, and/or cet1 gene (or homolog thereof). An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axehead structures, provided the molecule cleaves RNA. Further, gene silencing can be used to reduce the activity of abd1, ceg1, and/or cet1.

Any method can be used to introduce an exogenous nucleic acid molecule into a fungal cell, for example to genetically inactivate abd1, ceg1, and/or cet1 (or homolog thereof). For example, chemical mediated-protoplast transformation, electroporation, Agrobacterium-mediated transformation, fusion of protoplasts, and biolistic delivery are common methods for introducing nucleic acid into fungal cells. (See, e.g., Ito et al., J. Bacteriol. 153:163-8, 1983; Durrens et al., Curr. Genet. 18:7-12, 1990; Sambrook et al., Molecular cloning: A laboratory manual, Cold Spring Harbour Laboratory Press, New York, USA, third edition, 2001; and Becker and Guarente, Methods in Enzymology 194:182-7, 1991). An exogenous nucleic acid molecule contained within a particular cell of the disclosure can be maintained within that cell in any form. For example, exogenous nucleic acid molecules can be integrated into the genome of the cell or maintained in an episomal state. That is, the recombinant fungi cells can be a stable or transient transformant.

A fungus having an inactivated abd1, ceg1, and/or cet1 gene (or homolog thereof) can be identified using any known method. For example, PCR and nucleic acid hybridization techniques, such as Northern and Southern analysis, can be used to confirm that a fungus has an inactivated abd1, ceg1, and/or cet1 gene (or homolog thereof). Alternatively, real-time reverse transcription PCR (qRT-PCR) can be used for detection and quantification of targeted messenger RNA, such as mRNA of abd1, ceg1, and/or cet1 gene (or homolog thereof) in the parent and mutant strains as grown at the same culture conditions. Immunohisto-chemical and biochemical techniques can also be used to determine if a cell expresses abd1, ceg1, and/or cet1 (or homolog thereof) by detecting the expression of the Abd1, Ceg1, and/or Cet1 protein(s) (or homolog thereof) encoded by abd1, ceg1, and/or cet1 (or homolog thereof). For example, an antibody having specificity for Abd1, Ceg1, and/or Cet1 (or homolog thereof) can be used to determine whether or not a particular fungus contains a functional nucleic acid encoding Abd1, Ceg1, and/or Cet1 protein (or homolog thereof). Further, biochemical techniques can be used to determine if a cell contains a particular gene inactivation by detecting a product produced as a result of the expression of the peptide.

Abd1, Ceg1, and/or Cet1 protein and nucleic acid sequences are publicly available and specific examples are provided herein. In addition, abd1, ceg1, and/or cet1 sequences, as well as the sequences of homologs thereof, can be identified using routine molecular biology methods. Exemplary abd1, ceg1, and cet1 nucleic acid sequences that can be genetically inactivated are shown in SEQ ID NOs: 35, 37, and 39 respectively. Variant sequences may contain a single insertion, a single deletion, a single substitution, multiple insertions, multiple deletions, multiple substitutions, or any combination thereof (e.g., single deletion together with multiple insertions). In addition, the degeneracy of the code permits multiple nucleic acid sequences to encode the same protein. In some examples, the abd1, ceg1, and/or cet1 gene that is genetically inactivated includes a nucleic acid molecule having at least 80%, at least 90%, at least 95%, at least 97%, at least 98%, at least 99%, or 100% sequence identity to SEQ ID NO: 35, 37, and 39, respectively prior to the inactivation.

Additional abd1, ceg1, and/or cet1 sequences (and homologs thereof) can be identified using any method such as those described herein. For example, abd1, ceg1, and/or cet1 nucleic acid molecules (and homologs thereof) that encode an Abd1, Ceg1p, or Cet1p protein can be identified and obtained using molecular cloning or chemical nucleic acid synthesis procedures and techniques, including PCR. In addition, nucleic acid sequencing techniques and software programs that translate nucleic acid sequences into amino acid sequences based on the genetic code can be used to determine whether or not a particular nucleic acid has any sequence homology with known abd1, ceg1, and/or cet1 sequences. Sequence alignment software such as MEGALIGN (DNASTAR, Madison, W I, 1997) can be used to compare various sequences.

Nucleic acid hybridization techniques can be used to identify and obtain a nucleic acid molecule that encodes an Abd1, Ceg1p, and/or Cet1p protein. Briefly, any known abd1, ceg1, and/or cet1 nucleic acid molecule, or fragment thereof, can be used as a probe to identify similar nucleic acid molecules (including homologs thereof) by hybridization under conditions of moderate to high stringency. Such similar nucleic acid molecules then can be isolated, sequenced, and analyzed to determine whether the encoded protein is an Abd1, Ceg1p, and/or Cet1p protein.

### Heterologous, Native, and Non-Native Gene Expression

Expression of heterologous genes can be performed using any acceptable method. In some examples the heterologous gene is a native gene, or a non-native gene. In some examples a strain of Saccharomyces is transformed with a heterologous methyltransferase. In some examples a strain of Saccharomyces is transformed with a heterologous guanylyltransferase. In some examples a strain of Saccharomyces is transformed with a heterologous RNA triphosphatase. The heterologous methyltransferase, heterologous guanylyltransferase, or heterologous RNA triphosphatase can include vertebrate methyltransferases, guanylyltransferases, or RNA triphosphatases. The heterologous methyltransferase, heterologous guanylyltransferase, or heterologous RNA triphosphatase can include bacterial methyltransferases, guanylyltransferases, or RNA triphosphatases. The heterologous methyltransferase, heterologous guanylyltransferase, or heterologous RNA triphosphatase can include fungal methyltransferases, guanylyltransferases, or RNA triphosphatases. The heterologous methyltransferase, heterologous guanylyltransferase, or heterologous RNA triphosphatase can include viral methyltransferases, guanylyltransferases, or RNA triphosphatases. Expression of the heterologous genes disclosed is herein.

In some examples, the heterologous methyltransferase is abd1, RNMT, nsp14, d1, d12, d1 and d12, NP868R, and/or ns5. In some examples, the heterologous abd1 methyltransferase is from a different strain of fungus than the yeast. In some examples the heterologous guanylyltransferase is ceg1, MCE1, nsp12, d1, d12, d1 and d12, NP868R, and/or ns5. In some examples, the heterologous ceg1 guanylyltransferase is from a different strain of fungus than the yeast. In some examples the heterologous RNA triphosphatase is cet1, MCE1, nsp9, d1, d12, d1 and d12, and/or NP868R. In some examples, the heterologous cet1 RNA triphosphatase is from a different strain of fungus than the yeast.

In one example, a strain of yeast is transformed with a vector that introduces a heterologous methyltransferase, guanylyltransferase, or RNA triphosphatase. This can be done by introducing one or more coding sequences for a heterologous methyltransferase, guanylyltransferase, or RNA triphosphatase, optionally including sequences such as promoters, operably linked to the heterologous sequences. A heterologous methyltransferase, guanylyltransferase, or RNA triphosphatase can be expressed in a yeast cell by introduction of a vector that includes one or more heterologous sequences (such as 1, 2, 3, 4, 5, 6, 7, 8, 9 or 10 heterologous methyltransferase sequences or copies of such sequences, such as 1, 2, 3, 4, 5, 6, 7, 8, 9 or 10 copies of nsp14 sequences or copies of such sequences) into the desired yeast cell. In some examples, the heterologous sequences are from the same species, in some examples there are multiple copies from different species, or combinations thereof, such as heterologous methyltransferase sequences from at least 2, 3, 4, 5, 6, 7, 8, 9, or 10 different species. In some examples, the heterologous sequence(s) introduced into the yeast is optimized for codon usage.

In some examples, a wild type sequences, such as a wild type RNMT, MCE1, MCE1, nsp14, nsp12, nsp9, D1, D12, NP868R, or ns5 sequence further includes an N-terminal M.

The disclosure in some examples provides transformed yeast that include at least one exogenous nucleic acid molecule which includes a nsp14 gene or coding sequence (such as a nucleic acid encoding a protein with at least 80%, at least 85%, at least 90%, at least 95%, at least 97%, at least 98%, or at least 99% sequence identity to SEQ ID NOs: 4 or 289). In some examples, the nsp14 gene or coding sequence further includes an N-terminal M, e.g., as shown in SEQ ID NO: 4. In some examples, provided is transformed yeast that include at least one exogenous nucleic acid molecule which includes a D1 gene or coding sequence (such as a nucleic acid encoding a protein with at least 80%, at least 85%, at least 90%, at least 95%, at least 97%, at least 98%, or at least 99% sequence identity to SEQ ID NOs: 6 or 290). In some examples, the D1 gene or coding sequence further includes an N-terminal M, e.g., as shown in SEQ ID NO: 6. In some examples, provided is transformed yeast that include at least one exogenous nucleic acid molecule which includes a D12 gene or coding sequence (such as a nucleic acid encoding a protein with at least 80%, at least 85%, at least 90%, at least 95%, at least 97%, at least 98%, or at least 99% sequence identity to SEQ ID NO:8). In some examples, provided is transformed yeast that include at least one exogenous nucleic acid molecule which includes a NP868R gene or coding sequence (such as a nucleic acid encoding a protein with at least 80%, at least 85%, at least 90%, at least 95%, at least 97%, at least 98%, or at least 99% sequence identity to SEQ ID NO:10). In some examples, provided is transformed yeast that include at least one exogenous nucleic acid molecule which includes a ns5 gene or coding sequence (such as a nucleic acid encoding a protein with at least 80%, at least 85%, at least 90%, at least 95%, at least 97%, at least 98%, or at least 99% sequence identity to SEQ ID NOs: 31 or 292). In some examples the ns5 gene or coding sequence further includes an N-terminal M, e.g., as shown in SEQ ID NO: 31. In some examples, provided is transformed yeast that include at least one exogenous nucleic acid molecule which includes a nsp12 gene or coding sequence (such as a nucleic acid encoding a protein with at least 80%, at least 85%, at least 90%, at least 95%, at least 97%, at least 98%, or at least 99% sequence identity to SEQ ID NOs: 29 or 291). In some examples the nsp12 gene or coding sequence further includes an N-terminal M, e.g., as shown in SEQ ID NO: 29. In some examples, provided is transformed yeast that include at least one exogenous nucleic acid molecule which includes a nsp9 gene or coding sequence (such as a nucleic acid encoding a protein with at least 80%, at least 85%, at least 90%, at least 95%, at least 97%, at least 98%, or at least 99% sequence identity to SEQ ID NO: 34). In some examples, provided is transformed yeast that include at least one exogenous nucleic acid molecule which includes a RNMT gene or coding sequence (such as a nucleic acid encoding a protein with at least 80%, at least 85%, at least 90%, at least 95%, at least 97%, at least 98%, or at least 99% sequence identity to SEQ ID NO: 14). In some examples, provided is transformed yeast that include at least one exogenous nucleic acid molecule which includes a MCE1 gene or coding sequence (such as a nucleic acid encoding a protein with at least 80%, at least 85%, at least 90%, at least 95%, at least 97%, at least 98%, or at least 99% sequence identity to SEQ ID NO: 33). In some examples, provided is transformed yeast that include at least one exogenous nucleic acid molecule which includes a Abd1 gene or coding sequence (such as a nucleic acid encoding a protein with at least 80%, at least 85%, at least 90%, at least 95%, at least 97%, at least 98%, or at least 99% sequence identity to SEQ ID NO: 36). In some examples, provided is transformed yeast that include at least one exogenous nucleic acid molecule which includes a Ceg1p gene or coding sequence (such as a nucleic acid encoding a protein with at least 80%, at least 85%, at least 90%, at least 95%, at least 97%, at least 98%, or at least 99% sequence identity to SEQ ID NO: 38). In some examples, provided is transformed yeast that include at least one exogenous nucleic acid molecule which includes a Cet1p gene or coding sequence (such as a nucleic acid encoding a protein with at least 80%, at least 85%, at least 90%, at least 95%, at least 97%, at least 98%, or at least 99% sequence identity to SEQ ID NO: 40).

Transformation can be accomplished by any suitable method, such as the use of spheroplasts, LiAc, electroporation, bombardment, or glass beads. To accomplish a LiAc transformation, briefly, yeast cells are grown to mid-log phase and then suspended in a solution containing lithium acetate (LiAc). The cells are then incubated with a plasmid, followed by exposure to PEG, and a heat shock, resulting in transformation. For detailed methods, see Kawai et al., Transformation of Saccharomyces cerevisiae and other fungi Bioeng Bugs (2010) 1:395-403, incorporated by reference herein in its entirety.

In another technique, the cre-lox system is used for site specific recombination of DNA (for example see Steiger et al., Appl. Environ. Microbiol. 77(1):114, 2011). The system includes 34 base pair lox sequences that are recognized by the bacterial cre recombinase gene. If the lox sites are present in the DNA in an appropriate orientation, DNA flanked by the lox sites will be excised by the cre recombinase, resulting in the deletion of all sequences except for one remaining copy of the lox sequence. Using standard recombination techniques, a targeted gene can be deleted in the yeast genome and replaced with one or more copies of a non-native sequence (for example in S. cerevisiae, replacing one or both S. cerevisiae abd1 sequences with RNMT, nsp14, d1, d12, d1 and d12, NP868R, and/or ns5. In another example, heterologous genes such as RNMT, MCE1, nsp14, nsp12, nsp9, d1, d12, d1 and d12, NP868R, and/or ns5 is inserted replacing a non-protein coding sequence, or a sequence coding for a selectable marker. In some examples, the non-native sequence or heterologous genes additional include a selectable marker. Transient expression (by electroporation of a suicide plasmid containing the cre gene under control of a promoter that functions in yeast) of the cre recombinase should result in efficient elimination of the lox flanked marker. This process will produce a yeast containing the desired insertion mutation and one copy of the lox sequence.

In one example, one or more heterologous genes are introduced into yeast cells by chemical mediated protoplast transformation in combination of yeast-gap repairing method for transgene expression construction. In one example, one or more heterologous genes are introduced into yeast cells by a CRISPR/Cas system. CRISPR/Cas9 mediated insertion of genes in yeast can be accomplished by methods such as Novarina et al., A user-friendly and streamlined protocol for CRISPR/Cas9 genome editing in budding yeast, STAR Protocols (2022) 3:101358. Additional methods for expressing heterologous, native, and non-native genes in yeast are explained in Romanos et al., Foreign Gene Expression in Yeast: A Review, Yeast (1992) 8:423-88. In some examples, the heterologous gene is introduced via a plasmid. Plasmids can be constructed by any appropriate method, such as the methods of Gibson, D. G.; Young, L.; Chuang, R.-Y.; Venter, J. C.; Hutchison, C. A.; Smith, H. O. Enzymatic Assembly of DNA Molecules up to Several Hundred Kilobases. Nat Methods 2009, 6 (5), 343-345.

In one example a plasmid is generated and expressed in the desired yeast cell, such as a yeast cell with genetically inactivated Abd1, Ceg1p, and/or Cet1p. In some examples, the plasmid encodes a heterologous methyltransferase, such as heterologous abd1, RNMT, nsp14, d1, d12, d1 and d12, NP868R, and/or ns5. In some examples, the heterologous methyltransferase in the plasmid is operably linked to a promoter, such as a pTpl1 promoter. In one example, the pTpl1 promoter has at least 80%, at least 85%, at least 90%, at least 95%, at least 97%, at least 98%, or at least 99% sequence identity to SEQ ID NO: 13. In some examples, the heterologous methyltransferase in the plasmid is fused to an RNA polymerase complex targeting gene, such as a Mce1 GTase domain gene. In some examples, the Mce1 GTase domain gene encodes a protein with at least 80%, at least 85%, at least 90%, at least 95%, at least 97%, at least 98%, or at least 99% sequence identity to SEQ ID NO: 1. In some examples the heterologous methyltransferase is fused to the 5′ aspect of the RNA polymerase complex targeting gene. In some examples the heterologous methyltransferase is fused to the 3′ aspect of the RNA polymerase complex targeting gene. In some examples, the heterologous methyltransferase and RNA polymerase complex targeting gene form a fusion product is by RNA splicing or post translational modifications. In some examples the plasmid has at least 80%, at least 85%, at least 90%, at least 95%, at least 97%, at least 98%, or at least 99% sequence identity to at least one of SEQ ID NOs: 16, 17, 18, 19, 21, 22, and/or 23.

In some examples the plasmid encodes a heterologous abd1 gene. In some examples, the plasmid includes a yeast ura3 gene. In some examples, the plasmid is curable. In some examples the plasmid has at least 80%, at least 85%, at least 90%, at least 95%, at least 97%, at least 98%, or at least 99% sequence identity to SEQ ID NO: 3.

In some examples, the plasmid encodes a heterologous guanylyltransferase, such as heterologous ceg1, MCE1, nsp12, d1, d12, d1 and d12, NP868R, and/or ns5. In some examples, the heterologous guanylyltransferase in the plasmid is operably linked to a promoter, such as a pTpl1 promoter. In one example, the pTpl1 promoter has at least 80%, at least 85%, at least 90%, at least 95%, at least 97%, at least 98%, or at least 99% sequence identity to SEQ ID NO: 13. In some examples, the heterologous guanylyltransferase in the plasmid is fused to an RNA polymerase complex targeting gene, such as a Mce1 GTase domain gene. In some examples, the Mce1 GTase domain gene encodes a protein with at least 80%, at least 85%, at least 90%, at least 95%, at least 97%, at least 98%, or at least 99% sequence identity to SEQ ID NO: 1. In some examples the heterologous guanylyltransferase is fused to the 5′ aspect of the RNA polymerase complex targeting gene. In some examples the heterologous guanylyltransferase is fused to the 3′ aspect of the RNA polymerase complex targeting gene. In some examples, the heterologous guanylyltransferase and RNA polymerase complex targeting gene form a fusion product is by RNA splicing or post translational modifications. In some examples the plasmid has at least 80%, at least 85%, at least 90%, at least 95%, at least 97%, at least 98%, or at least 99% sequence identity to at least one of SEQ ID NOs: 26 and/or 27.

In some examples the plasmid encodes a heterologous ceg1 gene. In some examples, the plasmid includes a yeast ura3 gene. In some examples, the plasmid is curable. In some examples the plasmid has at least 80%, at least 85%, at least 90%, at least 95%, at least 97%, at least 98%, or at least 99% sequence identity to SEQ ID NO: 28.

In some examples, the plasmid encodes a heterologous RNA Triphosphatase, such as heterologous cet1, MCE1, nsp9, d1, d12, d1 and d12, and/or NP868R. In some examples, the heterologous RNA Triphosphatase in the plasmid is operably linked to a promoter, such as a pTpl1 promoter. In one example, the pTpl1 promoter has at least 80%, at least 85%, at least 90%, at least 95%, at least 97%, at least 98%, or at least 99% sequence identity to SEQ ID NO: 13. In some examples, the heterologous RNA Triphosphatase in the plasmid is fused to an RNA polymerase complex targeting gene, such as a Mce1 GTase domain gene. In some examples, the Mce1 GTase domain gene has at least 80%, at least 85%, at least 90%, at least 95%, at least 97%, at least 98%, or at least 99% sequence identity to SEQ ID NO: 1.

In some examples the plasmid encodes a heterologous cet1 gene. In some examples, the plasmid includes a yeast ura3 gene. In some examples, the plasmid is curable.

In some examples, the heterologous methyltransferase is abd1, RNMT, nsp14, d1, d12, d1 and d12, NP868R, and/or ns5. In some examples, the heterologous abd1 methyltransferase is from a different strain of fungus than the yeast. In some examples the heterologous guanylyltransferase is ceg1, MCE1, nsp12, d1, d12, d1 and d12, NP868R, and/or ns5. In some examples, the heterologous ceg1 guanylyltransferase is from a different strain of fungus than the yeast. In some examples the heterologous RNA triphosphatase is cet1, MCE1, nsp9, d1, d12, d1 and d12, and/or NP868R. In some examples, the heterologous cet1 RNA triphosphatase is from a different strain of fungus than the yeast.

Thus, for example, a yeast cell can be engineered to express heterologous genes, such as heterologous abd1, heterologous ceg1, heterologous cet1, RNMT, MCE1, nsp14, nsp12, nsp9, d1, d12, d1 and d12, NP868R, and/or ns5.

### V. Methods of Using the Modified Yeast

The present disclosure provides for isolated, yeast having genetically inactivated Abd1, Ceg1p, and/or Cet1p. In some examples the isolated yeast incorporates a heterologous methyltransferase, heterologous guanylyltransferase, and/or heterologous RNA triphosphatase.

### Identifying Attenuation Mutations

In one example, yeasts of this disclosure can be used to identify attenuation mutations in the heterologous methyltransferase, heterologous guanylyltransferase, and/or heterologous RNA triphosphatase. For example, for a heterologous methyltransferase, a methyltransferase gene from a pathogenic organism can be selected, and subjected to mutagenesis. Mutagenesis can be accomplished by any acceptable method, such as alanine scanning, or constructing a library targeting a residue of interest, using oligonucleotides. The resulting mutants can be incorporated into plasmids, forming a library of heterologous methyltransferases. This library can be incorporated into yeast having genetically inactivated Abd1, and the growth rate of the mutants compared. In one example, yeast containing a mutant methyltransferase that does not grow could be identified as an inactivation mutation. In one example, the growth of yeast containing mutant methyltransferases could be compared to a control, such as a yeast containing an unmanipulated methyltransferase, or a historical control. In one example, yeast containing a mutant methyltransferase grows slower than a control, such as at least 10% slower, at least 20% slower, at least 30% slower, at least 40% slower, at least 50% slower, at least 60% slower, at least 70% slower, at least 80% slower, or at least 90% slower could be identified as yeast having an attenuation mutation. In one example, yeast containing a mutant methyltransferase grows slower than a control, such as at most 10% slower, at most 20% slower, at most 30% slower, at most 40% slower, at most 50% slower, at most 60% slower, at most 70% slower, at most 80% slower, or at most 90% slower could be identified as yeast having an attenuation mutation. A similar protocol could be used to identify attenuated guanylyltransferase genes, or attenuated RNA triphosphatase genes.

In some examples, the yeasts disclosed herein are used in a method of identifying one or more attenuating mutations in the heterologous viral methyltransferase; heterologous viral guanylyltransferase; or heterologous viral RNA triphosphatase of the yeast. In some examples, the method includes the steps of altering one or more amino acid residues in the heterologous viral methyltransferase; heterologous viral guanylyltransferase; or heterologous viral RNA triphosphatase of the isolated non-native yeast to generate an experimental strain, culturing the experimental strain, measuring growth of the experimental strain, and comparing the growth of the experimental strain to a control. In some examples, the experimental strain growing slower than the control indicates the presence of an attenuating mutation in the experimental strain. Culturing the yeasts disclosed herein can be accomplished by any suitable methods, such as culturing on solid or liquid media, such as YPD or chemically defined media, such as media containing a selection agent. Measuring the growth of the yeast disclosed herein can be accomplished by any suitable method, such as dilution plating, or use of spectrophotometry, or by using a yeast strain which incorporates a reporter, such as a fluorescent reporter, which can be measured by flow cytometry.

Identifying Methyltransferases, Guanylyltransferase and/or RNA Triphosphatases Inhibitors

In one example, yeasts of this disclosure are used to identify compounds which inhibit Methyltransferases, Guanylyltransferase and/or RNA Triphosphatases, such as Methyltransferases, Guanylyltransferase and/or RNA Triphosphatases expressed by pathogens. For example, a non-native yeast having genetically inactivated Abd1, Ceg1p, and/or Cet1p, and also expressing a heterologous methyltransferase, heterologous guanylyltransferase, and/or heterologous RNA triphosphatase, can be grown in media which contains a compound which may inhibit the heterologous genes. The growth rate of the yeast can be measured, and compared to a control such as a similar strain of yeast begin grown in the absence of the compound which may inhibit the heterologous genes, or a historical control. In one example, if the yeast grown in the presence of the compound grows slower than the control, such as at least 10% slower, at least 20% slower, at least 30% slower, at least 40% slower, at least 50% slower, at least 60% slower, at least 70% slower, at least 80% slower, or at least 90% slower, this could identify the compound as an inhibitor of the activity of the heterologous gene. In one example, if the yeast grown in the presence of the compound grows slower than the control such as at most 10% slower, at most 20% slower, at most 30% slower, at most 40% slower, at most 50% slower, at most 60% slower, at most 70% slower, at most 80% slower, or at most 90% slower this could identify the compound as an inhibitor of the activity of the heterologous gene.

In one example, yeasts of this disclosure are used to identify compounds which selectively inhibit Methyltransferases, Guanylyltransferase and/or RNA Triphosphatases expressed by pathogens, as compared to vertebrate Methyltransferases, Guanylyltransferase and/or RNA Triphosphatases, mammalian, Methyltransferases, Guanylyltransferase and/or RNA Triphosphatases, or human Methyltransferases, Guanylyltransferase and/or RNA Triphosphatases. For example, a non-native yeast having genetically inactivated Abd1, Ceg1p, and/or Cet1p, and also expressing a heterologous methyltransferase, heterologous guanylyltransferase, and/or heterologous RNA triphosphatase which is derived from a pathogen of interest, can be grown in media which contains a compound which may inhibit the heterologous genes. At the same time, a non-native yeast having genetically inactivated Abd1, Ceg1p, and/or Cet1p, and also expressing a heterologous methyltransferase, heterologous guanylyltransferase, and/or heterologous RNA triphosphatase derived from a control, such as a human methyltransferase, guanylyltransferase, and/or RNA triphosphatase, can be grown in media which contains the same compound which may inhibit the heterologous genes. The relative growth of the yeast having the heterologous gene derived from a pathogen could be compared the relative growth of the yeast having the heterologous gene derived from a control, such as a human. In some examples the growth could be directly compared. In some examples, the growth of each yeast would be normalized against a historic control, and the change in growth rates would be compared. When the yeast expressing a heterologous pathogen enzyme has more growth impairment than the yeast expressing the heterologous control enzyme, such as a human enzyme, this could identify the compound as selectively inhibiting the pathogen enzyme. When the yeast expressing the heterologous control enzyme, such as a human enzyme, has more growth impairment than the yeast expressing a heterologous pathogen enzyme, this could identify the compound as selectively inhibiting the control enzyme.

In some examples, growth of the yeast expressing a pathogen enzyme would be identified as a selective inhibitor if it grows at least 10% slower, at least 20% slower, at least 30% slower, at least 40% slower, at least 50% slower, at least 60% slower, at least 70% slower, at least 80% slower, or at least 90% slower as compared to a control, such as the control enzyme, or similar yeasts grown in the absence of the compound, or a historical control. In some examples, growth of the yeast expressing a pathogen enzyme would be identified as a selective inhibitor if it grows at most 10% slower, at most 20% slower, at most 30% slower, at most 40% slower, at most 50% slower, at most 60% slower, at most 70% slower, at most 80% slower, or at most 90% slower as compared to a control, such as the control enzyme, or similar yeasts grown in the absence of the compound, or a historical control.

In some examples, yeasts disclosed herein are used in a method of screening for methyltransferase, guanylyltransferase, or RNA triphosphatase inhibitors. This method may include steps of culturing a first strain of yeast in media including at least one screening compound, measuring growth of the first strain, and comparing the growth of the first strain to a control. In some examples, when the first strain grows slower than the control, this indicates the at least one screening compound is a methyltransferase inhibitor, a guanylyltransferase inhibitor, or an RNA triphosphatase inhibitor. In some examples, when the first strain growing slower than both the control and the second strain indicates the at least one screening compound is a specific methyltransferase inhibitor, a specific guanylyltransferase inhibitor, or a specific RNA triphosphatase inhibitor.

Culturing the yeasts disclosed herein can be accomplished by any suitable method, such as culturing on solid or liquid media, such as YPD or chemically defined media, such as media containing a selection agent. Measuring the growth of the yeast disclosed herein can be accomplished by any suitable method, such as dilution plating, or se of spectrophotometry, or by using a yeast strain which incorporates a reporter, such as a fluorescent reporter, which can be measured by flow cytometry.

### VI. Attenuated Viruses

Disclosed herein are mutations that are useful in producing recombinant strains of SARS-CoV-2, MERS-CoV, ASFV, MPV, and/or WNV exhibiting a range of attenuation phenotypes and are suitable for use as attenuated, live vaccines in humans. As reported herein, particular combinations of mutations to wt SARS-CoV-2, MERS-CoV, ASFV, MPV, and/or WNV result in a live, attenuated virus that elicits a superior immune response.

Further disclosed herein are methods and compositions related to the expression of the disclosed viruses. For example, isolated polynucleotide molecules that include a nucleic acid sequence encoding the genome or antigenome of the described viruses are disclosed.

The recombinant SARS-CoV-2, MERS-CoV, ASFV, MPV, and/or WNV provided herein include a genome or antigenome containing modifications or mutations as described in detail herein relative to wild-type SARS-CoV-2, MERS-CoV, ASFV, MPV, and/or WNV that attenuate the recombinant SARS-CoV-2, MERS-CoV, ASFV, MPV, and/or WNV. The wild type SARS-CoV-2 genome is encoded by GenBank accession #MT318827.1. The wild type MERS-CoV genome is encoded by GenBank accession #NC_019843.3. The wild type ASFV genome is encoded by GenBank accession #NC_001659.2. The wild type MPV genome is encoded by GenBank accession #MT903343.1. The wild type WNV genome is encoded by GenBank accession #NC_009942.1. The wild type SARS-CoV-2 genome encodes the proteins nsp14, nsp12, and nsp9. The wild type MERS-CoV genome encodes the proteins nsp14, nsp12, and nsp9. The wild type MPV genome encodes the proteins D1 and D12. The wild type ASFV genome encodes the protein NP858R. The wild type WNV genome encodes the protein ns5.

In some examples, the attenuated virus is a virus expressing SARS-CoV-2 nsp14, with one or more of the following substitutions: W293F, F368N, F368L, D353T, and/or D353A where the substitutions refer to SEQ ID NO: 4. In some examples, the attenuated virus is a virus expressing SARS-CoV-2 nsp14, with one or more of the following substitutions: V381L, A394V, P46L, P412H, L157F or I42V where the substitutions refer to SEQ ID NO: 289. In some examples, the attenuated virus is a virus expressing MERS-CoV nsp14 with the following substitution: F365G where the substitution refer to SEQ ID NO: 24. In some examples, the attenuated virus is a virus expressing ASFV NP868R with one or more of the following substitutions: Y714L, F71IL, F711W, D680L, K647Y, and/or S604A where the substitutions refer to SEQ ID NO: 10. In some examples, the attenuated virus is a virus expressing MPV Dl with one or more of the following substitutions: Y555F, R655A, D545A, D598A, Y683V, or R548A, R548K where the substitutions refer to SEQ ID NO: 290. In some examples the attenuated virus is a virus expressing SARS-CoV-2 nsp12 with one or more of the following substitutions: K73A, D218A, D760A, V30A, R33A, R33L, R33K, R55A, R55L, R55K, K50A, C53A, R116A, V71A, N209A, L119A, and/or Y217F wherein the substitutions refer to SEQ ID NO: 291. In some examples the attenuated virus is a virus expressing WNV ns5 with one or more of the following substitutions: F24L, F24E, and/or S150C where the substitutions refer to SEQ ID NO: 292. As discussed in the examples below, the novel combination of these modifications to the native SARS-CoV-2, MERS-CoV, ASFV, MPV, and/or WNV genome results in a recombinant SARS-CoV-2, MERS-CoV, ASFV, MPV, and/or WNV that elicits a superior immune response.

As used herein, and unless context indicates otherwise, virus names are descriptive rather than limiting. The full set of modifications or mutations in each virus is not necessarily listed fully in the name. Additionally, unless context indicates otherwise, the order of appearance of modifications/mutations in a virus name can vary. The examples of recombinant SARS-CoV-2, MERS-CoV, ASFV, MPV, and/or WNV disclosed herein are infectious, attenuated, and self-replicating.

Unless context indicates otherwise, the numbering used in this disclosure for substitutions in SARS-CoV-2 nsp14 is based on SEQ ID NO: 4 or SEQ ID NO: 289. Unless context indicates otherwise, the numbering used in this disclosure for substitutions in MERS-CoV nsp14 is based on SEQ ID NO: 24. Unless context indicates otherwise, the numbering used in this disclosure for substitutions in ASFV NP868R is based on SEQ ID NO: 10. Unless context indicates otherwise, the numbering used in this disclosure for substitutions in MPV D1 is based on SEQ ID NO: 290. Unless context indicates otherwise, the numbering used in this disclosure for substitutions in MPV D12 is based on SEQ ID NO: 8. Unless context indicates otherwise, the numbering used in this disclosure for substitutions in SARS-CoV-2 Nsp12 is based on SEQ ID NO: 291. Unless context indicates otherwise, the numbering used in this disclosure for substitutions in WNV ns5 is based on SEQ ID NO: 292. Unless context indicates otherwise, the numbering used in this disclosure for substitutions in SARS-CoV-2 nsp9 is based on SEQ ID NO: 34. With regard to sequence numbering of nucleotide and amino acid sequence positions for the described viruses, a convention was used whereby each nucleotide or amino acid residue in a given viral sequence retained the sequence position number that it has in the above referenced SEQ ID NOs: 4, 289, 24, 10, 290, 8, 291, and 292, irrespective of any modifications. Thus, although a genome could contain deletions and/or insertions that cause changes in nucleotide length, and in some cases amino acid length, the numbering of all of the other residues (nucleotide or amino acid) in the genome and encoded proteins remains unchanged. Corresponding sequence positions between viral genomes or proteins that might differ in length can also be identified by sequence alignments as well as the positions of open reading frames, RNA features such as gene-start and gene-end signals, and amino acid sequence features.

In addition to the above-described modifications to recombinant SARS-CoV-2, MERS-CoV, ASFV, MPV, and/or WNV, different or additional modifications in SARS-CoV-2, MERS-CoV, ASFV, MPV, and/or WNV clones can be made to facilitate manipulations, such as the insertion of unique restriction sites in various intergenic regions or elsewhere. Nontranslated gene sequences can be removed to increase capacity for inserting foreign sequences.

Introduction of the foregoing, defined mutations into an infectious SARS-CoV-2, MERS-CoV, ASFV, MPV, and/or WNV clone can be achieved by a variety of methods. By “infectious clone” is meant cDNA or its product, synthetic or otherwise, which can be transcribed into genomic or antigenomic RNA capable of producing an infectious virus. The term “infectious” refers to a virus or viral structure that is capable of replicating in a cultured cell or animal or human host to produce progeny virus or viral structures capable of the same activity. Thus, defined mutations can be introduced by known techniques (e.g., site-directed mutagenesis) into a cDNA copy of the genome or antigenome. The use of antigenome or genome cDNA subfragments to assemble a complete antigenome or genome cDNA has the advantage that each region can be manipulated separately (smaller cDNAs are easier to manipulate than large ones) and then readily assembled into a complete cDNA. Thus, the complete antigenome or genome cDNA, or any subfragment thereof, can be used as template for oligonucleotide-directed mutagenesis. A mutated subfragment can then be assembled into the complete antigenome or genome cDNA. Mutations can vary from single nucleotide changes to replacement of large cDNA pieces containing one or more genes or genome regions.

In some examples, the disclosed recombinant SARS-CoV-2, MERS-CoV, ASFV, MPV, and/or WNV can be produced using a recombinant DNA-based technique called reverse genetics (Collins, et al. 1995. Proc Natl Acad Sci USA 92:11563-11567). This system allows de novo recovery of infectious virus entirely from cDNA in a qualified cell substrate under defined conditions. Reverse genetics provides a means to introduce predetermined mutations into the SARS-CoV-2, MERS-CoV, ASFV, MPV, and/or WNV genome via the cDNA intermediate. Derivation of vaccine viruses from cDNA minimizes the risk of contamination with adventitious agents and helps to keep the passage history brief and well documented. Once recovered, the engineered virus strains propagate in the same manner as a biologically derived virus. As a result of passage and amplification, the vaccine viruses do not contain recombinant DNA from the original recovery.

Recombinant SARS-CoV-2, MERS-CoV, ASFV, MPV, and/or WNV may be produced by the intracellular coexpression of a cDNA that encodes the SARS-CoV-2, MERS-CoV, ASFV, MPV, and/or WNV genomic RNA, together with those viral proteins necessary to generate a transcribing, replicating nucleocapsid. Plasmids encoding other SARS-CoV-2, MERS-CoV, ASFV, MPV, and/or WNV proteins may also be included with these essential proteins. Alternatively, RNA may be synthesized in in vitro transcription reactions and transfected into cultured cells.

To propagate a SARS-CoV-2, MERS-CoV, ASFV, MPV, and/or WNV virus for vaccine use and other purposes, a number of cell lines which allow for SARS-CoV-2, MERS-CoV, ASFV, MPV, and/or WNV growth may be used. SARS-CoV-2, MERS-CoV, ASFV, MPV, and/or WNV grows in a variety of human and animal cells. Exemplary cell lines for propagating attenuated SARS-CoV-2, MERS-CoV, ASFV, MPV, and/or WNV virus for vaccine use include DBSFRhL-2, MRC-5, and Vero cells. Cells are typically inoculated with virus at a multiplicity of infection ranging from about 0.001 to 1.0, or more, and are cultivated under conditions permissive for replication of the virus, e.g., at about 30-37° C. and for about 3-10 days, or as long as necessary for virus to reach an adequate titer. Temperature-sensitive viruses often are grown using 32° C. as the “permissive temperature.” Virus is removed from cell culture and separated from cellular components, typically by well-known clarification procedures, e.g., centrifugation, and may be further purified as desired using any suitable procedure.

SARS-CoV-2, MERS-CoV, ASFV, MPV, and/or WNV which has been attenuated as described herein can be tested in various in vitro and in vivo models to confirm adequate attenuation, resistance to phenotypic reversion, and immunogenicity for vaccine use. In in vitro assays, the modified virus, which can be a multiply attenuated, biologically derived or recombinant SARS-CoV-2, MERS-CoV, ASFV, MPV, and/or WNV, is tested for temperature sensitivity of virus replication or “ts phenotype,” and for the small plaque phenotype. Modified virus also may be evaluated in an in vitro human airway epithelium (HAE) model, which appears to provide a means of ranking viruses in the order of their relative attenuation in non-human primates and humans (Zhang et al 2002 J Virol 76:5654-5666; Schaap-Nutt et al 2010 Vaccine 28:2788-2798; Ilyushina et al 2012 J Virol 86:11725-11734). Modified viruses are further tested in animal models of SARS-CoV-2, MERS-CoV, ASFV, MPV, and/or WNV infection. A variety of animal models (e.g., murine, cotton rat, and primate) have been described.

Recombinant viruses can be evaluated in cell culture, rodents and non-human primates for infectivity, replication kinetics, yield, efficiency of protein expression, and genetic stability. While these semi-permissive systems may not reliably detect every difference in replication, substantial differences in particular may be detected. Recombinant strains may be also evaluated successively in adults, seropositive children, and seronegative children. In some cases, where a previous similar strain has already been shown to be well-tolerated in seronegative children, a new strain may be evaluated directly in seronegative children. Evaluation can be done, for example, in groups of 10 vaccine recipients and 5 placebo recipients, which is a small number that allows simultaneous evaluation of multiple candidates. Candidates can be evaluated in the period immediately post-immunization for vaccine virus infectivity, replication kinetics, shedding, tolerability, immunogenicity, and genetic stability, and the vaccinees may be subjected to surveillance during the following SARS-CoV-2, MERS-CoV, ASFV, MPV, and/or WNV season for safety, SARS-CoV-2, MERS-CoV, ASFV, MPV, and/or WNV disease, and changes in SARS-CoV-2, MERS-CoV, ASFV, MPV, and/or WNV-specific serum antibodies, as described in Karron, et al. 2015, Science Transl Med 2015 7(312):312ra175. Thus, analysis of selected representative viruses provides for relatively rapid triage to narrow down candidates to identify the most optimal.

Also provided herein are isolated polynucleotides that encode the described mutated viruses, make up the described genomes or antigenomes, express the described genomes or antigenomes, or encode various proteins useful for making recombinant SARS-CoV-2, MERS-CoV, ASFV, MPV, and/or WNV in vitro. The nucleic acid sequences of exemplary polynucleotides are also provided. Included within the examples provided herein are polynucleotides comprising sequences that consist or consist essentially of any of the aforementioned nucleic acid sequences. Further included are polynucleotides that possess at least 90, 91, 92, 93, 94, 95, 96, 97, 98, 99 percent or more identity, or any number in between, to any of the aforementioned sequences or SEQ ID NOs provided herein, as well as polynucleotides that hybridize to, or are the complements of the aforementioned molecules.

These polynucleotides can be included within or expressed by vectors to produce a recombinant SARS-CoV-2, MERS-CoV, ASFV, MPV, and/or WNV. Accordingly, cells transfected with the isolated polynucleotides or vectors are also included.

In additional examples, compositions (e.g., isolated polynucleotides and vectors incorporating a SARS-CoV-2, MERS-CoV, ASFV, MPV, and/or WNV-encoding cDNA) and methods are provided for producing a recombinant SARS-CoV-2, MERS-CoV, ASFV, MPV, and/or WNV. Also provided are novel, isolated polynucleotide molecules and vectors incorporating such molecules that comprise a SARS-CoV-2, MERS-CoV, ASFV, MPV, and/or WNV genome or antigenome which is modified as described herein. Also provided is the same or different expression vector comprising one or more isolated polynucleotide molecules encoding the SARS-CoV-2, MERS-CoV, ASFV, MPV, and/or WNV proteins. These proteins also can be expressed directly from the genome or antigenome cDNA. The vector(s) are preferably expressed or coexpressed in a cell or cell-free lysate, thereby producing an infectious mutant SARS-CoV-2, MERS-CoV, ASFV, MPV, and/or WNV particle or subviral particle.

Also provided are methods for producing one or more purified SARS-CoV-2, MERS-CoV, ASFV, MPV, and/or WNV protein(s) which involves infecting a host cell permissive of SARS-CoV-2, MERS-CoV, ASFV, MPV, and/or WNV infection with a recombinant SARS-CoV-2, MERS-CoV, ASFV, MPV, and/or WNV strain under conditions that allow for SARS-CoV-2, MERS-CoV, ASFV, MPV, and/or WNVv propagation in the infected cell. After a period of replication in culture, the cells are lysed and recombinant SARS-CoV-2, MERS-CoV, ASFV, MPV, and/or WNV is isolated therefrom. One or more desired SARS-CoV-2, MERS-CoV, ASFV, MPV, and/or WNV protein(s) is purified after isolation of the virus, yielding one or more SARS-CoV-2, MERS-CoV, ASFV, MPV, and/or WNV protein(s) for vaccine, diagnostic and other uses.

The above methods and compositions yield infectious viral or subviral particles, or derivatives thereof. An infectious virus is comparable to the authentic SARS-CoV-2, MERS-CoV, ASFV, MPV, and/or WNV virus particle and is infectious as is. It can directly infect fresh cells. An infectious subviral particle typically is a subcomponent of the virus particle which can initiate an infection under appropriate conditions. Subviral particles include viral particles which lack one or more protein(s), protein segment(s), or other viral component(s) not essential for infectivity.

In other examples the disclosure provides a cell or cell free lysate containing an expression vector which comprises an isolated polynucleotide molecule encoding mutant SARS-CoV-2, MERS-CoV, ASFV, MPV, and/or WNV genome or antigenome as described above, and an expression vector (the same or different vector) which includes one or more isolated polynucleotide molecules proteins of SARS-CoV-2, MERS-CoV, ASFV, MPV, and/or WNV. One or more of these proteins also can be expressed from the genome or antigenome cDNA. Upon expression the genome or antigenome, the proteins combine to produce an infectious SARS-CoV-2, MERS-CoV, ASFV, MPV, and/or WNV viral or sub-viral particle.

### VII. Immunogenic Compositions

Immunogenic compositions including one or more disclosed recombinant SARS-CoV-2, MERS-CoV, ASFV, MPV, and/or WNV and a pharmaceutically acceptable carrier are also provided. Such compositions can be administered to a subject by a variety of modes, for example, by injection, or by an intranasal route. Methods for preparing administrable immunogenic compositions are described, for example, in such publications as Remingtons Pharmaceutical Sciences, 19^{th} Ed., Mack Publishing Company, Easton, Pennsylvania, 1995.

Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

The immunogenic composition can contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic composition can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition can optionally include an adjuvant to enhance the immune response of the host. Suitable adjuvants include, for example, toll-like receptor agonists, alum, AlPO4, alhydrogel, Lipid-A and derivatives or variants thereof, oil-emulsions, saponins, neutral liposomes, liposomes containing the recombinant virus, and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), among many other suitable adjuvants can be used as an adjuvant (Newman et al., 1998, Critical Reviews in Therapeutic Drug Carrier Systems 15:89-142). These adjuvants help to stimulate the immune system in a non-specific way, thus enhancing the immune response to a pharmaceutical product.

In some instances, it may be desirable to combine the immunogenic composition including the recombinant SARS-CoV-2, MERS-CoV, ASFV, MPV, and/or WNV, with other pharmaceutical products (e.g., vaccines) which induce protective responses to other viral agents, particularly those causing other childhood illnesses. For example, a composition including a recombinant SARS-CoV-2, MERS-CoV, ASFV, MPV, and/or WNV as described herein can also include other vaccines recommended by the Advisory Committee on Immunization Practices (ACIP; cdc.gov/vaccines/acip/index.html) for the targeted age group (e.g., infants from approximately one to six months of age). As such, a recombinant SARS-CoV-2, MERS-CoV, ASFV, MPV, and/or WNV as described herein may be administered simultaneously with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some examples, the immunogenic composition can be provided in unit dosage form for use to induce an immune response in a subject, for example, to prevent SARS-CoV-2, MERS-CoV, ASFV, MPV, and/or WNV infection in the subject. A unit dosage form contains a suitable single preselected dosage for administration to a subject, or suitable marked or measured multiples of two or more preselected unit dosages, and/or a metering mechanism for administering the unit dose or multiples thereof.

### VIII. Methods of Eliciting an Immune Response

Provided herein are methods of eliciting an immune response in a subject by administering an immunogenic composition provided herein that contains one or more disclosed recombinant SARS-CoV-2, MERS-CoV, ASFV, MPV, and/or WNV to the subject. Upon immunization, the subject responds by producing antibodies specific for SARS-CoV-2, MERS-CoV, ASFV, MPV, and/or WNV. In addition, innate and cell-mediated immune responses are induced, which can provide antiviral effectors as well as regulating the immune response. As a result of the immunization the host becomes at least partially or completely immune to SARS-CoV-2, MERS-CoV, ASFV, MPV, and/or WNV infection, or resistant to developing moderate or severe SARS-CoV-2, MERS-CoV, ASFV, MPV, and/or WNV disease.

In additional examples, a method of eliciting an immune response in a subject includes administering to the subject an immunogenic composition containing a recombinant SARS-CoV-2 nsp14 with one or more of the following substitutions: W293F, F368N, F368L, D353T, and/or D353A. In some examples, the method of eliciting an immune response includes administering to the subject an immunogenic composition containing a recombinant MERS-CoV nsp14 with the following substitution: F365G. In some examples, the method of eliciting an immune response includes administering to the subject an immunogenic composition containing a recombinant ASFV NP868R with one or more of the following substitutions: Y714L, F711L, F711W, D680L, K647Y, and/or S604A. In some examples, the method of eliciting an immune response includes administering to the subject an immunogenic composition containing a recombinant SARS-CoV-2 nsp12 with one or more of the following substitutions: K73A, D218A, D760A, V30A, R33A, R33L, R33K, R55A, R55L, R55K, K50A, C53A, R116A, V71A, N209A, L119A, and/or Y217F. In some examples, the method of eliciting an immune response includes administering to the subject an immunogenic composition containing a recombinant WNV ns5 with one or more of the following substitutions: F24L, F24E, and/or S150C. In some examples, the immunogenic composition includes a recombinant SARS-CoV-2, MERS-CoV, ASFV, MPV, and/or WNV, which is infectious, attenuated, and self-replicating. In some examples, the immunogenic composition includes incorporating the recombinant proteins, such as SARS-CoV-2 nsp14, MERS-CoV nsp14, MPV D1, ASFV NP868R, WNV ns5, or SARS-CoV-2 nsp12 into a viral particle in which these proteins would not naturally be found, such as an influenza viral particle expressing SARS-CoV-2 nsp14. In some examples, the recombinant proteins are incorporated into a recombinant organism where they would not naturally be found, such as a bacterium expressing SARS-CoV-2 nsp14. Upon immunization, the subject responds by producing antibodies specific for SARS-CoV-2, MERS-CoV, ASFV, MPV, and/or WNV. In addition, innate and cell-mediated immune responses are induced, which can provide antiviral effectors as well as regulating the immune response. As a result of the immunization the host becomes at least partially or completely immune to SARS-CoV-2, MERS-CoV, ASFV, MPV, and/or WNV infection, or resistant to developing moderate or severe SARS-CoV-2, MERS-CoV, ASFV, MPV, and/or WNV disease.

Because nearly all humans are susceptible to SARS-CoV-2, MERS-CoV, MPV, and/or WNV, the entire population is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, family members of newborn infants or those still in utero, and subjects greater than 50 years of age. The scope of this disclosure is meant to include maternal immunization. In several examples, the subject is a human subject that is seronegative for SARS-CoV-2, MERS-CoV, MPV, and/or WNV specific antibodies. In additional examples, the subject is no more than one year old, such as no more than 6 months old, no more than 3 months, or no more than 1 month old. In some examples, a second immunization will be given following the first immunization, such as 2 weeks, 3 weeks, 4 weeks, 5 weeks, 6 weeks, 7 weeks, or 8 weeks following the first injection.

Because domestic swine and wild boars are susceptible to ASFV, the entire population of S. scrofa is a relevant population for immunization. In some examples, this could be done by immunizing subjects subcutaneously, or intramuscularly. In some examples, this could be done by immunizing subjects intranasally, or orally. In some examples, a withdrawal time will be indicated for the vaccination, such as 3 weeks, 4 weeks, or 5 weeks. In some examples, a second immunization will be given following the first immunization, such as 2 weeks, 3 weeks, 4 weeks, 5 weeks, 6 weeks, 7 weeks, or 8 weeks following the first injection. In some examples, the subject is no more than one year old, such as no more than 6 months old, no more than 3 months, or no more than 1 month old.

The immunogenic compositions containing the recombinant SARS-CoV-2, MERS-CoV, ASFV, MPV, and/or WNV are administered to a subject susceptible to or otherwise at risk of SARS-CoV-2, MERS-CoV, ASFV, MPV, and/or WNV infection in an “effective amount” which is sufficient to induce or enhance the individual's immune response capabilities against SARS-CoV-2, MERS-CoV, ASFV, MPV, and/or WNV. The immunogenic composition may be administered by any suitable method, including but not limited to, via injection, aerosol delivery, nasal spray, nasal droplets, oral inoculation, or topical application. In some examples, the vaccine may be administered intranasally or subcutaneously or intramuscularly. In some examples, it may be administered to the upper respiratory tract. This may be performed by any suitable method, including but not limited to, by spray, droplet or aerosol delivery. Often, the composition will be administered to an individual seronegative for antibodies to SARS-CoV-2, MERS-CoV, ASFV, MPV, and/or WNV or possessing transplacentally acquired maternal antibodies to SARS-CoV-2, MERS-CoV, ASFV, MPV, and/or WNV.

Upon immunization with an effective amount of a disclosed recombinant SARS-CoV-2, MERS-CoV, ASFV, MPV, and/or WNV, the subject responds by producing antibodies specific for SARS-CoV-2, MERS-CoV, ASFV, MPV, and/or WNV virus proteins, such as nsp14, nsp12, nsp9, d1, d12, NP868R, and/or ns5. In addition, innate and cell-mediated immune responses are induced, which can provide antiviral effectors as well as regulating the immune response. As a result of the immunization the host becomes at least partially or completely immune to SARS-CoV-2, MERS-CoV, ASFV, MPV, and/or WNV infection, or resistant to developing moderate or severe SARS-CoV-2, MERS-CoV, ASFV, MPV, and/or WNV disease, particularly of the lower respiratory tract.

The precise amount of immunogen administered and the timing and repetition of administration will be determined by various factors, including the patient's state of health and weight, the mode of administration, the nature of the formulation, etc. Dosages ins some examples range from about 3.0 log₁₀ to about 6.0 log₁₀ plaque forming units (“PFU”) or more of virus per patient, more commonly from about 4.0 log₁₀ to 5.0 log₁₀ PFU virus per patient. In one example, about 5.0 log₁₀ to 6.0 log₁₀ PFU per patient may be administered during infancy, such as between 1 and 6 months of age, and one or more additional booster doses could be given 2-6 months or more later. In another example, young infants could be given a dose of about 5.0 log₁₀ to 6.0 log₁₀ PFU per patient at approximately 2, 4, and 6 months of age, which is the recommended time of administration of a number of other childhood vaccines. In yet another example, an additional booster dose could be administered at approximately 10-15 months of age.

The examples of recombinant SARS-CoV-2, MERS-CoV, ASFV, MPV, and/or WNV described herein, and immunogenic compositions thereof, are administered to a subject in an amount effective to induce or enhance an immune response against the SARS-CoV-2, MERS-CoV, ASFV, MPV, and/or WNV antigens in the recombinant SARS-CoV-2, MERS-CoV, ASFV, MPV, and/or WNV in the subject. An effective amount will allow some growth and proliferation of the virus, to produce the desired immune response, but will not produce viral-associated symptoms or illnesses.

The resulting immune response can be characterized by a variety of methods. These include taking samples of nasal washes or sera for analysis of SARS-CoV-2, MERS-CoV, ASFV, MPV, and/or WNV-specific antibodies, which can be detected by tests including, but not limited to, complement fixation, plaque neutralization, enzyme-linked immunosorbent assay, luciferase-immunoprecipitation assay, and flow cytometry. In addition, immune responses can be detected by assay of cytokines in nasal washes or sera, ELISPOT of immune cells from either source, quantitative RT-PCR or microarray analysis of nasal wash or serum samples, and restimulation of immune cells from nasal washes or serum by re-exposure to viral antigen in vitro and analysis for the production or display of cytokines, surface markers, or other immune correlates measures by flow cytometry or for cytotoxic activity against indicator target cells displaying SARS-CoV-2, MERS-CoV, ASFV, MPV, and/or WNV antigens. In this regard, individuals are also monitored for signs and symptoms of upper respiratory illness.

A desired immune response is to inhibit subsequent infection with SARS-CoV-2, MERS-CoV, ASFV, MPV, and/or WNV. The SARS-CoV-2, MERS-CoV, ASFV, MPV, and/or WNV infection does not need to be completely inhibited for the method to be effective. For example, administration of an effective amount of a disclosed recombinant SARS-CoV-2, MERS-CoV, ASFV, MPV, and/or WNV can decrease subsequent SARS-CoV-2, MERS-CoV, ASFV, MPV, and/or WNV infection (for example, as measured by infection of cells, or by number or percentage of subjects infected by SARS-CoV-2, MERS-CoV, ASFV, MPV, and/or WNV) by a desired amount, for example by at least 10%, at least 20%, at least 50%, at least 60%, at least 70%, at least 80%, at least 90%, at least 95%, at least 98%, or even at least 100% (prevention of detectable SARS-CoV-2, MERS-CoV, ASFV, MPV, and/or WNV infection), as compared to a suitable control.

Determination of effective dosages is typically based on animal model studies followed up by human clinical trials, where applicable, and is guided by administration protocols that significantly reduce the occurrence or severity of targeted disease symptoms or conditions in the subject, or that induce a desired response in the subject (such as a neutralizing immune response). Suitable models in this regard include, for example, murine, rat, hamster, cotton rat, bovine, ovine, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of a targeted disease).

An immunogenic composition including one or more of the disclosed recombinant SARS-CoV-2, MERS-CoV, ASFV, MPV, and/or WNV viruses can be used in coordinate (or prime-boost) immunization protocols or combinatorial formulations. It is contemplated that there can be several boosts, and that each boost can be a different disclosed immunogen. It is also contemplated in some examples that the boost may be the same immunogen as another boost, or the prime. In certain examples, novel combinatorial immunogenic compositions and coordinate immunization protocols employ separate immunogens or formulations, each directed toward eliciting an anti-viral immune response, such as an immune response to SARS-CoV-2, MERS-CoV, ASFV, MPV, and/or WNV proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

### IX. Kits

Also provided are compositions and kits that can be used with the disclosed methods. In some examples, the kits are used for methods of administering an immunogenic composition including: a recombinant SARS-CoV-2 nsp14 with one or more of the following substitutions: W293F, F368N, F368L, D353T, and/or D353A; a recombinant MERS-CoV nsp14 with the following substitution: F365G; a recombinant ASFV NP868R with one or more of the following substitutions: Y714L, F711L, F711W, D680L, K647Y, and/or S604A; a recombinant SARS-CoV-2 nsp12 with one or more of the following substitutions: K73A, D218A, D760A, V30A, R33A, R33L, R33K, R55A, R55L, R55K, K50A, C53A, R116A, V71A, N209A, L119A, and/or Y217F; and/or a recombinant WNV ns5 with one or more of the following substitutions: F24L, F24E, and/or S150C. In some examples, the kit includes a container including viral particles which include the recombinant proteins described herein. In some examples, the kit includes a containing including purified proteins with the substitutions described herein. In some examples, the kit includes nucleotides encoding the recombinant proteins described herein. In some examples, the kit includes a cell line configured to express the recombinant proteins described herein. In some examples, the examples described herein are inside a container, which in some examples is a syringe. In some examples, the kit optionally includes instructions for use.

In some examples, the kit includes a plasmid including a heterologous methyltransferase gene, a heterologous guanylyltransferase gene, and/or a heterologous RNA triphosphatase gene. In some examples, the kit includes a yeast strain with genetically inactivated Abd1, Ceg1p, and/or Cet1p. In some examples, the kit includes instructions for using the kit. In some examples, the kit includes a library of screening compounds. In some examples, the kit includes a plasmid with a selectable marker. In some examples, the kit includes a selectable agent, optionally which corresponds to the selectable marker in the plasmid in the kit. In some examples, the kit includes medium for growing the yeast of the kit, optionally including an antibiotic or selectable agent.

In some examples, the kit includes a plasmid including a methyltransferase, guanylyltransferase, and/or RNA triphosphatase, which can be incorporated into a strain of yeast, which optionally has inactivated Abd1, Ceg1p, and/or Cet1p. In some examples the kit includes the yeasts disclosed herein. In some examples, the kit further includes a curable Abd1, Ceg1p, and/or Cet1p plasmid. In some examples, the kit includes a culture medium, optionally including G418, optionally lacking uracil, and optionally including 5-FOA.

In some examples, the kit includes a first strain of yeast disclosed herein, such as yeast incorporating a plasmid including a methyltransferase, guanylyltransferase, and/or RNA triphosphatase. In some examples, the kit includes a second strain of yeast as described herein, where the second strain of the isolated non-native yeast includes a vertebrate heterologous methyltransferase, heterologous guanylyltransferase, or heterologous RNA triphosphatase. In some examples, the kit includes a control compound or small molecule which inhibits activity of the heterologous methyltransferase, the heterologous guanylyltransferase, or the heterologous RNA triphosphatase of the first strain, and which inhibits activity of the heterologous methyltransferase, the heterologous guanylyltransferase, or the heterologous RNA triphosphatase of the second strain. In some examples, the kit includes a control compound or small molecule which inhibits activity of the heterologous methyltransferase, the heterologous guanylyltransferase, or the heterologous RNA triphosphatase of the first strain, and which does not inhibit activity of the heterologous methyltransferase, the heterologous guanylyltransferase, or the heterologous RNA triphosphatase of the second strain. In some examples, the kit includes a control compound or small molecule which does not inhibit activity of the heterologous methyltransferase, the heterologous guanylyltransferase, or the heterologous RNA triphosphatase of the first strain, and which inhibits activity of the heterologous methyltransferase, the heterologous guanylyltransferase, or the heterologous RNA triphosphatase of the second strain. In some examples, the kit includes a control compound or small molecule which does not inhibit activity of the heterologous methyltransferase, the heterologous guanylyltransferase, or the heterologous RNA triphosphatase of the first strain, and which does not inhibit activity of the heterologous methyltransferase, the heterologous guanylyltransferase, or the heterologous RNA triphosphatase of the second strain. In some examples, the kit includes a library of small molecules or compounds.

### X. Additional Aspects

Clause 1. An isolated non-native yeast, comprising:
a) a genetically inactivated Abd1 gene and a heterologous methyltransferase;
b) a genetically inactivated Ceg1p gene and a heterologous guanylyltransferase; or
c) a genetically inactivated Cet1p and a heterologous RNA triphosphatase.

Clause 2. The isolated non-native yeast of clause 1, wherein the Abd1 gene is replaced by an antibiotic resistance gene.

Clause 3. The isolated non-native yeast of clause 1 or clause 2, wherein the heterologous methyltransferase is a full-length methyltransferase, a functional methyltransferase fragment, or a codon optimized methyltransferase for expression in yeast.

Clause 4. The isolated non-native yeast of any one of the preceding clauses, wherein the heterologous methyltransferase is encoded by a plasmid.

Clause 5. The isolated non-native yeast of clause 4, wherein the plasmid encoding the heterologous methyltransferase comprises a promoter operably linked to the heterologous methyltransferase.

Clause 6. The isolated non-native yeast of clause 5, wherein the plasmid encoding the heterologous methyltransferase comprises or consists of at least 80%, at least 85%, at least 90%, at least 95%, at least 96%, at least 97%, at least 98%, at least 99%, or 100% sequence identity to SEQ ID NO: 16, 17, 18, 19, 21, 22, or 23.

Clause 7. The isolated non-native yeast of clause 4 or clause 5, wherein the heterologous methyltransferase is fused to an RNA polymerase complex targeting gene.

Clause 8. The isolated non-native yeast of clause 7, wherein the RNA polymerase complex targeting gene comprises a Mce1 GTase domain gene.

Clause 9. The isolated non-native yeast of clause 8, wherein the Mce1 GTase domain gene is a full length Mce1 GTase domain, a functional Mce1 GTase domain fragment, or a codon optimized Mce1 GTase domain gene for expression in yeast.

Clause 10. The isolated non-native yeast of any one of clause 8 or clause 9, wherein the amino acid sequence of the Mce1 GTase domain gene comprises or consists of at least 80%, at least 85%, at least 90%, at least 95%, at least 96%, at least 97%, at least 98%, at least 99%, or 100% sequence identity to SEQ ID NO: 1; or

wherein the nucleotide sequence of the Mce1 GTase domain gene comprises or consists of at least 80%, at least 85%, at least 90%, at least 95%, at least 96%, at least 97%, at least 98%, at least 99%, or 100% sequence identity to SEQ ID NO: 2.

Clause 11. The isolated non-native yeast of any one of the preceding clauses, further comprising an Adb1 plasmid comprising a second Abd1 gene operably linked to a promoter.

Clause 12. The isolated non-native yeast of clause 11, wherein the Adb1 plasmid further comprises a yeast Ura3 gene; and wherein the Adb1 plasmid is curable.

Clause 13. The isolated non-native yeast of clause 11 or clause 12, wherein the nucleotide sequence of the Adb1 plasmid comprises or consists of at least 80%, at least 85%, at least 90%, at least 95%, at least 96%, at least 97%, at least 98%, at least 99%, or 100% sequence identity to SEQ ID NO: 3.

Clause 14. The isolated non-native yeast of any one of the preceding clauses, wherein the heterologous methyltransferase comprises nsp14, D1, D12, D1 and D12, NP868R, ns5, or mRNA cap methyltransferase (RNMT).

Clause 15. The isolated non-native yeast of clause 14, wherein:
the nsp14 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 4 or 24;
the nsp14 comprises or consists of a protein sequence comprising or consisting of V381L, A394V, P46L, P412H, L157F or I42V substitutions within SEQ ID NO: 289;
the nsp14 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 5 or 25;
the D1 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 6;
the D1 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 7;
the D12 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 8;
the D12 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 9;
the NP868R comprises or consists of a protein sequence comprising or consisting of at least 80%, at least 85%, at least 90%, at least 95%, at least 96%, at least 97%, at least 98%, at least 99%, or 100% sequence identity to SEQ ID NO: 10;
the NP868R is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 11;
the ns5 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 12;
the RNMT comprises or consists of a protein sequence comprising or consisting of at least 80%, at least 85%, at least 90%, at least 95%, at least 96%, at least 97%, at least 98%, at least 99%, or 100% sequence identity to SEQ ID NOs: 14, 285, or 286; or
the RNMT is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 15.

Clause 16. The isolated non-native yeast of any one of the preceding clauses, wherein the heterologous methyltransferase comprises a Coronaviridae methyltransferase, a Poxviridae methyltransferase, an Asfarviridae methyltransferase, a Flaviviridae methyltransferase, a Filoviridae methyltransferase, a vertebrate methyltransferase, or a mammalian methyltransferase.

Clause 17. The isolated non-native yeast of any one of the preceding clauses, wherein the heterologous methyltransferase is nsp14; wherein the nsp14 comprises a catalytically inactive ExoN domain.

Clause 18. The isolated non-native yeast of clause 1, wherein the Ceg1p gene is replaced by an antibiotic resistance gene.

Clause 19. The isolated non-native yeast of clause 1 or clause 18, wherein the heterologous guanylyltransferase is a full length guanylyltransferase, a functional guanylyltransferase fragment, or a codon optimized guanylyltransferase for expression in yeast.

Clause 20. The isolated non-native yeast of any one of clauses 1, 18, or 19, wherein the heterologous guanylyltransferase is encoded by a plasmid.

Clause 21. The isolated non-native yeast of clause 20, wherein the plasmid encoding the heterologous guanylyltransferase comprises a promoter operably linked to the heterologous guanylyltransferase.

Clause 22. The isolated non-native yeast of clause 20, wherein the plasmid encoding the heterologous guanylyltransferase comprises or consists of at least 80%, at least 85%, at least 90%, at least 95%, at least 96%, at least 97%, at least 98%, at least 99%, or 100% sequence identity to SEQ ID NOs: 17, 18, 19, 26 or 27.

Clause 23. The isolated non-native yeast of clause 20 or clause 21, wherein the heterologous guanylyltransferase is fused to an RNA polymerase complex targeting gene.

Clause 24. The non-native yeast of clause 23, wherein the RNA polymerase complex targeting gene comprises a Mce1 GTase domain gene.

Clause 25. The isolated non-native yeast of clause 24, wherein the Mce1 GTase domain gene is a full length Mce1 GTase domain, a functional Mce1 GTase domain fragment, or a codon optimized Mce1 GTase domain gene for expression in yeast.

Clause 26. The isolated non-native yeast of clause 24 or clause 25, wherein the amino acid sequence of the Mce1 GTase domain gene comprises or consists of at least 80%, at least 85%, at least 90%, at least 95%, at least 96%, at least 97%, at least 98%, at least 99%, or 100% sequence identity to SEQ ID NO: 1; or
wherein the nucleotide sequence of the mce1 GTase domain gene comprises or consists of at least 80%, at least 85%, at least 90%, at least 95%, at least 96%, at least 97%, at least 98%, at least 99%, or 100% sequence identity to SEQ ID NO: 2.

Clause 27. The isolated non-native yeast of any one of the preceding clauses, further comprising a Ceg1p plasmid comprising a second Ceg1p gene operably linked to a promoter.

Clause 28. The isolated non-native yeast of clause 27, wherein the Ceg1p plasmid further comprises a yeast ura3 gene; and wherein the Ceg1p plasmid is curable.

Clause 29. The isolated non-native yeast of clause 27 or clause 28 wherein the nucleotide sequence of the Ceg1p plasmid comprises or consists of at least 80%, at least 85%, at least 90%, at least 95%, at least 96%, at least 97%, at least 98%, at least 99%, or 100% sequence identity to SEQ ID NO: 28.

Clause 30. The isolated non-native yeast of any one of the preceding clauses, wherein the heterologous guanylyltransferase comprises nsp12, ns5, NP868R, D1, D12, D1 and D12, or MCE1.

Clause 31. The isolated non-native yeast of clause 30, wherein:
the nsp12 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 29;
the nsp12 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 30;
the ns5 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 31;
the ns5 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 32;
the D1 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 6;
the D1 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 7;
the D12 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 8;
the D12 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 9;
the NP868R comprises or consists of a protein sequence comprising or consisting of at least 80%, at least 85%, at least 90%, at least 95%, at least 96%, at least 97%, at least 98%, at least 99%, or 100% sequence identity to SEQ ID NO: 10;
the NP868R is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 11; or
the MCE1 comprises or consists of a protein sequence comprising or consisting of at least 80%, at least 85%, at least 90%, at least 95%, at least 96%, at least 97%, at least 98%, at least 99%, or 100% sequence identity to SEQ ID NOs: 33 or 287.

Clause 31. The isolated non-native yeast of any one of the preceding clauses, wherein the heterologous guanylyltransferase comprises a Coronaviridae guanylyltransferase, a Poxviridae guanylyltransferase, an Asfarviridae guanylyltransferase, a Flaviviridae guanylyltransferase, a Filoviridae guanylyltransferase, a vertebrate guanylyltransferase, or a mammalian guanylyltransferase.

Clause 32. The isolated non-native yeast of clause 1, wherein the Cet1p gene is replaced by an antibiotic resistance gene.

Clause 33. The isolated non-native yeast of clause 1 or clause 32, wherein the heterologous RNA triphosphatase is a full length RNA triphosphatase, a functional RNA triphosphatase fragment, or a codon optimized RNA triphosphatase for expression in yeast.

Clause 34. The isolated non-native yeast of any one of clauses 1, 32, or 33, wherein the heterologous RNA triphosphatase is encoded by a plasmid.

Clause 35. The isolated non-native yeast of clause 34, wherein the plasmid encoding the heterologous RNA triphosphatase comprises a promoter operably linked to the heterologous RNA triphosphatase.

Clause 36. The isolated non-native yeast of clause 20, wherein the plasmid encoding the heterologous RNA triphosphatase comprises or consists of at least 80%, at least 85%, at least 90%, at least 95%, at least 96%, at least 97%, at least 98%, at least 99%, or 100% sequence identity to SEQ ID NOs: 17, 18, or 19.

Clause 37. The isolated non-native yeast of clause 34 or clause 35, wherein the heterologous RNA triphosphatase is fused to an RNA polymerase complex targeting gene.

Clause 38. The non-native yeast of clause 37, wherein the RNA polymerase complex targeting gene comprises a Mce1 GTase domain gene.

Clause 39. The isolated non-native yeast of clause 38, wherein the Mce1 GTase domain gene is a full length Mce1 GTase domain, a functional Mce1 GTase domain fragment, or a codon optimized Mce1 GTase domain gene for expression in yeast.

Clause 40. The isolated non-native yeast of clause 38 or clause 39, wherein the amino acid sequence of the Mce1 GTase domain gene comprises or consists of at least 80%, at least 85%, at least 90%, at least 95%, at least 96%, at least 97%, at least 98%, at least 99%, or 100% sequence identity to SEQ ID NO: 1; or
wherein the nucleotide sequence of the mce1 GTase domain gene comprises or consists of at least 80%, at least 85%, at least 90%, at least 95%, at least 96%, at least 97%, at least 98%, at least 99%, or 100% sequence identity to SEQ ID NO: 2.

Clause 41. The isolated non-native yeast of any one of the preceding clauses, further comprising a Cet1p plasmid comprising a second Cet1p gene operably linked to a promoter.

Clause 42. The isolated non-native yeast of clause 41, wherein the Cet1p plasmid further comprises a yeast Ura3 gene; and wherein the Cet1p plasmid is curable.

Clause 43. The isolated non-native yeast of any one of the preceding clauses, wherein the heterologous RNA triphosphatase comprises nsp9, NP868R, D1, D12, D1 and D12, or MCE1.

Clause 44. The isolated non-native yeast of clause 43, wherein:
the nsp9 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 34;
the D1 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 6;
the D1 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 7;
the D12 comprises or consists of a protein sequence comprising or consisting of at least 80%, at least 85%, at least 90%, at least 95%, at least 96%, at least 97%, at least 98%, at least 99%, or 100% sequence identity to SEQ ID NO: 8;
the D12 is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 9;
the NP868R comprises or consists of a protein sequence comprising or consisting of at least 80%, at least 85%, at least 90%, at least 95%, at least 96%, at least 97%, at least 98%, at least 99%, or 100% sequence identity to SEQ ID NO: 10;
the NP868R is encoded by a nucleic acid sequence comprising or consisting of at least 80%, at least 85%, at least 90%, at least 95%, at least 96%, at least 97%, at least 98%, at least 99%, or 100% sequence identity to SEQ ID NO: 11; or
the MCE1 comprises or consists of a protein sequence comprising or consisting of at least 80%, at least 85%, at least 90%, at least 95%, at least 96%, at least 97%, at least 98%, at least 99%, or 100% sequence identity to SEQ ID NOs: 33 or 287.

Clause 45. The isolated non-native yeast of any of the preceding clauses, wherein the heterologous methyltransferase; heterologous guanylyltransferase; or heterologous RNA triphosphatase is from a virus which encodes an RNA capping enzyme.

Clause 46. The isolated non-native yeast of clause 45, wherein the virus which encodes the RNA capping enzyme is a Coronavirus, an African Swine Fever Virus, a Pox virus, a Flavivirus, a Coronaviridae virus, an Asfarviridae virus, a Poxviridae virus, a Filoviridae virus, or a Flaviviridae virus.

Clause 47. The isolated non-native yeast of clause 46, wherein:
the Coronavirus or the Coronaviridae virus is SARS-CoV-1, SARS-CoV-2, MERS-Cov, 229E, NL63, OC43, or HKU1;
the African Swine Fever Virus or the Asfarviridae virus is African Swine Fever Virus;
the Poxvirus or the Poxviridae virus is smallpox virus, vaccinia virus, monkeypox virus, molluscum contagiosum virus, milker's nodes virus, Orf virus, cowpox virus, or tanapox virus;
the Filoviridae virus is Ebola virus or Marburg virus; or
the Flavivirus or the Flaviviridae virus is dengue virus, yellow fever virus, Zika virus, Japanese encephalitis virus, West Nile virus, Kyasanur forest disease, Alkhurma disease, or Omsk hemorrhagic fever.

Clause 48. The isolated non-native yeast of any of the preceding clauses, wherein the yeast is S. cerevisiae.

Clause 49. The isolated non-native yeast of any of the preceding clauses, wherein the isolated non-native yeast is haploid.

Clause 50. A composition comprising the isolated non-native yeast of any of the preceding clauses and a carrier.

Clause 51. The composition of clause 50, wherein the carrier is a solid or liquid medium.

Clause 52. The composition of clause 50 or clause 51, wherein the carrier comprises synthetic defined media, optionally wherein the synthetic defined media comprises yeast nitrogen base comprising, glucose, lysine, methionine, histidine, and leucine, but without amino acids.

Clause 53. The composition of any one of clauses 50-52, further comprising 5-FOA.

Clause 54. A kit comprising:
a) a plasmid comprising a methyltransferase gene, wherein incorporating the plasmid into a yeast comprising genetically inactivated Abd1 results in the isolated non-native yeast of clause 1;
b) a plasmid comprising a guanylyltransferase gene, wherein incorporating the plasmid into a yeast comprising genetically inactivated Ceg1p results in the isolated non-native yeast of clause 1; or
c) a plasmid comprising an RNA triphosphatase gene, wherein incorporating the plasmid into a yeast comprising genetically inactivated Cet1p results in the isolated non-native yeast of clause 1; and
optionally, further comprising the yeast comprising genetically inactivated Abd1, the yeast comprising genetically inactivated Ceg1p, or the yeast comprising genetically inactivated Cet1p; and
optionally, further comprising instructions for using the kit.

Clause 55. A kit comprising a first strain of the isolated non-native yeast of any one of clauses 1-49, and one or more of: a culture medium and/or instructions for using the kit.

Clause 56. A method of identifying one or more attenuating mutations in the heterologous viral methyltransferase; heterologous viral guanylyltransferase; or heterologous viral RNA triphosphatase of the isolated non-native yeast of clauses 1-49 comprising:
altering one or more amino acid residues in the heterologous viral methyltransferase; heterologous viral guanylyltransferase; or heterologous viral RNA triphosphatase of the isolated non-native yeast to generate an experimental strain;
culturing the experimental strain;
measuring growth of the experimental strain;
comparing the growth of the experimental strain to a control, optionally wherein the control comprises historical non-native yeast growth data or the isolated non-native yeast; and
wherein the experimental strain growing slower than the control indicates the presence of an attenuating mutation in the experimental strain.

Clause 57. A method of screening for methyltransferase, guanylyltransferase, or RNA triphosphatase inhibitors using the isolated non-native yeast of clauses 1-49, comprising:
culturing a first strain of the isolated non-native yeast in media comprising at least one screening compound;
measuring growth of the first strain;
comparing the growth of the first strain to a control, optionally, wherein the control comprises historical non-native yeast growth data, the isolated non-native yeast grown in the absence of the screening compound, or the isolated non-native yeast grown in the presence of a control compound;
optionally, further comprising culturing a second strain of the isolated non-native yeast in the media comprising the screening compound, the second strain comprising a vertebrate methyltransferase gene, a vertebrate guanylyltransferase gene, or a vertebrate RNA triphosphatase gene; measuring the growth of the second strain, and comparing the growth of the second strain to a control, or to the growth of the first strain;
optionally, wherein the media further comprises 5-FOA or G418, and optionally wherein the media lacks uracil;
wherein the first strain growing slower than the control indicates the at least one screening compound is a methyltransferase inhibitor, a guanylyltransferase inhibitor, or an RNA triphosphatase inhibitor; and
optionally, wherein the first strain growing slower than both the control and the second strain indicates the at least one screening compound is a specific methyltransferase inhibitor, a specific guanylyltransferase inhibitor, or a specific RNA triphosphatase inhibitor.

Clause 58. The method of clause 57, wherein the vertebrate methyltransferase gene comprises a human methyltransferase gene; the vertebrate guanylyltransferase gene comprises a human guanylyltransferase gene; the vertebrate RNA triphosphatase gene comprises a human RNA triphosphatase gene; wherein the vertebrate methyltransferase gene comprises a pig methyltransferase gene; the vertebrate guanylyltransferase gene comprises a pig guanylyltransferase gene; or the vertebrate RNA triphosphatase gene comprises a pig RNA triphosphatase gene.

Clause 59. An isolated virus comprising:
a) SARS-CoV-2 nsp14 comprising one or more of the following substitutions: nsp14 W293F, F368N, F368L, D353T, or D353A, wherein the substitutions refer to SEQ ID NO: 4;
b) MERS-CoV nsp14 comprising the following substitution: F365G, wherein the substitution refers to SEQ ID NO: 24;
c) ASFV NP868R comprising one or more of the following substitutions: Y714L, F711L, F711W, D680L, K647Y, or S604A, wherein the substitutions refer to SEQ ID NO: 10;
d) MPV D1 comprising one or more of the following substitutions: Y555F, R655A, D545A, D598A, Y683V, or R548A, R548K wherein the substitutions refer to SEQ ID NO: 290;
e) SARS-CoV-2 nsp12 comprising one or more of the following substitutions: K73A, D218A, D760A, V30A, R33A, R33L, R33K, R55A, R55L, R55K, K50A, C53A, R116A, V71A, N209A, L119A, or Y217F wherein the substitutions refer to SEQ ID NO: 291; or
f) WNV ns5 comprising one or more of the following substitutions: F24L, F24E, or S150C wherein the substitutions refer to SEQ ID NO: 292.

Clause 60. The isolated virus of clause 59, wherein:
a) the isolated virus comprising SARS-CoV-2 nsp14 is SARS-CoV-2;
b) the isolated virus comprising MERS-CoV nsp14 is MERS-CoV;
c) the isolated virus comprising ASFV NP868R is ASFV;
d) the isolated virus comprising MPV D1 is MPV;
e) the isolated virus comprising SARS COV 2 nsp12 is SARS-CoV-2; or
f) the isolated virus comprising WNV ns5 is WNV.

Clause 61. A composition comprising the isolated virus of clause 59 or clause 60, and a carrier.

Clause 62. A composition comprising the isolated virus of clause 59 or clause 60, and an adjuvant.

Clause 63. A method of stimulating an immune response in a subject, comprising:
administering a therapeutically effective amount of the isolated virus of clause 59 or 60, or the composition of clause 61 or 62, to the subject, thereby stimulating the immune response in the subject.

### EXAMPLES

The following examples are provided to illustrate particular features of certain aspects of the disclosure, but the scope of the claims should not be limited to those features exemplified.

### Example 1

### Material and Methods for Results in Example 1 Through Example 6

This example illustrates the methods used for Example 1 to Example 6.

Strains commercially purchased. Saccharomyces cerevisiae strains derived from YBR236C BY4743, homozygous diploid MATa/MATalpha his3 delta1/his3 delta1 leu2 delta0/leu2 delta0 lys2 delta0/+met15 delta0/+ura3 delta0/ura3 delta0 deltaABD1 (ATCC 4033376).

Sporulation and germination procedure. To produce haploid S. cerevisiae ΔABD1 pMO1 from diploid S. cerevisiae ΔABD1 pMO1, diploid S. cerevisiae ΔABD1 pMO1 was subject to sporulation conditions (incubation in 0.3% KOAc at 23° C.) for 5 days⁶⁴. The sporulated cells were then treated with zymolyase and plated on synthetic defined media lacking uracil. Several colonies were screened for their ploidy using oligonucleotides AM965/AM966/AM967 amplifying the S. cerevisiae MATa allele.

Growth media conditions. All Saccharomyces cerevisiae cultures were shaken aerobically at 30° C. and 250 rpm in synthetic defined medium containing 0.67% nitrogen base without amino acids, 2% glucose, 0.02 mg/mL lysine, 0.02 mg/mL methionine, 0.02 mg/mL histidine, and 0.1 mg/ml carbenicillin. Leucine 0.012 mg/mL was added when S. cerevisiae Δabd1 pMO1 haploid was cultured.

Plasmid curing procedure. Plasmids containing ura3 marker were cured by growing cultures in synthetic defined medium containing 1 mg/mL 5-fluoroorotic acid (5FOA) and uracil 0.02 mg/mL for 48 hours or by plating onto synthetic media agar plates containing 1 mg/mL 5-fluoroorotic acid (5FOA) and uracil 0.02 mg/mL and incubating at 30° C. for 48 hours.

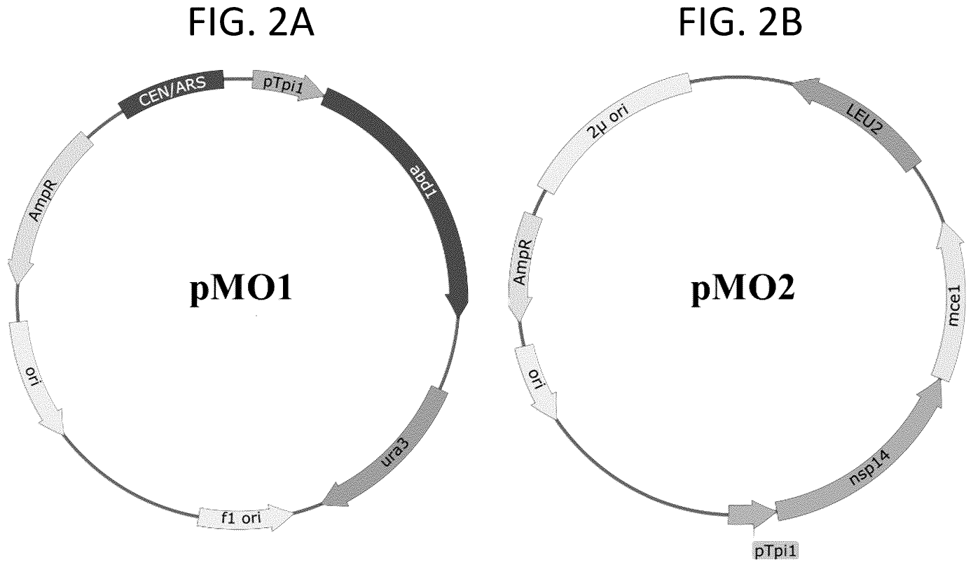

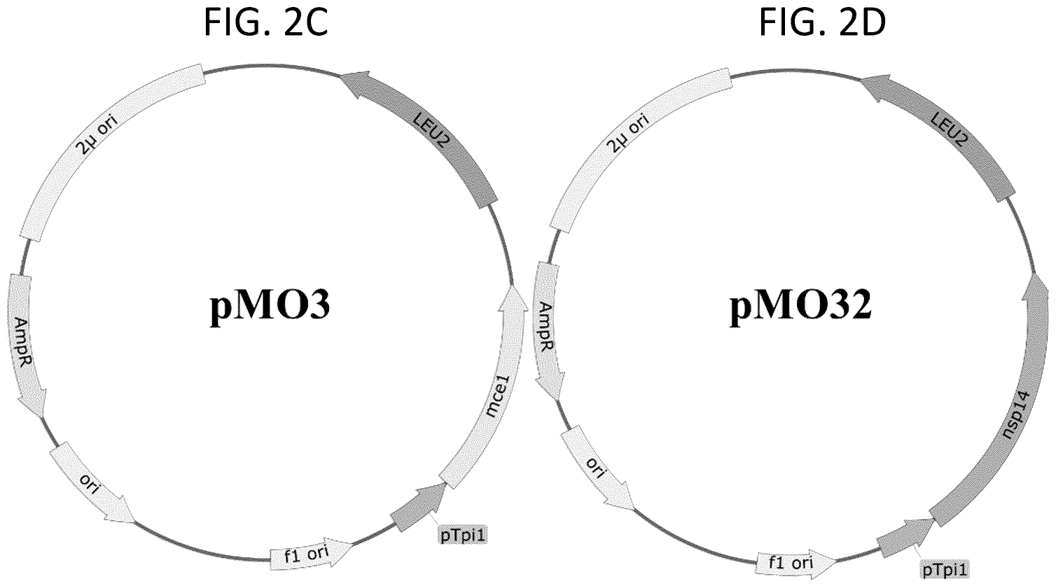

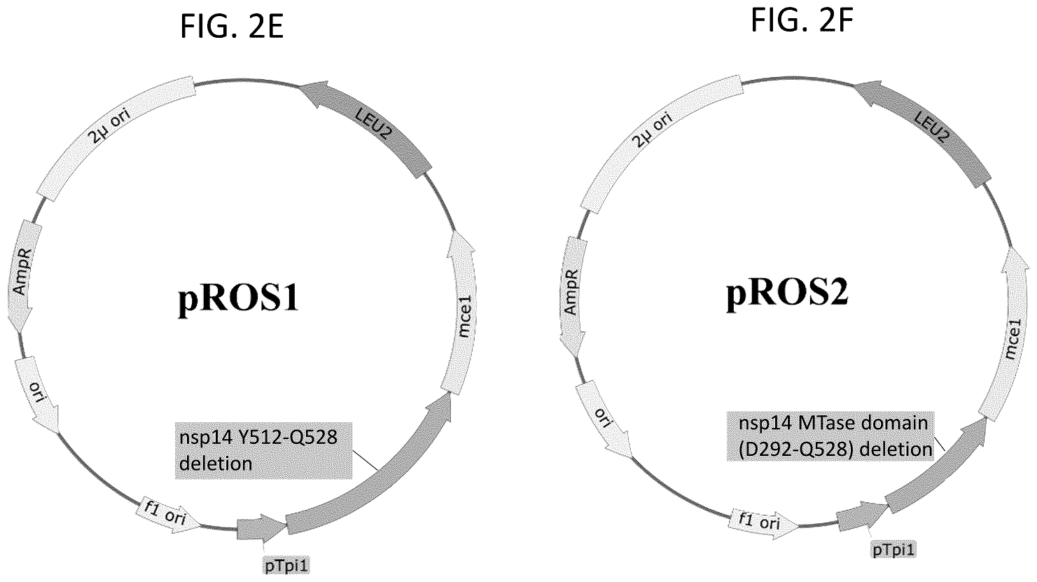

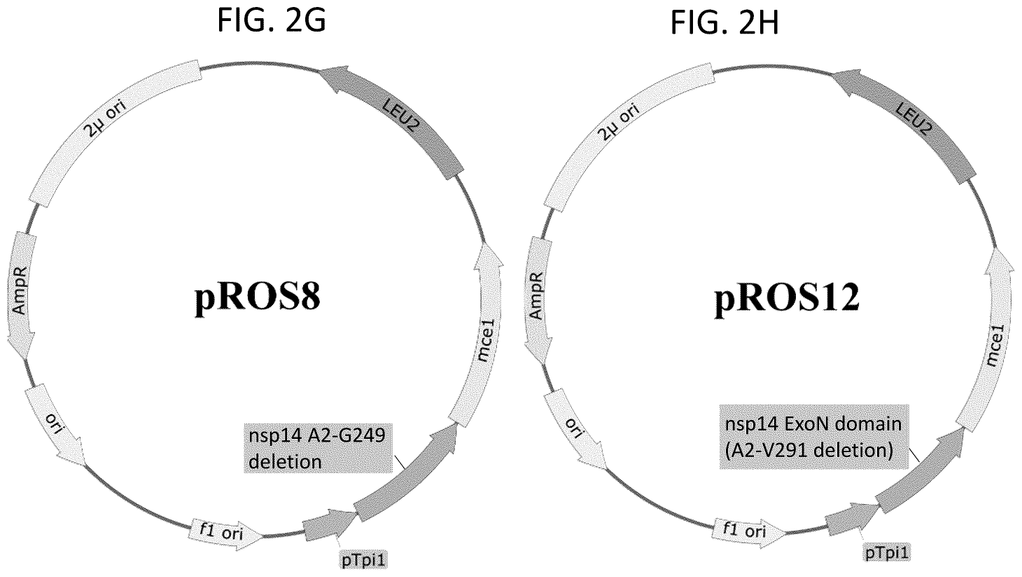

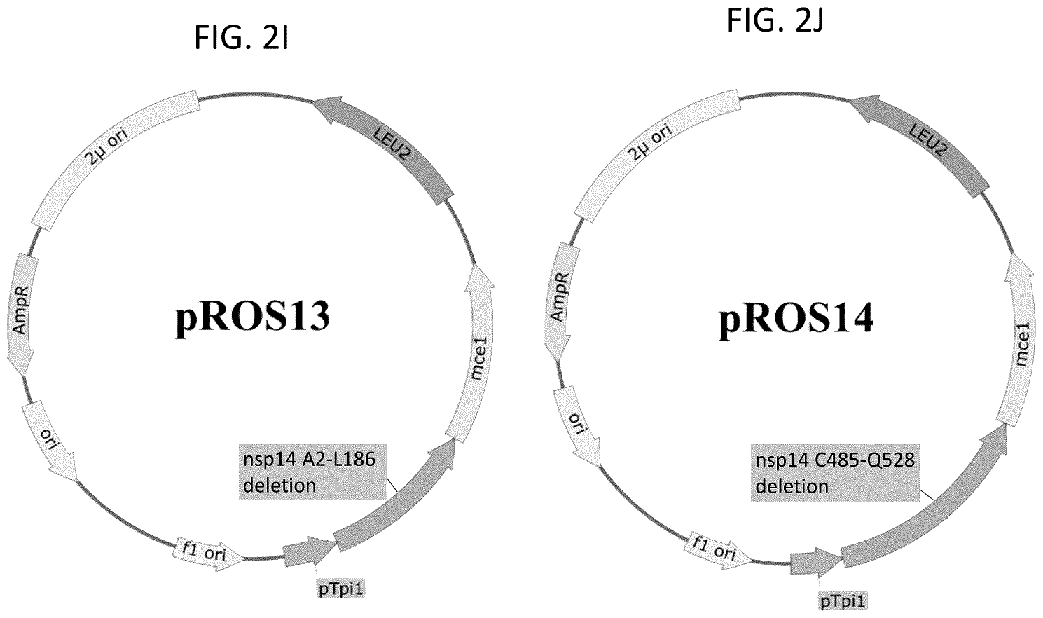

Construction of plasmids. Plasmid maps are included in FIG. 2. Correspondence between sequence numbering and plasmid names are provided in Table 1. All single-stranded and double-stranded DNA oligonucleotide fragments were purchased from Integrated DNA Technologies (IDT). Single-stranded oligonucleotides sequences are listed in Table 2. Double-stranded oligonucleotide sequences (gblocks) used for Gibson assembly are listed in Table 3. Genomic DNA fragments used in the construction of plasmids are listed in Table 4. When noted, coding sequences were codon-optimized for S. cerevisiae expression using IDT codon optimization software.

pMO1: The promoter sequence, pTPI1, was amplified from the gDNA of S. cerevisiae YPH500 using the oligonucleotides M041/MO42. The gene corresponding to ABD1 was amplified from the gDNA of S. cerevisiae YPH500 using the oligonucleotides M045/MO46. pRS416 ura3 was linearized using the oligonucleotides M044/MO43. pTPI1 and abd1 gene fragments were inserted into linearized pRS416 ura3 by Gibson assembly⁶⁵ to afford pMO1.

pMO2: double-stranded nsp14 and mce1 gBlocks codon optimized for S. cerevisiae was purchased from Integrated DNA Technologies (IDT). Mce1 gBlock was amplified using oligonucleotides JC136/M04. Nsp14 gBlock was amplified using oligonucleotides M057/M075. pRS425 leu2 pTPI1 was linearized using the oligonucleotides MO58/JC135. Nsp14 and mce1 fragments were inserted into linearized pRS425 leu2 by Gibson assembly to afford pMO2.

pMO3: pMO2 was linearized using oligonucleotides M0215/MO132. The linearized fragment was ligated by KLD reaction (NEB #M0554S) to afford pMO3.

pMO4: point mutation D331A was introduced into pMO2 by linearizing pMO2 using the oligonucleotides M069/M070. The plasmid was ligated by KLD reaction to afford pMO4.

pROS1: the C-terminal 17 amino acids of nsp14 were removed from pMO2 by amplifying nsp14 using oligonucleotides M0186/MO57. pMO2 was linearized to exclude the C-terminal 17 amino acids of nsp14 using oligonucleotides M0132/MO58. The nsp14 domain fragment was inserted into the linearized pMO2 by Gibson assembly to afford pROS1.

pROS2: the methyltransferase domain of nsp14 was removed from pMO2 by amplifying the exonuclease domain of nsp14 using oligonucleotides MO189/M057. pMO2 was linearized to exclude the methyltransferase domain of nsp14 using oligonucleotides MO132/MO58. The exonuclease domain fragment was inserted into the linearized pMO2 by Gibson assembly to afford pROS2.

pROS8: the exonuclease domain of nsp14 was removed from pMO2 by amplification of the methyltransferase domain of nsp14 using oligonucleotides ROS17/ROS16. pMO2 was linearized to exclude the exonuclease domain of nsp14 using oligonucleotides ROS7/ROS8. The methyltransferase domain fragment was inserted into the linearized pMO2 by Gibson assembly to afford pROS8.

pROS9: point mutation D243A mutation was introduced into pMO2 by linearizing pMO2 using the oligonucleotides ROS18/ROS19. The plasmid was ligated by KLD reaction to afford pMO4.

pROS12: pMO2 was linearized using oligonucleotides ROS21/ROS30 to delete amino acids A2-G249. The linearized fragment was ligated by KLD reaction to afford pROS12.

pROS13: pMO2 was linearized using oligonucleotides ROS21/ROS31 to delete amino acids A2-L186. The linearized fragment was ligated by KLD reaction to afford pROS13.

pROS14: pMO2 was linearized using oligonucleotides ROS8/ROS32 to delete amino acids C485-Q528. The linearized fragment was ligated by KLD reaction to afford pROS14.

pMO15: point mutation V381L was introduced into nsp14 by linearizing pMO2 using oligonucleotides M0246/M0247. The linearized fragment was ligated by KLD reaction to afford pMO15.

pMO16: point mutation A394V was introduced into nsp14 by linearizing pMO2 using oligonucleotides M0248/M0249. The linearized fragment was ligated by KLD reaction to afford pMO16.

pMO17: point mutation P46L was introduced into nsp14 by linearizing pMO2 using oligonucleotides M0242/M0243. The linearized fragment was ligated by KLD reaction to afford pMO17.

pMO18: point mutation P412H was introduced into nsp14 by linearizing pMO2 using oligonucleotides M0250/M0251. The linearized fragment was ligated by KLD reaction to afford pMO18.

C388X/Y369X/A354X library construction: a hydrophobic amino acid focused library was made by randomizing codons at positions C388, Y369, and A354. This was done by amplifying pMO2 using oligonucleotides MO151/M0156 to create a library insert. A second amplification using oligonucleotides MO151/MO157 were performed on this fragment to extend the insert. The insert was ligated to a linearized backbone fragment that was made by amplifying pMO2 with oligonucleotides M0126/M0127. The insert and backbone fragments were ligated using Gibson assembly to afford the library.

W293X/F368X library construction: a library at positions W293 and F368 was made by amplifying pMO2 with oligonucleotides M0221/MO212 to create a library insert. The insert was ligated to a linearized backbone fragment that was made by amplifying pMO2 with oligonucleotides M0218/M0211. The insert and backbone fragments were ligated using Gibson assembly to afford the library.

pMO32: pMO2 was linearized using oligonucleotides M0264/DS001 remove mcel. The linearized fragment was ligated by KLD reaction to afford pMO32.

pMO27: point mutation W293F was introduced into nsp14 by linearizing pMO2 using oligonucleotides M0258/M0259. The linearized fragment was ligated by KLD reaction to afford pMO27.

pMO28: point mutation F368N was introduced into nsp14 by linearizing pMO2 using oligonucleotides M0260/M0261. The linearized fragment was ligated by KLD reaction to afford pMO28.

pMO33: point mutation 142V was introduced into nsp14 by linearizing pMO2 using oligonucleotides M02651M0266. The linearized fragment was ligated by KLD reaction to afford pMO33.

D353X library construction: a library at positions D353 made by amplifying pMO2 with oligonucleotides M0262/JC136 to create a library insert. The insert was ligated to a linearized backbone fragment that was made my amplifying pMO2 with oligonucleotides M0263/JC135. The insert and backbone fragments were ligated using Gibson assembly to afford the library.

TABLE 1
Correspondence between plasmids and sequence numbering.

| Plasmid | Plasmid SEQ ID NO |
|---|---|
| pMO1 | SEQ ID NO: 3 |
| pMO2 | SEQ ID NO: 16 |
| pMO3 | SEQ ID NO: 49 |
| pMO4 | |
| pROS1 | SEQ ID NO: 50 |
| pROS2 | SEQ ID NO: 51 |
| pROS8 | SEQ ID NO: 52 |
| pROS12 | SEQ ID NO: 53 |
| pROS13 | SEQ ID NO: 54 |
| pROS14 | SEQ ID NO: 55 |
| pMO14 | |
| pMO15 | |
| pMO16 | |
| pMO17 | |
| pMO18 | |
| pMO32 | SEQ ID NO: 23 |

TABLE 2
DNA oligos used in the construction of plasmids.

| | SEQ | |
|---|---|---|
| | ID | |
| Name | NO | Sequence |
| MO41 | 56 | ACAGCTTGTCACACCCCTTTTCTGGCA |
| MO42 | 57 | TGGTTGACATTTTTAGTTTATGTATGTGTTTTTTGTAGTT |
| | | ATAG |
| MO45 | 58 | TAAACTAAAAATGTCAACCAAACCAGAGAAGCCAATATG |
| MO46 | 59 | TCCGCTTACATCAGTTGGGCTTTACGCTTTCC |
| MO44 | 60 | GCCCAACTGATGTAAGCGGATGCCGGG |
| MO43 | 61 | AAAGGGGTGTGACAAGCTGTGACCGTCTCC |
| JC136 | 62 | TCTTGAATCAGGTTGGCCGATGCAGTCTTTTG |
| MO4 | 63 | ACGTCTACAGGAACCAGGGTCAAGTGCATCCTTTA |
| JC128 | 64 | AAAGGGGTGTCCAAACAACCAATTACTTGTTGAGAAATAG |
| | | AG |
| JC135 | 65 | ATCGGCCAACCTGATTCAAGAAATATCTTGACCGCAG |
| MO56 | 66 | ACCCTGGTTCGTTAATAGATCAAAAATCATCGCTTCGCTG |
| MO2 | 67 | TCGGCCAACCGAGCGGTCTAAGGCGCC |
| MO55 | 68 | ATCTATTAACGAACCAGGGTCAAGTGCATCC |
| MO1 | 69 | TAGACCGCTCGGTTGGCCGATGCAGTCTTTTG |
| MO69 | 70 | GCTATCGGTAATCCGAAAGCCATAAAATG |
| MO70 | 71 | GTGTAGAACAGGGAATTTGTCCG |
| MO106 | 72 | GAAGCAACAGGAACCAGGGTCAAGTGC |
| MO96 | 73 | AGTTGGCCATTTTTAGTTTATGTATGTGTTTTTTGTAGTT |
| | | ATAGA |
| MO105 | 74 | ACCCTGGTTCCTGTTGCTTCTCAAAGGC |
| MO113 | 75 | TAAACTAAAAATGGCCAACTCCGC |
| MO186 | 76 | AATCCATAATGAGAATCCCGCG |
| MO57 | 77 | TAAACTAAAAATGGCAGAGAATGTAACCGGG |
| MO132 | 78 | GAACCAGGGTCAAGTGCATC |
| MO58 | 79 | TCTCTGCCATTTTTAGTTTATGTATGTGTTTTTTGTAGTT |
| | | ATAGA |
| MO189 | 80 | GATGCACTTGACCCTGGTTCGACACGTTTGACGAAACATT |
| | | CATGT |
| AM965 | 81 | AGTCACATCAAGATCGTTTATGG |
| AM966 | 82 | GCACGGAATATGGGACTACTTCG |
| AM967 | 83 | ACTCCACTTCAAGTAAGAGTTTG |
| MO215 | 84 | TTTTAGTTTATGTATGTGTTTTTTGTAGTTATAGA |
| MO132 | 85 | GAACCAGGGTCAAGTGCATC |
| ROS17 | 86 | ACTACAAAAAACACATACATAAACTAAAAATGGATTGGAG |
| | | CGTGGAATACCC |
| ROS16 | 87 | GATGCACTTGACCCTGGTTCCTGTAGCCTTGTAAAGGTAT |
| | | TCCA |
| ROS7 | 88 | TTTTAGTTTATGTATGTGTTTTTTGTAGT |
| ROS8 | 89 | GAACCAGGGTCAAGTGCATC |
| ROS21 | 90 | TTTTAGTTTATGTATGTGTTTTTTGTAGTTATAGATTTAA |
| | | GC |
| ROS30 | 91 | ATGTTTACGGGTAACCTTCAGTC |
| ROS31 | 92 | ATGTGGGCACACGGCTTT |
| ROS32 | 93 | GACAGCTCCACCAAGATTGC |
| MO246 | 94 | TACAGATGGTCTTTGTTTGTTCT |
| MO247 | 95 | AACTTATCATGGTGGGTCGC |
| MO248 | 96 | TAGATACCCTGTCAACGCCATAG |
| MO249 | 97 | TCCACGTTACAGTTCCAGAACAAAC |
| MO243 | 98 | ATGTCGACACAAAGTCCTTCAGTC |
| MO242 | 99 | ACCTGGGATTCTGAAGGACATGA |
| MO250 | 100 | TCTGAACCTTCACGGGTGTGATG |
| MO251 | 101 | TTACTCAGTACTCTCGTGTCGAATCTG |
| MO133 | 102 | AGAGATAAAACAGATGGCAGAGAATGTAACCGG |
| MO134 | 103 | CACCTCTAGTACTCTGTAGCCTTGTAAAGGTATTCCAT |
| MO135 | 104 | TCTCTGCCATCTGTTTTATCTCTTGTCCCC |
| MO136 | 105 | AAGGCTACAGAGTACTAGAGGTGATGGG |
| MO53 | 106 | CTTCCTTCTGTTCGGAGATTACCGAATC |
| MO54 | 107 | CGGTGATGACGGTGAAAACCTC |
| MO67 | 108 | AAATTTAGGTTTCAATGATCTTCCC |
| MO68 | 109 | TTCGAATCAATAGTTTCCATACCTC |
| MO126 | 110 | AACGTGGATAGATACCCTGC |
| MO151 | 111 | GGCGTTGGCAGGGTATCTATCCACGTTSVVGTTCCAGAAC |
| | | AAAC |
| MO156 | 112 | TGATAAAGCCTACAAGATCGAAGAGTTGTTCBBSTCTTAC |
| | | GCGACCCACC |
| MO157 | 113 | ATGGAAGTTTTACGACBBSCAACCTTGCTCTGATAAAGCC |
| | | TACAAGATCGAAGAGT |
| MO127 | 114 | GTCGTAAAACTTCCATTCGACTTCT |
| MO218 | 115 | CAACTCTTCGATCTTGTAGGCTTT |
| MO221 | 116 | AAAGCCTACAAGATCGAAGAGTTGNNKTATTCTTACGCGA |
| MO211 | 117 | ATCGACACGTTTGACGAAACA |
| MO212 | 118 | TGTTTCGTCAAACGTGTCGATNNKAGCGTGGAATACC |
| MO264 | 119 | TGATTCAAGAAATATCTTGACCGCAG |
| DS001 | 120 | CTGTAGCCTTGTAAAGGTATTCCATAAA |
| ROS18 | 121 | CCATTGTTGTACCGCTATCATAAATGG |
| ROS19 | 122 | CCATTTATGATAGCGGTACAACAATGG |
| MO258 | 123 | TGTTTCGTCAAACGTGTCGATTTTAGCGTGGAATACC |
| MO259 | 124 | TTCATGTACTGCCAAACATCTAGTCATTATGGCGTCA |
| MO260 | 125 | AAAGCCTACAAGATCGAAGAGTTGAATTATTCTTACGCGA |
| MO261 | 126 | ATCAGAGCAAGGTTGGGCGTCGTAA |
| MO262 | 127 | GCAGAAGTCGAATGGAAGTTTTACNNKGCCCAACCTTGCT |
| MO263 | 128 | GTAAAACTTCCATTCGACTTCTGC |
| MO265 | 129 | CTTTGTGTCGACGTACCTGGGATTCCG |
| MO266 | 130 | TCCTTCAGTCTTGAACTTGATATCGACA |

In the sequences of Table 2, the character S can include G or C. The character V can include A, C, or G. The character B can include G, C, or T. The character N can include A, C, G, or T. The character K can include G or T. The character M can include A or C.

TABLE 3
Sequences of gBlocks used in the construction of plasmids.

| | SEQ ID | |
|---|---|---|
| gblock | NO: | Sequence |
| nsp14 | 131 | ATGGCAGAGAATGTAACCGGGCTATTCAAGGATTGCAGCAAAATAATTACCGGGCT |
| | | TCATCCGACGCAAGCACCCACACACCTGAGTGTCGATATCAAGTTCAAGACTGAAG |
| | | GACTTTGTGTCGACATACCTGGGATTCCGAAGGACATGACCTATCGTAGACTAATT |
| | | TCTATGATGGGATTCAAGATGAATTATCAAGTTAACGGCTATCCAAATATGTTCAT |
| | | AACTAGGGAGGAAGCAATACGTCATGTACGTGCTTGGATAGGATTCGACGTAGAAG |
| | | GATGTCATGCCACACGTGACGCTGTGGGTACTAACTTACCATTACAATTAGGCTTT |
| | | AGCACCGGGGTTAACTTAGTTGCAGTTCCCACCGGCTACGTCGACACCGAGAATAA |
| | | CACGGAGTTTACAAGGGTAAATGCCAAGCCGCCGCCAGGTGATCAGTTCAAGCACT |
| | | TGATCCCTTTGATGTACAAGGGACTACCCTGGAATGTAGTAAGAATCAAGATAGTA |
| | | CAAATGCTATCCGACACCCTAAAAGGATTATCCGACCGTGTTGTTTTCGTCTTGTG |
| | | GGCACACGGCTTTGAACTAACGTCAATGAAATACTTTGTCAAAATTGGACCAGAGA |
| | | GAACGTGCTGCCTTTGTGATAAGCGTGCGACTTGTTTTAGCACCAGCTCTGACACC |
| | | TATGCATGTTGGAATCATTCTGTAGGGTTTGACTACGTATACAACCCATTTATGAT |
| | | AGACGTACAACAATGGGGTTTTACGGGTAACCTTCAGTCTAATCACGACCAACATT |
| | | GTCAAGTACACGGCAATGCGCACGTCGCATCCTGTGACGCCATAATGACTAGATGT |
| | | TTGGCAGTACATGAATGTTTCGTCAAACGTGTCGATTGGAGCGTGGAATACCCGAT |
| | | TATTGGTGATGAACTGAGGGTCAACTCTGCTTGTAGGAAAGTACAGCACATGGTAG |
| | | TTAAATCCGCGCTTCTAGCGGACAAATTCCCTGTTCTACACGACATCGGTAATCCG |
| | | AAAGCCATAAAATGTGTACCACAAGCAGAAGTCGAATGGAAGTTTTACGACGCCCA |
| | | ACCTTGCTCTGATAAAGCCTACAAGATCGAAGAGTTGTTCTATTCTTACGCGACCC |
| | | ACCATGATAAGTTTACAGATGGTGTTTGTTTGTTCTGGAACTGTAACGTGGATAGA |
| | | TACCCTGCCAACGCCATAGTTTGCAGATTCGACACGAGAGTACTGAGTAATCTGAA |
| | | CCTTCCCGGGTGTGATGGTGGGAGTTTATACGTGAACAAACATGCCTTTCATACAC |
| | | CGGCCTTTGATAAGAGCGCATTTACCAACTTAAAACAACTGCCATTTTTTTATTAC |
| | | TCAGGTAGTCCGTGCGAGAGTCATGGGAAACAGGTAGTCAGTGATATAGATTACGT |
| | | ACCGCTGAAGTCAGCAACTTGTATAACCAGATGCAATCTTGGTGGAGCTGTCTGTC |
| | | GTCATCATGCTAACGAATACCGTCAATACTTAGACGCATACAACATGATGATTTCC |
| | | GCGGGATTCTCATTATGGATTTATAAACAATTTGATACATACAATTTATGGAATAC |
| | | CTTTACAAGGCTACAG |
| mcel | 132 | GAACCAGGGTCAAGTGCATCCTTTAGTAAAAGGAGAAAAGAGCGGTTAAAACTGGG |
| | | AGCTATTTTCTTGGAAGGCATAACTGTTAAAGGTGTAACTCAAGTAACAACTCAAC |
| | | CCAAGTTAGGAGAGGTGCAGCAGAAATGTCATCAGTTCTGTGGCTGGGAAGGGTCT |
| | | GGGTTCCCTGGAGCACAGCCTGTCTCCATGGACAAGCAAAATATTAGACTTTTAGA |
| | | GCAGAAGCCTTATAAAGTAAGCTGGAAAGCAGACGGCACTCGTTACATGATGTTGA |
| | | TTGATGGCACAAATGAAGTTTTTATGATTGACAGAGATAATTCCGTTTTTCATGTT |
| | | TCAAATCTGGAATTTCCATTTCGTAAAGATCTTCGCATGCATTTATCAAATACTCT |
| | | TTTGGATGGGGAAATGATCATTGACAAAGTAAATGGACAGGCCGTCCCAAGATATT |
| | | TGATCTATGACATAATTAAGTTCAATGCACAACCAGTTGGAGACTGTGATTTTAAT |
| | | ATTCGTCTACAGTGTATTGAACGTGAAATTATAAGTCCACGACATGAAAAAATGAA |
| | | GACTGGACTCATTGACAAAACACAGGAACCATTTAGTGTCAGACCTAAACAATTTT |
| | | TTGACATCAATATTTCAAGAAAGCTTCTGGAAGGAAATTTTGCCAAAGAAGTCAGC |
| | | CATGAAATGGATGGACTTATTTTTCAGCCTATTGGAAAATACAAGCCTGGTCGATG |
| | | TGATGACATTTTGAAATGGAAACCTCCCAGTCTAAACTCTGTGGATTTTCGACTTA |
| | | AGATAACAAGAATGGGAGGAGAAGGGTTGCTTCCACAGAATGTTGGCCTTCTCTAT |
| | | GTTGGAGGTTATGAAAGACCCTTTGCACAAATCAAGGTGACAAAAGAACTAAAACA |
| | | GTATGACAACAAAATTATAGAATGCAAATTTGAGAACAATAGCTGGGTCTTCATGA |
| | | GACAGAGGATAGACAAAAGTTTCCCAAATGCCTACAACACAGCCATGGCTGTGTGC |
| | | AATAGCATCTCGAACCCTGTCACCAAGGAGATGCTGTTTGAATTCATTGACAGATG |
| | | TGCAGCAGCCGCCCAGGGACAGAAGCGGAAGTATCCCCTGGACCCTGACACGGAGC |
| | | TCATGCCGCCCCCACCGCCCAAAAGACTGCATCGGCCAACC |

TABLE 4
Genomic DNA fragments amplified from S. cerevisiae
YPH500 used in the construction of plasmids.

| | SEQ ID | |
|---|---|---|
| gblock | NO | Sequence |
| abd1 | 133 | ATGTCAACCAAACCAGAGAAGCCAATATGGATGTCACAAGAAGACTATGACCGTCA |
| | | GTACGGCTCAATTACTGGTGATGAATCGTCCACAGTCTCAAAAAAAGACTCCAAAG |
| | | TTACTGCCAATGCGCCTGGTGATGGTAATGGATCATTACCAGTTTTGCAAAGCAGC |
| | | TCTATCCTTACTTCTAAGGTGTCTGATCTACCCATTGAGGCAGAATCCGGGTTTAA |
| | | AATCCAAAAGAGAAGACATGAAAGGTATGATCAAGAGGAAAGATTACGCAAACAGC |
| | | GTGCTCAAAAATTGAGAGAAGAGCAACTAAAGAGACATGAAATAGAGATGACTGCA |
| | | AATAGATCCATAAATGTTGACCAGATTGTCCGAGAGCACTATAATGAACGTACAAT |
| | | AATCGCCAATCGCGCTAAGAGGAATCTATCGCCTATTATTAAGCTTCGTAATTTTA |
| | | ACAATGCGATTAAGTATATGTTAATTGACAAATATACAAAACCTGGAGATGTCGTT |
| | | TTAGAACTCGGATGTGGCAAAGGTGGTGATTTAAGAAAATATGGTGCTGCAGGTAT |
| | | TTCACAGTTTATTGGTATTGACATTTCCAATGCCTCTATCCAAGAAGCTCATAAGA |
| | | GATACCGGTCCATGAGAAATTTGGACTACCAAGTTGTGCTAATTACTGGGGATTGC |
| | | TTTGGCGAATCATTAGGTGTTGCAGTAGAGCCCTTCCCAGACTGTAGATTTCCGTG |
| | | TGATATCGTTTCGACGCAATTTTGCTTGCATTATGCTTTTGAGACCGAAGAAAAAG |
| | | CAAGGAGAGCTTTACTGAATGTCGCCAAATCACTCAAAATTGGTGGTCACTTTTTT |
| | | GGCACGATACCAGATTCAGAATTCATTCGTTATAAACTGAATAAATTTCCCAAAGA |
| | | AGTGGAAAAGCCATCTTGGGGAAACTCCATTTATAAAGTGACATTTGAGAATAATT |
| | | CATACCAAAAGAATGATTATGAGTTCACATCGCCTTATGGTCAAATGTATACTTAT |
| | | TGGCTAGAAGATGCTATAGATAATGTTCCTGAATATGTTGTGCCATTCGAAACCTT |
| | | AAGAAGTTTGGCTGATGAATACGGTTTGGAACTAGTATCGCAAATGCCTTTTAATA |
| | | AATTTTTTGTCCAAGAAATTCCCAAATGGATTGAGAGGTTTTCGCCTAAAATGAGG |
| | | GAAGGTTTACAAAGATCTGATGGTAGATATGGTGTTGAAGGGGATGAAAAGGAAGC |
| | | GGCATCATATTTCTATACCATGTTTGCATTCCGTAAGGTAAAACAGTATATCGAAC |
| | | CGGAAAGCGTAAAGCCCAACTGA |
| tpil | 134 | ACACCCCTTTTCTGGCATCCAGTTTTTAATCTTCAGTGGCATGTGAGATTCTCCGA |
| promoter | | AATTAATTAAAGCAATCACACAATTCTCTCGGATACCACCTCGGTTGAAACTGACA |
| | | GGTGGTTTGTTACGCATGCTAATGCAAAGGAGCCTATATACCTTTGGCTCGGCTGC |
| | | TGTAACAGGGAATATAAAGGGCAGCATAATTTAGGAGTTTAGTGAACTTGCAACAT |
| | | TTACTATTTTCCCTTCTTACGTAAATATTTTTCTTTTTAATTCTAAATCAATCTTT |
| | | TTCAATTTTTTGTTTGTATTCTTTTCTTGCTTAAATCTATAACTACAAAAAACACA |
| | | TACATAAACTAAAA |

### Example 2

### Screening for RNA Cap 0 Methyltransferase Activity

The example illustrates methods employed to design YeRCOM and engineer yeast strains for functional screening of RNA cap 0 methyltransferase activity.

Yeast is a model system for functional screening of various eukaryotic enzymes, and yeast-based platforms are used for phenotypic screening to understand the mechanisms of various disease states (like cancer) and develop therapeutics.^{44,45} In addition to this, yeast has also been used as a model organism to characterize the functional activities of a variety of viral enzymes.⁴⁶⁻⁵⁰ yeast-based platform that can functionally characterize and screening of the methyltransferase activity of SARS-CoV-2 nsp14 was designed. Coronavirus nsp14 usually has dual activity, i.e., proof-reading exonuclease activity and mRNA cap guanine-N7 methyltransferase activity.^{24,30} To design an in vivo yeast-based complementation platform for the characterization of SARS-CoV-2 nsp14 N7-MTase activity, Saccharomyces cerevisiae strains that contained a chromosomal deficiency in an essential gene corresponding to S. cerevisiae mRNA cap-0 N7-MTase⁵¹ were engineered. Then, it was tested whether this yeast deficiency could be complemented by plasmid-based expression of SARS-CoV-2 nsp14. The S. cerevisiae mutant strains were expected to survive only if the nsp14 was able to catalyze mRNA cap-0 N7-MTase reaction; on the other hand, S. cerevisiae strains would be inviable in the absence of such activity. This phenotypic platform would allow simple yeast phenotypic screening to characterize the N7-MTase activity of SARS-CoV-2 nsp14, facilitate identification of a methyltransferase domain of SARS-CoV-2 nsp14, facilitate identification of important catalytic residues, and enable screening for inhibitors of SARS-CoV-2 nsp14 methyltransferase activity.

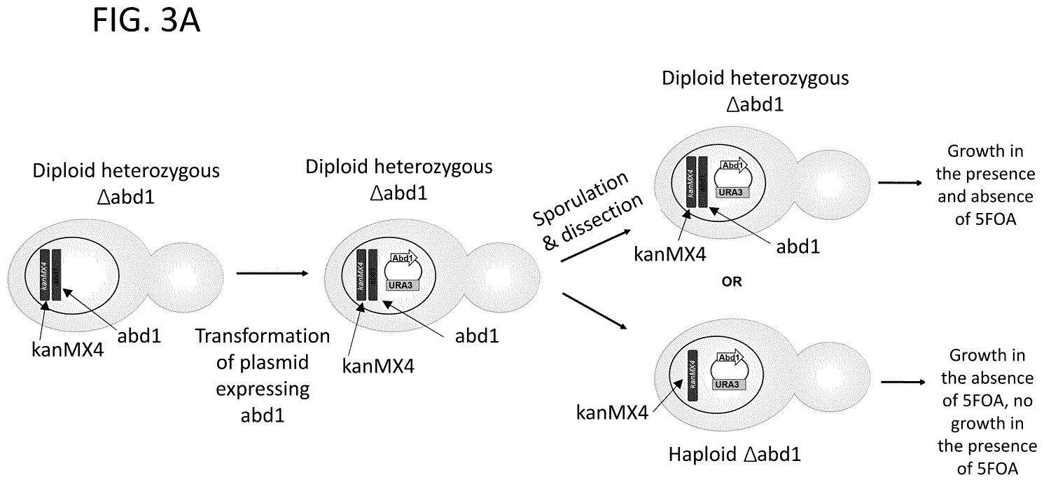

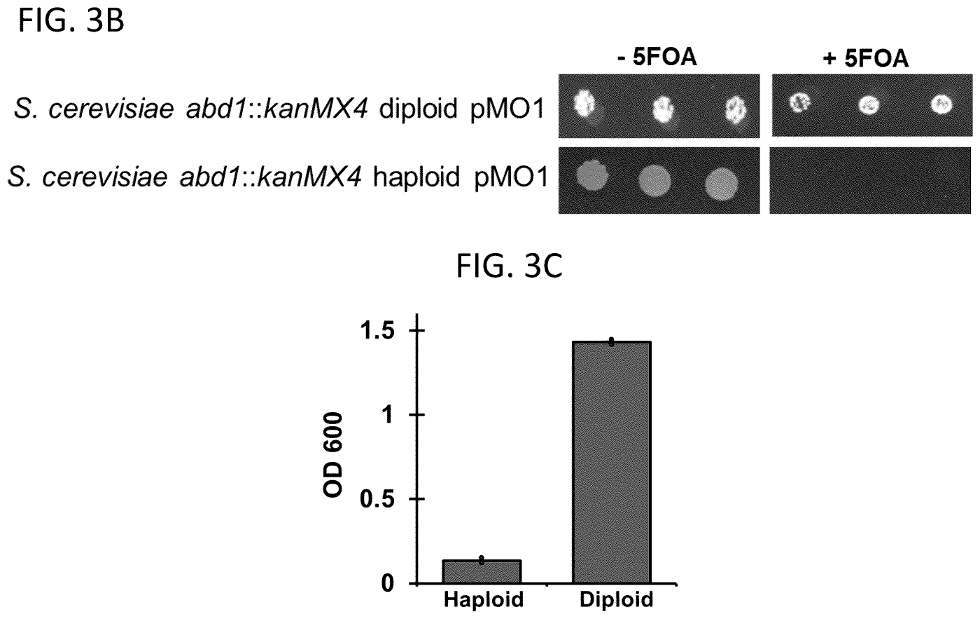

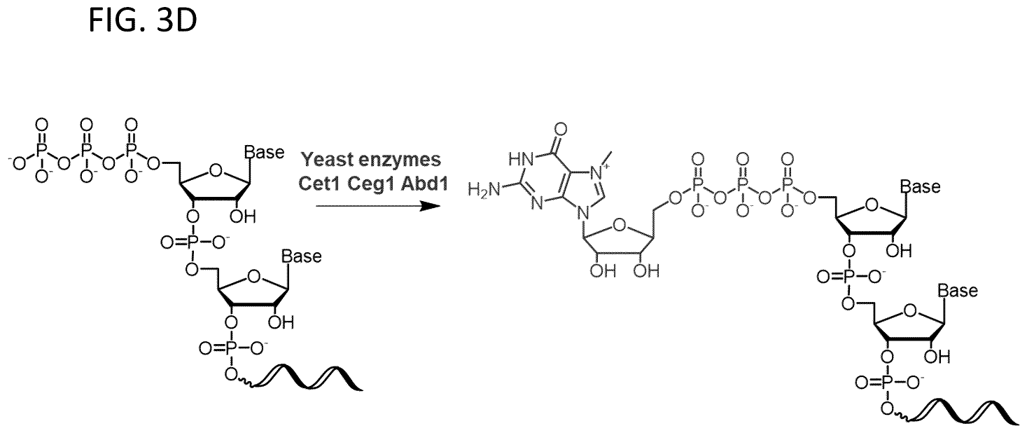

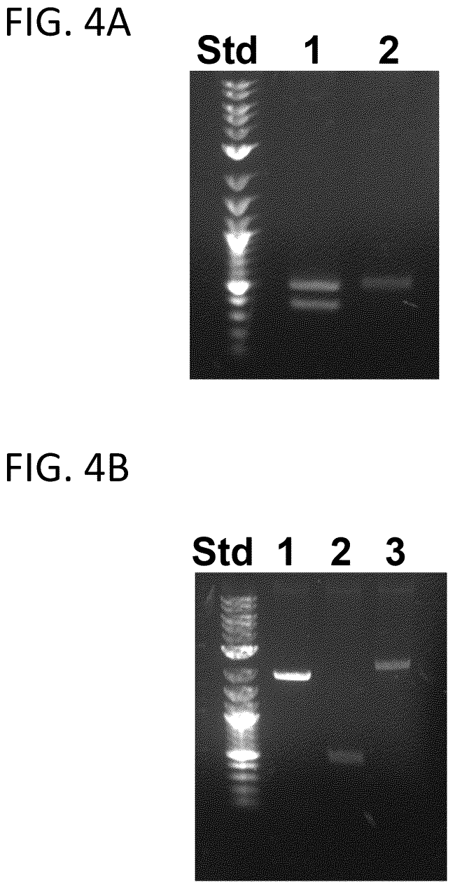

Commercially available S. cerevisiae strain (ATCC #4023376)⁵² was used to engineer this complementation platform as a starting yeast strain. This is a diploid strain of S. cerevisiae where abd1 gene on one of the copies of chromosome II is replaced by G418 resistance marker (referred to as S. cerevisiae abd1::kanMX4 diploid). The gene product corresponding to abd1 is the yeast mRNA cap-0 (guanine-N7)-methyltransferase which is an essential enzyme for the viability of S. cerevisiae. The first goal was to isolate haploid strains of yeast with a single copy of chromosome II containing abd1 gene deletion. Because abd1 is an essential gene⁵¹, complementation of abd1 deficiency by plasmid-based expression of abd1 was attempted. To accomplish this, a curable plasmid pMO1 which contains abd1 expression cassette under pTPI1 promoter and ura3 gene as the selection marker and transformed S. cerevisiae abd1::kanMX4 diploid strain with pMO1 plasmid was constructed (Table 5). These strains were then subjected to sporulation for 5 days and recovered on selection medium lacking uracil but containing G418. The resulted in the growth of two possible clonal populations (FIG. 3A): (i) S. cerevisiae abd1::kanMX4 diploid strain containing plasmid pMO1 and (ii) S. cerevisiae abd1::kanMX4 haploid strain containing plasmid pMO1. As per the YeRCOM design, the next goal was to isolate the haploid strain as the starting host strain. To this end, we screened surviving colonies to identify S. cerevisiae abd1::kanMX4 haploid strain containing plasmid pMO1. Total genomic DNA from a subset of individual colonies was isolated and analyzed using primers specific to the S. cerevisiae mating allele (MAT) to identify if the strains contained both MATa and MATα (indicating diploid strains) or just one of them (indicating haploid strain). Through this screening S. cerevisiae abd1::kanMX4 haploid strain containing plasmid pMO1 was identified (FIG. 4). The resulting haploid strains had a replacement of abd1 gene by G418 resistance cassette on chromosome II (FIG. 4). In these strains the chromosomal deficiency of the essential S. cerevisiae gene, abd1, was rescued by the pMO1 expressing functional ABDI (Strain name: S. cerevisiae abd1::kanMX4 pMO1 haploid). Since pMO1 has a ura3 marker, it can be cured in presence of 5-fluorooratic acid (5-FOA) which is converted to 5-fluorouracil which is a toxic molecule.⁵¹ So, in case of S. cerevisiae abd1::kanMX4 pMO1 haploid strains, the addition of 5-FOA would be expected to result in loss of cell viability due to curing of pMO1 resulting in the lack of complete lack of nuclear and plasmid-based abd1 expression which is lethal to S. cerevisiae strains. It was confirmed that S. cerevisiae abd1::kanMX4 pMO1 haploid strains are not viable when cultured in synthetic defined medium containing 5-FOA. This can be seen from FIGS. 3B-3C, no cell growth is observed in the presence of 5-FOA indicating that the lack of ABD1 expression is lethal to the cells. On the other hand, growth is observed in the control strains S. cerevisiae abd1::kanMX4 diploid pMO1 in presence of 5-FOA as one of the copies of chromosomes II in the diploid strain still possess native abd1 gene. Taken together, these data indicate that the S. cerevisiae abd1::kanMX4 pMO1 haploid strain can be used for functional screening of N7-MTase activity. This haploid strain was used for developing subsequent YeRCOM platforms. Table 5 has a list of plasmids constructed and used. FIG. 2 and Table 1 have plasmid map details and Table 6 has a list of the strains engineered and used.

TABLE 5
Details of plasmids used in the instant disclosure.

| | | Plasmid | Plasmid |
|---|---|---|---|
| Plasmid | P_{TPI1} driven expression cassette | marker | backbone |
| pMO1 | abd1 | URA3 | pRS416 |
| pMO2 | nsp 14 - mce1 | LEU2 | pRS425 |
| pMO3 | mce1 | LEU2 | pRS425 |
| pMO4 | nsp14 (D331A) - mce1 | LEU2 | pRS425 |
| pROS1 | nsp14 (Y512-Q528 deletion) - mce1 | LEU2 | pRS425 |
| pROS2 | nsp14 MTase domain (D292-Q528) deletion - mce1 | LEU2 | pRS425 |
| pROS9 | nsp14 (D243A) - mce1 | LEU2 | pRS425 |
| pROS8 | nsp14 ExoN domain (A2-V291) deletion - mce1 | LEU2 | pRS425 |
| pROS12 | nsp14 (A2 - G249 deletion) - mce1 | LEU2 | pRS425 |
| pROS13 | nsp14 (A2 - L186 deletion) - mce1 | LEU2 | pRS425 |
| pROS14 | nsp14 (C485-Q528 deletion) - mce1 | LEU2 | pRS425 |
| pMO15 | nsp14 (V381L) - mce1 | LEU2 | pRS425 |
| pMO16 | nsp 14 (A394V) - mce1 | LEU2 | pRS425 |
| pMO17 | nsp14 (P46L) - mce1 | LEU2 | pRS425 |
| pMO18 | nsp14 (P412H) - mce1 | LEU2 | pRS425 |
| pMO32 | nsp14 | LEU2 | pRS425 |
| pMO27 | nsp 14 (W293F) - mce1 | LEU2 | pRS425 |
| pMO28 | nsp14 (F368N) - mce1 | LEU2 | pRS425 |
| pMO33 | nsp14 (142V) - mce1 | LEU2 | pRS425 |

TABLE 6
YeRCOM strains engineered in the instant disclosure.

| | P_{TPI1} driven expression | | |
|---|---|---|---|
| | cassettes present in these | | |
| Strain Name | strains | Mating allele | Source |
| S. cerevisiae abd1::kanMX4 | NA | MATa MATα | ATCC |
| diploid | | | 4023376 |
| S. cerevisiae abd1::kanMX4 | abd1 | MATa | This |
| pMO1 haploid | | | study |
| S. cerevisiae abd1::kanMX4 | abd1, | MATa | This |
| pMO1 pMO2 haploid | nsp14 - mce1 | | study |
| S. cerevisiae abd1::kanMX4 | abd1, | MATa | This |
| pMO1 pMO3 haploid | mce1 | | study |
| S. cerevisiae abd1::kanMX4 | abd1, | MATa | This |
| pMO1 pMO4 haploid | nsp14 (D331A) - mce1 | | study |
| S. cerevisiae abd1::kanMX4 | abd1, | MATa | This |
| pMO1 pROS1 haploid | nsp14 (Y512-Q528 deletion) | | study |
| | - mce1 | | |
| S. cerevisiae abd1::kanMX4 | abd1, | MATa | This |
| pMO1 pROS2 haploid | nsp14 MTase domain | | study |
| | (D292-Q528) deletion - | | |
| | mce1 | | |
| S. cerevisiae abd1::kanMX4 | abd1, | MATa | This |
| pMO1 pROS9 haploid | nsp14 (D3243A) - mce1 | | study |
| S. cerevisiae abd1::kanMX4 | abd1, | MATa | This |
| pMO1 pROS8 haploid | nsp14 ExoN domain (A2- | | study |
| | V291) deletion - mce1 | | |
| S. cerevisiae abd1::kanMX4 | abd1, | MATa | This |
| pMO1 pROS12 haploid | nsp14 (A2 - G249 deletion) - | | study |
| | mce1 | | |
| S. cerevisiae abd1::kanMX4 | abd1, | MATa | This |
| pMO1 pROS13 haploid | nsp14 (A2 - L186 deletion) - | | study |
| | mce1 | | |
| S. cerevisiae abd1::kanMX4 | abd1, | MATa | This |
| pMO1 pROS14 haploid | nsp14 (C485-Q528 deletion) | | study |
| | - mce1 | | |
| S. cerevisiae abd1::kanMX4 | abd1, | MATa | This |
| pMO1 pMO15 haploid | nsp14 (V381L) - mce1 | | study |
| S. cerevisiae abd1::kanMX4 | abd1, | MATa | This |
| pMO1 pMO16 haploid | nsp14 (A394V) - mce1 | | study |
| S. cerevisiae abd1::kanMX4 | abd1, | MATa | This |
| pMO1 pMO17 haploid | nsp14 (P46L) - mce1 | | study |
| S. cerevisiae abd1::kanMX4 | abd1, | MATa | This |
| pMO1 pMO18 haploid | nsp14 (P412H) - mce1 | | study |
| S. cerevisiae abd1::kanMX4 | abd1, | MATa | This |
| pMO1 pMO32 haploid | nsp14 | | study |
| S. cerevisiae abd1::kanMX4 | abd1, | MATa | This |
| pMO1 pMO27 haploid | nsp14 (W293F) - mce1 | | study |
| S. cerevisiae abd1::kanMX4 | abd1, | MATa | This |
| pMO1 pMO28 haploid | nsp14 (F368N) - mce1 | | study |
| S. cerevisiae abd1::kanMX4 | abd1, | MATa | This |
| pMO1 pMO33 haploid | nsp14 (142V) - mce1 | | study |

### Example 3

### YeRCOM-nsp14

This example illustrates building YeRCOM-nsp14 for SARS-CoV-2 nsp14 N7-MTase activity.

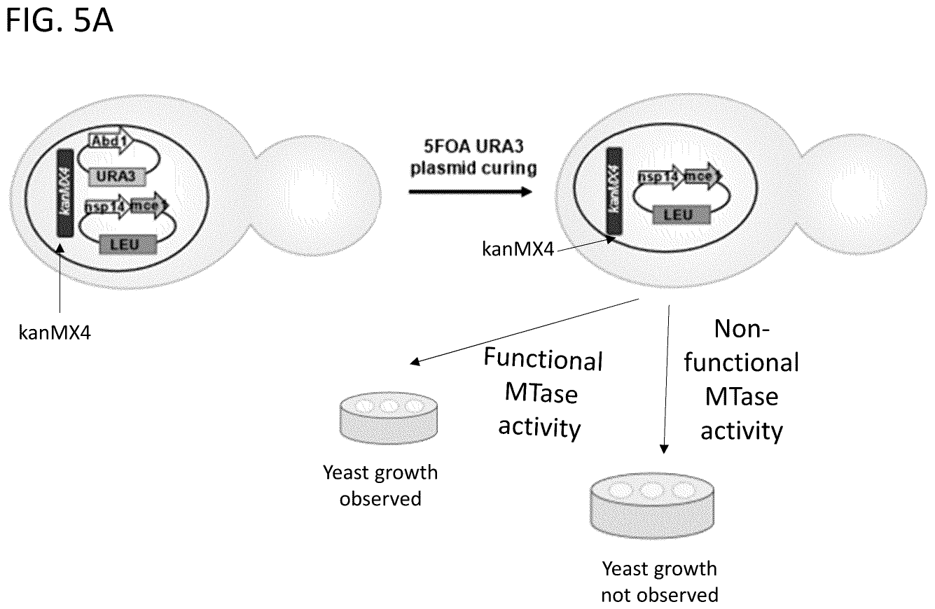

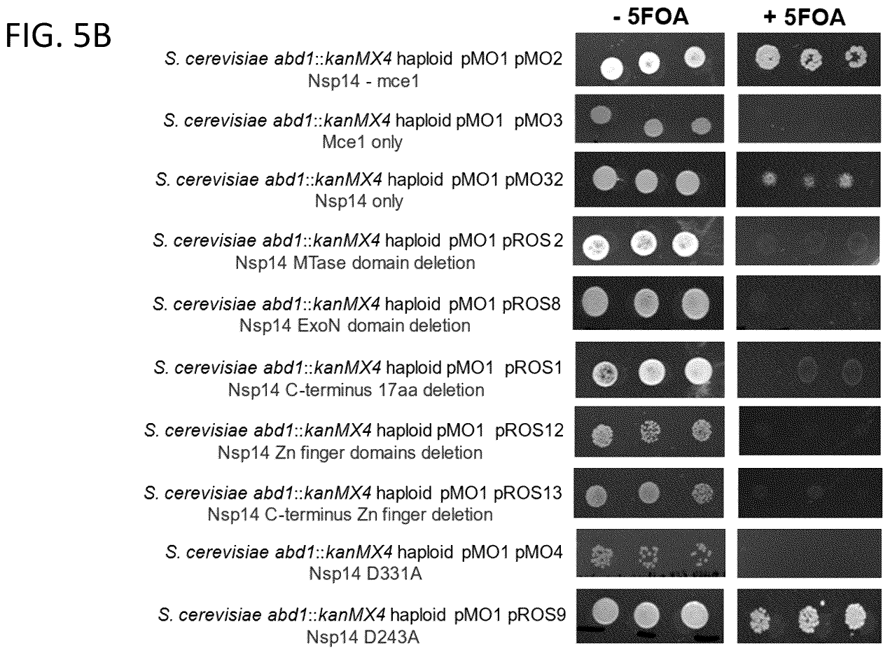

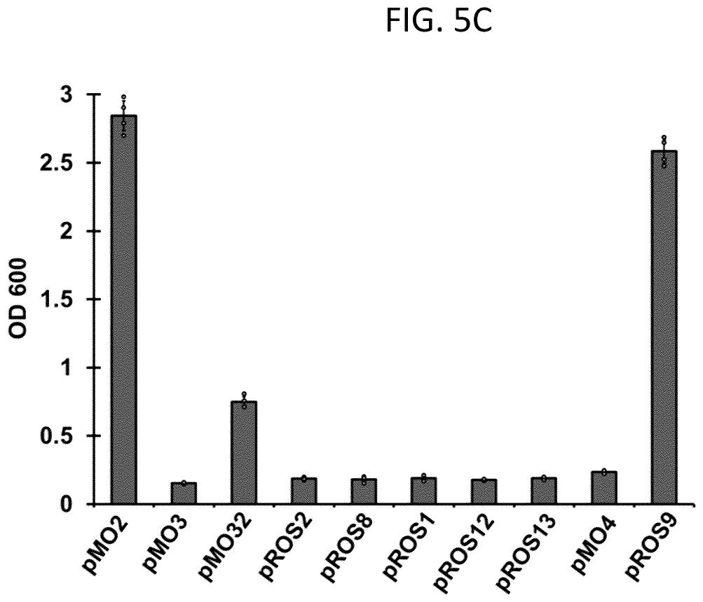

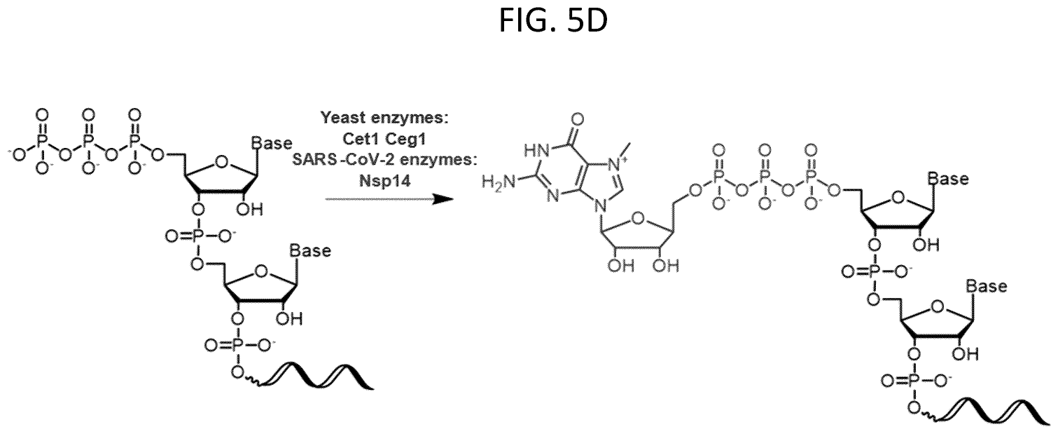

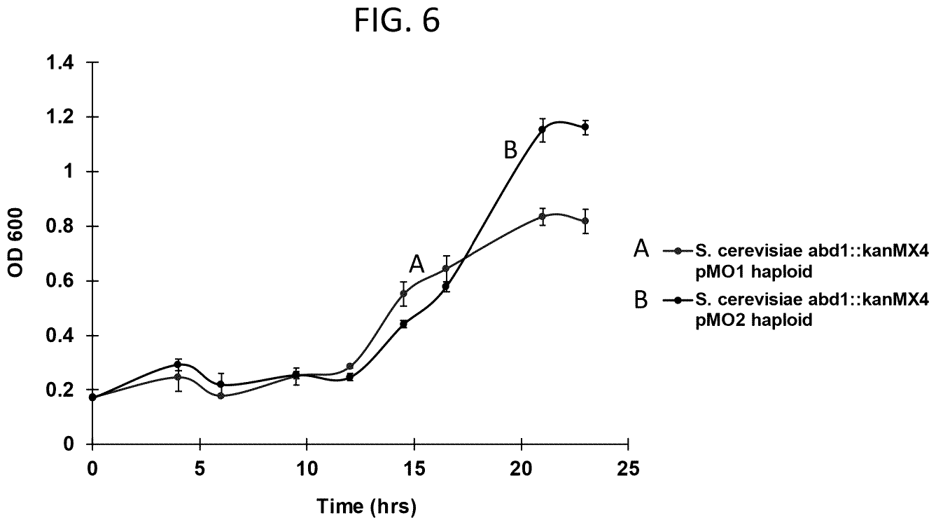

The use of the S. cerevisiae abd1::kanMX4 pMO1 haploid-based platform to functionally characterize SARS-CoV-2 nsp14 N7-MTase activity was investigated. Particularly, it was investigated whether S. cerevisiae abd1::kanMX4 haploid abd1 deficiency can be rescued by the expression of SARS-CoV-2 RNA-cap-0 N7-MTase, nsp14. In order to screen for this possibility, a fusion between SARS-CoV-2 nsp14 sequence and human mce1 gene was generated to direct the viral nsp14 to RNA polymerase 11 complex⁵¹ and it was incorporated into a plasmid with PTPn1 promoter and leu2 marker (plasmid name: pMO2). A control plasmid lacking nsp14 and expressing only mec1 (plasmid pMO3) was also constructed. pMO2 and pMO3 were transformed into S. cerevisiae abd1::kanMX4 pMO1 haploid to generate S. cerevisiae abd1::kanMX4 pMO1 pMO2 haploid and S. cerevisiae abd1::kanMX4 pMO1 pMO3 haploid respectively. If the SARS-CoV-2 nsp14-MEC1 fusion catalyzes the RNA cap 0 N7-methylation of the native yeast mRNAs, then S. cerevisiae abd1::kanMX4 pMO1 pMO2 haploid will survive in presence of 5-FOA where the abd1 expressing plasmid pMO1 is cured. This would indicate that the SARS-CoV-2 nsp14-MEC1 fusion was able to functionally rescue abd1 deficiency in S. cerevisiae abd1::kanMX4 haploid strains. On the other hand, if no growth was observed for S. cerevisiae abd1::kanMX4 pMO1 pMO2 haploid strains in presence of 5-FOA, this would indicate that the SARS-CoV-2 nsp14-MEC1 fusion was unable to functionally rescue abd1 deficiency in S. cerevisiae abd1::kanMX4 haploid strains. As depicted in FIGS. 5B-5C, the S. cerevisiae abd1::kanMX4 pMO1 pMO2 haploid was able to survive in presence of 5-FOA indicating the that the SARS-CoV-2 nsp14-MEC1 fusion was able to rescue complete lack of nuclear and plasmid expression of abd1. On the other hand, the control plasmid lacking nsp14 was unable to rescue the growth of S. cerevisiae abd1::kanMX4 pMO1 pMO3 haploid in the presence of 5-FOA indicating that human mce1 alone was unable to rescue the abd1 deficiency in S. cerevisiae abd1::kanMX4 haploid strains. Taken together, these data indicate that SARS-CoV-2 nsp14 was active (which is consistent with the in vitro enzyme assays⁴²′4¹) and was able to catalyze the N7-methylation of the RNA cap 0 of S. cerevisiae mRNAs resulting in the survival of S. cerevisiae abd1::kanMX4 in the absence of abd1. It was further investigated if instead of using nsp14-mce1 fusion, nsp14 could be used by itself (plasmid: pMO32) for functional rescue of S. cerevisiae abd1::kanMX4 pMO1 haploid strain in presence of 5-FOA. As indicated in FIGS. 5B-5C, S. cerevisiae abd1::kanMX4 pMO1 pMO32 haploid strains were able to grow in presence of 5-FOA, albeit to a lower level. This indicated that though the mce1 fusion to nsp14 was not necessary, it was certainly helpful for high-efficiency complementation of S. cerevisiae abd1::kanMX4 pMO1 haploid strain in presence of 5-FOA. Therefore, analysis was performed with variants of nsp14 fused to mce1 targeting domain. These set of experiments suggested that this engineered phenotypic platform could be used to for functional screening of SARS-CoV-2 nsp14 N7-MTase activity. S. cerevisiae abd1::kanMX4 pMO2 haploid strain (YeRCOM-nsp14 strain) was generated by two rounds of growth of S. cerevisiae abd1::kanMX4 pMO1 pMO2 haploid strain in the presence of 5-FOA which resulted in complete curing of pMO1 from S. cerevisiae abd1::kanMX4 pMO1 pMO2 haploid strain. The growth rate of S. cerevisiae abd1::kanMX4 pMO1 haploid was characterized in comparison to S. cerevisiae abd1::kanMX4 pMO2 haploid (FIG. 6).

### Example 4

SARS-CoV-2 nsp14 N7-MTase Characterization

This example illustrates using YeRCOM-nsp14 platform to determine relevant domains in SARS-CoV-2 nsp14 N7-MTase.

The YeRCOM-nsp14 platform was used to determine the relevant domains and amino acid residues in SARS-CoV-2 nsp14 related to N7-MTase activity. To demonstrate the functional relevance of the putative methyltransferase domain of nsp14^{54,55} to its N7-MTase activity, a truncation variant of SARS-CoV-2 nsp14 that lacked the N7-MTase domain was generated (deletions from amino acids 292-528, plasmid pROS2 containing nsp14 A292-528). Next, S. cerevisiae abd1::kanMX4 pMO1 haploid was transformed with pROS2 to generate S. cerevisiae abd1::kanMX4 pMO1 pROS2 haploid strain.

To determine if nsp14 A292-528 truncation possessed N7-MTase activity, S. cerevisiae abd1::kanMX4 pMO1 pROS2 haploid strain was treated with 5-FOA and as shown in FIGS. 5B-5C, these strains were unable to survive upon 5-FOA treatment indicating that nsp14 A292-528 truncation was catalytically inactive. This suggested that the MTase domain of nsp14 was involved in its N7-MTase activity. Next, the relevance of exonuclease domain was for the N7-MTase activity of nsp14 was investigated. A plasmid pROS3 containing a variant of nsp14 that lacked the ExoN domain (amino acids 2-292, nsp14 A2-292 truncation) was constructed. S. cerevisiae abd1::kanMX4 pMO1 haploid strain was then transformed with pROS8 to generate S. cerevisiae abd1::kanMX4 pMO1 pROS8 haploid strain. As before 5-FOA treatment was performed and as shown in FIG. 5B-5C, surprisingly, the S. cerevisiae abd1::kanMX4 pMO1 pROS8 haploid was unable to survive in presence of 5-FOA indicating that fusion of methyltransferase domain of nsp14 alone to mce1, was not sufficiently for efficient RNA cap-0 N7-MTase activity. It was hypothesized that the ExoN domain of nsp14 likely plays a structural role for efficient N7-MTase activity and predicted that the functionally inactive exonuclease domain along with MTase domain of nsp14 fused to mce1 would be able to complement abd1 deficiency. To test this hypothesis, a catalytically inactive variant of ExoN⁵⁶ was generated (nsp14 D243A, plasmid pROS9), and transformed into host cells to generate S. cerevisiae abd1::kanMX4 pMO1 pROS4 haploid. S. cerevisiae abd1::kanMX4 pMO1 pROS9 haploid was able to survive in presence of 5-FOA (FIG. 5B-5C). This observation was consistent with the hypothesis that while it may not be necessary to have a catalytically active ExoN domain, the structural features of the ExoN domain of nsp14 could play a role in efficient catalysis of the nsp14 N7-MTase activity.

Next, the roles of several zinc finger domains in SARS-CoV-2 nsp14 were investigated. Nsp14 has two zinc finger domains near the N-terminus and one zinc finger domain near the C-terminus which could play a role in binding of the RNA substrate. To investigate the importance of these domains for N7-MTase activity, four truncation variants of nsp14 were made: (i) C-terminal truncation (nsp14 A485-528, plasmid pRSO14) (ii) C-terminal 17 amino acid truncation (nsp14 A512-528, plasmid pROS1) (iii) nsp14 A2-186 containing a deletion of one zinc finger domain (plasmid pROS12) and (iv) nsp14 A2-249 containing a deletion of two zinc finger domains (plasmid pROS13). Plasmids corresponding to each of these variants were transformed into S. cerevisiae abd1::kanMX4 pMO1 haploid to generate corresponding haploid strains. As shown in FIG. 5B-5C, none of the haploid strains were able to survive in presence of 5-FOA indicating that the importance of zinc finger domains to nsp14 activity.

### Example 5

SARS-CoV-2 nsp14 Variants

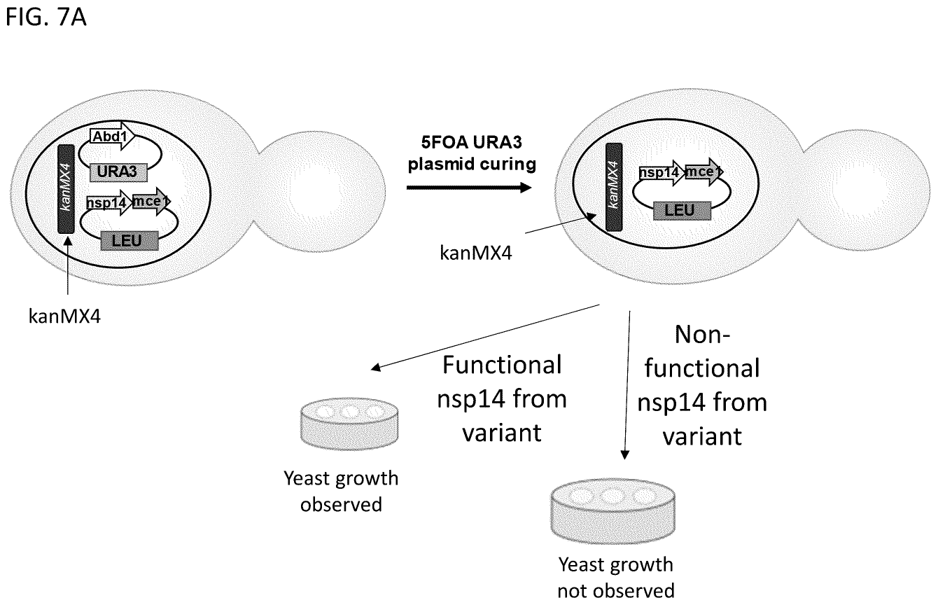

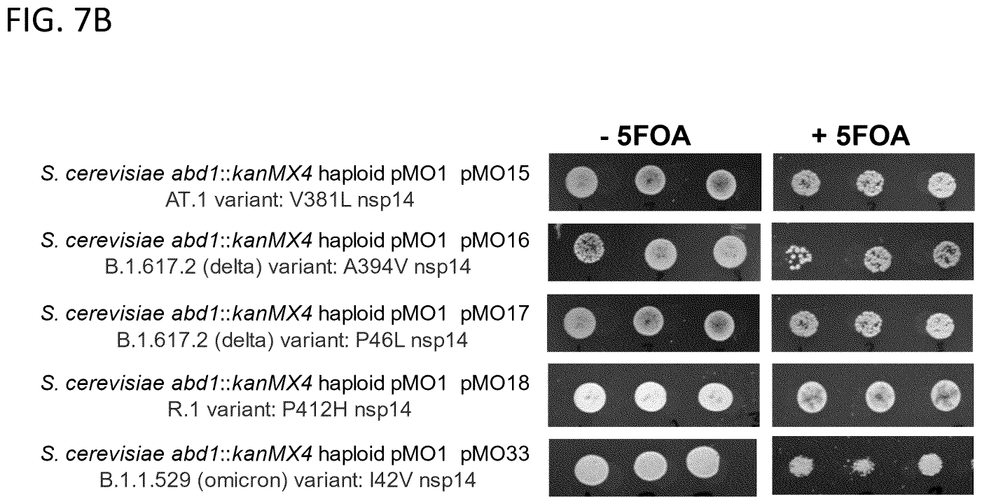

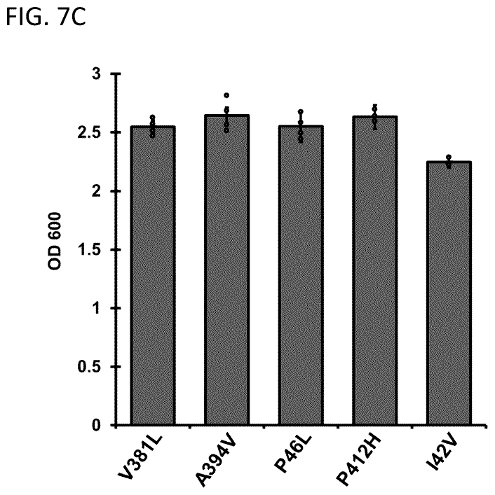

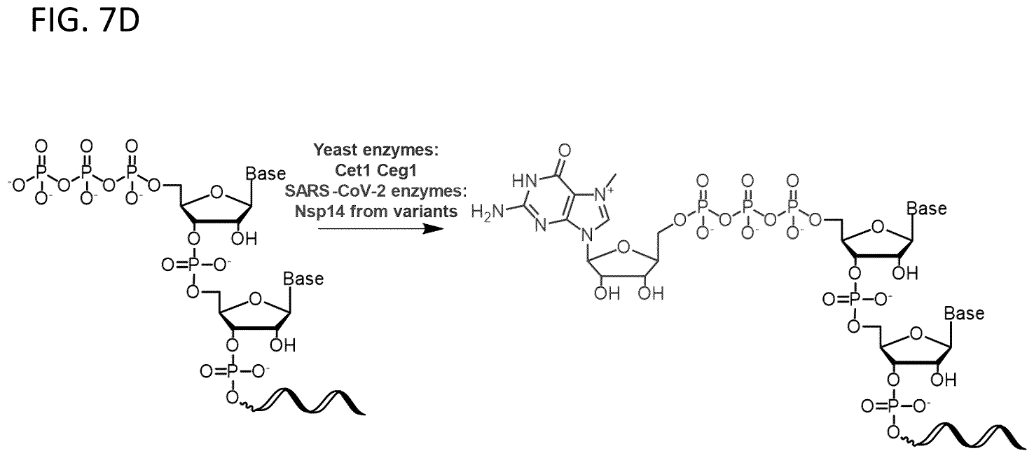

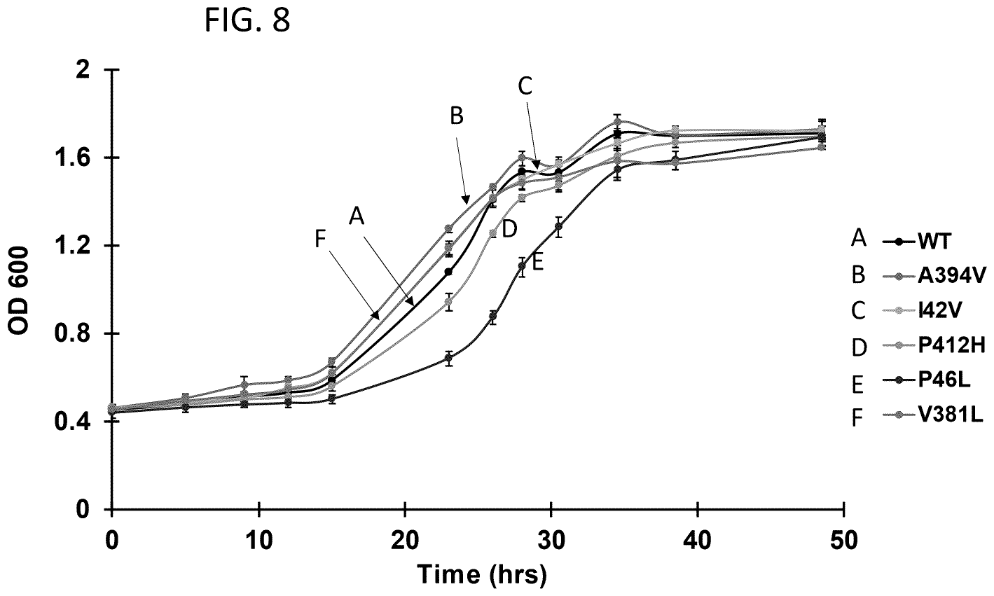

This example illustrates expanding YeRCOM-nsp14 platforms for nsp14 mutations observed in variants of SARS-CoV-2 nsp14 activity. Since COVID-19 was declared as a pandemic, remarkable efforts are ongoing to track various variants of SARS-CoV-2. Some of the notable variants of SARS-CoV-2 include: B.1.1.529 (or omicron), B.1.617.2 (delta), AZ.5, and C.1.2 among others. Significant efforts have been focused on understanding the mutations observed in the S gene and their correlation to the escape from vaccine-induced immune response. Several of the SARS-CoV-2 variants of concern also have mutations in the gene encoding nsp14. The B.1.1.529 (omicron) variants encode for nsp14 I42V. B.1.617.2 (delta) variants encode for two different variants of nsp14 (A394V and P46L). The AT.1 variant encodes for nsp14 V381L. The B.1.616 1 variant encodes for nsp14 L157F. The R.1 variant encodes for P412H. Importantly, along with variations in the ExoN domain of nsp14, variations are also observed in the N7-MTase domain of nsp14. In order to understand the effect of these mutations on nsp14 N7-MTase activity, plasmids were constructed corresponding to each of these variants of nsp14 and transformed into our starting yeast strains, S. cerevisiae abd1::kanMX4 pMO1 haploid. Upon treatment with 5-FOA, similar growth of the corresponding yeast strains was observed compared to the yeast strains expressing wildtype nsp14 (i.e., S. cerevisiae abd1::kanMX4 pMO1 pMO2 haploid, FIGS. 7B-7C). As before, S. cerevisiae abd1::kanMX4 pMO15 haploid, S. cerevisiae abd1::kanMX4 pMO16 haploid, S. cerevisiae abd1::kanMX4 pMO17 haploid, S. cerevisiae abd1::kanMX4 pMO18 haploid and S. cerevisiae abd1::kanMX4 pMO33 haploid strains were generated by two rounds of growth in the presence of 5-FOA which resulted in complete curing of pMO1. These strains can be readily used for phenotypic screening purposes for evaluating the activities of inhibitors targeting notable nsp14 variants. The growth rates of each of these strains in comparison to S. cerevisiae abd1::kanMX4 pMO2 haploid were characterized (FIG. 8).

### Example 6

Inactivation and Attenuation of SARS-CoV-2 nsp14

This example illustrates the use of YeRCOM-nsp14 to identify inactivation and attenuation variants of SARS-CoV-2 nsp14.

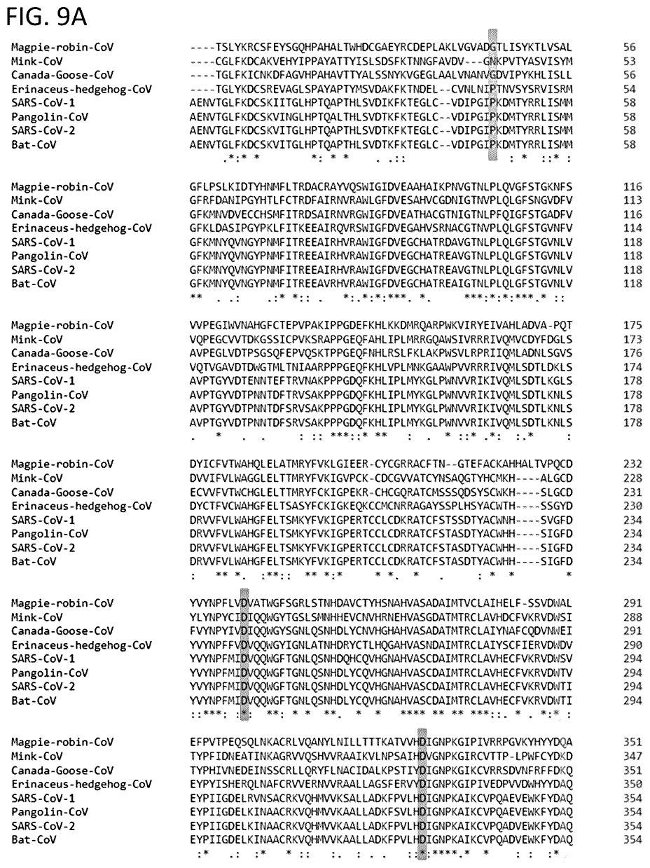

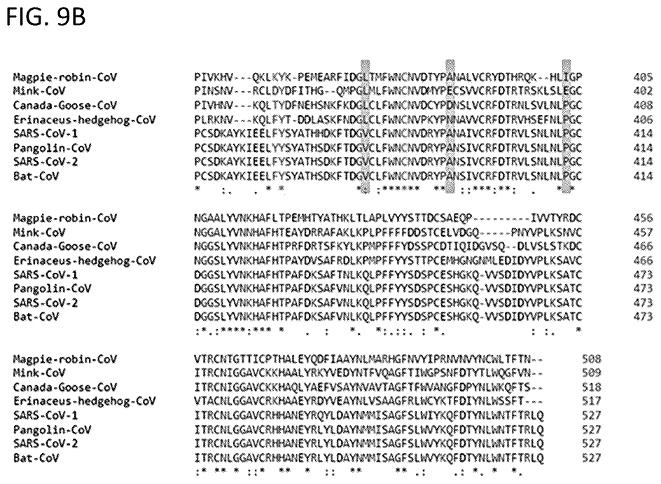

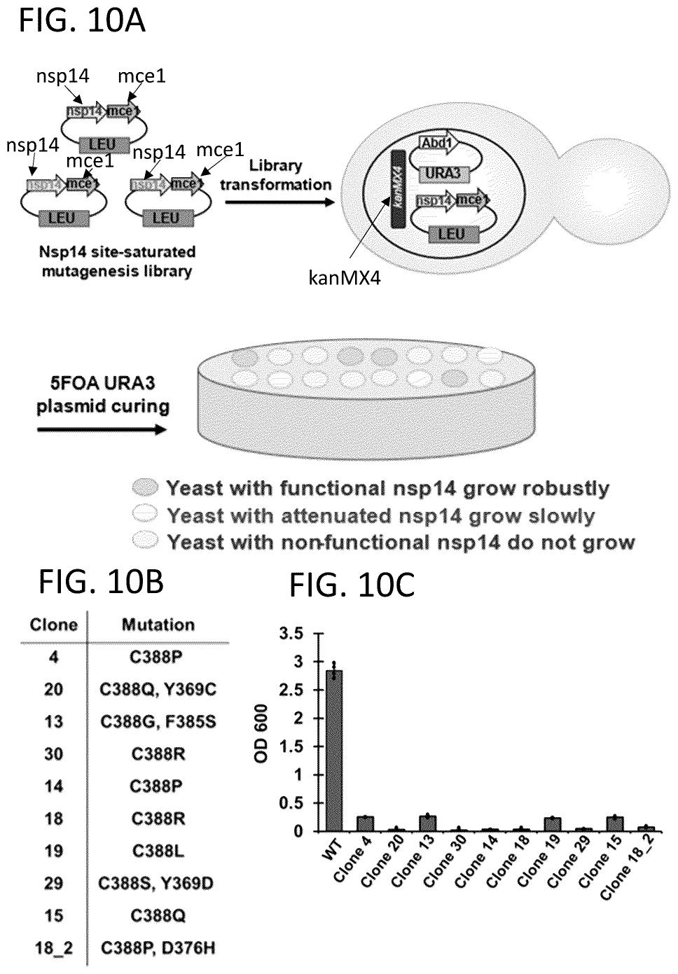

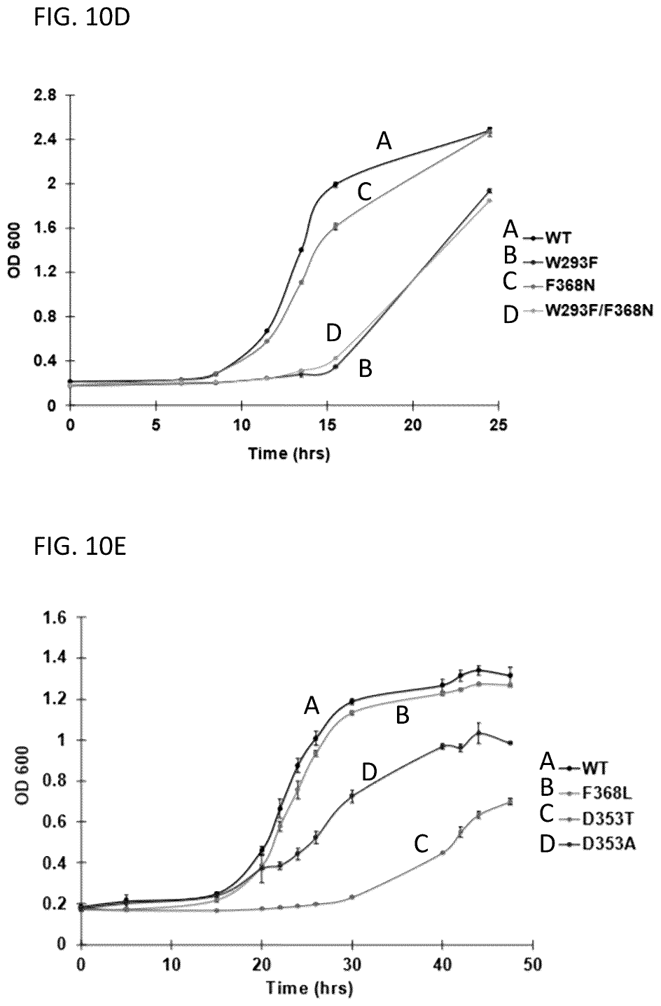

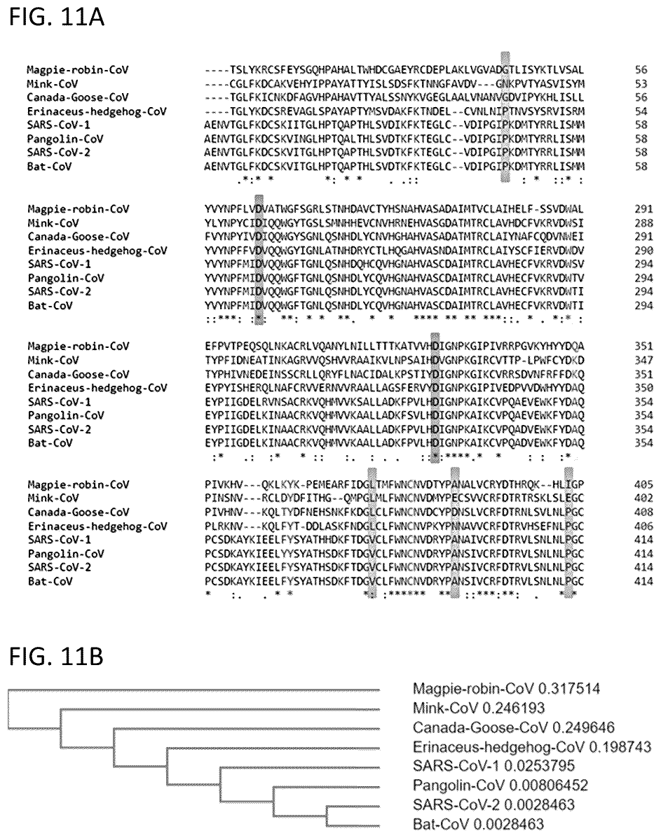

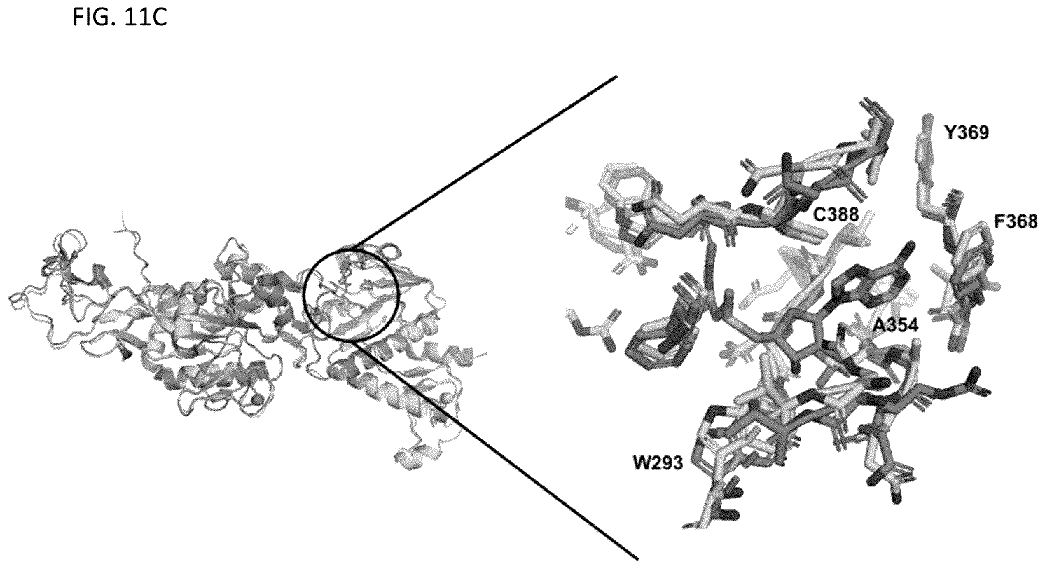

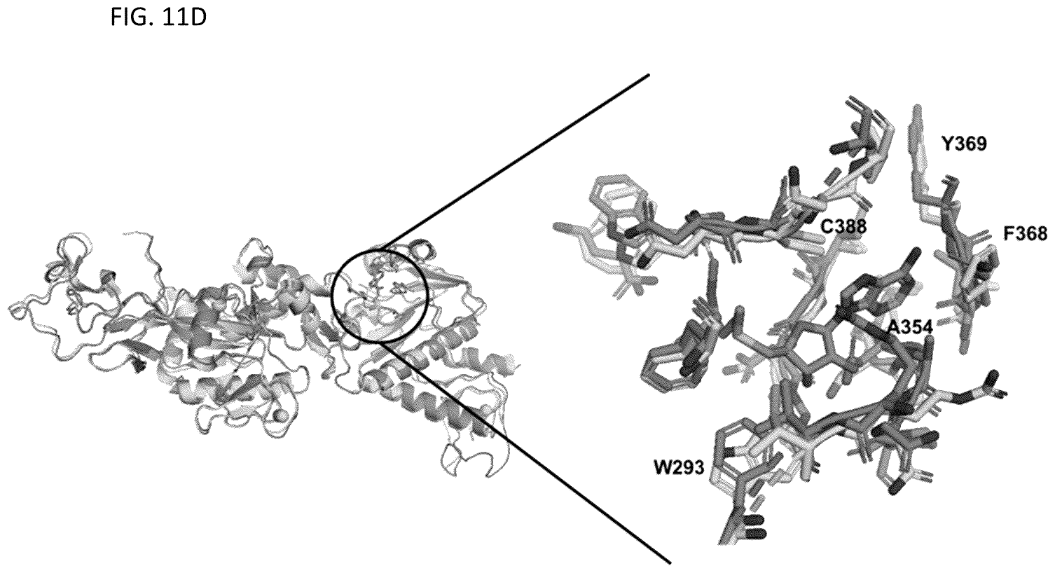

Essential viral enzymes are often targeted to develop attenuated strains of viruses as potential live attenuated vaccine strains.^{21,22} Inactivation and attenuation mutations in SARS-CoV-2 nsp14 were identified. Based on the available crystal structure^{54,55}, literature precedence⁴⁸, and nsp14 sequence analysis from diverse coronaviruses (FIG. 9)⁵⁷, proof-of-concept experiments were performed to demonstrate the importance of D331 residue nsp14 to N7-MTase activity.^{54,55} Plasmid pMO4, derived from pMO2, was constructed, replacing residue D331 with alanine. pMO4 was transformed into S. cerevisiae abd1::kanMX4 pMO4 haploid and it was observed that the corresponding strain was unable to survive in presence of 5-FOA indicating that this point mutation resulted in a functionally inactive variant of nsp14. Since S-adenosylmethionine (AdoMet) is a necessary coenzyme for N7-MTase activity, the AdoMet binding pocket was targeted to identify variants of nsp14 that could have attenuated activity. Because this approach was compatible with high-throughput phenotypic screening, site-saturated mutagenesis/screening approach was used to determine inactivating mutations in SARS-CoV-2 nsp14. Such identification would facilitate precise understanding of the functional relevance of highly conserved (FIG. 9) non-catalytic residues to the activity of nsp14. To this end, the YeRCOM-nsp14 platform for directed evolution of SARS-CoV-2 nsp14 was used.⁵⁸ First, a randomized site saturation mutagenesis library at residues A354, C388, and Y369 was generated. Then, this library was transformed into S. cerevisiae abd1::kanMX4 pMO1 haploid and through replica plating in the presence and absence of 5-FOA, residue changes in the AdoMet binding pocket that result in inactivation of nsp14 were identified (FIG. 10B). Notably, every inactivated mutant identified had position C388 mutated (FIG. 10B). The list of inactivating mutations identified using this approach are listed in FIG. 10B. The next goal was to identify attenuated variants of SARS-CoV-2 nsp14. To identify attenuated variants of nsp14, a randomized site saturation mutagenesis library at residues W293 and F368, in the AdoMet binding pocket was constructed. Sequence alignments suggested that while W293 is highly conserved residue (FIGS. 9, 11A, and 11B), F368 is not as highly conserved. S. cerevisiae abd1::kanMX4 pMO1 haploid strains were transformed with W293X-F368X nsp14 library and ˜130 colonies were screened through replica plating in the presence and absence of 5-FOA. Through this screening an attenuated mutant of nsp14 was identified (FIG. 10D) which resulted in significantly slower growth of S. cerevisiae abd1::kanMX4 haploid strains in presence of complementation plasmid expressing nsp14 library variant. This nsp14 variant was sequenced, and it was observed that this variant had the following changes: W293F and F368N. In order to elucidate the relevance of each of individual alterations, plasmids expressing nsp14-W293F (pMO27) and nsp14-F368N (pMO28) were generated and transformed individually into S. cerevisiae abd1::kanMX4 haploid strains. The growth of the corresponding strains was monitored over 48 hours (FIG. 10D) in presence of 5-FOA. It was observed that W293F was responsible for most of the attenuated activity (FIG. 10D). Upon further screening of the W293X/F368X and D353X libraries, several additional attenuation mutations of nsp14 were identified: F368L, D353T, and D353A (FIG. 10E).

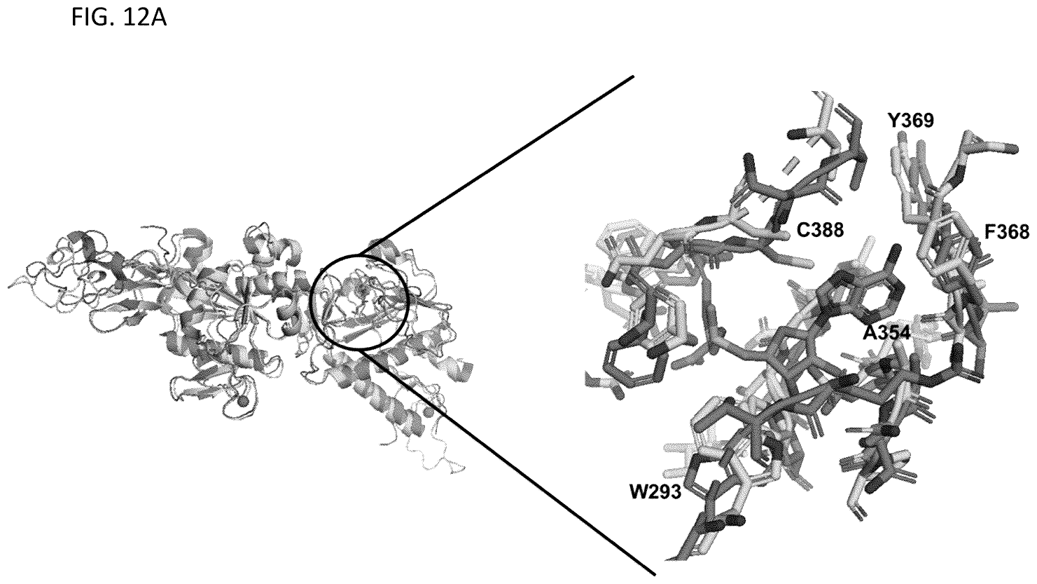

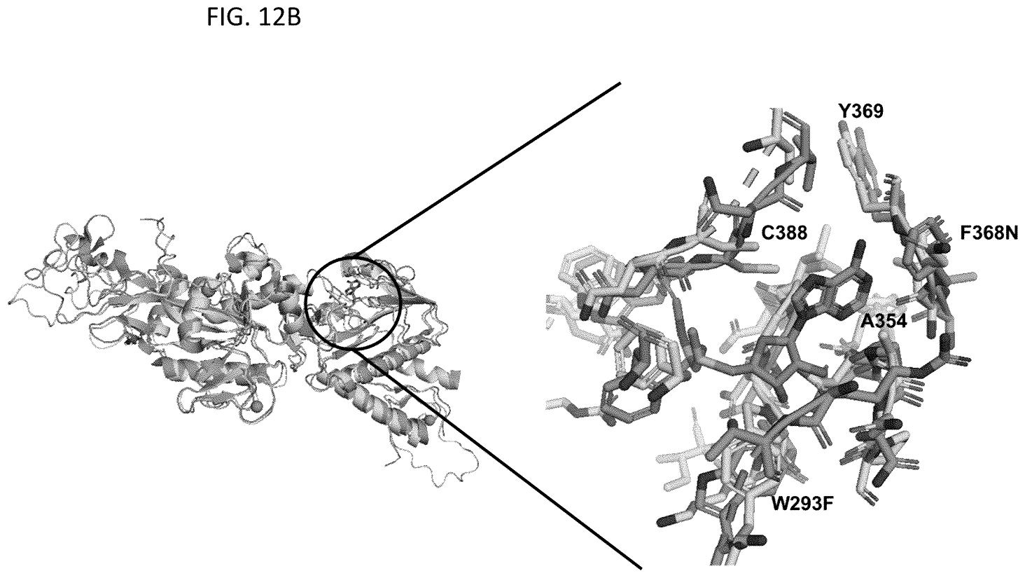

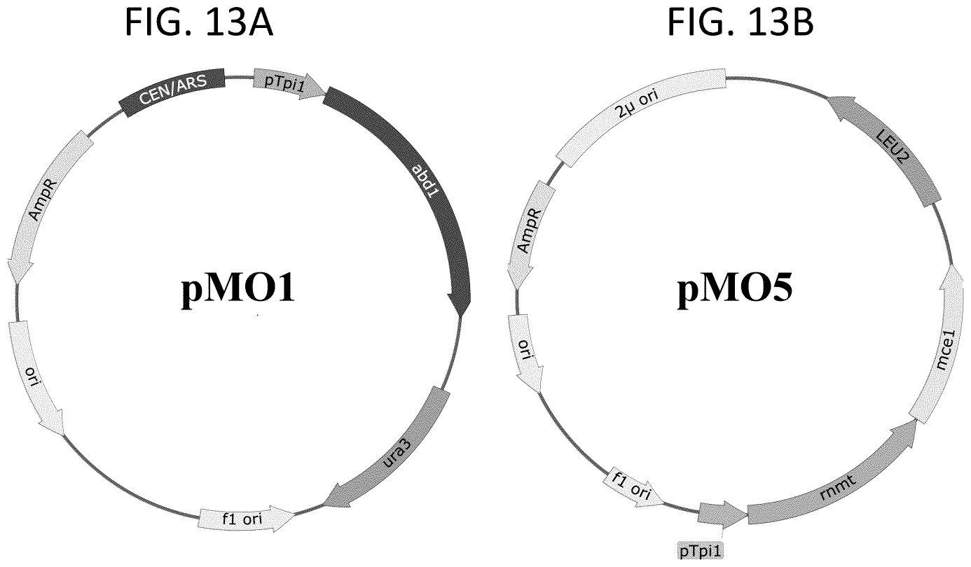

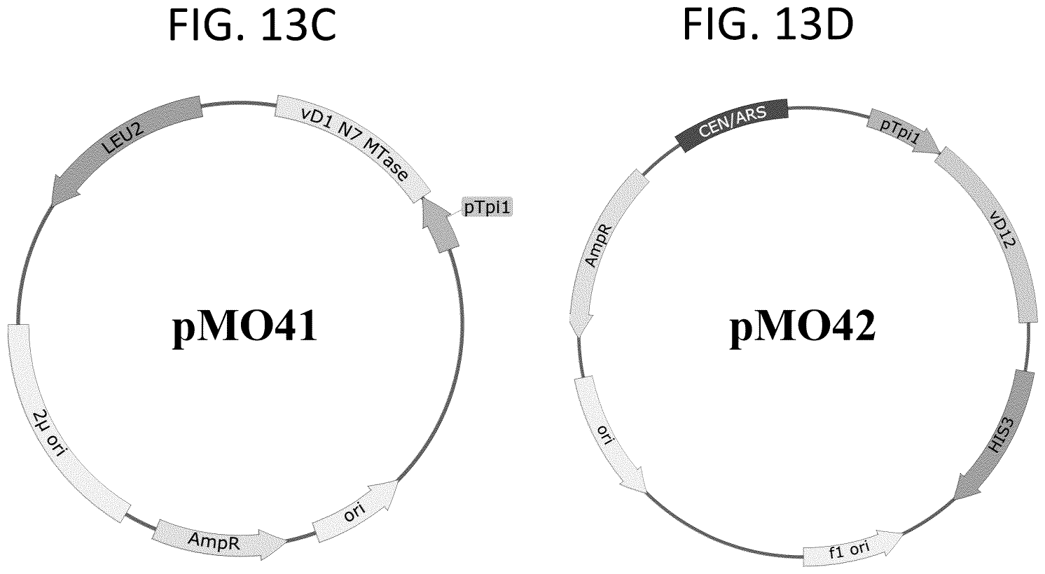

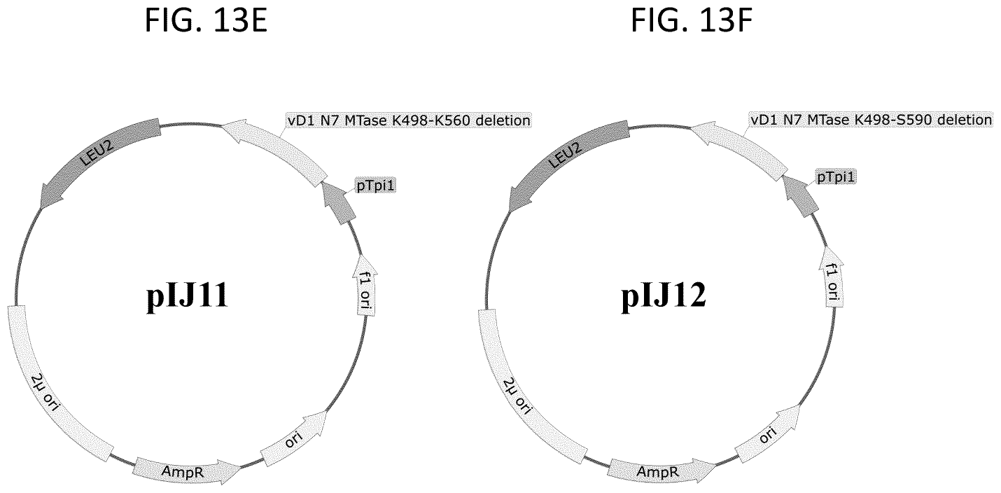

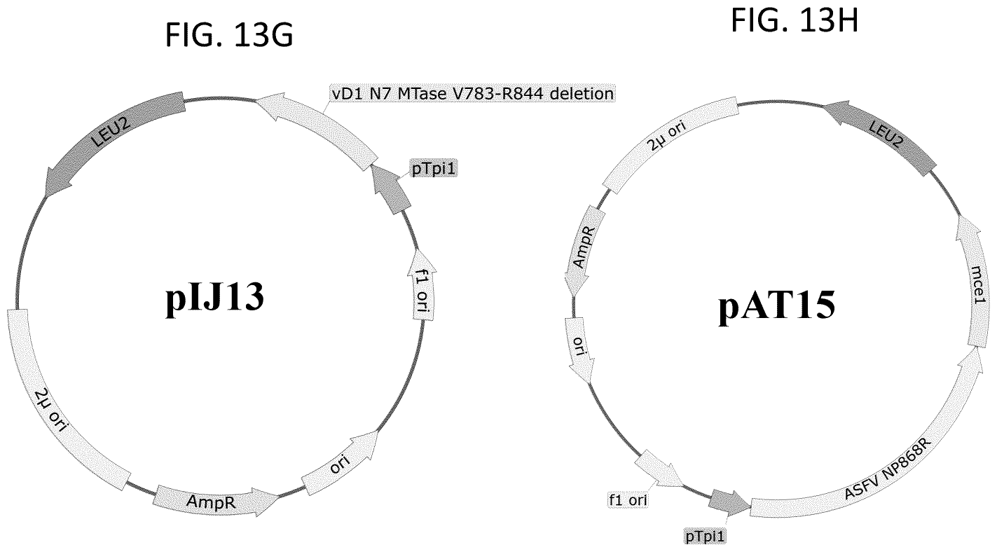

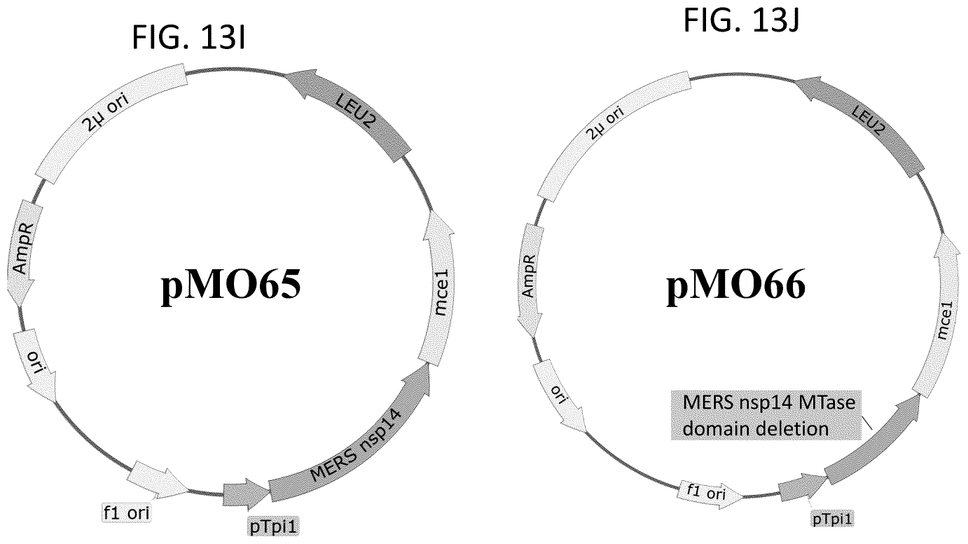

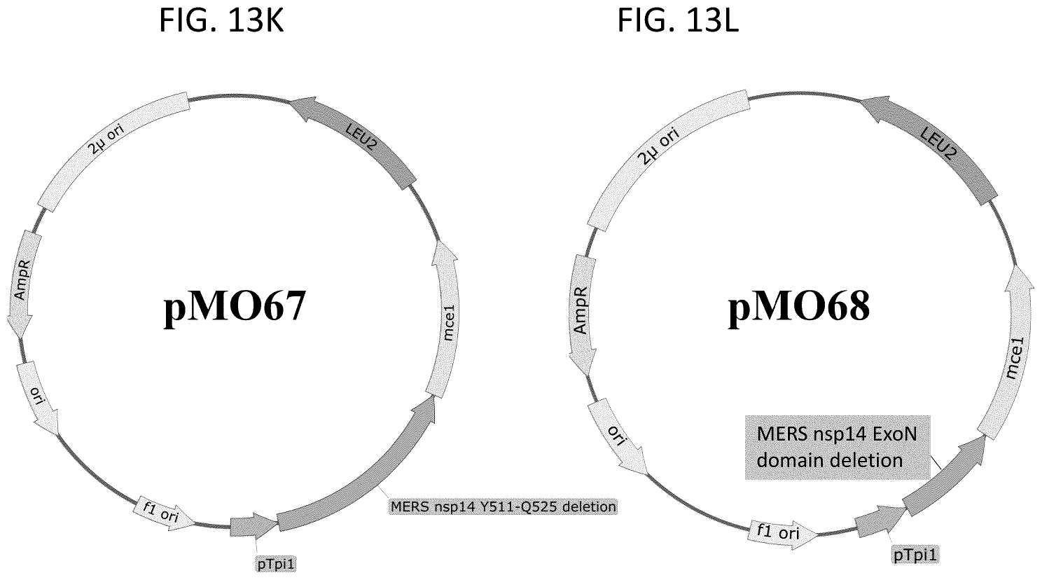

In order the understand the biochemical basis of the identified attenuation double mutant, SWISS-MODEL⁵⁹ (FIGS. 11C, 11D) and AlphaFold⁶⁰ (FIG. 12) were used to predict the structure of both WT nsp14 and attenuated nsp14 in order to compare it to the crystal structure of nsp14 (PDB 5C8T). W293F opens up the AdoMet binding region the active site which may have alter the binding of coenzyme AdoMet to nsp14. Both models predict a slight shift in the positions of C388 and Y369 further opening up the AdoMet binding pocket in nsp14.

### REFERENCES FOR EXAMPLE 1 THROUGH EXAMPLE 6

(21) Lauring, A. S.; Jones, J. O.; Andino, R. Rationalizing the Development of Live Attenuated Virus Vaccines. Nat Biotechnol 2010, 28 (6), 573-579.
(22) Yeh, M. T.; Bujaki, E.; Dolan, P. T.; Smith, M.; Wahid, R.; Konz, J.; Weiner, A. J.; Bandyopadhyay, A. S.; Van Damme, P.; De Coster, I.; Revets, H.; Macadam, A.; Andino, R. Engineering the Live-Attenuated Polio Vaccine to Prevent Reversion to Virulence. Cell Host & Microbe 2020, 27 (5), 736-751.e8.
(24) Eckerle, L. D.; Becker, M. M.; Halpin, R. A.; Li, K.; Venter, E.; Lu, X.; Scherbakova, S.; Graham, R. L.; Baric, R. S.; Stockwell, T. B. Infidelity of SARS-CoV Nsp14-Exonuclease Mutant Virus Replication Is Revealed by Complete Genome Sequencing. PLoS pathogens 2010, 6 (5).
(30) Sawicki, S. G. Coronavirus Genome Replication. Viral Genome Replication 2008, 25-39.
(42) Otava, T.; Sila, M.; Li, F.; Fanfrlik, J.; Devkota, K.; Perveen, S.; Chau, I.; Pakarian, P.; Hobza, P.; Vedadi, M.; Boura, E.; Nencka, R. The Structure-Based Design of SARS-CoV-2 Nsp14 Methyltransferase Ligands Yields Nanomolar Inhibitors. ACS Infect. Dis. 2021, 7 (8), 2214-2220.
(43) Devkota, K.; Schapira, M.; Perveen, S.; Khalili Yazdi, A.; Li, F.; Chau, I.; Ghiabi, P.; Hajian, T.; Loppnau, P.; Bolotokova, A.; Satchell, K. J. F.; Wang, K.; Li, D.; Liu, J.; Smil, D.; Luo, M.; Jin, J.; Fish, P. V.; Brown, P. J.; Vedadi, M. Probing the SAM Binding Site of SARS-CoV-2 Nsp14 In Vitro Using SAM Competitive Inhibitors Guides Developing Selective Bisubstrate Inhibitors. SLAS DISCOVERY: Advancing the Science of Drug Discovery 2021, 26 (9), 1200-1211.
(44) Ferreira, R.; Limeta, A.; Nielsen, J. Tackling Cancer with Yeast-Based Technologies. Trends in Biotechnology 2019, 37 (6), 592-603. https://doi.org/10.1016/j.tibtech.2018.11.013.
(45) Simon, J. A.; Szankasi, P.; Nguyen, D. K.; Ludlow, C.; Dunstan, H. M.; Roberts, C. J.; Jensen, E. L.; Hartwell, L. H.; Friend, S. H. Differential Toxicities of Anticancer Agents among DNA Repair and Checkpoint Mutants of Saccharomyces Cerevisiae. Cancer Research 2000, 60 (2), 328-333.
(46) Alves-Rodrigues, I.; Galão, R. P.; Meyerhans, A.; Diez, J. Saccharomyces Cerevisiae: A Useful Model Host to Study Fundamental Biology of Viral Replication. Virus Res 2006, 120 (1), 49-56.
(47) Saha, N.; Shuman, S.; Schwer, B. Yeast-Based Genetic System for Functional Analysis of Poxvirus MRNA Cap Methyltransferase. Journal of Virology 2003, 77 (13), 7300-7307.
(48) Chen, Y.; Cai, H.; Xiang, N.; Tien, P.; Ahola, T.; Guo, D. Functional Screen Reveals SARS Coronavirus Nonstructural Protein Nsp14 as a Novel Cap N7 Methyltransferase. Proceedings of the National Academy of Sciences 2009, 106 (9), 3484-3489.
(49) Alalam, H.; Sigurdardóttir, S.; Bourgard, C.; Tiukova, I.; King, R. D.; Gretli, M.; Sunnerhagen, P. A Genetic Trap in Yeast for Inhibitors of SARS-CoV-2 Main Protease. mSystems 6 (6), e01087-21.
(50) Cottier, V.; Barberis, A.; Luthi, U. Novel Yeast Cell-Based Assay to Screen for Inhibitors of Human Cytomegalovirus Protease in a High-Throughput Format. Antimicrob Agents Chemother 2006, 50 (2), 565-571.
(51) Mao, X.; Schwer, B.; Shuman, S. Yeast MRNA Cap Methyltransferase Is a 50-Kilodalton Protein Encoded by an Essential Gene. Molecular and Cellular Biology 1995, 15 (8), 4167-4174.
(52) Winzeler, E. A.; Shoemaker, D. D.; Astromoff, A.; Liang, H.; Anderson, K.; Andre, B.; Bangham, R.; Benito, R.; Boeke, J. D.; Bussey, H.; Chu, A. M.; Connelly, C.; Davis, K.; Dietrich, F.; Dow, S. W.; Bakkoury, M. E.; Foury, F.; Friend, S. H.; Gentalen, E.; Giaever, G.; Hegemann, J. H.; Jones, T.; Laub, M.; Liao, H.; Liebundguth, N.; Lockhart, D. J.; Lucau-Danila, A.; Lussier, M.; M'Rabet, N.; Menard, P.; Mittmann, M.; Pai, C.; Rebischung, C.; Revuelta, J. L.; Riles, L.; Roberts, C. J.; Ross-MacDonald, P.; Scherens, B.; Snyder, M.; Sookhai-Mahadeo, S.; Storms, R. K.; Véronneau, S.; Voet, M.; Volckaert, G.; Ward, T. R.; Wysocki, R.; Yen, G. S.; Yu, K.; Zimmermann, K.; Philippsen, P.; Johnston, M.; Davis, R. W. Functional Characterization of the S. Cerevisiae Genome by Gene Deletion and Parallel Analysis. Science 1999, 285 (5429), 901-906.
(53) Boeke, J. D.; Trueheart, J.; Natsoulis, G.; Fink, G. R. 5-Fluoroorotic Acid as a Selective Agent in Yeast Molecular Genetics. Methods Enzymol 1987, 154, 164-175.
(54) Lin, S.; Chen, H.; Chen, Z.; Yang, F.; Ye, F.; Zheng, Y.; Yang, J.; Lin, X.; Sun, H.; Wang, L.; Wen, A.; Dong, H.; Xiao, Q.; Deng, D.; Cao, Y.; Lu, G. Crystal Structure of SARS-CoV-2 Nsp10 Bound to Nsp14-ExoN Domain Reveals an Exoribonuclease with Both Structural and Functional Integrity. Nucleic Acids Research 2021, 49 (9), 5382-5392.
(55) Krafcikova, P.; Silhan, J.; Nencka, R.; Boura, E. Structural Analysis of the SARS-CoV-2 Methyltransferase Complex Involved in RNA Cap Creation Bound to Sinefungin. Nat Commun 2020, 11 (1), 3717.
(56) Saramago, M.; Bárria, C.; Costa, V. G.; Souza, C. S.; Viegas, S. C.; Domingues, S.; Lousa, D.; Soares, C. M.; Arraiano, C. M.; Matos, R. G. New Targets for Drug Design: Importance of Nsp14/Nsp10 Complex Formation for the 3′-5′ Exoribonucleolytic Activity on SARS-CoV-2. The FEBS Journal 2021, 288 (17), 5130-5147.
(57) Madeira, F.; Park, Y. M.; Lee, J.; Buso, N.; Gur, T.; Madhusoodanan, N.; Basutkar, P.; Tivey, A. R.; Potter, S. C.; Finn, R. D. The EMBL-EBI Search and Sequence Analysis Tools APIs in 2019. Nucleic acids research 2019, 47 (W1), W636-W641.
(58) Romero, P. A.; Arnold, F. H. Exploring Protein Fitness Landscapes by Directed Evolution. Nature Reviews Molecular Cell Biology 2009, 10 (12), 866-876.
(59) Waterhouse, A.; Bertoni, M.; Bienert, S.; Studer, G.; Tauriello, G.; Gumienny, R.; Heer, F. T.; de Beer, T. A. P.; Rempfer, C.; Bordoli, L. SWISS-MODEL: Homology Modelling of Protein Structures and Complexes. Nucleic acids research 2018, 46 (WI), W296-W303.
(60) Jumper, J.; Evans, R.; Pritzel, A.; Green, T.; Figurnov, M.; Ronneberger, O.; Tunyasuvunakool, K.; Bates, R.; Z̆idek, A.; Potapenko, A. Highly Accurate Protein Structure Prediction with AlphaFold. Nature 2021, 596 (7873), 583-589.
(64) Paulissen, S. M.; Huang, L. S. Efficient Sporulation of Saccharomyces Cerevisiae in a 96 Multiwell Format. JoVE (Journal of Visualized Experiments) 2016, No. 115, e54584.
(65) Gibson, D. G.; Young, L.; Chuang, R.-Y.; Venter, J. C.; Hutchison, C. A.; Smith, H. O. Enzymatic Assembly of DNA Molecules up to Several Hundred Kilobases. Nat Methods 2009, 6 (5), 343-345.

### Example 7

### Material and Methods for Results in Example 7 Through Example 12

This example illustrates the methods used for Example 7 to Example 12.

Growth media conditions: All Saccharomyces cerevisiae cultures were shaken aerobically at 30° C. and 250 rpm in synthetic defined medium containing 0.67% nitrogen base without amino acids, 2% glucose, 0.02 mg/mL lysine, 0.02 mg/mL methionine, 0.02 mg/mL histidine, and 0.1 mg/ml carbenicillin. Leucine 0.012 mg/mL was added when S. cerevisiae Δabd1 pMO1 haploid was cultured. Histidine was not included in the media for strains expressing vD12 from the pRS413-tpi1-vD12 plasmid.

Plasmid curing procedure: Plasmids containing ura3 marker were cured by growing cultures in synthetic defined medium containing 1 mg/mL 5-fluoroorotic acid (5-FOA) and uracil 0.02 mg/mL for 48 hours or by plating onto synthetic media agar plates containing 1 mg/mL 5-fluoroorotic acid (5-FOA) and uracil 0.02 mg/mL and incubating at 30° C. for 48 hours.

Disulfiram Screening conditions: Saccharomyces cerevisiae cured cultures were grown in synthetic defined medium containing uracil in the presence of 0, 25, and 50 uM disulfiram for 48 hours. Disulfiram was dissolved in DMSO (final DMSO concentrations in all cultures was 1%).

Construction of plasmids: Plasmid maps are included in FIG. 12. Plasmid map Benchling links are provided in Table 7. All single-stranded and double-stranded DNA oligonucleotide fragments were purchased from Integrated DNA Technologies (IDT). Single-stranded oligonucleotides sequences are listed in Table 8. Double-stranded oligonucleotide sequences (gblocks) used for Gibson assembly are listed in Table 9. Genomic DNA fragments used in the construction of plasmids are listed in Table 10. When noted, coding sequences were codon-optimized for S. cerevisiae expression using IDT codon optimization software.

pMO1: The promoter sequence, pTPI1, was amplified from the gDNA of S. cerevisiae YPH500 using the oligonucleotides M041/MO42. The gene corresponding to ABD1 was amplified from the gDNA of S. cerevisiae YPH500 using the oligonucleotides M045/MO46. pRS416 ura3 was linearized using the oligonucleotides M044/MO43. pTPI1 and abd1 gene fragments were inserted into linearized pRS416 ura3 by Gibson assembly⁴⁵ to afford pMO1.

pMO65: double-stranded MERS nsp14 gBlock codon optimized for S. cerevisiae was purchased from Integrated DNA Technologies (IDT). MERS nsp14 gBlock was amplified using oligonucleotides ROS6/ROS5. pRS425-pTPI1-mce1 was linearized using the oligonucleotides ROS7/ROS8. MERS nsp14 fragment was inserted into linearized pRS425-pTPI1-mce1 by Gibson assembly to afford pMO65.

pMO66: MERS nsp14 ExoN domain was amplified using oligonucleotides ROS10/ROS5. pMO65 was linearized using oligonucleotides ROS8/ROS7. MERS nsp14 ExoN domain fragment was inserted into linearized pMO65 by Gibson assembly to afford pMO66.

pMO67: MERS nsp14 was amplified using oligonucleotides ROS5/ROS9 to exclude the C-terminal 17 amino acids. pMO65 was linearized using oligonucleotides ROS8/ROS7. MERS nsp14 fragment was inserted into linearized pMO65 by Gibson assembly to afford pMO67.

pMO68: MERS nsp14 MTase domain was amplified using oligonucleotides ROS6/ROS11. pMO65 was linearized using oligonucleotides ROS8/ROS7. MERS nsp14 MTase domain fragment was inserted into linearized pMO65 by Gibson assembly to afford pMO68.

pMO70: point mutation D243A was introduced into pMO65 by amplifying nsp14 fragment using ROS14/ROS5. pMO65 was linearized using oligonucleotides ROS15/ROS7. MERS nsp14 fragment was inserted into linearized pMO65 by Gibson assembly to afford pMO70.

pMO69: point mutation D331A was introduced into pMO65 by amplifying nsp14 fragment using ROS12/ROS5. pMO65 was linearized using oligonucleotides ROS13/ROS7. MERS nsp14 fragment was inserted into linearized pMO65 by Gibson assembly to afford pMO69.

pIJ31: point mutation F365L was introduced into pMO65 by linearizing pMO65 using oligonucleotides M0399/M0376. The linearized fragment was ligated by KLD reaction to afford pIJ31.

pIJ19: point mutation F365G was introduced into pMO65 by linearizing pMO65 using oligonucleotides M0371/M0376. The linearized fragment was ligated by KLD reaction to afford pIJ19.

pIJ29: point mutation F365D was introduced into pMO65 by linearizing pMO65 using oligonucleotides M0400/M0376. The linearized fragment was ligated by KLD reaction to afford pIJ29.

pIJ30: point mutation F365N was introduced into pMO65 by linearizing pMO65 using oligonucleotides M0398/M0376. The linearized fragment was ligated by KLD reaction to afford pIJ30.

pIJ17: point mutation W293F was introduced into pMO65 by linearizing pMO65 using oligonucleotides M0372/M0377. The linearized fragment was ligated by KLD reaction to afford pIJ17.

pIJ18: point mutation D353A was introduced into pMO65 by linearizing pMO65 using oligonucleotides M0373/M0375. The linearized fragment was ligated by KLD reaction to afford pIJ18.

pIJ16: point mutation D353T was introduced into pMO65 by linearizing pMO65 using oligonucleotides M0374/M0375. The linearized fragment was ligated by KLD reaction to afford pIJ16.

pMO41: double-stranded MPV vD1 (aa 498-844) gBlock codon optimized for S. cerevisiae was purchased from Integrated DNA Technologies (IDT). MPV vD1 (aa 498-844) gBlock was amplified using oligonucleotides M0324/M0322. pRS425-tpi1 backbone was linearized using the oligonucleotides M0321/M0323. MPV vD1 (aa 498-844) fragment was inserted into linearized pRS425-tpil backbone by Gibson assembly to afford pMO41.

pMO42: double-stranded MPV vD12 gBlock codon optimized for S. cerevisiae was purchased from Integrated DNA Technologies (IDT). MPV vD12 gBlock was amplified using oligonucleotides M0318/M0320. pRS413-tpi1 backbone was linearized using the oligonucleotides M0317/M0319. MPV vD12 fragment was inserted into linearized pRS413-tpi1 backbone by Gibson assembly to afford pMO42.

pMO54: point mutation D620A was introduced into pMO41 by linearizing pMO41 using oligonucleotides M0362/M0363. The linearized fragment was ligated by KLD reaction to afford pMO54.

pMO55: point mutation R655A was introduced into pMO41 by linearizing pMO41 using oligonucleotides M0364/M0365. The linearized fragment was ligated by KLD reaction to afford pMO55.

pMO48: point mutation Y555F was introduced into pMO41 by linearizing pMO41 using oligonucleotides M0359/M0357. The linearized fragment was ligated by KLD reaction to afford pMO48.

pMO47: point mutation Y555A was introduced into pMO41 by linearizing pMO41 using oligonucleotides M0358/M0357. The linearized fragment was ligated by KLD reaction to afford pMO47.

pMO50: point mutation D598A was introduced into pMO41 by linearizing pMO41 using oligonucleotides M0360/M0361. The linearized fragment was ligated by KLD reaction to afford pMO50.

pMO56: point mutation Y683A was introduced into pMO41 by linearizing pMO41 using oligonucleotides M0366/M0367. The linearized fragment was ligated by KLD reaction to afford pMO56.

pMO53: point mutation D545A was introduced into pMO41 by linearizing pMO41 using oligonucleotides M0353/M0354. The linearized fragment was ligated by KLD reaction to afford pMO53.

pMO49: point mutation F556A was introduced into pMO41 by linearizing pMO41 using oligonucleotides M0356/M0357. The linearized fragment was ligated by KLD reaction to afford pMO49.

pIJI1: residues K498-K560 were deleted from MPV vD1 (aa 498-844) by linearizing pMO41 using oligonucleotides M0351/M0349. The linearized fragment was ligated by KLD reaction to afford pIJ11.

pIJ12: residues K498-S590 were deleted from MPV vD1 (aa 498-844) by linearizing pMO41 using oligonucleotides M0368/M0349. The linearized fragment was ligated by KLD reaction to afford pIJ12.

pIJ13: residues V783-R844 were deleted from MPV vD1 (aa 498-844) by linearizing pMO41 using oligonucleotides M0264/M0352. The linearized fragment was ligated by KLD reaction to afford pIJ13.

MPV Y683X library construction: a library at position Y683 was made by amplifying pMO41 with oligonucleotides M0329/M0322 to create a library insert. The insert was ligated to a linearized backbone fragment that was made my amplifying pMO41 with oligonucleotides M0330/MO321. The insert and backbone fragments were ligated using Gibson assembly to afford the library.

MPV R548X library construction: a library at position R548 was made by amplifying pMO41 with oligonucleotides M0327/M0324 to create a library insert. The insert was ligated to a linearized backbone fragment that was made my amplifying pMO41 with oligonucleotides M0328/MO323. The insert and backbone fragments were ligated using Gibson assembly to afford the library.

pMO5: double-stranded RNMT gBlock codon optimized for S. cerevisiae was purchased from Integrated DNA Technologies (IDT). pMO2 was linearized to exclude nsp14 using oligonucleotides M0106/M096. RNMT gBlock was amplified using oligonucleotides M0105/M0113 and inserted into the linearized fragment by Gibson assembly to afford pMO5.

pAT15: double-stranded ASFV NP868R gBlock codon optimized for S. cerevisiae was purchased from Integrated DNA Technologies (IDT). ASFV NP868R gBlock was amplified using oligonucleotides ROS33/ROS34. pMO3 was linearized using the oligonucleotides ROS7/ROS8. ASFV NP868R fragment was inserted into linearized pRS425-pTPI1-mce1 by Gibson assembly to afford pAT15.

pIJ22: point mutation Y714A was introduced into ASFV NP868R by linearizing pAT15 using oligonucleotides M0392/M0394. The linearized fragment was ligated by KLD reaction to afford pIJ22.

pIJ23: point mutation Y714L was introduced into ASFV NP868R by linearizing pAT15 using oligonucleotides M0393/M0394. The linearized fragment was ligated by KLD reaction to afford pIJ23.

pIJ24: point mutation F711A was introduced into ASFV NP868R by linearizing pAT15 using oligonucleotides M0389/M0391. The linearized fragment was ligated by KLD reaction to afford pIJ24.

pIJ25: point mutation F71IL was introduced into ASFV NP868R by linearizing pAT15 using oligonucleotides M0390/M0391. The linearized fragment was ligated by KLD reaction to afford pIJ25.

pIJ26: point mutation F711W was introduced into ASFV NP868R by linearizing pAT15 using oligonucleotides M0395/M0391. The linearized fragment was ligated by KLD reaction to afford pIJ26.

pIJ27: point mutation D680L was introduced into ASFV NP868R by linearizing pAT15 using oligonucleotides M0396/M0397. The linearized fragment was ligated by KLD reaction to afford pIJ27.

pMO64: point mutation K647Y was introduced into ASFV NP868R by linearizing pAT15 using oligonucleotides AT139/AT140. The linearized fragment was ligated by KLD reaction to afford pMO64.

pAT19: point mutation S604A was introduced into ASFV NP868R by linearizing pAT15 using oligonucleotides AT124/AT125. The linearized fragment was ligated by KLD reaction to afford pAT19.

pAT20: point mutation D646A was introduced into ASFV NP868R by linearizing pAT15 using oligonucleotides AT141/AT140. The linearized fragment was ligated by KLD reaction to afford pAT20.

pAT21: point mutation D646E was introduced into ASFV NP868R by linearizing pAT15 using oligonucleotides AT142/AT140. The linearized fragment was ligated by KLD reaction to afford pAT21.

pAT22: point mutation D680N was introduced into ASFV NP868R by linearizing pAT15 using oligonucleotides AT137/AT136 The linearized fragment was ligated by KLD reaction to afford pAT22.

pAT23: point mutation D680A was introduced into ASFV NP868R by linearizing pAT15 using oligonucleotides AT135/AT136 The linearized fragment was ligated by KLD reaction to afford pAT23.

pAT24: point mutation D680E was introduced into ASFV NP868R by linearizing pAT15 using oligonucleotides AT138/AT136 The linearized fragment was ligated by KLD reaction to afford pAT24.

ASFV F711X library construction: a library at position F711 was made by amplifying pAT15 with oligonucleotides AT113/ROS36 to create a library insert. The insert was ligated to a linearized backbone fragment that was made my amplifying pAT15 with oligonucleotides AT114/ROS35. The insert and backbone fragments were ligated using Gibson assembly to afford the library.

ASFV Y714X library construction: a library at position Y714 was made by amplifying pAT15 with oligonucleotides AT112/ROS36 to create a library insert. The insert was ligated to a linearized backbone fragment that was made my amplifying pAT15 with oligonucleotides AT111/ROS35. The insert and backbone fragments were ligated using Gibson assembly to afford the library.

TABLE 7
Correspondence between plasmids and sequence numbering.

| Plasmid | SEQ ID NO |
|---|---|
| pMO1 | SEQ ID NO: 3 |
| pMO5 | SEQ ID NO: 21 |
| pMO41 | SEQ ID NO: 17 |
| pMO42 | SEQ ID NO: 18 |
| pIJ11 | SEQ ID NO: 135 |
| pIJ12 | SEQ ID NO: 136 |
| pIJ13 | SEQ ID NO: 137 |
| pAT15 | SEQ ID NO: 19 |
| pMO65 | SEQ ID NO: 22 |
| pMO66 | SEQ ID NO: 138 |
| pMO67 | SEQ ID NO: 139 |
| pMO68 | SEQ ID NO: 140 |

TABLE 8
DNA oligos used in the construction of plasmids.

| | SEQ | |
|---|---|---|
| | ID | |
| Name | NO | Sequence |
| MO41 | 141 | ACAGCTTGTCACACCCCTTTTCTGGCA |
| MO42 | 142 | TGGTTGACATTTTTAGTTTATGTATGTGTTTTTTGTAGTTATAG |
| MO45 | 143 | TAAACTAAAAATGTCAACCAAACCAGAGAAGCCAATATG |
| MO46 | 144 | TCCGCTTACATCAGTTGGGCTTTACGCTTTCC |
| MO44 | 145 | GCCCAACTGATGTAAGCGGATGCCGGG |
| MO43 | 146 | AAAGGGGTGTGACAAGCTGTGACCGTCTCC |
| MO106 | 147 | GAAGCAACAGGAACCAGGGTCAAGTGC |
| MO96 | 148 | AGTTGGCCATTTTTAGTTTATGTATGTGTTTTTTGTAGTTATAGA |
| MO105 | 149 | ACCCTGGTTCCTGTTGCTTCTCAAAGGC |
| MO113 | 150 | TAAACTAAAAATGGCCAACTCCGC |
| ROS5 | 151 | ACTACAAAAAACACATACATAAACTAAAAATGTCCCAAATTGTCACTGG |
| ROS6 | 152 | GATGCACTTGACCCTGGTTCCTGAACTTTTGTAAACGTACTCCAA |
| ROS7 | 153 | TTTTAGTTTATGTATGTGTTTTTTGTAGT |
| ROS8 | 154 | GAACCAGGGTCAAGTGCATC |
| ROS9 | 155 | GATGCACTTGACCCTGGTTCGCACCACAGACGGAATCC |
| ROS10 | 156 | GATGCACTTGACCCTGGTTCCACCCTTTCTATGAAGCAAGAATGG |
| ROS11 | 157 | ACTACAAAAAACACATACATAAACTAAAAATGGACTGGGATATTGAATACCCGTAC |
| ROS14 | 158 | CACTGCTGTACCGCTACAAAGAAT |
| ROS15 | 159 | ATTCTTTGTAGCGGTACAGCAGTG |
| ROS12 | 160 | AGGGTTCCCTATCGCATACACTTTAT |
| ROS13 | 161 | ATAAAGTGTATGCGATAGGGAACCCT |
| ROS33 | 162 | GATGCACTTGACCCTGGTTCATTCTTCCTTAAGCATATAAACCCGA |
| ROS34 | 163 | ACTACAAAAAACACATACATAAACTAAAAATGGCTAGTCTTGAGAATCTGGT |
| MO321 | 164 | GCTATTAATGTATTTCATTTTTAGTTTATGTATGTGTTTTTTGTAGTTATAGA |
| MO322 | 165 | CACATACATAAACTAAAAATGAAATACATTAATAGCGAAGATTATTACGG |
| MO323 | 166 | TATTCAGCAAGAGGTGATTCAAGAAATATCTTGACCGCAG |
| MO324 | 167 | TATTTCTTGAATCACCTCTTGCTGAATACGTAAACTACA |
| MO317 | 168 | TGACAATCTCATCCATTTTTAGTTTATGTATGTGTTTTTTGTAGTTATAGA |
| MO318 | 169 | CATACATAAACTAAAAATGGATGAGATTGTCAAGAATATTCGTG |
| MO319 | 170 | CTAGTTAAATTACTGCTATGATTTTTCTATTACTCTTGGCCTCCT |
| MO320 | 171 | AGAGTAATAGAAAAATCATAGCAGTAATTTAACTAGCCTACGC |
| MO362 | 172 | CTGGTAGCAACAGCCCCCGATGCTGAT |
| MO363 | 173 | CAGCGCGATTTCCCCGTAAAAGTAT |
| MO364 | 174 | CAGGAAACAATTGCTTCTGACACGTTC |
| MO365 | 175 | TATGTAATCAAACTTATAATATTTCGTTTTTATTCC |
| MO359 | 176 | CCGGAAGTAAGTTTCTTCACGAATAAACG |
| MO357 | 177 | ATTAAGTCTAAATTTGTCATTGTTTGCG |
| MO358 | 178 | CCGGAAGTAAGTGCCTTCACGAATAAACG |
| MO360 | 179 | GTATTAGCCATCGCCTTCGGAAACGGT |
| MO361 | 180 | TTTTCTTTTGTTAGAATCGTCTAAGAATGT |
| MO366 | 181 | TTTGCCATCCATGCCTCATTTCATCCG |
| MO367 | 182 | CTGCCAGTCGATGATATTGAACTTA |
| MO353 | 183 | GCCGCAAACAATGCCAAATTTAGACTT |
| MO354 | 184 | GTATTGATGTCCAACGTCACTCA |
| MO356 | 185 | CCGGAAGTAAGTTACGCCACGAATAAACGT |
| MO321 | 186 | GCTATTAATGTATTTCATTTTTAGTTTATGTATGTGTTTTTTGTAGTTATAGA |
| MO322 | 187 | CACATACATAAACTAAAAATGAAATACATTAATAGCGAAGATTATTACGG |
| MO329 | 188 | ATGGATGGCAAACTGCCAGT |
| MO330 | 189 | ACTGGCAGTTTGCCATCCATNNKTCATTTCATCCG |
| MO323 | 190 | TATTCAGCAAGAGGTGATTCAAGAAATATCTTGACCGCAG |
| MO324 | 191 | TATTTCTTGAATCACCTCTTGCTGAATACGTAAACTACA |
| MO327 | 192 | CGCCGCAAACAATGACAAATTTNNKCTTAATCCGGAAGTA |
| MO328 | 193 | AAATTTGTCATTGTTTGCGGCG |
| MO392 | 194 | CAATCTGTTTATACATGCTCTGATGAAGTCCAGCC |
| MO393 | 195 | CAATCTGTTTATACATTTACTGATGAAGTCCAGCC |
| MO394 | 196 | CTTACTATACTACTAGCTCCTTCTTTGGG |
| MO389 | 197 | GTATAGTAAGCAATCTGGCTATACATTATCTGATG |
| MO390 | 198 | GTATAGTAAGCAATCTGTTAATACATTATCTGATG |
| MO391 | 199 | TACTAGCTCCTTCTTTGGGAAACC |
| MO395 | 200 | GTATAGTAAGCAATCTGTGGATACATTATCTGATG |
| MO396 | 201 | GTATTGCACCAACTCTTGGCGGAACC |
| MO397 | 202 | ATAGATGTTCGTCGCGTGCTTG |
| AT124 | 203 | GCGCAGACGGCGTTGATCGCTTTTATAAAACAAGAAATT |
| AT125 | 204 | ACGATAGATGCCGGTCTTCGCCCCA |
| AT135 | 205 | TATGTATTGCACCAAGCATTGGCGGAACCTGCTA |
| AT136 | 206 | GATGTTCGTCGCGTGCTTGTGCTG |
| AT137 | 207 | TATGTATTGCACCAAAATTTGGCGGAACCTGCTA |
| AT138 | 208 | TATGTATTGCACCAAGAATTGGCGGAACCTGCTA |
| AT139 | 209 | ACATCTGGTTGGTATTGATTATGACCAGACCGCATTGGCG |
| AT140 | 210 | CTAATTCCTGCATCCAGGTAACGCCCT |
| AT141 | 211 | ACATCTGGTTGGTATTGCTAAAGACCAGACCGCAT |
| AT142 | 212 | ACATCTGGTTGGTATTGAAAAAGACCAGACCGCAT |
| MO264 | 213 | TGATTCAAGAAATATCTTGACCGCAG |
| MO349 | 214 | CATTTTTAGTTTATGTATGTGTTTTTTGTAGTTAT |
| MO351 | 215 | CGTACACGTGGGCCGTTAGGTAT |
| MO352 | 216 | CAATACAAATCCATACTCGTTGAAAACT |
| MO368 | 217 | AACAAAAGAAAAGTATTAGCCATCGACT |
| MO371 | 218 | GAAAGGTTCAACAGTTAGGTTACACCGAAGATATG |
| MO372 | 219 | CATAGAAAGGGTGGACTTCGATATTGAATACC |
| MO373 | 220 | GATTGGCACTACTTTGCTGCACAACCACTAAC |
| MO374 | 221 | GATTGGCACTACTTTACTGCACAACCACTAAC |
| MO375 | 222 | TACCACCGGATCATCCACAATG |
| MO376 | 223 | TTGTTAGTGGTTGTGCATCAAAGTAG |
| MO377 | 224 | AAGCAAGAATGGATCGCCAG |
| MO398 | 225 | GAAAGGTTCAACAGTTAAATTACACCGAAGATATG |
| MO399 | 226 | GAAAGGTTCAACAGTTATTATACACCGAAGATATG |
| MO400 | 227 | GAAAGGTTCAACAGTTAGATTACACCGAAGATATG |
| ROS35 | 228 | CGTGTAGCCAGGACACCA |
| ROS36 | 229 | TGGTGTCCTGGCTACACG |
| AT111 | 230 | AAGAAGGAGCTAGTAGTATAGTAAGCAATCTGNNKATACATTATCTG |
| AT112 | 231 | GATTGCTTACTATACTACTAGCTCCTTCTTTGGGAAACCCA |
| AT113 | 232 | CTCAACCTGTTGGCTGGACTTCATCAGMNNATGTATAAACAG |
| AT114 | 233 | AAGTCCAGCCAACAGGTTGAGAACTTAGCGGTAC |

In the sequences of Table 8, the character S can include G or C. The character V can include A, C, or G. The character B can include G, C, or T. The character N can include A, C, G, or T. The character K can include G or T. The character M can include A or C.

TABLE 9
Sequences of gBlocks used in the construction of plasmids.

| | SEQ | |
|---|---|---|
| gblock | ID NO | Sequence |
| mce1 | 234 | GAACCAGGGTCAAGTGCATCCTTTAGTAAAAGGAGAAAAGAGCGGTTAAAACTGGG |
| | | AGCTATTTTCTTGGAAGGCATAACTGTTAAAGGTGTAACTCAAGTAACAACTCAAC |
| | | CCAAGTTAGGAGAGGTGCAGCAGAAATGTCATCAGTTCTGTGGCTGGGAAGGGTCT |
| | | GGGTTCCCTGGAGCACAGCCTGTCTCCATGGACAAGCAAAATATTAGACTTTTAGA |
| | | GCAGAAGCCTTATAAAGTAAGCTGGAAAGCAGACGGCACTCGTTACATGATGTTGA |
| | | TTGATGGCACAAATGAAGTTTTTATGATTGACAGAGATAATTCCGTTTTTCATGTT |
| | | TCAAATCTGGAATTTCCATTTCGTAAAGATCTTCGCATGCATTTATCAAATACTCT |
| | | TTTGGATGGGGAAATGATCATTGACAAAGTAAATGGACAGGCCGTCCCAAGATATT |
| | | TGATCTATGACATAATTAAGTTCAATGCACAACCAGTTGGAGACTGTGATTTTAAT |
| | | ATTCGTCTACAGTGTATTGAACGTGAAATTATAAGTCCACGACATGAAAAAATGAA |
| | | GACTGGACTCATTGACAAAACACAGGAACCATTTAGTGTCAGACCTAAACAATTTT |
| | | TTGACATCAATATTTCAAGAAAGCTTCTGGAAGGAAATTTTGCCAAAGAAGTCAGC |
| | | CATGAAATGGATGGACTTATTTTTCAGCCTATTGGAAAATACAAGCCTGGTCGATG |
| | | TGATGACATTTTGAAATGGAAACCTCCCAGTCTAAACTCTGTGGATTTTCGACTTA |
| | | AGATAACAAGAATGGGAGGAGAAGGGTTGCTTCCACAGAATGTTGGCCTTCTCTAT |
| | | GTTGGAGGTTATGAAAGACCCTTTGCACAAATCAAGGTGACAAAAGAACTAAAACA |
| | | GTATGACAACAAAATTATAGAATGCAAATTTGAGAACAATAGCTGGGTCTTCATGA |
| | | GACAGAGGATAGACAAAAGTTTCCCAAATGCCTACAACACAGCCATGGCTGTGTGC |
| | | AATAGCATCTCGAACCCTGTCACCAAGGAGATGCTGTTTGAATTCATTGACAGATG |
| | | TGCAGCAGCCGCCCAGGGACAGAAGCGGAAGTATCCCCTGGACCCTGACACGGAGC |
| | | TCATGCCGCCCCCACCGCCCAAAAGACTGCATCGGCCAACC |
| MERS | 235 | ATGTCCCAAATTGTCACTGGACTGTTCAAAGACTGCTCCCGTGAGACTTCAGGCTT |
| nsp14 | | GTCACCAGCGTATGCACCAACGTACGTCAGCGTTGACGATAAGTATAAAACTTCAG |
| | | ATGAATTGTGTGTGAATCTAAATCTTCCAGCGAATGTGCCTTATAGTAGAGTTATA |
| | | TCACGTATGGGCTTTAAACTGGACGCAACTGTTCCAGGCTATCCGAAACTATTTAT |
| | | TACAAGGGAGGAAGCAGTGCGTCAAGTGAGGTCATGGATAGGTTTCGACGTAGAAG |
| | | GAGCCCATGCGAGTAGAAACGCCTGCGGAACCAATGTTCCGCTTCAACTTGGGTTC |
| | | TCCACCGGGGTAAACTTCGTGGTTCAGCCTGTTGGTGTCGTTGATACTGAATGGGG |
| | | AAACATGTTAACGGGGATAGCCGCGAGGCCTCCGCCCGGAGAGCAATTTAAGCACC |
| | | TGGTCCCGCTTATGCATAAAGGGGCTGCTTGGCCAATCGTCCGTAGGCGTATCGTT |
| | | CAGATGTTGAGCGATACCCTTGACAAGCTTTCCGATTACTGCACATTTGTATGTTG |
| | | GGCCCATGGGTTTGAACTAACGTCAGCCTCCTACTTTTGCAAAATTGGGAAGGAGC |
| | | AAAAGTGTTGTATGTGTAATCGTAGAGCTGCAGCATACTCAAGCCCCTTGCAGAGT |
| | | TACGCCTGTTGGACTCATTCATGTGGCTACGACTACGTATACAATCCATTCTTTGT |
| | | AGACGTACAGCAGTGGGGCTACGTTGGCAATTTGGCAACAAATCATGACAGATATT |
| | | GTTCTGTGCATCAAGGCGCTCATGTTGCGTCCAATGATGCTATAATGACTAGATGC |
| | | CTGGCGATCCATTCTTGCTTCATAGAAAGGGTGGACTGGGATATTGAATACCCGTA |
| | | CATATCACACGAAAAGAAACTTAATTCTTGTTGCCGTATCGTGGAGAGGAATGTAG |
| | | TGAGGGCAGCTTTGCTGGCGGGATCATTTGATAAAGTGTATGACATAGGGAACCCT |
| | | AAGGGTATCCCCATTGTGGATGATCCGGTGGTAGATTGGCACTACTTTGATGCACA |
| | | ACCACTAACAAGAAAGGTTCAACAGTTATTTTACACCGAAGATATGGCATCCAGAT |
| | | TTGCTGATGGCCTTTGTCTATTCTGGAACTGTAATGTCCCCAAATACCCCAACAAT |
| | | GCGATCGTCTGTCGTTTTGACACGAGAGTCCATTCCGAGTTTAATTTGCCAGGGTG |
| | | TGACGGTGGGTCACTGTACGTGAATAAGCATGCCTTCCATACACCAGCCTATGACG |
| | | TTTCCGCCTTCAGAGATTTGAAACCGCTACCATTTTTCTACTACTCTACGACTCCG |
| | | TGCGAGGTACATGGTAACGGCAGTATGATCGAAGATATAGATTATGTTCCTCTGAA |
| | | AAGCGCCGTTTGCATAACAGCTTGCAACCTTGGGGGAGCCGTTTGCAGAAAACATG |
| | | CCACAGAGTACAGGGAATATATGGAAGCCTATAATCTTGTCTCAGCCTCTGGATTC |
| | | CGTCTGTGGTGCTATAAAACTTTTGATATATATAACCTTTGGAGTACGTTTACAAA |
| | | AGTTCAG |
| ASFV | 236 | ATGGCTAGTCTTGAGAATCTGGTAGCACGTTATCAGAGATGTTTCAACGATCAGTC |
| NP868R | | TCTGAAAAATTCAACCATCGAATTTGAAATCAGGTTCCAGCACATTAACTTTTTAT |
| | | TGTTTAAGACGGTGTATGAAGCTCTAGTCGCACAAGAAATCCCAAGTACTATCAGC |
| | | CACTCTATCAGGTGTATAAAGAAGGTCCACCATGAAAATCACTGCAGAGAAAAAAT |
| | | CCTTCCGTCTGACAATTTCTACTTCAAGAAGCAACCGCTTATGTTTTTTAAATTTA |
| | | GTGAGCCTGCTTCCTTGGGATGCAAAGTTAGCCTAGCCATCGAGCAACCAATTCGT |
| | | AAGTTCATCTTAGACTCTTCTGTGCTGGTCAGGTTAAAAAATAGAACCACATTCCA |
| | | GATTAGCGAACTTTGGAAGATCGAACTGACCGTCGTCAAACAGCTTATGGGCTCTG |
| | | AAGTCAGCGCAAAATTAACTGCCTTCAAGACTCTTTTGTTTGACACACCGGAGCAA |
| | | CAGACTGCAAAGAATATGATGACCTTGATTAATCCTGACGATGAGTACTTGTACGA |
| | | GATCGAGATAGAATACACAGGGAAACCTGAAAGTTTGACTGCTGCAGACGTGATCA |
| | | AGATCAAAAACACCGTTCTAACCTTAATATCACCTAATCATCTTATGCTGACAGCA |
| | | TATCATCAGGCTATCGAGTTCATTGCTTCACATATTCTTAGTTCTGAGATTCTTCT |
| | | GGCTAGAATAAAATCTGGAAAGTGGGGGTTAAAAAGATTGCTGCCTCAGGTAAAAT |
| | | CCATGACTAAAGCGGATTATATGAAATTCTACCCACCGGTAGGATACTATGTTACA |
| | | GATAAAGCGGATGGGATACGTGGCATTGCTGTCATTCAGGATACCCAAATGTACGT |
| | | GGTCGCAGATCAATTATATTCACTGGGAACGACCGGTATAGAACCCCTAAAGCCCA |
| | | CTATTCTTGACGGCGAATTCATGCCTGAAAAGAAAGAATTTTATGGATTCGATGTG |
| | | ATTATGTACGAGGGCAATCTGCTAACGCAGCAGGGTTTTGAGACGAGAATAGAAGC |
| | | ACTTAACAAAGGCATCAAAGTTTTGCAGGCCTTTAATATAAAGGCAGAAATGAAGC |
| | | CCTTCATAAGTCTTACCAGCGCAGACCCCAATGTCCTGTTAAAAAATTTTGAAAGC |
| | | GTTTTCAAAAAAAAAACTAGACCGTACTCAATAGACGGCATAATACTGGTCGAGCC |
| | | GGGAAACAGTTATTTAAACACCAATACCTTCAAATGGAAACCTACATGGGACAATA |
| | | CCTTGGACTTCCTGGTCAGAAAATGTCCCGAGTCCCTAAACGTCCCTGAGTATGCA |
| | | CCTAAAAAGGGCTTCTCCCTGCATCTGCTATTTGTCGGAATCAGTGGGGAGTTATT |
| | | CAAGAAATTGGCGCTAAACTGGTGTCCTGGCTACACGAAGTTATTCCCTGTTACCC |
| | | AACGTAATCAGAACTATTTCCCGGTCCAATTCCAGCCGTCCGATTTTCCATTGGCT |
| | | TTTTTATACTATCATCCCGACACGAGTAGTTTCAGTGACATAGATGGAAAGGTACT |
| | | GGAAATGAGATGCCTAAAAAGGGAGGTAAATTACGTCTCATGGGAAATCGTCAAGA |
| | | TTCGTGAAGATAGACAACAAGATCTAAAGACTGGGGGTTATTTCGGTAACGATTTT |
| | | AAGACTGCAGAGCTGACATGGTTGAATTACATGGACCCGTTCAGCTTTGAGGAATT |
| | | AGCCAAAGGACCATCCGGCATGTACTTTGCTGGGGCGAAGACCGGCATCTATCGTG |
| | | CGCAGACGGCGTTGATCAGTTTTATAAAACAAGAAATTATACAGAAAATCAGCCAT |
| | | CAAAGTTGGGTCATAGATCTGGGAATCGGAAAGGGTCAGGATCTAGGGCGTTACCT |
| | | GGATGCAGGAATTAGACATCTGGTTGGTATTGATAAAGACCAGACCGCATTGGCGG |
| | | AATTAATATATAGGAAATTCAGTCATGCTACGACCCGTCAGCACAAGCACGCGACG |
| | | AACATCTATGTATTGCACCAAGACTTGGCGGAACCTGCTAAAGAAATCTCTGAAAA |
| | | GGTGCATCAAATATATGGGTTTCCCAAAGAAGGAGCTAGTAGTATAGTAAGCAATC |
| | | TGTTTATACATTATCTGATGAAGTCCAGCCAACAGGTTGAGAACTTAGCGGTACTA |
| | | TGTCATAAGTTGTTGCAGCCTGGCGGGATGGTATGGTTCACAACCATGCTGGGTGA |
| | | GCGTGTCCTGGAATTATTACATGAGAATAGGGTCGAGCTGAACGAAGTCTGGGAAG |
| | | CAAGGGAAAATGAAGTGGTTAAGTTTGCCATTAAGAGGCTTTTTAAAGAGGATGTA |
| | | CTGCAAGAGACGGGGCAAGAAATAGGTGTACTGCTTCCGTTCTCTAACGGCGATTT |
| | | CTATAACGAATATCTGGTAAACACAGCTTTCCTAATCAAGATCTTCAAGCACCACG |
| | | GCTTTAGCTTGGTCCAAATGCAAAGTTTTAAGGATTGGATACCCGAATTTCAGACG |
| | | TTCAGCAAATCACTTTATAAGATACTGACGGAGGCGGACAAAACGTGGACGTCCCT |
| | | GTTCGGGTTTATATGCTTAAGGAAGAAT |
| MPV | 237 | ATGAAATACATTAATAGCGAAGATTATTACGGGAATCAACACAATATCATCGTAGA |
| vDI | | GCATCTAAGGGACCAATCCATAAAGATCGGGGATGTCTTCAACGAAGATAAACTGA |
| (1-545) | | GTGACGTTGGACATCAATACGCCGCAAACAATGACAAATTTAGACTTAATCCGGAA |
| | | GTAAGTTACTTCACGAATAAACGTACACGTGGGCCGTTAGGTATCTTATCTAACTA |
| | | TGTGAAGACTCTATTGATCTCCATGTATTGCTCAAAAACATTCTTAGACGATTCTA |
| | | ACAAAAGAAAAGTATTAGCCATCGACTTCGGAAACGGTGCTGACTTGGAGAAATAC |
| | | TTTTACGGGGAAATCGCGCTGCTGGTAGCAACAGACCCCGATGCTGATGCAATTGC |
| | | CAGAGGCAACGAAAGATACAACAAGCTGAATTCAGGAATAAAAACGAAATATTATA |
| | | AGTTTGATTACATACAGGAAACAATTCGTTCTGACACGTTCGTGTCTAGCGTTAGA |
| | | GAAGTATTCTACTTTGGTAAGTTCAATATCATCGACTGGCAGTTTGCCATCCATTA |
| | | CTCATTTCATCCGAGGCATTATGCGACAGTAATGAATAATCTGTCTGAATTAACTG |
| | | CTAGCGGCGGGAAAGTGCTTATTACTACGATGGACGGGGACAAGCTAAGTAAGTTG |
| | | ACGGATAAGAAGACTTTTATCATCCATAAGAACTTGCCTAGTTCCGAGAACTACAT |
| | | GAGCGTTGAAAAAATCGCCGACGACCGTATAGTAGTATATAATCCATCCACGATGT |
| | | CTACACCCATGACTGAATACATAATCAAGAAAAATGATATTGTAAGAGTTTTCAAC |
| | | GAGTATGGATTTGTATTGGTCGACAACGTAGATTTTGCTACCATAATAGAACGTTC |
| | | CAAAAAGTTCATAAACGGTGCATCAACAATGGAAGACAGACCTAGTACTAGAAACT |
| | | TTTTCGAACTTAACCGTGGCGCCATAAAATGCGAGGGCTTGGATGTAGAGGATTTA |
| | | CTTTCATATTATGTAGTTTACGTATTCAGCAAGAGG |
| MPV | 238 | ATGGATGAGATTGTCAAGAATATTCGTGAGGGAACGCACGTGTTGCTGCCTTTCTA |
| vD12 | | CGAGACTTTGCCGGAATTGAACCTTTCCCTTGGAAAGAGCCCTCTTCCCAGTCTAG |
| | | AATATGGAGCAAATTACTTCCTTCAGATTAGTAGAGTAAACGATCTGAACAGGATG |
| | | CCAACCGATATGTTGAAGCTATTTACACACGATATTATGTTACCTGAATCCGATTT |
| | | AGACAAAGTTTACGAAATCCTGAAAATTAACTCAGTGAAGTATTACGGCCGTAGCA |
| | | CGAGGGCCGACGCCGTAGTTGCAGATCTAAGTGCTAGGAACAAGCTATTCAAGCGT |
| | | GAGAGAGACGCTATAAAAAGTAACAACCACCTGACCGAGAACAATCTTTACATAAG |
| | | TGACTATAAGATGCTTACCTTCGACGTCTTTAGACCATTATTCGACTTTGTGAATG |
| | | AAAAATATTGCATCATAAAGTTACCGACGCTTTTTGGAAGAGGGGTTATAGACACA |
| | | ATGAGAATATACTGCTCACTTTTCAAGAATGTGAGACTATTAAAGTGTGTCTCAGA |
| | | CAGTTGGCTGAAAGACTCCGCAATTATGGTTGCATCAGATGTTTACAAGAAGAACT |
| | | TAGACCTATTTATGTCTCACGTTAAGTCTGTTACGAAGTCCAGTAGTTGGAAAGAT |
| | | GTAAACACAGTTCAATTCTCCATTTTGAACGATCCCGTAGACACGGAATTTATTAA |
| | | CAAATTCCTTGAGTTCTCTAATAGAGTCTACGAAGCCTTGTACTATGTTCACAGCC |
| | | TTTTATACTCCAGTATGACCAGTGATTCAAAAAGTATAGAAAATAAACATCAGCGT |
| | | AGGCTAGTTAAATTACTGCTA |

TABLE 10
Genomic DNA fragments amplified from S. cerevisiae YPH500
used in the construction of plasmids.

| | SEQ | |
|---|---|---|
| gblock | ID NO | Sequence |
| abd1 | 239 | ATGTCAACCAAACCAGAGAAGCCAATATGGATGTCACAAGAAGACTATGACCGTCA |
| | | GTACGGCTCAATTACTGGTGATGAATCGTCCACAGTCTCAAAAAAAGACTCCAAAG |
| | | TTACTGCCAATGCGCCTGGTGATGGTAATGGATCATTACCAGTTTTGCAAAGCAGC |
| | | TCTATCCTTACTTCTAAGGTGTCTGATCTACCCATTGAGGCAGAATCCGGGTTTAA |
| | | AATCCAAAAGAGAAGACATGAAAGGTATGATCAAGAGGAAAGATTACGCAAACAGC |
| | | GTGCTCAAAAATTGAGAGAAGAGCAACTAAAGAGACATGAAATAGAGATGACTGCA |
| | | AATAGATCCATAAATGTTGACCAGATTGTCCGAGAGCACTATAATGAACGTACAAT |
| | | AATCGCCAATCGCGCTAAGAGGAATCTATCGCCTATTATTAAGCTTCGTAATTTTA |
| | | ACAATGCGATTAAGTATATGTTAATTGACAAATATACAAAACCTGGAGATGTCGTT |
| | | TTAGAACTCGGATGTGGCAAAGGTGGTGATTTAAGAAAATATGGTGCTGCAGGTAT |
| | | TTCACAGTTTATTGGTATTGACATTTCCAATGCCTCTATCCAAGAAGCTCATAAGA |
| | | GATACCGGTCCATGAGAAATTTGGACTACCAAGTTGTGCTAATTACTGGGGATTGC |
| | | TTTGGCGAATCATTAGGTGTTGCAGTAGAGCCCTTCCCAGACTGTAGATTTCCGTG |
| | | TGATATCGTTTCGACGCAATTTTGCTTGCATTATGCTTTTGAGACCGAAGAAAAAG |
| | | CAAGGAGAGCTTTACTGAATGTCGCCAAATCACTCAAAATTGGTGGTCACTTTTTT |
| | | GGCACGATACCAGATTCAGAATTCATTCGTTATAAACTGAATAAATTTCCCAAAGA |
| | | AGTGGAAAAGCCATCTTGGGGAAACTCCATTTATAAAGTGACATTTGAGAATAATT |
| | | CATACCAAAAGAATGATTATGAGTTCACATCGCCTTATGGTCAAATGTATACTTAT |
| | | TGGCTAGAAGATGCTATAGATAATGTTCCTGAATATGTTGTGCCATTCGAAACCTT |
| | | AAGAAGTTTGGCTGATGAATACGGTTTGGAACTAGTATCGCAAATGCCTTTTAATA |
| | | AATTTTTTGTCCAAGAAATTCCCAAATGGATTGAGAGGTTTTCGCCTAAAATGAGG |
| | | GAAGGTTTACAAAGATCTGATGGTAGATATGGTGTTGAAGGGGATGAAAAGGAAGC |
| | | GGCATCATATTTCTATACCATGTTTGCATTCCGTAAGGTAAAACAGTATATCGAAC |
| | | CGGAAAGCGTAAAGCCCAACTGA |
| tpil | 240 | ACACCCCTTTTCTGGCATCCAGTTTTTAATCTTCAGTGGCATGTGAGATTCTCCGA |
| pro- | | AATTAATTAAAGCAATCACACAATTCTCTCGGATACCACCTCGGTTGAAACTGACA |
| moter | | GGTGGTTTGTTACGCATGCTAATGCAAAGGAGCCTATATACCTTTGGCTCGGCTGC |
| | | TGTAACAGGGAATATAAAGGGCAGCATAATTTAGGAGTTTAGTGAACTTGCAACAT |
| | | TTACTATTTTCCCTTCTTACGTAAATATTTTTCTTTTTAATTCTAAATCAATCTTT |
| | | TTCAATTTTTTGTTTGTATTCTTTTCTTGCTTAAATCTATAACTACAAAAAACACA |
| | | TACATAAACTAAAA |

TABLE 11
YeRCOM strains engineered in this disclosure.

| | P_{TPI1} driven expression | | |
|---|---|---|---|
| | cassettes present in these | | |
| Strain Name | strains | Mating allele | Source |
| S. cerevisiae abd1::kanMX4 | NA | MATa MATα | ATCC |
| diploid | | | 4023376 |
| S. cerevisiae abd1::kanMX4 | abd1 | MATa | This study |
| pMO1 haploid | | | |
| S. cerevisiae abd1::kanMX4 | abd1, | MATa | This study |
| pMO1 pMO65 haploid | MERS nsp14 - mce1 | | |
| S. cerevisiae abd1::kanMX4 | abd1, | MATa | This study |
| pMO1 pMO66 haploid | MERS nsp14 MTase domain | | |
| | (D292-Q525) deletion - | | |
| | mce1 | | |
| S. cerevisiae abd1::kanMX4 | abd1, | MATa | This study |
| pMO1 pMO67 haploid | MERS nsp14 (Y511-Q525 | | |
| | deletion) - mce1 | | |
| S. cerevisiae abd1::kanMX4 | abd1, | MATa | This study |
| pMO1 pMO68 haploid | MERS nsp14 ExoN domain | | |
| | (S2-V291) deletion - mce1 | | |
| S. cerevisiae abd1::kanMX4 | abd1, | MATa | This study |
| pMO1 pMO70 haploid | MERS nsp14 (D243A) - | | |
| | mce1 | | |
| S. cerevisiae abd1::kanMX4 | abd1, | MATa | This study |
| pMO1 pMO69 haploid | MERS nsp14 (D331A) - | | |
| | mce1 | | |
| S. cerevisiae abd1::kanMX4 | abd1, | MATa | This study |
| pMO1 pIJ31 haploid | MERS nsp14 (F365L) - | | |
| | mce1 | | |
| S. cerevisiae abd1::kanMX4 | abd1, | MATa | This study |
| pMO1 pIJ19 haploid | MERS nsp14 (F365G) - | | |
| | mce1 | | |
| S. cerevisiae abd1::kanMX4 | abd1, | MATa | This study |
| pMO1 pIJ29 haploid | MERS nsp14 (F365D) - | | |
| | mce1 | | |
| S. cerevisiae abd1::kanMX4 | abd1, | MATa | This study |
| pMO1 pIJ30 haploid | MERS nsp14 (F365N) - | | |
| | mce1 | | |
| S. cerevisiae abd1::kanMX4 | abd1, | MATa | This study |
| pMO1 pIJ17 haploid | MERS nsp14 (W293F) - | | |
| | mce1 | | |
| S. cerevisiae abd1::kanMX4 | abd1, | MATa | This study |
| pMO1 pIJ18 haploid | MERS nsp14 (D353A) - | | |
| | mce1 | | |
| S. cerevisiae abd1::kanMX4 | abd1, | MATa | This study |
| pMO1 pIJ16 haploid | MERS nsp14 (D353T) - | | |
| | mce1 | | |
| S. cerevisiae abd1::kanMX4 | abd1, | MATa | This study |
| pMO1 pAT15 haploid | ASFV NP868R - mce1 | | |
| S. cerevisiae abd1::kanMX4 | abd1, | MATa | This study |
| pMO1 pIJ22 | ASFV NP868R (Y714A) - | | |
| | mce1 | | |
| S. cerevisiae abd1::kanMX4 | abd1, | MATa | This study |
| pMO1 pIJ23 | ASFV NP868R (Y714L) - | | |
| | mce1 | | |
| S. cerevisiae abd1::kanMX4 | abd1, | MATa | This study |
| pMO1 pIJ24 | ASFV NP868R (F711A) - | | |
| | mce1 | | |
| S. cerevisiae abd1::kanMX4 | abd1, | MATa | This study |
| pMO1 pIJ25 | ASFV NP868R (F711L) - | | |
| | mce1 | | |
| S. cerevisiae abd1::kanMX4 | abd1, | MATa | This study |
| pMO1 pIJ26 | ASFV NP868R (F711W) - | | |
| | mce1 | | |
| S. cerevisiae abd1::kanMX4 | abd1, | MATa | This study |
| pMO1 pIJ27 | ASFV NP868R (D680L) - | | |
| | mce1 | | |
| S. cerevisiae abd1::kanMX4 | abd1, | MATa | This study |
| pMO1 pMO64 | ASFV NP868R (K647Y) - | | |
| | mce1 | | |
| S. cerevisiae abd1::kanMX4 | abd1, | MATa | This study |
| pMO1 pAT19 | ASFV NP868R (S604A) - | | |
| | mce1 | | |
| S. cerevisiae abd1::kanMX4 | abd1, | MATa | This study |
| pMO1 pAT20 | ASFV NP868R (D646A) - | | |
| | mce1 | | |
| S. cerevisiae abd1::kanMX4 | abd1, | MATa | This study |
| pMO1 pAT21 | ASFV NP868R (D646E) - | | |
| | mce1 | | |
| S. cerevisiae abd1::kanMX4 | abd1, | MATa | This study |
| pMO1 pAT22 | ASFV NP868R (D680N) - | | |
| | mce1 | | |
| S. cerevisiae abd1::kanMX4 | abd1, | MATa | This study |
| pMO1 pAT23 | ASFV NP868R (D680A) - | | |
| | mce1 | | |
| S. cerevisiae abd1::kanMX4 | abd1, | MATa | This study |
| pMO1 pAT24 | ASFV NP868R (D680E) - | | |
| | mce1 | | |
| S. cerevisiae abd1::kanMX4 | abd1, | MATa | This study |
| pMO1 pMO41 pMO42 haploid | vD1 (aa 498-844) + vD12 | | |
| S. cerevisiae abd1::kanMX4 | abd1, | MATa | This study |
| pMO1 pMO41 haploid | vD1 (aa 498-844) | | |
| S. cerevisiae abd1::kanMX4 | abd1, | MATa | This study |
| pMO1 pMO42 haploid | vD12 | | |
| S. cerevisiae abd1::kanMX4 | abd1, | MATa | This study |
| pMO1 pMO54 pMO42 haploid | vD1 (aa 498-844) D620A + | | |
| | vD12 | | |
| S. cerevisiae abd1::kanMX4 | abd1, | MATa | This study |
| pMO1 pMO55 pMO42 haploid | vD1 (aa 498-844) R655A + | | |
| | vD12 | | |
| S. cerevisiae abd1::kanMX4 | abd1, | MATa | This study |
| pMO1 pMO48 pMO42 haploid | vD1 (aa 498-844) Y555F+ | | |
| | vD12 | | |
| S. cerevisiae abd1::kanMX4 | abd1, | MATa | This study |
| pMO1 pMO47 pMO42 haploid | vD1 (aa 498-844) Y555A + | | |
| | vD12 | | |
| S. cerevisiae abd1::kanMX4 | abd1, | MATa | This study |
| pMO1 pMO50 pMO42 haploid | vD1 (aa 498-844) D598A + | | |
| | vD12 | | |
| S. cerevisiae abd1::kanMX4 | abd1, | MATa | This study |
| pMO1 pMO56 pMO42 haploid | vD1 (aa 498-844) Y683A + | | |
| | vD12 | | |
| S. cerevisiae abd1::kanMX4 | abd1, | MATa | This study |
| pMO1 pMO53 pMO42 haploid | vD1 (aa 498-844) D545A + | | |
| | vD12 | | |
| S. cerevisiae abd1::kanMX4 | abd1, | MATa | This study |
| pMO1 pMO49 pMO42 haploid | vD1 (aa 498-844) F556A + | | |
| | vD12 | | |
| S. cerevisiae abd1::kanMX4 | abd1, | MATa | This study |
| pMO1 pMO46 pMO42 haploid | vD1 (aa 498-844) R548A + | | |
| | vD12 | | |
| S. cerevisiae abd1::kanMX4 | abd1, | MATa | This study |
| pMO1 pIJ21 pMO42 haploid | vD1 (aa 498-844) N550L + | | |
| | vD12 | | |
| S. cerevisiae abd1::kanMX4 | abd1, | MATa | This study |
| pMO1 pIJ12 pMO42 haploid | vD1 (aa 498-844) K498- | | |
| | S590 deletion + vD12 | | |
| S. cerevisiae abd1::kanMX4 | abd1, | MATa | This study |
| pMO1 pIJ13 pMO42 haploid | vD1 (aa 498-844) V783- | | |
| | R844 deletion + vD12 | | |
| S. cerevisiaeabd1::kanMX4 | abd1, | MATa | This study |
| pMO1 pIJ11 pMO42 haploid | vD1 (aa 498-844) K498- | | |
| | K560 deletion + vD12 | | |
| S. cerevisiae abd1::kanMX4 | abd1, | MATa | This study |
| pMO1 pMO5 haploid | rnmt-mce1 | | |

### Example 8

### Development of YeRC0M-MERS

This example illustrates engineering a yeast complementation platform for RNA capping enzymes from Middle East respiratory syndrome-related coronavirus.

Examples 1-6 describe S. cerevisiae abd1::kanMX4 pMO1 complementation platform to functionally characterize SARS-CoV-2 nsp14 N7-MTase activity (Yeast platform for RNA Cap-0 N7-Methyltransferase—YeRCOM).¹² This strain has a genomic deletion of the S. cerevisiae N7-methyltransferase (abd1), making S. cerevisiae survival dependent on viral N7-MTase activity. Next, the S. cerevisiae abd1::kanMX4 pMO1 strains were used as starting strains to investigate engineering strains for RNA capping enzymes from Middle East respiratory syndrome-related coronavirus (MERS-CoV). Plasmid pMO65 was constructed encoding the wild type MERS nsp14 gene fused to the human mce1 gene which directs viral nsp14 to the S. cerevisiae RNA Polymerase II complex where S. cerevisiae mRNA capping occurs¹³. If the MERS nsp14-mce1 fusion catalyzes the RNA cap-0 N7-methylation of S. cerevisiae mRNAs, then S. cerevisiae abd1::kanMX4 pMO1 pMO65 haploid will survive in the presence of 5-FOA which cures the S. cerevisiae abd1 expressing plasmid, pMO1 (FIG. 14A-14D). As shown in FIGS. 14B-14C, S. cerevisiae abd1::kanMX4 pMO1 pMO65 haploid survived upon 5-FOA treatment. This suggested that a functional MERS-CoV nsp14 was able to complement the loss of abd1 expression in S. cerevisiae abd1::kanMX4. This functional complementation platform was named YeRCOM-MERS.

The next goal was to use YeRCOM-MERS for functional characterization of MERS-CoV nsp14. Truncations of MERS nsp14 were created lacking the N7-MTase domain (pMO66), the ExoN domain (pMO68), and the C-terminal 17 amino acids (pMO67). S. cerevisiae abd1::kanMX4 pMO1 haploid was transformed with these plasmids and these strains were subjected to 5-FOA treatment and selection process. None of these truncations were able to recover growth of S. cerevisiae abd1::kanMX4 pMO1 haploid (FIGS. 141B-14C), indicating that these truncations resulted in a functionally inactive or highly inefficient MERS nsp14 in these complementation platforms. Next the effect of point mutation D331A, known to abolish SARS-CoV-2 and MERS-CoV nsp14 activity in vitro was studied¹⁴. Consistent with the in vitro data, this point mutation was found to abolish MERS-CoV nsp14 activity in vivo as well (FIGS. 14B-14C). Lastly, the effect of point mutation D243A, known to abolish nsp14 ExoN activity, on MERS nsp14 N7-MTase activity was studied to determine if MERS nsp14 ExoN domain activity is necessary for efficient MERS-CoV nsp14 N7-MTase activity. This point mutation did not reduce MERS nsp14 N7-MTase activity in vivo as observed by the phenotypic recovery of the yeast strains up on 5-FOA treatment (FIGS. 14B-14C). These data indicated that while the MERS nsp14 ExoN domain structure is important for N7-MTase activity, the ExoN domain nuclease activity is not critical for N7-MTase domain activity.

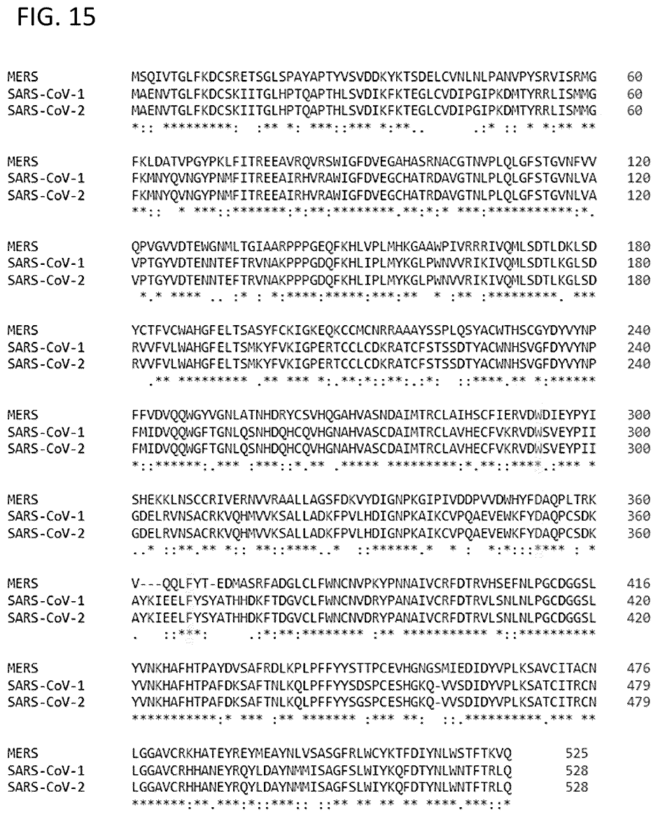

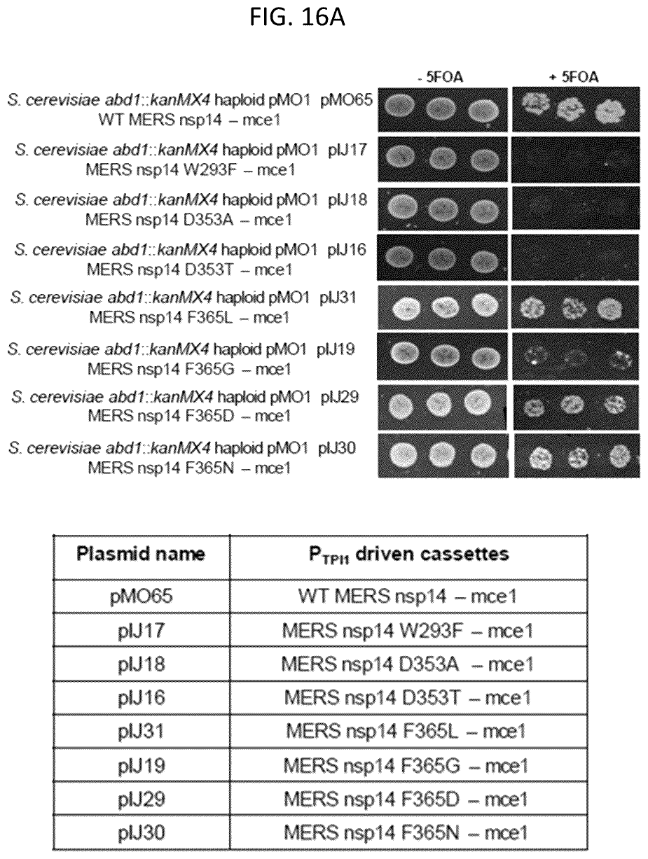

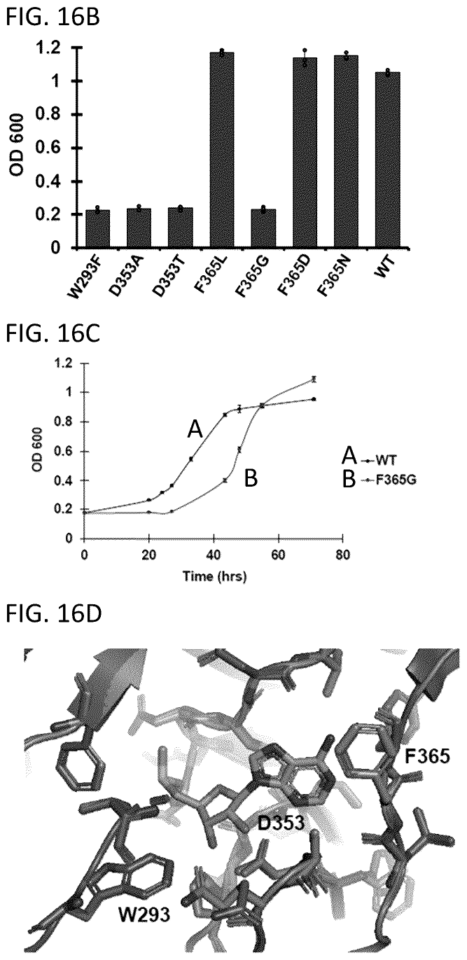

Since essential viral enzymes are often targeted for virus biocontainment as well as to identify attenuation mutations for live attenuated vaccine development perspective^{15,16}, it was next investigated if YeRCOM-MERS could be used to identify novel inactivation mutations as well as attenuation mutations in MERS-CoV-nsp14. Because MERS-CoV and SARS-CoV-2 nsp14 have high protein sequence homology (FIG. 15) (62.5% identity, 77.4% similarity), this disclosure's observations from SARS-CoV-2 were used as a starting point to identify attenuation mutations in MERS-CoV nsp14. Residue F368 in SARS-CoV-2 nsp14 (with reference to SEQ ID NO: 4) corresponds to Residue F365 in MERS-CoV nsp14 (with reference to SEQ ID NO: 24). Using YeRCOM for the directed evolution of SARS-CoV-2 nsp14, W293F, F368L, D353T, and D353A were identified as point mutations that resulted in attenuation of N7-methyltransferase activity. These mutations were mapped onto MERS-CoV nsp14 to see if they resulted in attenuation as they did for SARS-CoV-2 nsp14. Interestingly, unlike the SAR-CoV-2 nsp14 attenuated variants, the corresponding MERS-CoV nsp14 variants W293F, D353A and D353T abolished activity whereas F365L did not attenuate activity in comparison to wild-type MERS-CoV nsp14 (FIGS. 16A-16B). Next it was investigated whether other variants could b identified which may lead to attenuation. The F365 residue in the active-site was targeted, testing F365G, F365N, and F365D. It was found that F365N and F365D do not attenuate activity; however, F365G attenuated activity significantly in comparison to wild type MERS-CoV nsp14 (FIGS. 16A-16B). In addition to the smaller size of glycine, it is also possible that slight modifications in protein conformations due to glycine incorporation contribute to attenuation. Further, to understand why the attenuation mutations found in SARS-CoV-2 nsp14 did not map onto MERS nsp14, a homology model was generated between SARS-CoV and MERS nsp14 using SWISS model¹⁷ (FIGS. 15 and 16D). The active site between the two models appears almost identical, thus the differences in activity likely come from changes in global protein shape that differ between the two enzymes.

### Example 9

### Development of YeRCOM-MPV

This example illustrates engineering yeast complementation platform for RNA capping enzymes from human variants of Monkeypox virus:

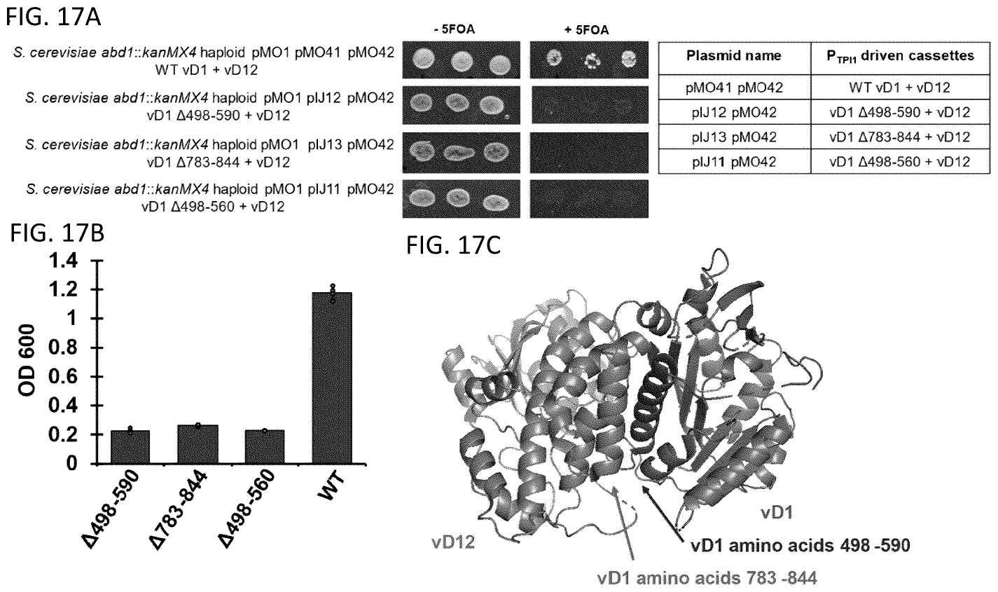

Monkeypox virus (MPV) related outbreaks continue to occur in human population with the 2022 outbreak resulting in more than 29,000 confirmed infections in the United States alone. Unlike coronaviruses, MPV belongs to a family of poxviridae which are double stranded DNA viruses. Interestingly, poxviridae DNA genomes typically encode highly conserved RNA capping enzymes for capping viral RNA transcripts. Therefore, it was investigated if YeRCOM could be engineered for RNA capping enzymes from this important family of zoonotic pathogenic DNA viruses, particularly for the human variant of MPV. Studies suggest that the 5′ m7GpppN cap structure of poxviruses is formed by sequential reactions of RNA triphosphatase, RNA guanylyl transferase, and RNA guanine-N7 methyltransferase^{18,19}. These enzymes lie within the 844-aa polypeptide encoded by the MPV D1 gene (vD1). The N-terminal domain of vD1 spanning amino acids 1-545 encodes an autonomous triphosphatase-guanylyltransferase domain²⁰⁻²², while the C-terminal domain encodes the methyltransferase domain which dimerizes with a 287-aa polypeptide encoded by the D12 gene (vD12)^{23,24}. vD1 encodes the methyltransferase active site but requires the vD12 stimulatory subunit for efficient activity (activity increases 30- to 50-fold with vD12 allosteric activation)²⁵. vD12 has no discernable homologs in eukaryotic organisms which makes it a great target for antiviral discovery/development²⁶. Shuman and coworkers have previously shown that co-expression of Vaccinia Virus (VACV) vD1+vD12 can functionally complement yeast abd1 mutants.²⁶ It is important to note that similar to MPV, Vaccinia Virus also belongs to the family of poxviridae. Therefore, it was investigated whether it was possible to complement lack of abd1 expression in the YeRCOM strains, S. cerevisiae abd1::kanMX4 pMO1, with co-expression of MPV vD1 and vD12. To test this, plasmids pMO41 and pMO42 were constructed for constitutive expression of MPV VD1 and MPV VD12 respectively. S. cerevisiae abd1::kanMX4 pMO1 was then transformed with pMO41 and pMO42. It was observed that co-expression of MPV vD1 (pMO41) and vD12 (pMO42) could recover S. cerevisiae abd1::kanMX4 pMO1 haploid growth in the presence of 5-FOA (FIGS. 17A-17B). It was found that mce1 fusion was not needed for MPV vD1/vD12 complementation. This indicated that a functional MPV vD1/vD12 co-expression alone was able to complement the loss of abd1 expression in S. cerevisiae abd1::kanMX4. This functional complementation platform was named YeRCOM-MPV.

Next, YeRCOM-MPV was used for biochemical characterization. Based on the crystal structure of the VACV capping enzyme²⁷ and studies in the literature²⁸, a series of truncations and point mutations in the vD1 gene were made to investigate protein domains and residues that are important for vD1 N7-MTase activity. First, vD1 truncations AK498-K560 (pIJ1l), AK498-S590 (pIJ12), ΔV783-R844 (pIJ13) were generated to investigate the importance of these regions for both substrate binding and interaction with vD12. Studies on VACV indicated that AK498-K560 and AK498-S590 variants of vD1 from VACV abolished the N7-MTase activity. Similar truncation mutations were generated in MPV VD1 by constructing plasmids pIJ11 and pIJ12. These plasmids were transformed into S. cerevisiae abd1::kanMX4 pMO1 and complementation assays were performed as before. It was found that these truncations were lethal to S. cerevisiae abd1::kanMX4 pMO1 pMO42 pIJI1 and S. cerevisiae abd1::kanMX4 pMO1 pMO42 pIJ12 haploid under 5-FOA selection conditions, indicating that these truncations abolished MPV vD1 N7-MTase activity (FIGS. 17A-17B). Based on the VACV vD1 crystal structure and previous studies on VACV vD1 N7-MTase, these residues have been hypothesized to be important for coenzyme S-adenosylmethionine (AdoMet) binding and vD12 interactions^{26,29}. It is also suggested that the C-terminus of VACV vD1, residues 783-815, also interacts with VACV vD12. A truncation variant of MPV vD1 was made that has this region deleted (ΔV783-R844, pIJ13). This truncation variant was tested using our YeRCOM-MPV complementation platform. It was found that this truncation also abolished MPV vD1 activity (FIGS. 17A-17B).

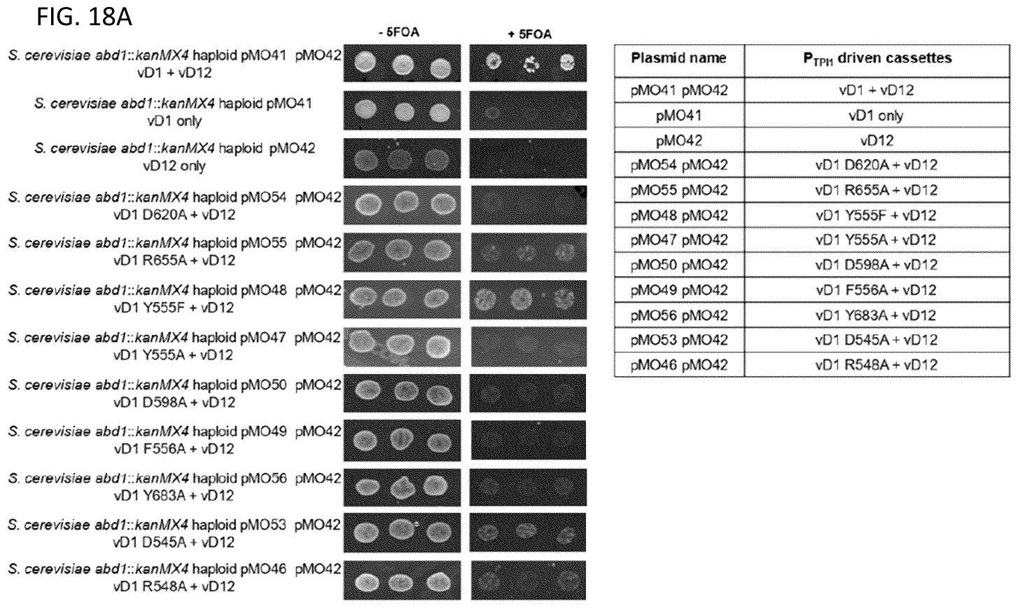

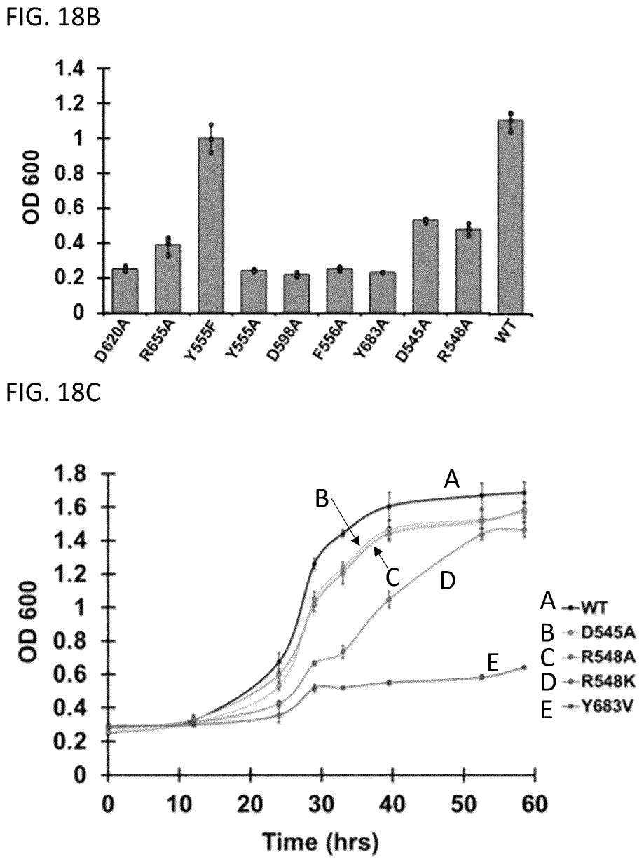

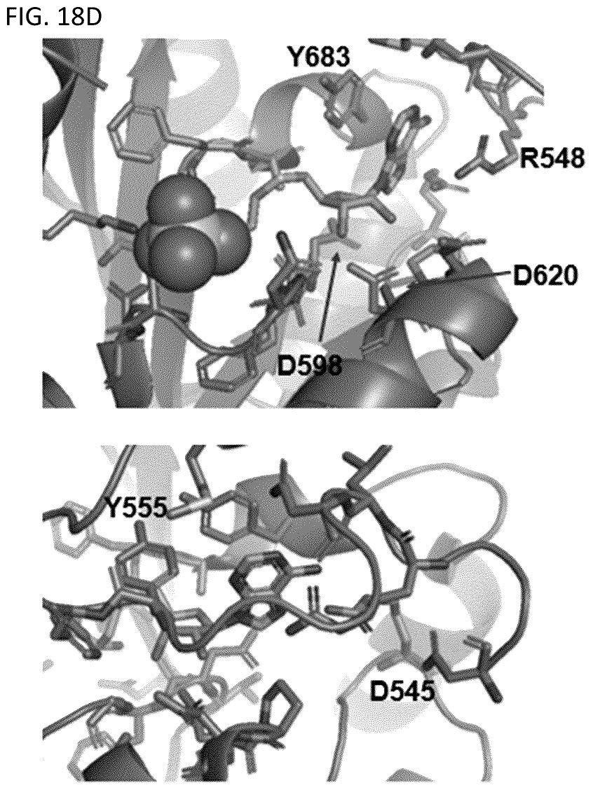

Next, variants of vD1 that contain mutations in the vicinity of the coenzyme AdoMet binding site were investigated. The role of residues responsible for binding of the adenine (R548, Y683, R655, D545), ribose (D620), and methionine (D598) moieties of AdoMet were investigated. Mutations were made to probe the role of residues hypothesized to be involved in the MPV vD1 N7-MTase catalytic mechanism (Y555, F556). R655 makes polar contact with the adenine N7, and this interaction is stabilized by the side chain of D545. Mutating either of these residues to alanine, R655A (pMO55) and D545A (pMO53), led to attenuation of vD1 activity (FIGS. 18A-18C). However, Y683A (pMO56) abolished vD1 function (FIGS. 18A-18B), likely due to the importance of pi-pi stacking interactions between the aromatic ring of Y683 and the aromatic rings of the adenine moiety of the AdoMet coenzyme to stabilize the rare syn-conformation of the adenine base²⁹. Residue D620 coordinates the 2′- and 3′-OH of the adenosine ribose and has been hypothesized to be critical for the binding and orientation of AdoMet so that its sulfonium center is aligned properly for attack by the cap guanine N7²⁸. The D620A (pMO54) mutation abolished vD1 activity (FIGS. 18A-18B), in alignment with previously observed effects of D260A mutation on VACV vD1 function in vivo³⁰. D598 makes polar contacts with both the adenosine and methionine moieties²⁸. In vitro analysis of D598A showed that this mutation only reduced vaccinia vD1 activity by a factor of two³¹, however, in vivo data demonstrated that mutation D598A in VACV vD1 precluded yeast growth at 30° C. with a small amount of growth observed at 19° C.³⁰ Here, it was found that MPV vD1 D598A (pMO50) prevented yeast growth at 30° C. after 2 days, however, growth was observed after 96 hours (FIG. 19). These data demonstrate the importance of developing in vivo systems to study enzyme function in tandem with in vitro analysis. Residues Y555 and F556 have been proposed to be involved in the VACV vD1 catalytic mechanism because mutations at these positions have not been shown to significantly affect methyl-donor binding or the ability of the enzyme to undergo a AdoMet-induced conformational change²⁹. Previous studies have shown that Y555A and F556A mutations abolish vD1 activity in vitro with Y555F still retaining some activity. It was observed that MPV vD1 Y555A (pMO47) and F556A (pMO49) abolish vD1 activity, however, Y555F (pMO48) conferred almost equal yeast growth as WT vD1 (FIGS. 18A-18B). This is an interesting observation because the hydroxyl group of Y555 is only 4 angstroms away from the sulfur atom of AdoMet²⁹. Because of its proximity to the methyl donor, this residue has been implicated in catalysis with a proposed role of forming a charge-dipole interaction with the positively charged sulfur atom of the AdoMet thus favoring methyl transfer³². Taken together, these results indicate that the major function of this residue is likely optimizing positioning of the methyl-donor through aromatic interactions (stacking against the ribose) rather than the charge-dipole interaction the hydroxyl group of the tyrosine makes with the sulfur atom of AdoMet. As demonstrated here, this extensive biochemical characterization also resulted in the identification of various attenuated variants of vD1.

Directed evolution experiments were performed on MPV vD1 to identify additional attenuated variants. Site saturated mutagenesis libraries were made at positions Y683 and R548; these residues interact with the adenine base moiety of AdoMet on opposite sides. 64 colonies were screened from both the Y683X and R548X libraries and one hit was found from each library: Y683V and R548K. Growth rates of these hits as well as the attenuated mutants identified through biochemical analysis were characterized (FIGS. 18A-18C). The attenuated variants of MPV vD1 identified in this study can be used in the development of live attenuated vaccine candidates for MPV.

### Example 10

### Development of YeRCOM-ASFV

This example illustrates engineering a yeast complementation platform for RNA capping enzymes expressed by African Swine Fever Virus.

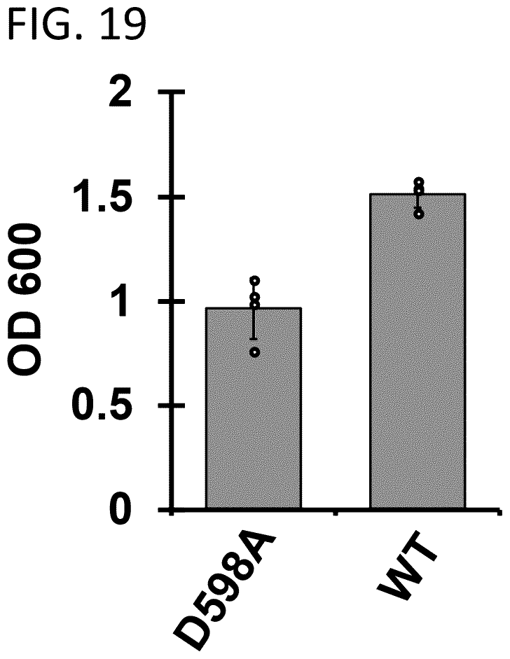

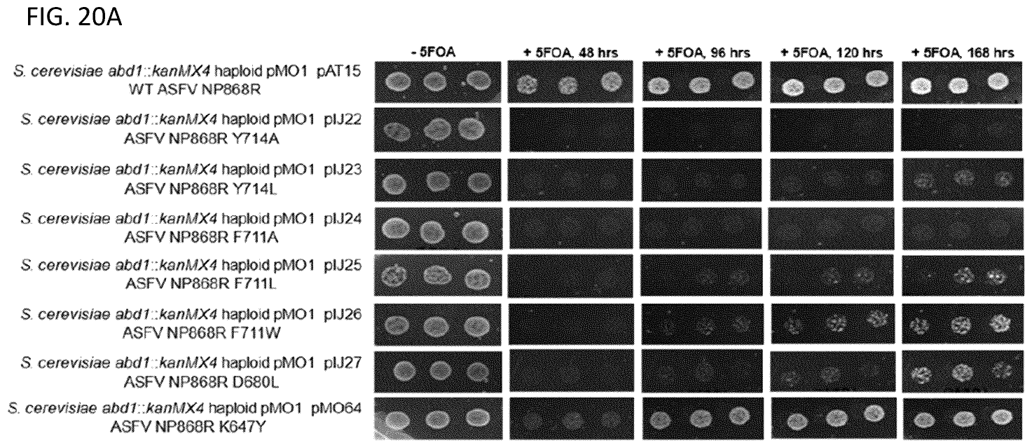

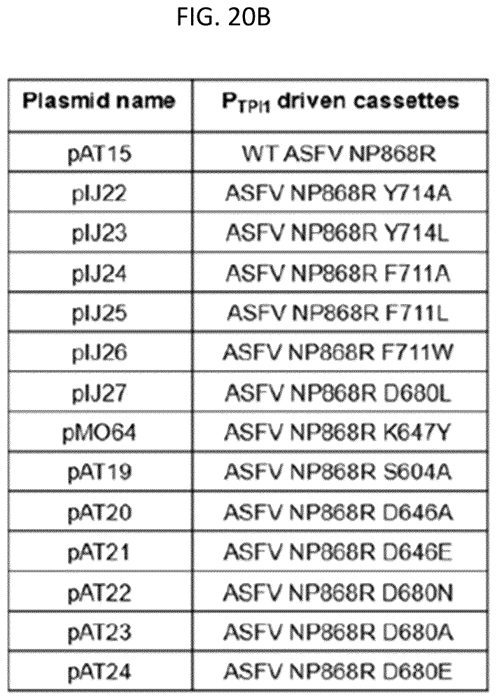

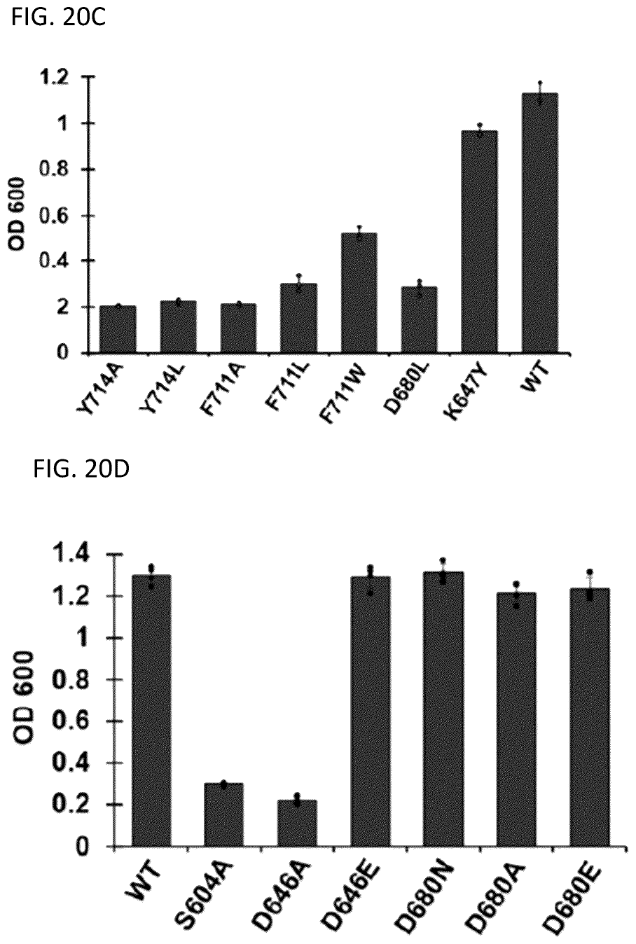

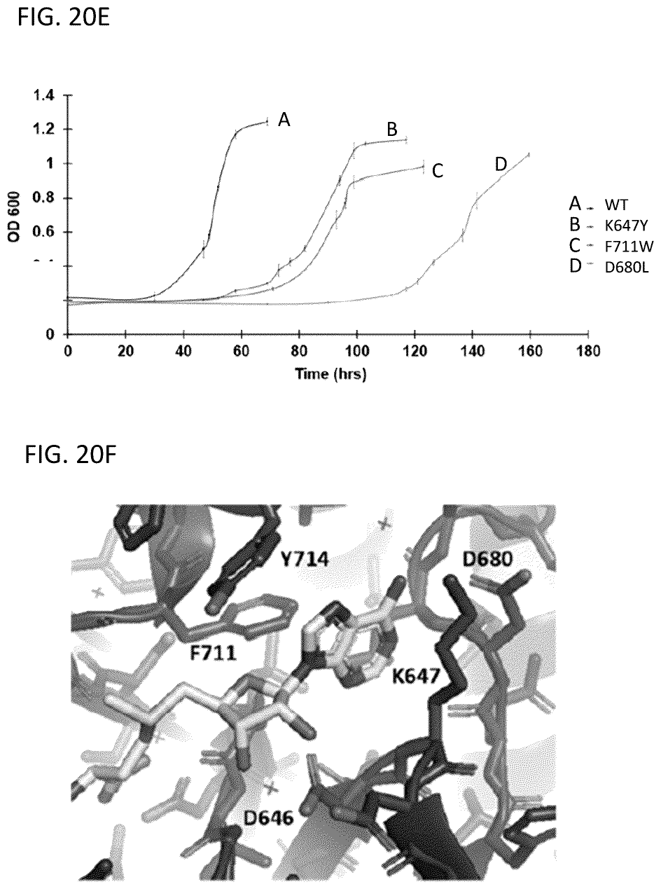

To expand the YeRCOM platform to animal viruses, African Swine Fever Virus (ASFV) was investigated. ASFV is a highly contagious virus that causes viral hemorrhagic disease in swine with high mortality rates. Due to its significant impact to the animal farming and the global food industry, efforts are directed to the development of novel vaccines, biotherapeutics and biocontainment strategies to control the spread of the disease. ASFV is a linear double stranded DNA virus in the Asfarviridae family. ASFV is classified as a nucleocytoplasmic large DNA viruses (NCLDV) because it replicates in the cytosol of mammalian cells, particularly monocyte/macrophage lineages of domestic and wild pigs³³. ASFV has a genome size of about 170-190 kb (depending on the strain) encoding 151-167 open reading frames with limited associated known or predicted functions.^{34,35} ASFV, which are double-stranded DNA viruses, are also predicted to encode RNA capping enzymes.³⁶ Given the importance of combating ASFV, it was investigated if a YeRCOM platform could be engineered for this pathogenic virus. The RNA capping enzyme from ASFV is predicted to have a similar domain organization as the D1 subunit of VACV, which is comprised of 3 domains with the following activities: RNA triphosphatase, guanylyl transferase³⁷, and N7-methyltransferase³³. However, the sequence similarity between the ASFV capping enzyme (NP868R) and the MTase domain of the VACV vD1 is only ˜20%³³. Further, ASFV NP868R does not require an allosteric activator for N7-MTase activity. Here, it is first shown that ASFV NP868R N7-MTase can recover S. cerevisiae abd1::kanMX4 growth by complementing S. cerevisiae abd1 function. Plasmid pAT15 was constructed encoding the WT ASFV NP868R gene fused to the human mce1 gene. As shown in FIG. 20A-20B, S. cerevisiae abd1::kanMX4 pMO1 pAT15 grew in the presence of 5-FOA after 48 hours, indicating successful RNA cap-0 N7-methylation complementation due to expression of ASFV NP868R. This functional complementation platform was named YeRCOM-ASFV.

Structure-based mutagenesis studies were performed to understand the role of various amino acids in the ASFV NP868R N7-MTase domain active site. As before, residues near the AdoMet binding site of ASFV NP868R were targeted. NP868R residue Y714A was previously shown to reduce ASFV N7-MTase activity in vitro to 14% of wild type activity but did not interfere with AdoMet binding³³. ASFV NP868R Y714A (pIJ22) was cloned and tested to recover growth of S. cerevisiae abd1::kanMX4 pMO1 haploid in vivo in presence of 5-FOA. It was found that this mutation was unable to recover yeast growth (FIGS. 20A-20B). However, when Y714L (pIJ23) was tested, it was found that incorporating a slightly larger amino acid residue at this position conferred yeast growth after 168 hours, indicating N7-MTase activity was attenuated significantly (FIGS. 20A-20B). Mutation F711A was previously shown to significantly reduce coenzyme AdoMet binding affinity³³, in alignment with the hypothesis that binding of AdoMet coenzyme is stabilized through hydrophobic interactions with this residue. It was found that mutation F711A was unable to recover S. cerevisiae abd1::kanMX4 haploid growth in vivo. However, when the ability of ASFV NP868R F711L (pIJ25) to recover S. cerevisiae abd1::kanMX4 haploid growth was tested, it was found that this mutation attenuated NP868R activity; growth was observed after 96 hours (FIGS. 20A-20C). Mutation F711W (pIJ26) was also tested to determine if incorporating a larger bicyclic aromatic amino acid would lead to higher activity than F711L, but less than WT NP868R. As shown in FIGS. 20A-20B, it was found this mutation attenuated NP868R activity, but it conferred faster growth than F711L. NP868R residue D646 stabilizes the ribose ring of AdoMet through the formation of several hydrogen bonds via its two side chains. This residue has been shown to be important for AdoMet binding³³. D646 is highly conserved in both cellular and poxvirus N7-MTases. D646A (pAT20) was transformed into S. cerevisiae abd1::kanMX4 pMO1 haploid and it was found that it did not recover growth in the presence of 5-FOA; in alignment with previous data demonstrating the importance of this aspartic acid (FIG. 20D). Residue D680 forms two hydrogen bonds with atoms N1 and N6 on the AdoMet adenine moiety. In theory, substituting a leucine for an aspartic acid at this position would lead to attenuation since leucine is the same chain length as aspartic acid, but the hydrogen bond interactions with AdoMet would be abolished. D680L (pIJ27) indeed led to attenuation of NP868R N7-MTase activity (FIGS. 20A-20D). The activity of D680E (pAT24) and D680N (pAT22) were also tested and it was found these mutations conferred equal activity to WT (FIG. 20D). Mutation D680A (pAT23) surprisingly had similar activity to ASFV WT NP868R activity in vivo (FIG. 20D). Therefore, it was hypothesized that the K647 residue might play a more important role in binding of AdoMet to the active site. K647 interacts with the adenine moiety of AdoMet, and it was thought that changing lysine to tyrosine may alter binding affinities of AdoMet and could lead to attenuation because this alteration would provide hydrophobic and polar interactions with AdoMet as well as a polar terminus. It was observed that indeed K647Y (pMO64) also led to attenuation of NP868R N7-MTase activity in vivo (FIG. 20A-20D). Lastly, position S604 interacts with the phosphate backbone of the RNA cap and previous data has shown that mutation S604A reduces N7-MTase activity to 45% of WT in vitro³³. It we observed that this observation translates to in vivo attenuation: S604A (pAT19) NP868R reduced S. cerevisiae abd1::kanMX4 haploid growth in comparison to WT NP868R (FIG. 20D).

Directed evolution experiments were also performed to identify attenuated variants of ASFV NP868R. A site saturated mutagenesis library at position F711 was created and 64 colonies were screened. Through this small screen, two attenuated hits were found: F711W and F711L. These attenuated variants had already been identified through the biochemical studies and rational design of attenuated variants, validating the rational design efforts.

### Example 11

### Development of YeRCOM-Human

This example illustrates engineering a yeast complementation platform for human RNA capping enzymes.

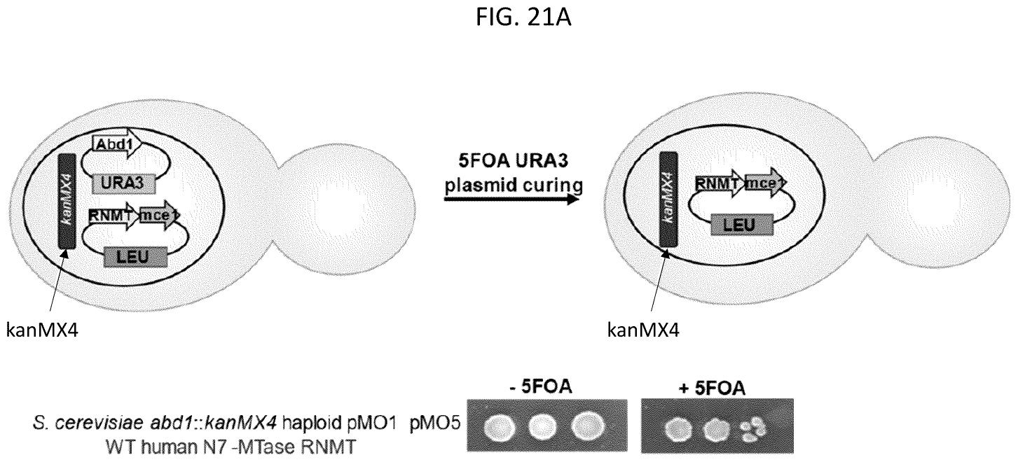

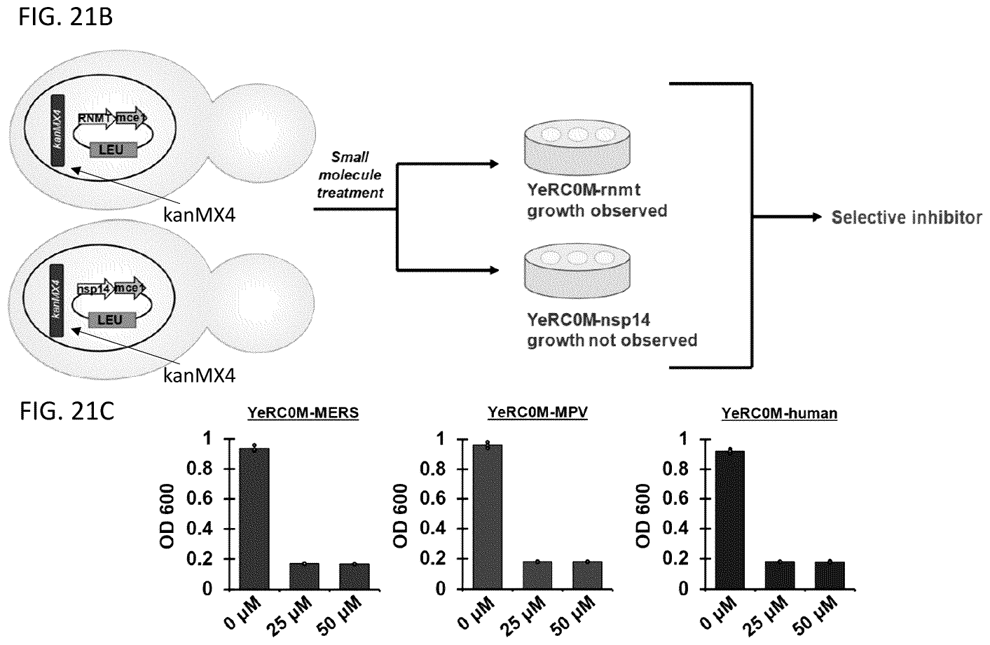

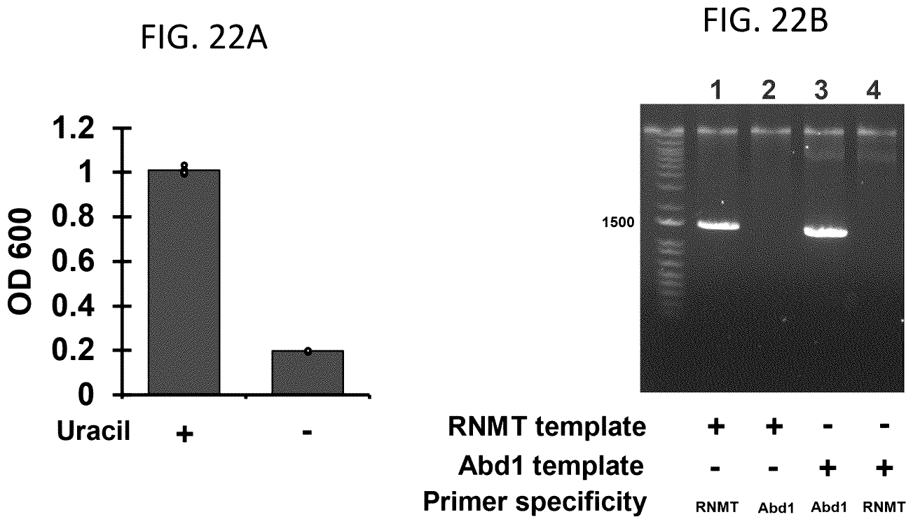

To make these platforms applicable to identify broad-spectrum inhibitors that selectively target viral RNA capping enzymes over human RNA capping enzymes, it was next sought to expand YeRCOM to human RNA capping enzymes.³⁸ First, it was tested if YeRCOM could be expanded to human RNMT; particularly, if S. cerevisiae abd1::kanMX4 pMO1 haploid could be complemented with human RNMT-MCE1 fusion.³⁹ Similar to pMO2, plasmid pMO5 was constructed which expressed human RNMT-MCE1 fusion, and pMOP5 was transformed into S. cerevisiae abd1::kanMX4 pMO1 haploid to generate S. cerevisiae abd1::kanMX4 pMO2 pMO5 haploid (FIG. 21A). The S. cerevisiae abd1::kanMX4 pMO1 pMO5 haploid was able to survive in the presence of 5-FOA indicating the that this yeast-based screen is compatible with human RNMT and can be used as a phenotypic platform to screen and optimize methyltransferase inhibitors that specifically target viral nsp14 over human RNMT. In order to develop starting strains containing only viral or human N7-MTase and not yeast N7-MTase, the pMO1 plasmid was cured from YeRCOM-MERS, YeRCOM-MPV and YeRCOM-ASFV by repeated culturing of these strains in presence of 5-FOA. The absence of pMO1 from corresponding strains was confirmed by growing them in absence of uracil and detecting no growth in absence of uracil, thereby suggesting that plasmid pMO1 containing ura3 marker were not present in each of these strains (FIG. 22A). The absence of plasmid pMO1 was confirmed by PCR analysis (FIG. 22B). These strains were designated as YeRCOM-MERS, YeRCOM-MPV, YeRCOM-ASFV and YeRCOM-human strains. All of these strains are compatible for high-throughput experimentation to identify selective inhibitors of corresponding viral enzymes over human enzymes.

### Example 12

### YeRCOM Phenotypic Screening

This example illustrates the utility of YeRCOM platforms for phenotypic screening approaches.

To demonstrate the utility of this phenotypic platform for screening and identifying nsp14 specific inhibitors), the activity of flunarizine dihydrochloride and tizanidine hydrochloride were assessed. Both of these compounds have been reported as selective inhibitors in a fluorescence-based screen against the SARS-CoV-2 nsp14 (⁴⁰ Based on this disclosure's observation regarding the functional relevance of the zinc finger domains of nsp14 for N7-MTase activity (FIGS. 17B-17C), the activity of a known zinc ejector, disulfiram was also evaluated. Disulfiram is an FDA-approved alcohol cessation drug and ongoing phase 2 clinical trial, ClinicalTrials.gov Identifier: NCT04485130, that has been shown to liberate zinc ions from isolated nsp14 in-vitro.⁴¹ As shown in FIG. 21C, non-selective inhibition was observed with disulfiram. These experiments suggest that YeRCOM can be readily used in combination with high-throughput screening, structure activity relationships and medicinal chemistry approaches to rapidly develop inhibitors of viral methyltransferase as potential antivirals.

### REFERENCES FOR EXAMPLE 7 THROUGH EXAMPLE 12

12. Ornelas, M. Y., Thomas, A. Y., Johnson Rosas, L. I., Scoville, R. O. & Mehta, A. P. Synthetic Platforms for Characterizing and Targeting of SARS-CoV-2 Genome Capping Enzymes. ACS Synth. Biol. 11, 3759-3771 (2022).
13. Chen, Y. et al. Functional screen reveals SARS coronavirus nonstructural protein nsp14 as a novel cap N7 methyltransferase. Proc. Natl. Acad. Sci. U.S.A 106, 3484-3489 (2009).
14. Ogando, N. S. et al. The Enzymatic Activity of the nsp14 Exoribonuclease Is Critical for Replication of MERS-CoV and SARS-CoV-2. J. Virol. 94, e01246-20 (2020).
15. Lauring, A. S., Jones, J. O. & Andino, R. Rationalizing the development of live attenuated virus vaccines. Nat. Biotechnol. 28, 573-579 (2010).
16. Yeh, M. T. et al. Engineering the Live-Attenuated Polio Vaccine to Prevent Reversion to Virulence. Cell Host Microbe 27, 736-751.e8 (2020).
17. Waterhouse, A. et al. SWISS-MODEL: homology modelling of protein structures and complexes. Nucleic Acids Res. 46, W296-W303 (2018).
18. Martin, S. & Moss, B. Modification of RNA by mRNA guanylyltransferase and mRNA (guanine-7-)methyltransferase from vaccinia virions. J. Biol. Chem. 250, 9330-9335 (1975).
19. Venkatesan, S., Gershowitz, A. & Moss, B. Modification of the 5′ end of mRNA. Association of RNA triphosphatase with the RNA guanylyltransferase-RNA (guanine-7-)methyltransferase complex from vaccinia virus. J. Biol. Chem. 255, 903-908 (1980).
20. Myette, J. R. & Niles, E. G. Domain Structure of the Vaccinia Virus mRNA Capping Enzyme: EXPRESSION IN ESCHERICHIA COLI OF A SUBDOMAIN POSSESSING THE RNA 5′-TRIPHOSPHATASE AND GUANYLYLTRANSFERASE ACTIVITIES AND A KINETIC COMPARISON TO THE FULL-SIZE ENZYME (*). J. Biol. Chem. 271, 11936-11944 (1996).
21. Gong, C. & Shuman, S. Mapping the active site of vaccinia virus RNA triphosphatase. Virology 309, 125-134 (2003).
22. Yu, L., Martins, A., Deng, L. & Shuman, S. Structure-function analysis of the triphosphatase component of vaccinia virus mRNA capping enzyme. J. Virol. 71, 9837-9843 (1997).
23. Cong, P. & Shuman, S. Methyltransferase and subunit association domains of vaccinia virus mRNA capping enzyme. J. Biol. Chem. 267, 16424-16429 (1992).
24. Higman, M. A., Bourgeois, N. & Niles, E. G. The vaccinia virus mRNA (guanine-N7-)-methyltransferase requires both subunits of the mRNA capping enzyme for activity. J. Biol. Chem. 267, 16430-16437 (1992).
25. Mao, X., Schwer, B. & Shuman, S. Yeast mRNA cap methyltransferase is a 50-kilodalton protein encoded by an essential gene. Mol. Cell. Biol. 15, 4167-4174 (1995).
26. Schwer, B. & Shuman, S. Genetic analysis of poxvirus mRNA cap methyltransferase: Suppression of conditional mutations in the stimulatory D12 subunit by second-site mutations in the catalytic D1 subunit. Virology 352, 145-156 (2006).
27. Kyrieleis, O. J. P., Chang, J., de la Pena, M., Shuman, S. & Cusack, S. Crystal Structure of Vaccinia Virus mRNA Capping Enzyme Provides Insights into the Mechanism and Evolution of the Capping Apparatus. Structure 22, 452-465 (2014).
28. Zheng, S. & Shuman, S. Structure-function analysis of vaccinia virus mRNA cap (guanine-N7) methyltransferase. RNA 14, 696-705 (2008).
29. Structural insights into the mechanism and evolution of the vaccinia virus mRNA cap N7 methyl-transferase. EMBO J. 26, 4913-4925 (2007).
30. Saha, N., Shuman, S. & Schwer, B. Yeast-Based Genetic System for Functional Analysis of Poxvirus mRNA Cap Methyltransferase. J. Virol. 77, 7300-7307 (2003).
31. Mao, X. & Shuman, S. Vaccinia Virus mRNA (Guanine-7-)Methyltransferase: Mutational Effects on Cap Methylation and AdoHcy-Dependent Photo-Cross-Linking of the Cap to the Methyl Acceptor Site. Biochemistry 35, 6900-6910 (1996).
32. Takata, Y. et al. Catalytic Mechanism of Glycine N-Methyltransferase, Biochemistry 42, 8394-8402 (2003).
33. Du, X., Gao, Z.-Q., Geng, Z., Dong, Y.-H. & Zhang, H. Structure and Biochemical Characteristic of the Methyltransferase (MTase) Domain of RNA Capping Enzyme from African Swine Fever Virus. J. Virol. JVI.02029-20 (2020) doi:10.1128/JVI.02029-20.
34. Ndlovu, S. et al. Genome sequences of three African swine fever viruses of genotypes I, III, and XXII from South Africa and Zambia, isolated from ornithodoros soft ticks. Microbiol. Resour. Announc. 9, e01376-19 (2020).
35. Bisimwa, P. N. et al. The first complete genome sequence of the African swine fever virus genotype X and serogroup 7 isolated in domestic pigs from the Democratic Republic of Congo. Virol. J. 18, 1-12 (2021).
36. Koonin, E. V. & Yutin, N. Origin and Evolution of Eukaryotic Large Nucleo-Cytoplasmic DNA Viruses. Intervirology 53, 284-292 (2010).
37. Pena, L., Yanez, R. J., Revilla, Y., Vinuela, E. & Salas, M. L. African Swine Fever Virus Guanylyltransferase. Virology 193, 319-328 (1993).
38. Tsukamoto, T., Shibagaki, Y., Niikura, Y. & Mizumoto, K. Cloning and Characterization of Three Human cDNAs Encoding mRNA (Guanine-7-)-methyltransferase, an mRNA Cap Methylase. Biochem. Biophys. Res. Commun. 251, 27-34 (1998).
39. Saha, N., Schwer, B. & Shuman, S. Characterization of Human, Schizosaccharomyces pombe, and Candida albicans mRNA Cap Methyltransferases and Complete Replacement of the Yeast Capping Apparatus by Mammalian Enzymes *. J. Biol. Chem. 274, 16553-16562 (1999).
40. Aouadi, W. et al. Toward the identification of viral cap-methyltransferase inhibitors by fluorescence screening assay. Antiviral Res. 144, 330-339 (2017).
41. Chen, T. et al. Synergistic Inhibition of SARS-CoV-2 Replication Using Disulfiram/Ebselen and Remdesivir. ACS Pharmacol. Transl. Sci. 4, 898-907 (2021).
45. Gibson, D. G. et al. Enzymatic assembly of DNA molecules up to several hundred kilobases. Nat. Methods 6, 343-345 (2009).

### Example 13

### Material and Methods for Results in Example 13 Through Example 15

This example illustrates the methods used for Example 13 to Example 15.

Growth media conditions. All Saccharomyces cerevisiae cultures were shaken aerobically at 30° C. and 250 rpm in synthetic defined medium containing 0.67% nitrogen base without amino acids, 2% glucose, 0.02 mg/mL lysine, 0.02 mg/mL methionine, 0.02 mg/mL histidine, and 0.1 mg/ml carbenicillin. Leucine 0.012 mg/mL was added when S. cerevisiae Δceg1 pAT4 haploid was cultured.

Plasmid curing procedure. Plasmids containing ura3 marker were cured by growing cultures in synthetic defined medium containing 1 mg/mL 5-fluoroorotic acid (5FOA) and uracil 0.02 mg/mL for 48 hours or by plating onto synthetic media agar plates containing 1 mg/mL 5-fluoroorotic acid (5FOA) and uracil 0.02 mg/mL and incubating at 30° C. for 48 hours.

Construction of plasmids. All single-stranded and double-stranded DNA oligonucleotide fragments were purchased from Integrated DNA Technologies (IDT). When noted, coding sequences were codon-optimized for S. cerevisiae expression using IDT codon optimization software. Table 12 provides SEQ ID NOs for plasmids. Table 13 contains the gblock sequences purchased from IDT. Table 14 contains the oligonucleotide sequences use to make each plasmid and mutations.

Library Screening. Libraries were generated using either NNK or MNN oligonucleotides purchased from IDT. The libraries were transformed into S. cerevisiae Aceg1 pAT4 haploid and screened using 5-fluororotic acid plasmid curing. Any colonies that showed reduce growth or no growth were further verified and sequenced.

pAT4: The gene corresponding to CEG1 was amplified from the gDNA of S. cerevisiae YPH500 using the oligonucleotides ROS42/ROS43. Previously made pMO1 which contains a ptpi1 promoter on pRS416 ura3 was linearized using the oligonucleotides AT14/AT16. CEG1 gene fragments were inserted into linearized pRS416 ura3 by Gibson assembly to afford pAT4.

pAM206: double-stranded WNV ns5 gblock codon optimized for S. cerevisiae was purchased from Integrated DNA Technologies (IDT). The gblock was inserted into a linearized backbone with oligonucleotides M093/M094 of pRS425 by Gibson Assembly. Mce1 was attached to the gblock by linearizing the backbone with oligonucleotides MO112/MO104. WNV ns5 was amplified using oligonucleotides MO111/MO103. The linearized fragments were Gibson together to afford pAM206.

pAM206-F24X Library: A site-specific library was generated on the 24 amino acid position of the WNV ns5 plasmid (pAM206). An MNN oligonucleotide, AT193, and AT195 were used to amplify the backbone of the plasmid while AT192/AT194 were used to amplify the remaining gene. The two linearized fragments were Gibson together to afford the F24X library.

pAM206-S150X Library: A site-specific library was generated on the 150 amino acid position of the WNV ns5 gene (pAM206). An NNK oligonucleotide, AT197, and AT194 were used to amplify the insert region of the plasmid while AT201/AT195 were used to amplify the remaining backbone of the plasmid. The two linearized fragments were Gibson together to afford the S150X library.

pAT8: double-stranded nsp12 gblock codon optimized for S. cerevisiae was purchased from Integrated DNA Technologies (IDT). The gblock was first cloned into puc19. The gene for nsp12 was amplified with oligonucleotides AT60/AT63. pRS425 backbone was linearized with oligonucleotides AT62/AT61.

pAT13: mce1 was removed from pAM206 by linearizing the plasmid with oligonucleotides AT101/AT102. The linearized fragment was ligated by KLD reaction to afford pAT37.

pMO72: K73A point mutation K73A was introduced into pAT8 by linearizing pAT8 using oligonucleotides M0414/M0415. The linearized fragment was ligated by KLD reaction to afford pMO72.

pMO73: point mutation D218A was introduced into pAT8 by linearizing pAT8 using oligonucleotides M0416/M0417. The linearized fragment was ligated by KLD reaction to afford pMO73.

pMO74: point mutation D760A was introduced into pAT8 by linearizing pAT8 using oligonucleotides M0418/M0419. The linearized fragment was ligated by KLD reaction to afford pMO74.

pMO93: point mutation V30A was introduced into pAT8 by linearizing pAT8 using oligonucleotides M0469/M0466. The linearized fragment was ligated by KLD reaction to afford pMO93.

pMO89: point mutation R33A was introduced into pAT8 by linearizing pAT8 using oligonucleotides M0465/M0466. The linearized fragment was ligated by KLD reaction to afford pMO89.

pMO90: point mutation R33L was introduced into pAT8 by linearizing pAT8 using oligonucleotides M0468/M0466. The linearized fragment was ligated by KLD reaction to afford pMO90.

pMO91: point mutation R33K was introduced into pAT8 by linearizing pAT8 using oligonucleotides M0467/M0466. The linearized fragment was ligated by KLD reaction to afford pMO91.

pMO86: point mutation R55A was introduced into pAT8 by linearizing pAT8 using oligonucleotides M0461/M0462. The linearized fragment was ligated by KLD reaction to afford pMO86.

pMO87: point mutation R55L was introduced into pAT8 by linearizing pAT8 using oligonucleotides M0464/M0462. The linearized fragment was ligated by KLD reaction to afford pMO87.

pMO88: point mutation R55K was introduced into pAT8 by linearizing pAT8 using oligonucleotides M0463/M0462. The linearized fragment was ligated by KLD reaction to afford pMO88.

pMO83: point mutation K50A was introduced into pAT8 by linearizing pAT8 using oligonucleotides M0453/M0454. The linearized fragment was ligated by KLD reaction to afford pMO83.

pMO85: point mutation C53A was introduced into pAT8 by linearizing pAT8 using oligonucleotides M0459/M0460. The linearized fragment was ligated by KLD reaction to afford pMO85.

pMO82: point mutation R116A was introduced into pAT8 by linearizing pAT8 using oligonucleotides M0451/MO452. The linearized fragment was ligated by KLD reaction to afford pMO82.

pMO92: point mutation V71A was introduced into pAT8 by linearizing pAT8 using oligonucleotides M0470/M0471. The linearized fragment was ligated by KLD reaction to afford pMO92.

pMO84: point mutation N209A was introduced into pAT8 by linearizing pAT8 using oligonucleotides M04571M0458. The linearized fragment was ligated by KLD reaction to afford pMO84.

pMO94: point mutation Li 19A was introduced into pAT8 by linearizing pAT8 using oligonucleotides M04551M0456. The linearized fragment was ligated by KLD reaction to afford pMO94.

pMO96: point mutation Y217F was introduced into pAT8 by linearizing pAT8 using oligonucleotides M04731M0474. The linearized fragment was ligated by KLD reaction to afford pMO96.

TABLE 12
Correspondence between plasmids and sequence numbering.

| Plasmid | SEQ ID NO |
|---|---|
| pAT4 | SEQ ID NO: 28 |
| pAT8 | SEQ ID NO: 26 |
| pAM206 | SEQ ID NO: 241 |
| pAT13 | SEQ ID NO: 27 |
| pAT37 | SEQ ID NO: 242 |
| pMO35 | SEQ ID NO: 243 |
| pMO37 | SEQ ID NO: 244 |

Plasmid pAT37 contains a WNV ns3 expression cassette operably under a pTPI1 promoter. Plasmid pMO35 which contains a cet1 expression cassette under a pTPI1 promoter with ura3 gene as the selection marker. Plasmid pMO37 contains a contains a cet1 expression cassette under a pTPI1 promoter and a cet1 expression cassette under a pTPI1 promoter with ura3 gene as the selection marker.

TABLE 13
Sequences of gBlocks used in the construction of plasmids.

| | SEQ | |
|---|---|---|
| gblock | ID NO | Sequence |
| mce1 | 245 | GAACCAGGGTCAAGTGCATCCTTTAGTAAAAGGAGAAAAGAGCGGTTAAAACTGGGA |
| | | GCTATTTTCTTGGAAGGCATAACTGTTAAAGGTGTAACTCAAGTAACAACTCAACCC |
| | | AAGTTAGGAGAGGTGCAGCAGAAATGTCATCAGTTCTGTGGCTGGGAAGGGTCTGGG |
| | | TTCCCTGGAGCACAGCCTGTCTCCATGGACAAGCAAAATATTAGACTTTTAGAGCAG |
| | | AAGCCTTATAAAGTAAGCTGGAAAGCAGACGGCACTCGTTACATGATGTTGATTGAT |
| | | GGCACAAATGAAGTTTTTATGATTGACAGAGATAATTCCGTTTTTCATGTTTCAAAT |
| | | CTGGAATTTCCATTTCGTAAAGATCTTCGCATGCATTTATCAAATACTCTTTTGGAT |
| | | GGGGAAATGATCATTGACAAAGTAAATGGACAGGCCGTCCCAAGATATTTGATCTAT |
| | | GACATAATTAAGTTCAATGCACAACCAGTTGGAGACTGTGATTTTAATATTCGTCTA |
| | | CAGTGTATTGAACGTGAAATTATAAGTCCACGACATGAAAAAATGAAGACTGGACTC |
| | | ATTGACAAAACACAGGAACCATTTAGTGTCAGACCTAAACAATTTTTTGACATCAAT |
| | | ATTTCAAGAAAGCTTCTGGAAGGAAATTTTGCCAAAGAAGTCAGCCATGAAATGGAT |
| | | GGACTTATTTTTCAGCCTATTGGAAAATACAAGCCTGGTCGATGTGATGACATTTTG |
| | | AAATGGAAACCTCCCAGTCTAAACTCTGTGGATTTTCGACTTAAGATAACAAGAATG |
| | | GGAGGAGAAGGGTTGCTTCCACAGAATGTTGGCCTTCTCTATGTTGGAGGTTATGAA |
| | | AGACCCTTTGCACAAATCAAGGTGACAAAAGAACTAAAACAGTATGACAACAAAATT |
| | | ATAGAATGCAAATTTGAGAACAATAGCTGGGTCTTCATGAGACAGAGGATAGACAAA |
| | | AGTTTCCCAAATGCCTACAACACAGCCATGGCTGTGTGCAATAGCATCTCGAACCCT |
| | | GTCACCAAGGAGATGCTGTTTGAATTCATTGACAGATGTGCAGCAGCCGCCCAGGGA |
| | | CAGAAGCGGAAGTATCCCCTGGACCCTGACACGGAGCTCATGCCGCCCCCACCGCCC |
| | | AAAAGACTGCATCGGCCAACC |
| SARS- | 246 | ATGAGTGCAGATGCTCAATCTTTCCTAAATAGGGTGTGTGGTGTGAGCGCCGCTAGA |
| CoV-2 | | CTTACACCTTGCGGGACGGGGACCTCTACGGATGTCGTGTATAGAGCGTTCGATATT |
| nsp12 | | TATAATGATAAAGTCGCAGGTTTTGCTAAGTTTCTGAAAACAAATTGTTGTAGGTTC |
| | | CAGGAGAAAGATGAAGATGACAACTTAATTGATTCATACTTCGTTGTTAAAAGGCAT |
| | | ACCTTCTCTAATTACCAACATGAAGAAACAATTTATAATTTGTTGAAGGACTGTCCA |
| | | GCGGTCGCAAAACACGATTTCTTTAAATTCAGGATAGATGGAGATATGGTACCACAC |
| | | ATCTCTAGACAGAGGCTTACGAAATATACAATGGCCGATCTAGTCTATGCATTACGT |
| | | CACTTCGATGAGGGAAACTGTGACACATTAAAGGAAATCTTAGTAACCTATAACTGT |
| | | TGCGATGATGATTATTTTAACAAGAAGGATTGGTACGATTTCGTTGAAAATCCGGAT |
| | | ATTTTGCGTGTTTACGCAAATTTGGGTGAAAGAGTTAGACAAGCCTTATTAAAGACC |
| | | GTCCAATTTTGCGACGCAATGCGAAACGCGGGTATTGTTGGCGTACTAACACTCGAT |
| | | AACCAAGATTTGAACGGTAATTGGTACGACTTTGGAGATTTCATTCAAACTACCCCA |
| | | GGTAGTGGTGTTCCAGTCGTAGATTCATATTACAGTCTATTGATGCCTATTTTAACC |
| | | CTTACAAGAGCACTCACTGCCGAAAGTCATGTTGATACAGACCTGACTAAGCCATAC |
| | | ATTAAATGGGATTTATTGAAGTATGACTTCACTGAAGAGAGGCTAAAACTTTTCGAC |
| | | AGATACTTTAAATATTGGGACCAAACTTACCATCCTAATTGTGTAAACTGTTTGGAT |
| | | GACAGATGTATATTGCACTGTGCCAACTTCAATGTCCTTTTTTCTACAGTCTTTCCC |
| | | CCCACAAGCTTTGGCCCACTGGTGCGCAAAATATTTGTTGACGGTGTGCCTTTCGTT |
| | | GTCTCCACAGGTTATCACTTCAGAGAATTAGGAGTAGTACATAATCAAGATGTGAAC |
| | | TTGCACTCATCTCGGTTATCGTTCAAAGAGTTACTAGTTTATGCTGCTGATCCTGCC |
| | | ATGCATGCAGCTTCTGGCAATCTATTATTGGATAAGCGTACTACTTGCTTTTCAGTT |
| | | GCCGCACTGACTAATAATGTTGCTTTTCAGACAGTCAAACCAGGCAATTTTAACAAG |
| | | GATTTTTATGACTTTGCTGTATCTAAAGGCTTCTTCAAGGAGGGTTCCTCCGTGGAA |
| | | TTAAAGCATTTCTTTTTTGCCCAGGATGGTAACGCCGCAATTTCTGACTATGATTAT |
| | | TATAGATACAATTTACCTACCATGTGCGACATCAGACAATTATTGTTTGTAGTTGAG |
| | | GTCGTGGATAAATACTTTGATTGTTACGATGGGGGCTGCATCAACGCTAATCAGGTC |
| | | ATAGTTAATAATTTAGACAAATCCGCGGGGTTTCCCTTTAATAAGTGGGGTAAAGCA |
| | | CGTCTTTACTATGATTCGATGTCTTATGAAGATCAAGATGCTTTGTTTGCATATACA |
| | | AAGCGTAATGTTATACCTACTATAACGCAAATGAACTTGAAATACGCTATTTCTGCC |
| | | AAAAATCGAGCTAGGACAGTTGCAGGTGTTTCAATATGCTCCACCATGACAAACCGC |
| | | CAATTTCATCAAAAACTGTTAAAGAGCATAGCGGCAACAAGAGGAGCCACGGTTGTG |
| | | ATCGGTACTTCTAAATTCTATGGTGGATGGCATAATATGTTGAAAACGGTGTACTCG |
| | | GATGTTGAAAATCCACATCTAATGGGCTGGGATTACCCTAAATGCGACAGGGCGATG |
| | | CCAAACATGTTGAGAATCATGGCCAGCTTAGTTCTTGCTAGAAAACATACGACTTGT |
| | | TGTAGCCTCTCACATAGATTTTATCGGCTCGCAAATGAATGCGCCCAAGTGTTGTCG |
| | | GAAATGGTTATGTGTGGGGGTTCATTATATGTAAAGCCAGGAGGCACGTCCAGTGGT |
| | | GATGCTACTACAGCTTATGCGAATTCTGTTTTTAATATCTGTCAGGCTGTAACTGCA |
| | | AATGTTAACGCACTATTAAGTACAGACGGTAATAAAATTGCTGACAAGTACGTCAGG |
| | | AACCTTCAGCATAGACTGTACGAATGTTTATATCGTAACCGAGACGTTGACACGGAC |
| | | TTTGTCAACGAGTTTTATGCTTATCTGAGAAAACACTTTTCAATGATGATTCTATCG |
| | | GACGATGCTGTAGTTTGTTTTAACTCCACTTATGCTAGTCAAGGCTTAGTAGCTTCA |
| | | ATCAAGAACTTTAAATCCGTGTTGTATTACCAAAACAATGTTTTTATGTCAGAAGCT |
| | | AAGTGCTGGACCGAAACTGACTTGACTAAAGGACCACATGAGTTCTGTTCTCAGCAC |
| | | ACCATGCTCGTAAAACAAGGAGACGATTACGTCTACTTACCCTATCCTGATCCAAGT |
| | | AGAATTCTTGGTGCAGGTTGCTTCGTGGATGACATAGTAAAAACTGACGGAACTCTT |
| | | ATGATCGAAAGATTCGTTTCACTGGCTATTGATGCTTACCCGCTGACCAAGCATCCG |
| | | AATCAGGAGTACGCCGATGTTTTTCACTTGTACCTACAATATATTAGAAAGTTGCAC |
| | | GATGAATTGACTGGTCATATGCTTGATATGTACAGCGTCATGTTGACTAATGATAAT |
| | | ACCAGTAGATATTGGGAACCGGAATTTTACGAAGCAATGTATACTCCACATACCGTG |
| | | TTGCAA |
| WNV | 247 | CATAAACTAAAAGGTGGAGCTAAGGGGAGGACATTGGGGGAAGTTTGGAAGGAGAGA |
| ns5 | | TTAAACCAAATGACCAAAGAGGAATTTACCCGTTACAGAAAAGAAGCCATCATCGAA |
| | | GTAGACCGTTCAGCTGCCAAGCATGCTAGAAAGGAGGGCAATGTAACGGGGGGCCAT |
| | | CCAGTGAGCAGAGGGACGGCTAAGTTGCGTTGGCTTGTTGAAAGAAGATTTCTTGAA |
| | | CCAGTGGGGAAAGTCATTGACTTGGGTTGTGGCAGGGGAGGTTGGTGCTACTACATG |
| | | GCGACACAGAAACGTGTCCAGGAAGTAAGGGGATATACTAAAGGCGGACCCGGACAC |
| | | GAAGAACCTCAGCTGGTTCAATCCTATGGCTGGAACATCGTCACGATGAAGTCAGGG |
| | | GTTGACGTGTTCTATCGTCCGTCAGAATGTTGCGACACCTTACTGTGTGATATAGGG |
| | | GAGAGCTCAAGCTCTGCGGAGGTGGAGGAGCATAGAACGATCCGTGTTCTGGAGATG |
| | | GTCGAGGATTGGTTGCATCGTGGCCCAAGAGAGTTCTGCGTCAAGGTCCTTTGTCCG |
| | | TACATGCCTAAGGTTATAGAAAAGATGGAGCTGTTACAACGTAGATATGGGGGCGGT |
| | | TTGGTAAGAAATCCCCTTAGCAGAAACAGCACGCACGAAATGTACTGGGTCAGCAGA |
| | | GCTTCAGGTAATGTGGTGCATAGCGTAAACATGACTAGCCAGGTACTGTTAGGCCGT |
| | | ATGGAGAAAAGGACATGGAAGGGACCGCAATATGAAGAGGATGTGAACCTTGGTTCT |
| | | GGAACAAGGGCGGTTGGTAAACCCCTGCTGAATTCTGACACTAGTAAAATTAAGAAC |
| | | CGTATAGAGAGGTTACGTAGGGAGTACAGCTCTACGTGGCACCATGATGAAAATCAC |
| | | GCCTGATTCA |

TABLE 14
DNA oligos used in the construction of plasmids.

| | SEQ | |
|---|---|---|
| | ID | |
| Name | NO | Sequence |
| ROS42 | 248 | ATGGTTTTAGCAATGGAAAGTAGAGTG |
| ROS43 | 249 | CTAATCCGACCAATCATCCTCGT |
| AT14 | 250 | CATTGCTAAAACCATTTTTAGTTTATGTATGTGTT |
| | | TTTTGTAGTTATAGATTT |
| AT16 | 251 | GTCGGATTAGTGTAAGCGGATGCCGGG |
| AT60 | 252 | GACCCTGGTTCTTGCAACACGGTATGTGGAGT |
| AT61 | 253 | CCGTGTTGCAAGAACCAGGGTCAAGTGCATCC |
| AT62 | 254 | AGCATCTGCACTCATTTTTAGTTTATGTATGTGTT |
| | | TTTTGTAGTT |
| AT63 | 255 | ATACATAAACTAAAAATGAGTGCAGATGCTCAATC |
| | | TT |
| AT101 | 256 | GCCTGATTCAAGAAATATCTTGACCGCA |
| AT102 | 257 | GTGATTTTCATCATGGTGCCACGTAGAG |
| AT192 | 258 | ACCCGTTACAGAAAAGAAGCCA |
| AT193 | 259 | TGGCTTCTTTTCTGTAACGGGTMNNTTCCTCTTTG |
| | | GTC |
| AT194 | 260 | GTGATTTTCATCATGGTGCCAC |
| AT195 | 261 | GTGGCACCATGATGAAAATCAC |
| AT197 | 262 | ACCTTACTGTGTGATATAGGGGAGNNKTCAAGCTC |
| | | TGCGG |
| AT201 | 263 | CTCCCCTATATCACACAGTAAGGTGTCG |
| MO93 | 264 | AAATCACTAAGCCTGATTCAAGAAATATCTTGACC |
| MO94 | 265 | TAGCTCCACCTTTTAGTTTATGTATGTGTTTTTTG |
| | | TAGTTATAGA |
| MO103 | 266 | ACCCTGGTTCGTGATTTTCATCATGGTGCC |
| MO104 | 267 | TGAAAATCACGAACCAGGGTCAAGTGC |
| MO111 | 268 | TAAACTAAAAATGGGTGGAGCTAAGGGG |
| MO112 | 269 | TAGCTCCACCCATTTTTAGTTTATGTATGTGTTTT |
| | | TTGTAGTTATAGA |
| MO469 | 270 | CTACGGATGTCGCGTATAGAGCGTTCG |
| MO466 | 271 | AGGTCCCCGTCCCGCAAGGTGTAAG |
| MO465 | 272 | CTACGGATGTCGTGTATGCAGCGTTCGATATTTAT |
| MO468 | 273 | CTACGGATGTCGTGTATTTAGCGTTCGATATTTAT |
| MO467 | 274 | CTACGGATGTCGTGTATAAAGCGTTCGATATTTAT |
| MO459 | 275 | GTTTCTGAAAACAAATGCTTGTAGGTTCCAGGAG |
| MO460 | 276 | TTAGCAAAACCTGCGACTTTATCATTATAAATATC |
| MO453 | 277 | GTTTTGCTAAGTTTCTGGCAACAAATTGTTGTAG |
| MO454 | 278 | CTGCGACTTTATCATTATAAATATCGAACG |
| MO461 | 279 | GAAAACAAATTGTTGTGCGTTCCAGGAGAAAGATG |
| MO462 | 280 | AGAAACTTAGCAAAACCTGCGACTT |
| MO463 | 281 | GAAAACAAATTGTTGTAAGTTCCAGGAGAAAGATG |
| MO464 | 282 | GAAAACAAATTGTTGTTTGTTCCAGGAGAAAGATG |
| MO470 | 283 | AATTGATTCATACTTCGCTGTTAAAAGGCATAC |
| MO471 | 284 | AAGTTGTCATCTTCATCTTTCTCCTGGAAC |

In the sequences of Table 8, the character S can include G or C. The character V can include A, C, or G. The character B can include G, C, or T. The character N can include A, C, G, or T. The character K can include G or T. The character M can include A or C.

### Example 14

### Yeast Platform for RNA Cap 0 N7 Guanylyltransferase: WNV Ns5

This example illustrates the ability of WNV ns5 to catalyze the formation of the guanine cap on yeast mRNAs.

Commercially available S. cerevisiae strain (Horizon Discovery Accession #: YGL130W was used to engineer this complementation platform as a starting yeast strain. This is a diploid strain of S. cerevisiae where ceg1 gene on one chromosome is replaced by a kanamycin cassette (referred to as S. cerevisiae ceg1::kanMX4 diploid). The gene product corresponding to ceg1 is the yeast mRNA Cap 0 guanylyltransferase, which is an essential enzyme for the viability of S. cerevisiae.

Ceg1p was complemented in ceg1::kanMX4 haploid yeast via plasmid pAT4 which contains a ceg1 expression cassette under a pTPI1 promoter with ura3 gene as the selection marker. Since pAT4 has a ura3 marker, it can be cured in presence of 5-fluorooratic acid (5-FOA) which is converted to 5-fluorouracil which is a toxic molecule. In S. cerevisiae ceg1::kanMX4 pAT4 haploid strains, exposure to 5-FOA is expected to result in loss of cell viability due to curing of pAT4 resulting in the lack of complete lack of nuclear and plasmid-based ceg1 expression which is lethal to S. cerevisiae strains.

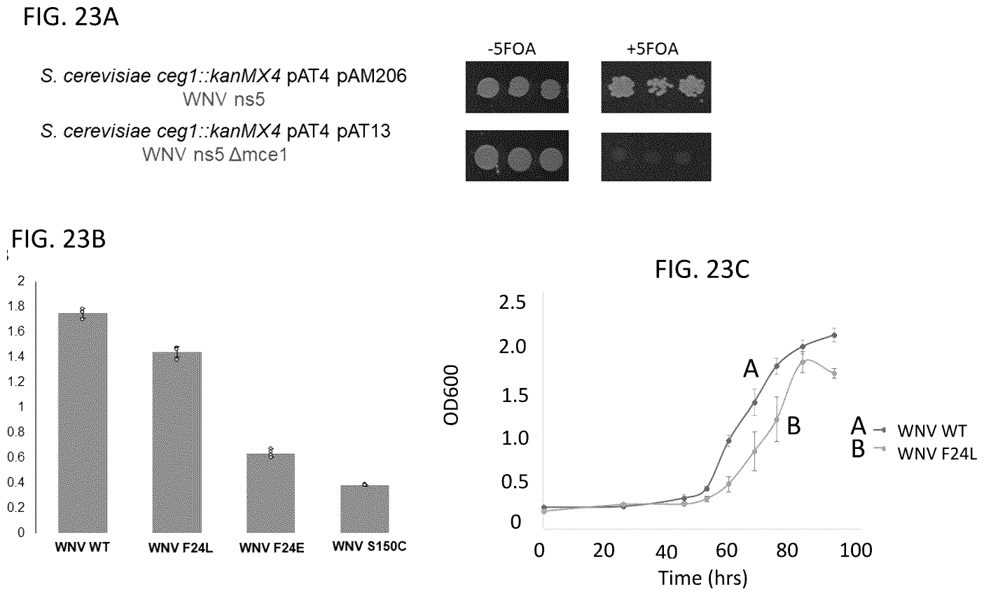

It was investigated whether S. cerevisiae ceg1::kanMX4 haploid ceg1 deficiency can be rescued by the expression of West Nile Virus (WNV) guanylyltransferase ns5. A fusion between WNV ns5 sequence and the human mce1 gene was generated, and incorporated into a plasmid with PTPL promoter, and leu2 marker (plasmid name: pM206). Another plasmid, similar to pM206, having WNV ns5, a PTPL promoter, and leu2 marker, but lacking human mce1, was also constructed (plasmid name: pAT13). Both pM206 and pAT13 were transformed into S. cerevisiae ceg1::kanMX4 pAT4 haploid to generate S. cerevisiae ceg1::kanMX4 pAT4 pM206 haploid and S. cerevisiae ceg1::kanMX4 pAT4 pAT13 haploid respectively. S. cerevisiae ceg1::kanMX4 pAT4 pM206 haploid and S. cerevisiae ceg1::kanMX4 pAT4 pAT13 haploid were then each grown in the presence and absence of 5-FOA. As depicted in FIG. 23A, yeast with the ceg1::kanMX4 pAT4 pM206 plasmid was able to survive in the presence of 5-FOA. However, as FIG. 23A also shows, yeast with the ceg1::kanMX4 pAT4 pAT13 had severely impaired growth.

Directed evolution experiments were performed on WNV ns5 to identify attenuated variants. Site saturated mutagenesis libraries were made at positions F24 and S150 by generating a pAM206-F24X Library and a pAM206-S150X Library. Colonies screened from these libraries resulted in F24L, F24E, and S150C being identified as attenuating mutations. The growth rates of F24L, F24E were characterized in liquid selection medium in the presence of 5-FOA (FIGS. 23B, 23C). The attenuated variants of WNV ns5 identified in this study can be used in the development of live attenuated vaccine candidates for WNV.

### Example 15

### Yeast Platform for RNA Cap 0 N7 Guanylyltransferase: SARS-CoV-2 Nsp12

This example illustrates the ability of SARS-CoV-2 nsp12 to catalyze the formation of the guanine cap on yeast mRNAs.

It was investigated whether S. cerevisiae ceg1::kanMX4 haploid ceg1 deficiency can be rescued by the expression of SARS-CoV-2 guanylyltransferase nsp12.

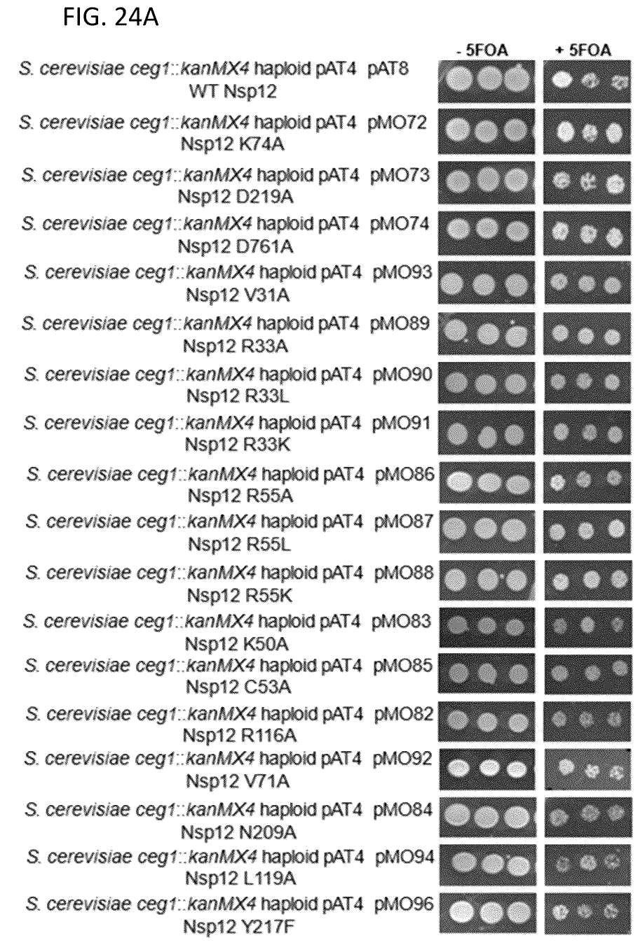

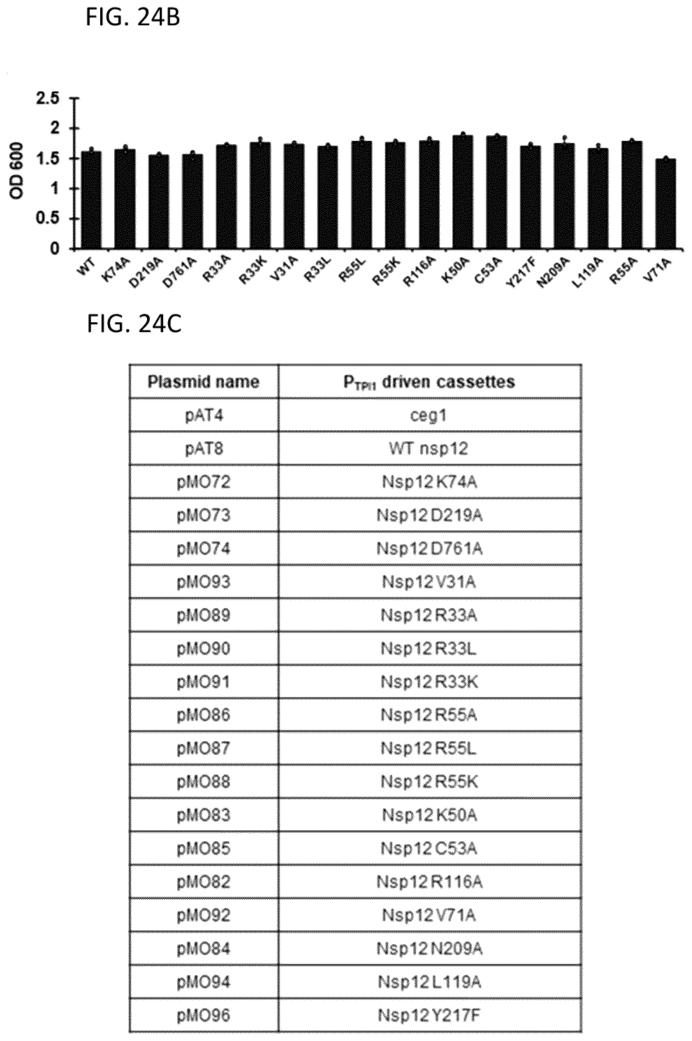

As in example 14, Ceg1p was complemented in ceg1::kanMX4 haploid yeast via curable plasmid pAT4 which contains a ceg1 expression cassette under a pTPI1 promoter with ura3 gene as the selection marker. A fusion between SARS-CoV-2 nsp12 sequence and the human mce1 gene was generated, and incorporated into a plasmid with P_{TPI1} promoter, and leu2 marker (plasmid name: pAT8). pAT8 was transformed into S. cerevisiae ceg1::kanMX4 pAT4 haploid to generate S. cerevisiae ceg1::kanMX4 pAT4 pT8 haploid, which was able to survive in the presence of 5-FOA (FIG. 24A).

Point mutations K73A (pMO72), D218A (pMO73), D760A (pMO74), V30A (pMO93), R33A (pMO89), R33L (pMO90), R33K (pMO91), R55A (pMO86), R55L (pMO87), R55K (pMO88), K50A (pMO83), C53A (pMO85), R116A (pMO82), V71A (pMO92), N209A (pMO84), L119A (pMO94), and Y217F (pMO96) were introduced into SARS-CoV-2 nsp12 and transformed into S. cerevisiae ceg1::kanMX4 pAT4 haploid. Growth was characterized in the presence of 5-FOA (FIG. 24A-24C).

It will be apparent that the precise details of the methods or compositions described may be varied or modified without departing from the spirit of the described aspects of the disclosure. We claim all such modifications and variations that fall within the scope and spirit of the claims below.

1. An isolated non-native yeast, comprising:
a) a genetically inactivated Abd1 gene and a heterologous methyltransferase;
b) a genetically inactivated Ceg1p gene and a heterologous guanylyltransferase; or
c) a genetically inactivated Cet1p and a heterologous RNA triphosphatase.

2. The isolated non-native yeast of claim 1:
a) wherein the heterologous methyltransferase is encoded by a plasmid and wherein the heterologous methyltransferase is a full-length methyltransferase, a functional methyltransferase fragment, or a codon optimized methyltransferase for expression in yeast;
b) wherein the heterologous guanylyltransferase is encoded by a plasmid and wherein the heterologous guanylyltransferase is a full length guanylyltransferase, a functional guanylyltransferase fragment, or a codon optimized guanylyltransferase for expression in yeast; or
c) wherein the heterologous RNA triphosphatase is encoded by a plasmid and wherein the heterologous RNA triphosphatase is a full length RNA triphosphatase, a functional RNA triphosphatase fragment, or a codon optimized RNA triphosphatase for expression in yeast.

3. The isolated non-native yeast of claim 2:
a) wherein the plasmid encoding the heterologous methyltransferase comprises at least 80%, at least 85%, at least 90%, at least 95%, at least 96%, at least 97%, at least 98%, at least 99%, or 100% sequence identity to SEQ ID NO: 16, 17, 18, 19, 21, 22, or 23;
b) wherein the plasmid encoding the heterologous guanylyltransferase comprises at least 80%, at least 85%, at least 90%, at least 95%, at least 96%, at least 97%, at least 98%, at least 99%, or 100% sequence identity to SEQ ID NOs: 17, 18, 19, 26 or 27;
c) wherein the plasmid encoding the heterologous RNA triphosphatase comprises at least 80%, at least 85%, at least 90%, at least 95%, at least 96%, at least 97%, at least 98%, at least 99%, or 100% sequence identity to SEQ ID NOs: 17, 18, or 19.

4. The isolated non-native yeast of claim 1:
a) wherein the heterologous methyltransferase is fused to an RNA polymerase complex targeting gene;
b) wherein the heterologous guanylyltransferase is fused to an RNA polymerase complex targeting gene; or
c) wherein the heterologous RNA triphosphatase is fused to an RNA polymerase complex targeting gene.

5. The isolated non-native yeast of claim 1:
a) further comprising an Adb1 plasmid comprising a second Abd1 gene operably linked to a promoter;
b) further comprising a Ceg1p plasmid comprising a second Ceg1p gene operably linked to a promoter; or
c) further comprising a Cet1p plasmid comprising a second Cet1p gene operably linked to a promoter.

6. The isolated non-native yeast of claim 5:
a) wherein the Adb1 plasmid further comprises a yeast Ura3 gene; and wherein the Adb1 plasmid is curable;
b) wherein the Ceg1p plasmid further comprises a yeast Ura3 gene; and wherein the Ceg1p plasmid is curable; or
c) wherein the Cet1p plasmid further comprises a yeast Ura3 gene; and wherein the Cet1p plasmid is curable.

7. The isolated non-native yeast of claim 1, wherein the heterologous methyltransferase comprises nsp14, D1, D12, D1 and D12, NP868R, ns5, or mRNA cap methyltransferase (RNMT).

8. The isolated non-native yeast of claim 1, wherein the heterologous guanylyltransferase comprises nsp12, ns5, NP868R, D1, D12, D1 and D12, or MCE1.

9. The isolated non-native yeast of claim 1, wherein the heterologous RNA triphosphatase comprises nsp9, NP868R, D1, D12, D1 and D12, or MCE1.

10. The isolated non-native yeast of claim 1, wherein the isolated non-native yeast is haploid.

11. A composition comprising the isolated non-native yeast of claim 1 and a carrier;
wherein the carrier comprises a solid or liquid medium; and
wherein the composition further comprises 5-FOA.

12. A kit comprising:
a) a plasmid comprising a methyltransferase gene, wherein incorporating the plasmid into a yeast comprising genetically inactivated Abd1 results in the isolated non-native yeast of claim 1;
b) a plasmid comprising a guanylyltransferase gene, wherein incorporating the plasmid into a yeast comprising genetically inactivated Ceg1p results in the isolated non-native yeast of claim 1; or
c) a plasmid comprising an RNA triphosphatase gene, wherein incorporating the plasmid into a yeast comprising genetically inactivated Cet1p results in the isolated non-native yeast of claim 1; and
further comprising the yeast comprising genetically inactivated Abd1, the yeast comprising genetically inactivated Ceg1p, or the yeast comprising genetically inactivated Cet1p.

13. A method of identifying one or more attenuating mutations in the heterologous viral methyltransferase; heterologous viral guanylyltransferase; or heterologous viral RNA triphosphatase of the isolated non-native yeast of claim 1 comprising:
altering one or more amino acid residues in the heterologous viral methyltransferase;
heterologous viral guanylyltransferase; or heterologous viral RNA triphosphatase of the isolated non-native yeast to generate an experimental strain;
culturing the experimental strain;
measuring growth of the experimental strain;
comparing the growth of the experimental strain to a control; and
wherein the experimental strain growing slower than the control indicates the presence of an attenuating mutation in the experimental strain.

14. A method of screening for methyltransferase, guanylyltransferase, or RNA triphosphatase inhibitors using the isolated non-native yeast of claim 1, comprising:
culturing a first strain of the isolated non-native yeast in media comprising at least one screening compound;
measuring growth of the first strain;
comparing the growth of the first strain to a control;
wherein the first strain growing slower than the control indicates the at least one screening compound is a methyltransferase inhibitor, a guanylyltransferase inhibitor, or an RNA triphosphatase inhibitor.

15. The method of screening for methyltransferase, guanylyltransferase, or RNA triphosphatase inhibitors of claim 14, further comprising culturing a second strain of the isolated non-native yeast in the media comprising the screening compound, the second strain comprising a vertebrate methyltransferase gene, a vertebrate guanylyltransferase gene, or a vertebrate RNA triphosphatase gene; measuring the growth of the second strain, and comparing the growth of the second strain to a control and to the growth of the first strain,
wherein the first strain growing slower than both the control and the second strain indicates the at least one screening compound is a specific methyltransferase inhibitor, a specific guanylyltransferase inhibitor, or a specific RNA triphosphatase inhibitor.

16. An isolated virus comprising:
a) SARS-CoV-2 nsp14 comprising one or more of the following substitutions: nsp14 W293F, F368N, F368L, D353T, or D353A, wherein the substitutions refer to SEQ ID NO: 4;
b) MERS-CoV nsp14 comprising the following substitution: F365G, wherein the substitution refers to SEQ ID NO: 24;
c) ASFV NP868R comprising one or more of the following substitutions: Y714L, F711L, F711W, D680L, K647Y, or S604A, wherein the substitutions refer to SEQ ID NO: 10;
d) MPV D1 comprising one or more of the following substitutions: Y555F, R655A, D545A, D598A, Y683V, or R548A, R548K wherein the substitutions refer to SEQ ID NO: 290;
e) SARS-CoV-2 nsp12 comprising one or more of the following substitutions: K73A, D218A, D760A, V30A, R33A, R33L, R33K, R55A, R55L, R55K, K50A, C53A, R116A, V71A, N209A, L119A, or Y217F wherein the substitutions refer to SEQ ID NO: 291; or
f) WNV ns5 comprising one or more of the following substitutions: F24L, F24E, or S150C wherein the substitutions refer to SEQ ID NO: 292.

17. The isolated virus of claim 16, wherein:
a) the isolated virus comprising SARS-CoV-2 nsp14 is SARS-CoV-2;
b) the isolated virus comprising MERS-CoV nsp14 is MERS-CoV;
c) the isolated virus comprising ASFV NP868R is ASFV;
d) the isolated virus comprising MPV D1 is MPV;
e) the isolated virus comprising SARS COV 2 nsp12 is SARS-CoV-2; or
f) the isolated virus comprising WNV ns5 is WNV.

18. A composition comprising the isolated virus of claim 16 and a carrier or an adjuvant.

19. A method of stimulating an immune response in a subject, comprising administering a therapeutically effective amount of the isolated virus of claim 16 to the subject, thereby stimulating the immune response in the subject.

20. A method of stimulating an immune response in a subject, comprising administering a therapeutically effective amount of the composition of claim 18, thereby stimulating the immune response in the subject.
